SEQUENCE LISTING (60 sequences)

SEQ ID NO: 1 - DNA, 24 bp, Artificial Sequence
  Primer GALC Forward
ctggatactctatggctccttgac

SEQ ID NO: 2 - DNA, 24 bp, Artificial Sequence
  Primer GALC Reverse
agtggtgagcgtaaatatctcgtc

SEQ ID NO: 3 - DNA, 24 bp, Artificial Sequence
  Primer CGT Forward
caataatcccagttatcggcagag

SEQ ID NO: 4 - DNA, 24 bp, Artificial Sequence
  Primer CGT Reverse
tccaataggtagtccgattgacag

SEQ ID NO: 5 - DNA, 21 bp, Artificial Sequence
  Primer RPLP0 Forward
cacgaagctaacgactatcgc

SEQ ID NO: 6 - DNA, 20 bp, Artificial Sequence
  Primer RPLP0 Reverse
ctctagggactcgttcgtgc

SEQ ID NO: 7 - RNA, 48 nt, Artificial Sequence
  siRNA Murine PP1
ccagaucguuuguacagaaaucucgagauuucuguacaaacgaucugg

SEQ ID NO: 8 - RNA, 49 nt, Artificial Sequence
  siRNA Murine PP1
gucgucgagucauccgcauugauaucuagcugaaauuaccgaguuaaga

SEQ ID NO: 9 - RNA, 49 nt, Artificial Sequence
  siRNA Murine PP1
ggcguccuugaaaguguuaaaucucgagauuuaacacuuucaaggacgc

SEQ ID NO: 10 - RNA, 49 nt, Artificial Sequence
  siRNA Murine PP1
gaaucugacccucgguagcaaugauagaguuuaacacgcuuucuguaga

SEQ ID NO: 11 - protein, 41 aa, Artificial Sequence
  Peptide RVG-9R
YTIWMPEBPRPGTPCDIFTNSRGKRASNGGGGRRRRRRRRR

SEQ ID NO: 12 - DNA, 1356 bp, Homo sapiens
gcggggccgcgggccgggggcggactggggcgggcggaaggagagccaggccggaaggaggctgccggagggcgggaggcaggagcgggccaggagctgctgggctggagcggcggcgccgccatgtccgacagcgagaagctcaacctggactcgatcatcgggcgcctgctggaaggtgacatacacggccagtactacgaccttctgcgactatttgagtatggcggtttccctcccgagagcaactacctctttctgggggactatgtggacaggggcaagcagtccttggagaccatctgcctgctgctggcctataagatcaagtaccccgagaacttcttcctgctccgtgggaaccacgagtgtgccagcatcaaccgcatctatggtttctacgatgagtgcaagagacgctacaacatcaaactgtggaaaaccttcactgactgcttcaactgcctgcccatcgcggccatagtggacgaaaagatcttctgctgccacggaggcctgtccccggacctgcagtctatggagcagattcggcggatcatgcggcccacagatgtgcctgaccagggcctgctgtgtgacctgctgtggtctgaccctgacaaggacgtgcagggctggggcgagaacgaccgtggcgtctcttttacctttggagccgaggtggtggccaagttcctccacaagcacgacttggacctcatctgccgagcacaccaggtggtagaagacggctacgagttctttgccaagcggcagctggtgacacttttctcagctcccaactactgtggcgagtttgacaatgctggcgccatgatgagtgtggacgagaccctcatgtgctctttccagatcctcaagcccgccgacaagaacaaggggaagtacgggcagttcagtggcctgaaccctggaggccgacccatcaccccaccccgcaattccgccaaagccaagaaatagcccccgcacaccaccctgtgccccagatgatggattgattgtacagaaatcatgctgccatgctgggggggggtcaccccgacccctcaggcccacctgtcacggggaacatggagccttggtgtatttttcttttctttttttaatgaatcaatagcagcgtccagtcccccagggctgcttcctgcctgcacctgcggtgactgtgagcaggatcctggggccgaggctgcagctcagggcaacggcaggccaggtcgtgggtctccagccgtgcttggcctcagggctggcagccggatcctggggcaacccatctggtctcttgaataaaggtcaaagctggattctcgcaaaaaaaaaaaaaaaaaa

SEQ ID NO: 13 - DNA, 1363 bp, Mus musculus
aggagagggcccggagctggtgggccggagcggcggcgccgccatgtccgacagcgagaagctcaacctggactccatcatcgggcgcctgctggaagtgcagggctcacggcctgggaagaacgtgcagctgacagagaacgagatccgtggtctgtgcctcaaatcccgggagattttcctgagccagcccattcttctggagcttgaggcgcccctcaagatctgtggtgacatccatggccagtactatgaccttctacggctgtttgagtatggtggcttccctccagagagcaactacctcttcttgggggattatgtagatcggggcaagcagtctttggagaccatctgcctgttgctggcctataagatcagatacccggagaatttctttctacttcgtgggaaccatgagtgtgccagcatcaaccgcatttatggcttctatgatgaatgcaagagaagatacaacatcaaactgtggaagacgttcactgactgcttcaactgcctgcccattgcagccattgtggatgagaagatcttctgctgccacgggggcctgtctccagacttgcaatccatggagcagattaggcgtattatgcggcccacagacgtgcctgaccagggcctactgtgtgatctcctgtggtctgaccctgacaaggatgttcaaggctggggcgagaatgaccgtggtgtctcctttacctttggggctgaggtggtagccaagttcctgcacaagcatgatttggacctcatctgcagagcacatcaggttgtagaagatggctatgagttctttgccaagagacagttggtgacactcttctcagctcccaactactgtggagagtttgacaatgctggtgccatgatgagtgtggatgagaccctcatgtgttccttccagatcctcaagcccgctgataagaataagggcaagtatgggcagttcagcggcctgaaccccggaggccggcccatcactccaccccgcaattctgccaaagccaagaaatagcctccatgtgctgcccttctgccccagatcgtttgtacagaaatcatgctgccatgggtcacactggcctctcaggcccacccgtcacggggaacacacagcgttaagtgtctttcctttattttttaaagaatcaatagcagcatctaatctcccagggctccctcccaccagcacctgtggtggctgcaagtggaatcctggggccaaggctgcagctcagggcaatggcagaccagattgtgggtctccagccttgcatggctggcagccagatcctggggcaacccatctggtctcttgaataaaggtcaaagctggattctc

SEQ ID NO: 14 - DNA, 4991 bp, Homo sapiens
ggcggcgcgcaagggacgtgcggagtgagtggcgctgcgggtggggccgtcggcggcgctggtgagctttgcggagctgggcggtgccgaggaggaggaggtggcggcctgggtctgacgcggccctgttcgagggggcctctcttgtttatttatttattttccgtgggtgcctccgagtgtgcgcgcgctctcgctacccggcggggagggggtggggggagggcccgggaaaagggggagttggagccggggtcgaaacgccgcgtgacttgtaggtgagagaacgccgagccgtcgccgcagcctccgccgccgagaagcccttgttcccgctgctgggaaggagagtctgtgccgacaagatggcggacggggagctgaacgtggacagcctcatcacccggctgctggaggtacgaggatgtcgtccaggaaagattgtgcagatgactgaagcagaagttcgaggcttatgtatcaagtctcgggagatctttctcagccagcctattcttttggaattggaagcaccgctgaaaatttgtggagatattcatggacaatatacagatttactgagattatttgaatatggaggtttcccaccagaagccaactatcttttcttaggagattatgtggacagaggaaagcagtctttggaaaccatttgtttgctattggcttataaaatcaaatatccagagaacttctttctcttaagaggaaaccatgagtgtgctagcatcaatcgcatttatggattctatgatgaatgcaaacgaagatttaatattaaattgtggaagaccttcactgattgttttaactgtctgcctatagcagccattgtggatgagaagatcttctgttgtcatggaggattgtcaccagacctgcaatctatggagcagattcggagaattatgagacctactgatgtccctgatacaggtttgctctgtgatttgctatggtctgatccagataaggatgtgcaaggctggggagaaaatgatcgtggtgtttcctttacttttggagctgatgtagtcagtaaatttctgaatcgtcatgatttagatttgatttgtcgagctcatcaggtggtggaagatggatatgaattttttgctaaacgacagttggtaaccttattttcagccccaaattactgtggcgagtttgataatgctggtggaatgatgagtgtggatgaaactttgatgtgttcatttcagatattgaaaccatctgaaaagaaagctaaataccagtatggtggactgaattctggacgtcctgtcactccacctcgaacagctaatccgccgaagaaaaggtgaagaaaggaattctgtaaagaaaccatcagatttgttaaggacatacttcataatatataagtgtgcactgtaaaaccatccagccatttgacaccctttatgatgtcacacctttaacttaaggagacgggtaaaggatcttaaatttttttctaatagaaagatgtgctacactgtattgtaataagtatactctgttatagtcaacaaagttaaatccaaattcaaaattatccattaaagttacatcttcatgtatcacaatttttaaagttgaaaagcatcccagttaaactagatgtgatagttaaaccagatgaaagcatgatgatccatctgtgtaatgtggttttagtgttgcttggttgtttaattattttgagcttgttttgtttttgtttgttttcactagaataatggcaaatacttctaatttttttccctaaacatttttaaaagtgaaatatgggaagagctttacagacattcaccaactattattttcccttgtttatctacttagatatctgtttaatcttactaagaaaactttcgcctcattacattaaaaaggaattttagagattgattgttttaaaaaaaaatacgcacattgtccaatccagtgattttaatcatacagtttgactgggcaaactttacagctgatagtgaatattttgctttatacaggaattgacactgatttggatttgtgcactctaatttttaacttattgatgctctattgtgcagtagcatttcatttaagataaggctcatatagtattacccaactagttggtaatgtgattatgtggtaccttggctttaggttttcattcgcacggaacaccttttggcatgcttaacttcctggtaacaccttcacctgcattggttttctttttcttttttctttctttttttttttttttttttttttgagttgttgtttgtttttagatccacagtacatgagaatccttttttgacaagccttggaaagctgacactgtctctttttcctccctctatacgaaggatgtatttaaatgaatgctggtcagtgggacattttgtcaactatgggtattgggtgcttaactgtctaatattgccatgtgaatgttgtatacgattgtaaggcttatgtcactaaagatttttattctgattttttcataatcaaaggtcatatgatactgtatagacaagctttgtagtgaagtatagtagcaataatttctgtacctgatcaagtttattgcagcctttcttttcctatttcttttttttaagggttagtattaacaaatggcaatgagtagaaaagttaacatgaagattttagaaggagagaacttacaggacacagatttgtgattctttgactgtgacactattggatgtgattctaaaagcttttattgagcattgtcaaatttgtaagcttcatagggatggacatcatatctataatgcccttctatatgtgctaccatagatgtgacatttttgaccttaatatcgtctttgaaaatgttaaattgagaaacctgttaacttacattttatgaattggcacattgtattacttactgcaagagatatttcattttcagcacagtgcaaaagttctttaaaatgcatatgtctttttttctaattccgttttgttttaaagcacattttaaatgtagttttctcatttagtaaaagttgtctaattgatatgaagcctgactgattttttttttccttacagtgagacatttaagcacacattttattcacatagatactatgtccttgacatattgaaatgattcttttctgaaagtattcatgatctgcatatgatgtattaggttaggtcacaaaggttttatctgaggtgatttaaataacttcctgattggagtgtgtaagctgagcgatttctaataaaattttagttgtacacttttagtagtcatagtgaagcaggtctagaaaataagcctttggcagggaaaaagggcaatgttgattaatctcagtattaaaccacattaatctgtatcccattgtctggcttttgtaaattcatccaggtcaagactaagtatgttggttaataggaatccttttttttttttttaaagactaaatgtgaaaaaataatcactacttaagctaattaatattggtcattaaatttaaaggatggaaatttatcatgtttaaaaattattcaagcactcttaaaaccacttaaacagcctccagtcataaaaatgtgttctttacaaatatttgcttggcaacacgacttgaaataaataaaactttgtttcttaggagaaaatgattctgtaattccagtgtcactaatttatattgttctttcctctgatttttttcaggttagtgatttttttgtatacaatttaatccaaatgttatgacattcagaaatcatgaaacacagtagatatctgttataatgtggtgtatcacatggattataaagcaaagttatggtcgatttctattcttgaaagaatcaactacagtgaatcctttgcatttgaagccttaacatgcattgctttaattttgcccagggacaaattttaataatcagcaagactggtttgtgcaaagcgttgagtcatcaggtatttagagcctagccagctacccagtatccatgctgccatatcccttcattgtaaaaagtacctaaacattcgtgaaatgattttttttagctgaaaaatgctggcaagaagaattttaaagcttaaaataggtggtaaatttgaagtatgagtgtgttcacgagaaacataggcttttcaaaaaaatttttattcaaggcaaagcaaggaacatcttgagatatgtctcaagaatataaagatgtattattttaagccaaggagctgaaatatatctcagtttataaattcaggtatattctttttgtctccatggcaaccataacttttgaaccaaaaaaaattgtttttacatctttatgctgaaaatgtgtttagattaggaatatggtcgggctgaatttgctgttgctccctaaccaaatccacctcttgttttccttgtgagtccatggctaaatcaaagctgcccctgagaagagacttaatccaagcctgattgtactagtggcatcacttagaagtaggctttccctcttcctagtagatctcaatgttttataattccttaaaacagctgaaaattgggacaacatactttacgcaatgaacagtagttaaataggaaataaactagttccatataagtatacacctagagttttaattacctttataatgtttcttaaaagtgaaacttagatacaattgtgattggatacttagatactaagtgaaacttagtgtaacaattttgatctgttaaattggattttacatgtacatttgaatgccagaatttctaaataaatcccctggttaggaaattttaaaagtcaaagcttgttttcttcaaccactaccttctacattggttgacttagaccgtaagctttttaagtttctcattgtaatttaccttctcatgcagattgctgatgttttattaaaccttatttttacaaaaatgaaaaaa

SEQ ID NO: 15 - DNA, 4024 bp, Mus musculus
gcggctaggcggcccgcaagggcggaggggagggagtgacgctgagggcggggctgtccgtggcgctgccgagctttgcggagctgggcggtgccgaggaggaggtggaggaggcggtggcggctggggtctgacgcggcccggttcctgacgcggcccggttcctgggggcctgcttgtttatttatttatttccagtgggcgccgccagcgtgtgcgcgcgctgtcgctgctcggcggggagggggtggggggagggcccgcgcccggggggagttggagccggggtcgaaacgccgcgtgactcgtaggtgagaacgccgagccgccgccgccgccgagaagccctgttaacgctttagggaggagagtctggtgccgacaagatggcggacggggagctgaacgtggacagcctcatcacccgcctgctggaggtacgaggatgtcgtccaggaaagattgtacaaatgactgaagcagaagtccgagggttgtgtatcaagtctcgtgaaatctttctcagccagcctattcttttggaattggaagcaccactgaagatttgtggagacattcatggacaatatacagacttactaagattatttgaatatggaggttttccaccagaagccaactatcttttcttaggagattatgtggacagaggaaagcagtctttggaaaccatctgtttgctattggcttacaaaatcaaatacccggagaacttctttcttctaagaggaaaccatgagtgtgctagcatcaatcgcatttatggattctacgatgagtgtaaacgaagatttaatattaaattgtggaagacattcactgattgttttaactgtctgcctatagctgctattgttgatgagaaaatcttctgttgtcatggaggactgtcaccagacctacaatctatggaacagattcggagaattatgagacccactgacgtacctgatacaggtttgctttgtgacttactgtggtccgacccagataaggatgtgcaaggctggggagaaaatgaccgtggtgtttcttttacttttggagctgatgtagtcagtaaatttctgaatcgtcatgatttagacttgatttgtcgagctcatcaggtggtggaagacggatatgaattttttgctaaacgacagttggtaaccttattttctgccccaaattactgtggcgagtttgacaatgctggtggtatgatgagtgtggatgagactttgatgtgttcattccagatattgaaaccatctgaaaagaaagctaagtaccagtatggtgggctgaattctggacgtcctgtcactccgcctcgaacagctaatccaccgaagaaaaggtgaagacaggaattctagaaagagaaaccatcagatttgttaaggacatacttcataatatataagtgtgcactgtaaaaccatccagccattcgacaccctttatgatgtcacacctttaacttaaggagacggtaaaggatcttaaatttttttctaatagaaagatgtgctacactgtattgtaataagtatactctgttataatattcaacaaaattaaatccaaattcaaaagtatccattaaagttctatcttctcatatcacagtttttaaagttgaaaagcatcccagttaaactagccctgttagtgacccagatgaaagcatgaagatccatctgtgtaatgtggttttagtggtgcttggttgtttcattattttgagcttgttttgttttgtttgtttttgctagaataatggcatctacttttcctatttttccctaaacatttttaaaagtgaaaatgggaagagctttaaagacattcaccaactattcttttcctttacttatctacttaagtaactgttggatcttactaagaaaacttacccctcattacagtaaaaaggaactttagaggtcgataggttttaaaaatatacaaactatctgatccattgattttaatcaaacagtttgactgggcaaactttgcagctgataatgagtatttcgctttttacaaaattgccactgatttggatttgtgcactctaatctttaatttattgatgctctattgtgcagtagcatttcatttaagataaggctcatatagtaatatccaaaactagttggtaatgtgattatgtggtactttggctttgggttctaattcgcacgaaacaccttttggcatgcttaactttctggtattaccctcacctgcattggttttgttttttggggtttttgttgtttgtttgtttttagattcacagaacatgagaatcctttttgacaagccttggatagctggctctcttctttccctctctctatgtgaaggatgtatttaaatgaacgctggtcagtgggacatttgtcagctctgaatattgggtgcttcactaataattgccatgtgaatgttgttttgactgtaaggctatgtcactaaagatttttactctgcgttttcataatcaaaggtcatgatgtgtatagacatgctttgtagtgaagtatagtagcaataatttctgtatgtgatcaagagtttattgcattatttctttccctgttctcttttttttttttttaagggttagcattaacaaatgtcaaggagtagcaaagtcaacaaagattttagaaggaggaggaactaagagcatacacagacttatgattctttggatgtgacacttattggatgtgattctaaagtcttttattgaacattgtcaaattcgtacgcttcataggatggacataatgtttatataatgcccttcttatgtgttaccatagatgtgtgaaaccttatagcgtccttgaaagtgttaaattgagaactctgttaacattttatggattgacacattatattactgcaagtaacatttgattttcagcacagtgcaaaagttctttaaaatgcatatgtctttttttttctaattccattttgtttaaagcacattttaaatgtagttttctcatttagtaaaagttgtctaattgatacaacgtctgagtgattattctgtgttgttttgttttacagtgagatatgtaagcacaagttgacatagactgaagcatagacagtctctgagctgtagccatgttctattaggtcacacatgcttttatttaatgcgattggataacttacatactagagtaaacgaacaattgtttactcaaacaattgctaataggattttagatgttatctctgagtaatcaatacttaaggtagctcaagaaaataagccttagtctcaatattagttaatgtacactaatttgtatcttaaactgttttgttttttgtaaatgttcattcaaattaaaactaggggcgaaaagtaagcaaattagtattggttgttaaaggatagacatttaccatgttggaaaattattcagacctcttaaaactactttacagcttctcatatataagtactcagtacatcatgtgctcctaagaatgataacacagattattaattatactagtttactgacaaagtcacaaagacaaacagtaaaatacaggctacctttaaccccatgattggtgaggcagacagagacaggtgaatctatgacttgaagcctagactacatgttacagtgtctgtttactagatgaaaagctacaagcaattgcagagaatttgggttcaattctttttttttttttttacacgtattttcctcaaattacatttccaatgctatctcaaaagtcccccacaccctgggttctattcttaacactcagaaggctgtctgtatggctaagccaaggggatctgctgttttatgccctctgagggcaccaggcacacacatggcacacatggtgggtacatagacatacatataggcaaaatcctcatacacataaaaataaaatgtttaaaggatttttttattaagtgttgagcaaataaaatgagttttttgattgg

SEQ ID NO: 16 - DNA, 1211 bp, Homo sapiens
agaaggccctgcgcgggcagacggggcggggctggaggctcaggtgccgcctcctctgcaacgccggggccagagtcttaaaaccgagggcccgcaggggtccccgcggccgccgcgatgcagaaatacgagaaactggaaaagattggggaaggcacctacggaactgtgttcaaggccaaaaaccgggagactcatgagatcgtggctctgaaacgggtgaggctggatgacgatgatgagggtgtgccgagttccgccctccgggagatctgcctactcaaggagctgaagcacaagaacatcgtcaggcttcatgacgtcctgcacagcgacaagaagctgactttggtttttgaattctgtgaccaggacctgaagaagtattttgacagttgcaatggtgacctcgatcctgagattgtaaagtcattcctcttccagctactaaaagggctgggattctgtcatagccgcaatgtgctacacagggacctgaagccccagaacctgctaataaacaggaatggggagctgaaattggctgattttggcctggctcgagcctttgggattcccgtccgctgttactcagctgaggtggtcacactgtggtaccgcccaccggatgtcctctttggggccaagctgtactccacgtccatcgacatgtggtcagccggctgcatctttgcagagctggccaatgctgggcggcctctttttcccggcaatgatgtcgatgaccagttgaagaggatcttccgactgctggggacgcccaccgaggagcagtggccctctatgaccaagctgccagactataagccctatccgatgtacccggccacaacatccctggtgaacgtcgtgcccaaactcaatgccacagggagggatctgctgcagaaccttctgaagtgtaaccctgtccagcgtatctcagcagaagaggccctgcagcacccctacttctccgacttctgtccgccctaggccccgggacccccggcctccaggctggggcctggcctatttaagccccctcttgagaggggtgagacagtgggggtgcctggtgcgctgtgctccagcagtgctgggcccagccggggtggggtgcctgagcccgaatttctcactccctttgtggactttatttaatttcataaattggctcctttcccacagtcaaaaaaaaaaaaaaaaaa

SEQ ID NO: 17 - DNA, 7095 bp, Homo sapiens
cgggcttgtgccgccgccgccgccgccgccgcccgggccaagtgacaaaggaaggaaggaagcgaggaggagccggccccgcagccgctgacagggctctgggctggggcaaagcgcggacacttcctgagcgggcaccgagcagagccgaggggcgggagggcggccgagctgttgccgcggacgggggagggggccccgagggacggaagcggttgccgggttcccatgtccccggcgaatggggaacagtcgaggagccgctgcctggggtctgaagggagctgcctccgccaccgccatggccgctggatccagccgccgcctgcagctgctcctggcgcaatgaggagaggagccgccgccaccgccaccgcccgcctctgactgactcgcgactccgccgccctctagttcgccgggcccctgccgtcagcccgccggatcccgcggcttgccggagctgcagcgtttcccgtcgcatctccgagccaccccctccctccctctccctccctcctacccatccccctttctcttcaagcgtgagactcgtgatccttccgccgcttcccttcttcattgactcggaaaaaaaatccccgaggaaaatataatattcgaagtactcattttcaatcaagtatttgcccccgtttcacgtgatacatatttttttaggatttgccctctcttttctctcctcccaggaaagggaggggaaagaattgtattttttcccaagtcctaaatcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaagagaaaaaaatcctgttcattttagtgttcaatttttttattattattgttgttcttatttaaccttgtaaaatatctataaatacaaaccaatttcattgtattctcactttgagggagatccagggggtgggaggggttgtggggagggggaaagcggagcactagaacatacaatctctctcccacgacaatctttttttattaaaagtctgctgttgtatactttaaaaacaggactcctgcctcatgccccttccacaaaagaagaaaacctttttctgtgctgatgggtttttttgaactttgttttcttttaaagtctagtgtgagactttggtatagtgcacagcttgaaattggttgggagcttagcaggtataactcaacggggacttaaatgtcacttgtaaaattaatccatatcttcgggtatttatagacttgcctttggcatgttggtggcaggtgtggcagacaaagaaatgtgtatcattcgtaacccagggaggtcaataaagtttggaactctacagggaagattcttagtagatttgttaaggttttgttttgctctcagttagtgctagtgatgtagaggcttgtacaggaggctgccagaggggaagcagcaagcaagactcaggcacacatgctctacaggtggctctttgtttgcctgaccaaagttctttgcaaatcttagcacagtttcaaactagtgacctgggaggagatggaaggggtgttgagcaggctgagctagctgctgaggtcaaaggctgatgagcccagaggaaggggacaggtcagggatacatctcaccactgtgaataagtttgtccagatttttttctaaagttacttcccttggaaagatacacttgagaggacattgtagttaaataatgtgaactgtaacagtcatctactggtttatttttcatattttttaattgaaaattgagcttgcagaaatagccacattctacacatagttctaattttaaatccaaatctagaatctgtatttaatttgttttttaacctcatgctttttacatttatttattgatgcatgtcagatggtagaaatattaaaaactacacatcagaatgatacagtcacttatacctgctgactttataggaaagctgatgatataaatgtgtgtatatatgttatatatacatatattcaatactgcctttttttttgtctacagtatcaaaattgactggttgaagcatgagaagaatgtttcccccacacccagttaagagtttttgtgtctgttttctttgtgtatcagtgaacgatgttaagaatcagtctctctttttgaagaaaaagcaatattccttggaaagcaaggagaattgaaggactatgtttgccgtgaggaaatagattttcatgactagtttgttttatacttttaaggttggcatctatgtgggccttatatactctaaaatgaactttagtcaccttggtgcttatgggccattacttgacctatgaatctttaaggcacaatcagttgtactttacatttaaagatcacttgagtgatggccgcctttccctcctacccgctccttccccacatgccttccaaggttagctggtaactgtagggctgcagagctgagcccatggttgtgtgtaacttgccctcaccctcctcattgccaccttaggtcactttatgggtctcgtcctccagagggttcggaagtggagtctgttggcagccctcctgcaggccctagcaccctgtcctgctccttaactgtgtgtgtgactctccaagagagttgtcctgcctgctgaagtgaaccagtacccagaaagacaactgtgagccatcttggttttcactcgctgtttagctgaggtcttgggccacaaaaggggtttcacaaacctctggatatatcagagtttatgagaaaggaaacatgctcagtcaaaccaaatcaaacaaattgaattttatgttttataaagtgcttctgaaagctaagatttgaaagaagtctgaaatcaaagtatttggcagcataactccttaaaggtagtggcgttgatagaccattttcagacagaatttataaagaatctgaaaaggcaggtctgtgatagagaaatggacctgcattcagatccaactgcccagcaagcgtttggatgcagacactgctctggacgtggtatactccccagagtccataaaaatcagtgcttattttaggaaacaggttgccccccacaactggggtaaaagaagagagaaaagtcacgcttttctctcatttcattgtgtgtgcatgtgtgcgtgtgtgtgtgtgtgtgtgtgtgctgagatgtgtgatttttctttctcaaggatcatggtgggatcacagaactcttttatacaagtgagatccaggtctctgaatatctttttgtatataataataataaaaagctcctcaccaaattcaagcttgtacattatattttctttctgtgtttttaaatttaagttttattgttttgtatgtaaatatgtggacccaggaactgttattaatgagcaaaaagttactgttcagggcagtgattctgtttaataatcagacaaaatgtagacgagctttttaaagccatatagttttaactctgtacagtaggtaccggcctgtattattgtaacaataactctagcaatgtatagtgtatctatatagtttggagtgccttcgcttccatgtgtttttttttttaatttgttcttttttaaattttaattggtttcctttatccatgtctccctgtccaccccctttccctttgaaataataactcactcataacagtatctttgccccttccacagttaagtttcagtgataccatactcaggagtgggaagaggaaatcatattcgtaatttcatttcgttgaagccctgcctttgttttggttctgaatgtctttcctcctcggtagcagtgagaccggtttcatttcatacttagtccattcagggacttagtgtagcaccagggagccctagagctggaggatatcgaatagattaaattttgctcgtctcttccacaagccctaaccatgggtcttaaaaacagcagattctgggagccttccatgctctctctctctcctcttttatctacttccctcccaaatgagagagtgacagagaattgtttttttataaatcgaagtttcttaatagtatcaggttttgatacgtcagtggtctaaaatgctatagtgcaattactagcagttactgcacggagtgccaccgtgccaatagaggactgttgttttaacaagggaactcttagcccatttcctccctcccgccatctctacccttgctcaatgaaatatcattttaatttcttttaaaaaaaatcagtttaattcttactgtgtgcccaacacgaaggccttttttgaaagaaaaatagaatgttttgcctcaaagtagtccatataaaatgtcttgaatagaagaaaaaactaccaaaccaaaggttactatttttgaaacatcgtgtgttcattccagcaaggcagaagactgcaccttctttccagtgacatgctgtgtcattttttttaagtcctcttaatttttagacacatttttggtttatgttttaacaatgtatgcctaaccagtcatcttgtctgcaccaatgcaaaggtttctgagaggagtattctctatccctgtggatatgaagacactggcatttcatctatttttccctttcctttttaaaggatttaactttggaatcttccaaaggaagtttggccaatgccagatccccaggaatttggggggttttctttcttttcaactgaaattgtatctgattcctactgttcatgttagtgatcatctaatcacagagccaaacacttttctcccctgtgtggaaaagtaggtatgctttacaataaaatctgtcttttctggtagaaacctgagccactgaaaataaaagagacaactagaagcacagtagagtcccagactgagatctacctttgagaggctttgaaagtaatccctggggtttggattattttcacaagggttatgccgttttattcaagtttgttgctccgttttgcacctctgcaataaaagcaaaatgacaaccagtacataaggggttagcttgacaaagtagacttccttgtgttaatttttaagtttttttttccttaactatatctgtctacaggcagatacagatagttgtatgaaaatctgcttgcctgtaaaatttgcatttataaatgtgttgccgatggatcacttgggcctgtacacataccaattagcgtgaccacttccatcttaaaaacaaacctaaaaaacaaaatttattatatatatatatatatatatataaaggactgtgggttgtatacaaactattgcaaacacttgtgcaaatctgtcttgatataaaggaaaagcaaaatctgtataacattattactacttgaatgcctctgtgactgatttttttttcattttaaatataaacttttttgtgaaaagtatgctcaatgttttttttccctttccccattcccttgtaaatacattttgttctatgtgacttggtttggaaatagttaactggtactgtaatttgcattaaataaaaagtaggttagcctggaaatgaaattaaaaaaaaaaaaaaaaaa

SEQ ID NO: 18 - DNA, 8787 bp, Homo Sapiens
ggccgcagctccccggcggaggcaagaggtggttgggggggaccatggctgacgttttcccgggcaacgactccacggcgtctcaggacgtggccaaccgcttcgcccgcaaaggggcgctgaggcagaagaacgtgcacgaggtgaaggaccacaaattcatcgcgcgcttcttcaagcagcccaccttctgcagccactgcaccgacttcatctgggggtttgggaaacaaggcttccagtgccaagtttgctgttttgtggtccacaagaggtgccatgaatttgttactttttcttgtccgggtgcggataagggacccgacactgatgaccccaggagcaagcacaagttcaaaatccacacttacggaagccccaccttctgcgatcactgtgggtcactgctctatggacttatccatcaagggatgaaatgtgacacctgcgatatgaacgttcacaagcaatgcgtcatcaatgtccccagcctctgcggaatggatcacactgagaagagggggcggatttacctaaaggctgaggttgctgatgaaaagctccatgtcacagtacgagatgcaaaaaatctaatccctatggatccaaacgggctttcagatccttatgtgaagctgaaacttattcctgatcccaagaatgaaagcaagcaaaaaaccaaaaccatccgctccacactaaatccgcagtggaatgagtcctttacattcaaattgaaaccttcagacaaagaccgacgactgtctgtagaaatctgggactgggatcgaacaacaaggaatgacttcatgggatccctttcctttggagtttcggagctgatgaagatgccggccagtggatggtacaagttgcttaaccaagaagaaggtgagtactacaacgtacccattccggaaggggacgaggaaggaaacatggaactcaggcagaaattcgagaaagccaaacttggccctgctggcaacaaagtcatcagtccctctgaagacaggaaacaaccttccaacaaccttgaccgagtgaaactcacggacttcaatttcctcatggtgttgggaaaggggagttttggaaaggtgatgcttgccgacaggaagggcacagaagaactgtatgcaatcaaaatcctgaagaaggatgtggtgattcaggatgatgacgtggagtgcaccatggtagaaaagcgagtcttggccctgcttgacaaacccccgttcttgacgcagctgcactcctgcttccagacagtggatcggctgtacttcgtcatggaatatgtcaacggtggggacctcatgtaccacattcagcaagtaggaaaatttaaggaaccacaagcagtattctatgcggcagagatttccatcggattgttctttcttcataaaagaggaatcatttatagggatctgaagttagataacgtcatgttggattcagaaggacatatcaaaattgctgactttgggatgtgcaaggaacacatgatggatggagtcacgaccaggaccttctgtgggactccagattatatcgccccagagataatcgcttatcagccgtatggaaaatctgtggactggtgggcctatggcgtcctgttgtatgaaatgcttgccgggcagcctccatttgatggtgaagatgaagacgagctatttcagtctatcatggagcacaacgtttcctatccaaaatccttgtccaaggaggctgtttctatctgcaaaggactgatgaccaaacacccagccaagcggctgggctgtgggcctgagggggagagggacgtgagagagcatgccttcttccggaggatcgactgggaaaaactggagaacagggagatccagccaccattcaagcccaaagtgtgtggcaaaggagcagagaactttgacaagttcttcacacgaggacagcccgtcttaacaccacctgatcagctggttattgctaacatagaccagtctgattttgaagggttctcgtatgtcaacccccagtttgtgcaccccatcttacagagtgcagtatgaaactcaccagcgagaacaaacacctccccagcccccagccctccccgcagtgggaagtgaatccttaaccctaaaattttaaggccacggccttgtgtctgattccatatggaggcctgaaaattgtagggttattagtccaaatgtgatcaactgttcagggtctctctcttacaaccaagaacattatcttagtggaagatggtacgtcatgctcagtgtccagtttaattctgtagaagttacgtctggctctaggttaacccttcctagaaagcaagcagactgttgccccattttgggtacaatttgatatactttccataccctccatctgtggatttttcagcattggaatcccccaaccagagatgttaaagtgagcctgtcccaggaaacatctccacccaagacgtctttggaatccaagaacaggaagccaagagagtgagcagggagggattgggggtgggggaggcctcaaaataccgactgcgtccattctctgcctccatggaaacagcccctagaatctgaaaggccgggataaacctaatcactgttcccaaacattgacaaatcctaacccaaccatggtccagcagttaccagtttaaacaaaaaaacctcagatgagtgttgggtgaatctgtcatctggtaccctccttggttgataactgtcttgatacttttcattctttgtaagaggccaaatcgtctaaggacgttgctgaacaagcgtgtgaaatcatttcagatcaaggataagccagtgtgtacatatgttcattttaatctctgggagattatttttccatccagggtgccatcagtaatcatgccactactcaccagtgttgttcgccaacacccacccccacacacaccaacattttgctgcctaccttgttatccttctcaagaagctgaagtgtacgccctctccccttttgtgcttatttatttaataggctgcagtgtcgcttatgaaagtacgatgtacagtaacttaatggaagtgctgactctagcatcagcctctaccgattgattttcctcccttctctagccctggatgtccacttagggataaaaagaatatggttttggttcccatttctagttcacgttgaatgacaggcctggagctgtagaatcaggaaacccggatgcctaacagctcaaagatgttttgttaatagaaggattttaatacgttttgcaaatgcatcatgcaatgaattttgcatgtttataataaaccttaataacaagtgaatctatattattgatataatcgtatcaagtataaagagagtattataataattttataagacacaattgtgctctatttgtgcaggttcttgtttctaatcctcttttctaattaagttttagctgaatcccttgcttctgtgctttccctccctgcacatgggcactgtatcagatagattactttttaaatgtagataaaatttcaaaaatgaatggctagtttacgtgatagattaggctcttactacatatgtgtgtgtatatatatgtatttgattctacctgcaaacaaatttttattggtgaggactatttttgagctgacactccctcttagtttcttcatgtcacctttcgtcctggttcctccgccactcttcctcttggggacaacaggaagtgtctgattccagtctgcctagtacgttggtacacacgtggcattgccgcagcacctgggctgacctttgtgtgtgcgtgtgtgtgtgtttccttcttcccttcagcctgtgactgttgctgactccaggggtgggagggatggggagactcccctcttgctgtgtgtactggacacgcaggaagcatgctgtcttgctgcctctgcaacgacctgtcgtttgctccagcatgcacaaacttcgtgagaccaacacagccgtgccctgcaggcaccagcacgtgcttttcagaggctgcggactttcttccagccattgtggcattggcctttccagtcttgggaggagcgcgctgctttggtgagacacccccatgcaaggtcctcagagtagccgggttctaccacaaacagaaacagaatgaaagtagctgtcagtccttgtagagagccgctctgtttcctcccagaagcatctcccagctaagctcgcattatttttctcctctggctgtttgcctgaagttcacagaacacacaaccatgaaaggctttttgaggtgagaggcccaggtggtcctggcaaccctgagtagaaggagagacggggtagggaacgggcccggccagaaaagaaccatttcttctgccatcttttatgcaccatagacatcgagactccagggggtcctggctcccctgtccctgcagccctgcaggtcagtgcatgatctgggttcgtgtcctgaccaggtgctcctcctttgatccgaggggaaagggactggtttatagaaagagcctaggagacaaaagggccagtccccctgcccagaatggagcagcagcaggacagacccccacgaggccccccagagaggaggaagatcccacggaggaacacatgaggttagggacccttgttcagcaccccaaacagcctgcctgtttaaagcaggcagcaggcttaggccttccctgcaaccccaacacccacaagtttgtttctctaggaaacacattcactgtctcagctggctgttactctctcagaccatatggcaaagttttccaagaaaatgccccgacaggggtgcccagcacactgcctgagggacaacagacatcagaacaaacccccagagagaaacagtcaaaatcagggcccggtgcagtgttgtcatgtggaacctgctttatccattgctgagtgttgaatgtgggtaatggttagggctttccagatctcagcagccaaagacagttattgttggaagactgtcatgtagataaccatgagcaatggctcgcctcagaatcagttcataaaattctatggtactggccccttcgtgggtattgtgtgaaatgagatggtggcgaggggtgcgctgtggaactgccgcagccacgcaggaggtccctgggggatgctttgggaagtccttgcccctgagcactgcctgattgccagggcctgtggaggtctaggccgcctggcagaatctagcaccgtccgaatccccgcaggacccatggagctatgaccacaccaggccattcaaatggctctgcattatcttcccttggaaggtggccactcctcggtggcagggcctttccctgaggctgcaggccgtgggctggcagcccgtctcttggcatttcaattgaaggtcaccaggtgctgggtttgaaaggaagtcactggagtgctgccaggggccgccctccaaggttaatgagaggcccacatccaggcaagaactaattcaaaaggcagatcagaaaccacaggagtcaaaattattgctccggcagtgcttcccttcctttcatccactggcctcgtgtggtccatgcagggccactgtctgccctttctgatgccacgtattaggctttcttactcagaattttgatagaaaaccatggggccaagagctctggaagcctggccggaaagaccaaggttcatgcagcccaacaaatgattgttgagcacctctcggagccaaagtccttaggcgagtgtggtgacttcctggaaggaggatgcagacttccagagagcccccccaacggacgtgctgagaagggagagggaggcgggggctgtagtcaggaaggagccagagaagaacagggtttgggtgcatccagaaatatgcctgcagtaggagggagaggaaggggtgccaccgtcaacggcttcccatcggaggtggttggtgcagatggaagtttctgtctgctggccctcaagagagtgttttgccagggacacagtctgttcctcctcagaaaacaccccccaaatgctaacaacatccccaccagctgctagaagcccctttcccctccccaccttgaagtagctcatagttctctgggcagagccagaccatccagtgtaccccagaggccagtaggttcctgcccattttcctctctggcttcctgccaagaattatggcagctgaggatgaatggagaagtaaaaacaactaacaccgcacaactaacaactaacaccgcagttcccacctgggttccacttagcaggagacatttcggagggttttttttgtttttgttcctgttttttttttttttgctggaatttgttttctcagtactgaaaagagaaaaagtgacaatcttgtatttttaaaagcctcggaaaggtgataccatctgacagtcattttctcacgttggtcttctaaagtcacctatttcttgtgtgtgcacatcacaccatttcctgtttctttataacccgacaagggtaggagtgcctgtttcccctgctgggcacaccagacaatcgtaatcacaaaacagacactgagccaggggcccaaagggtgtgatcatgagagttaccgggacagcagtaggcatgacagtcaccaggaaggacaagggtgctctgttgttagtggccacacaccaatttgacaaggagtgttgcgaaatttttatttatttatttatttattttgagatggagtttcactcttgttgcccaggctggagtgcggtggtacaatctcggctcactgcaacctccacctcccaggttcaagcgattctcctgcctcagcctcccaagtacctgggactacaggtgcgtgccaccacacccagctaaattttgtgtttttagtagagatggggtttcaccatgttggccaggatggtcttgaacccctgacctcatgatctgcctgcctcggcctcccaaagtgctgggattacaggcatgagccaccacgcccagccaaaatatttttttaaagtcattttccttaagctgcttgggctacatgtgaaatacactggacggtcaacattcctgtctcctcccatttgggctgatgcagcagatccagggaatgttacctgtttctgctgctagaagatccaggaaattgggaaggttacctgacgcacacatggatgaaggccatcatctagaaatggggtcaaccacaattgtgttaattccgtagtgtcagggattcttcgggaaggtcaacagtatgaaggattctgacccctgtgcctcccatttatgtgatcaggtgacagttaataaccgtggaggtcacactcagccatccaacagccttacagtgaccctacacaaaagcccccaaattccaaagactttttcttaacctaaaggaagaaattatttgttaattccagtagagcaactgaatatactgggctatttgtacttttttatagagaactttaataataattctttaaaaatgagtttttagaacaaagcaactgacgatttcctaagattccaatgccctggagcttgtaggaggacttagcctgggtcagctggagcacccccgacctgatctcccactgccagattttcccatgctcctagggtatggagtccacgtgggaatgactgcaagttcaggtggaacttggccgactgatgctctgcgagtttttaatagacactggggacaactgcttaaggtttagaaacttccaaaccacaggaaagacatttttagtgtcccccatccagaggcagccctggaataggattcccaggggtttctgggacccctttccttgctccgtgaggctctgtggccatcttttggcaggaggaggatgcttccttggctctgtgcccagacccgcctggtccccaggtctctcaccttgggtgaagattcagagatgccctgtaaggattttgcccactgggcaactcagaaatacttcgatctcccaagatataagaggcagcagcaaacgtgcctattgacgtctgtttcatagttaccacttacgcgagtagacagaactcggcttttcagaaaataggtgtcaagtccactttataagaacctttttttctaaaataagataaaaggtggctttgcattttctgattaaacgactgtgtctttgtcacctctgcttaactttaggagtatccattcctgtgattgtagacttttgttgatattcttcctggaagaatatcattcttttcttgaagggttggtttactagaatattcaaaatcaatcatgaaggcagttactattttgagtctaaaggttttctaaaaattaacctcacatcccttctgttagggtctttcagaatatcttttataaacagaagcatttgaagtcattgcttttgctacatgatttgtgtgtgtgaaggacataccacgtttaaatcattaattgaaaaacatcatataagccccaactttgtttggaggaagagacggaggttgaggtttttccttctgtataagcacctactgacaaaatgtagaggccattcaaccgtcaaacaccatttggttatatcgcagaggagacggatgtgtaaattactgcattgctttttttttcagtttgtataacctctaatctccgtttgcatgatacgctttgttagaaacattaattgtagtttggaagcaagtgtgtatgaataaagataatgatcattccaaaaaaaaaaaaaaaaa

SEQ ID NO: 19 - DNA, 1961 bp, Homo sapiens
accgcgcgtgccccgcctctctcccaagagctacgcggcggcggcggagcgcaggcctcgtgccgttacggccatcacggcggccgcagtggcgtcctggagccctcctcagtgctgaagctgctgaaagatggcagaagaagtggtggtagtagccaaatttgattatgtggcccaacaagaacaagagttggacatcaagaagaatgagagattatggcttctggatgattctaagtcctggtggcgagttcgaaattccatgaataaaacaggttttgtgccttctaactatgtggaaaggaaaaacagtgctcggaaagcatctattgtgaaaaacctaaaggataccttaggcattggaaaagtgaaaagaaaacctagtgtgccagattctgcatctcctgctgatgatagttttgttgacccaggggaacgtctctatgacctcaacatgcccgcttatgtgaaatttaactacatggctgagagagaggatgaattatcattgataaaggggacaaaggtgatcgtcatggagaaatgcagtgatgggtggtggcgtggtagctacaatggacaagttggatggttcccttcaaactatgtaactgaagaaggtgacagtcctttgggtgaccatgtgggttctctgtcagagaaattagcagcagtcgtcaataacctaaatactgggcaagtgttgcatgtggtacaggctctttacccattcagctcatctaatgatgaagaacttaatttcgagaaaggagatgtaatggatgttattgaaaaacctgaaaatgacccagagtggtggaaatgcaggaagatcaatggtatggttggtctagtaccaaaaaactatgttaccgttatgcagaataatccattaacttcaggtttggaaccatcacctccacagtgtgattacattaggccttcactcactggaaagtttgctggcaatccttggtattatggcaaagtcaccaggcatcaagcagaaatggcattaaatgaaagaggacatgaaggggatttcctcattcgtgatagtgaatcttcgccaaatgatttctcagtatcactaaaagcacaagggaaaaacaagcattttaaagtccaactaaaagagactgtctactgcattgggcagcgtaaattcagcaccatggaagaacttgtagaacattacaaaaaggcaccaatttttacaagtgaacaaggagaaaaattatatcttgtcaagcatttatcatgatactgctgaccagaagtgactgctgtgtagctgtaatttgtcatgtaattgaagactgagaaaatgttgggtccagtcgtgcttgattggaaattgttgtttctaaatctatatgagaattgacaataagtatttttattataactcagcccatacatatatactatgtatgcagtgcatctgcatagaacagttccttatccttggccttctgttttattgtttttttctttgctgttttccctttgcttctaatattacagttttgtattttgtaaacaaaaatcaaataatgcatatcagaatctttatatggaagaaatcctttattgcctttcctttgtttccttgtaaaggcaccctgttctgttatggtttttcattatataaaattattatatctatatatgacatatgctaaaatttcttggagagtgttaatcttttctgtgactaaatagcaataataagtggaaaattagaaattatttccaggtattatatttgtcacaggccattgtaaataccaagtatattgtgtctgccataatttttaaaaatacattcattgtcttcagtcatacagcaagacacatgagacatagattagaaaacatgttgtacaattttaatttacaactgttggaaataaaaatcacttaatttttttccaaaaaaaaaaaaaaaaaaaaaaaaa

SEQ ID NO: 20 - protein, 286 aa, Homo sapiens
MSDSEKLNLDSIIGRLLEGDIHGQYYDLLRLFEYGGFPPESNYLFLGDYVDRGKQSLETICLLLAYKIKYPENFFLLRGNHECASINRIYGFYDECKRRYNIKLWKTFTDCFNCLPIAAIVDEKIFCCHGGLSPDLQSMEQIRRIMRPTDVPDQGLLCDLLWSDPDKDVQGWGENDRGVSFTFGAEVVAKFLHKHDLDLICRAHQVVEDGYEFFAKRQLVTLFSAPNYCGEFDNAGAMMSVDETLMCSFQILKPADKNKGKYGQFSGLNPGGRPITPPRNSAKAKK

SEQ ID NO: 21 - protein, 330 aa, Mus musculus
MSDSEKLNLDSIIGRLLEVQGSRPGKNVQLTENEIRGLCLKSREIFLSQPILLELEAPLKICGDIHGQYYDLLRLFEYGGFPPESNYLFLGDYVDRGKQSLETICLLLAYKIRYPENFFLLRGNHECASINRIYGFYDECKRRYNIKLWKTFTDCFNCLPIAAIVDEKIFCCHGGLSPDLQSMEQIRRIMRPTDVPDQGLLCDLLWSDPDKDVQGWGENDRGVSFTFGAEVVAKFLHKHDLDLICRAHQVVEDGYEFFAKRQLVTLFSAPNYCGEFDNAGAMMSVDETLMCSFQILKPADKNKGKYGQFSGLNPGGRPITPPRNSAKAKK

SEQ ID NO: 22 - protein, 327 aa, Homo sapiens
MADGELNVDSLITRLLEVRGCRPGKIVQMTEAEVRGLCIKSREIFLSQPILLELEAPLKICGDIHGQYTDLLRLFEYGGFPPEANYLFLGDYVDRGKQSLETICLLLAYKIKYPENFFLLRGNHECASINRIYGFYDECKRRFNIKLWKTFTDCFNCLPIAAIVDEKIFCCHGGLSPDLQSMEQIRRIMRPTDVPDTGLLCDLLWSDPDKDVQGWGENDRGVSFTFGADVVSKFLNRHDLDLICRAHQVVEDGYEFFAKRQLVTLFSAPNYCGEFDNAGGMMSVDETLMCSFQILKPSEKKAKYQYGGLNSGRPVTPPRTANPPKKR

SEQ ID NO: 23 - protein, 327 aa, Mus musculus; identical to SEQ ID NO: 22

SEQ ID NO: 24 - protein, 292 aa, Homo sapiens
MQKYEKLEKIGEGTYGTVFKAKNRETHEIVALKRVRLDDDDEGVPSSALREICLLKELKHKNIVRLHDVLHSDKKLTLVFEFCDQDLKKYFDSCNGDLDPEIVKSFLFQLLKGLGFCHSRNVLHRDLKPQNLLINRNGELKLADFGLARAFGIPVRCYSAEVVTLWYRPPDVLFGAKLYSTSIDMWSAGCIFAELANAGRPLFPGNDVDDQLKRIFRLLGTPTEEQWPSMTKLPDYKPYPMYPATTSLVNVVPKLNATGRDLLQNLLKCNPVQRISAEEALQHPYFSDFCPP

SEQ ID NO: 25 - protein, 420 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 26 - protein, 672 aa, Homo Sapiens
MADVFPGNDSTASQDVANRFARKGALRQKNVHEVKDHKFIARFFKQPTFCSHCTDFIWGFGKQGFQCQVCCFVVHKRCHEFVTFSCPGADKGPDTDDPRSKHKFKIHTYGSPTFCDHCGSLLYGLIHQGMKCDTCDMNVHKQCVINVPSLCGMDHTEKRGRIYLKAEVADEKLHVTVRDAKNLIPMDPNGLSDPYVKLKLIPDPKNESKQKTKTIRSTLNPQWNESFTFKLKPSDKDRRLSVEIWDWDRTTRNDFMGSLSFGVSELMKMPASGWYKLLNQEEGEYYNVPIPEGDEEGNMELRQKFEKAKLGPAGNKVISPSEDRKQPSNNLDRVKLTDFNFLMVLGKGSFGKVMLADRKGTEELYAIKILKKDVVIQDDDVECTMVEKRVLALLDKPPFLTQLHSCFQTVDRLYFVMEYVNGGDLMYHIQQVGKFKEPQAVFYAAEISIGLFFLHKRGIIYRDLKLDNVMLDSEGHIKIADFGMCKEHMMDGVTTRTFCGTPDYIAPEIIAYQPYGKSVDWWAYGVLLYEMLAGQPPFDGEDEDELFQSIMEHNVSYPKSLSKEAVSICKGLMTKHPAKRLGCGPEGERDVREHAFFRRIDWEKLENREIQPPFKPKVCGKGAENFDKFFTRGQPVLTPPDQLVIANIDQSDFEGFSYVNPQFVHPILQSAV

SEQ ID NO: 27 - protein, 377 aa, Homo sapiens
MAEEVVVVAKFDYVAQQEQELDIKKNERLWLLDDSKSWWRVRNSMNKTGFVPSNYVERKNSARKASIVKNLKDTLGIGKVKRKPSVPDSASPADDSFVDPGERLYDLNMPAYVKFNYMAEREDELSLIKGTKVIVMEKCSDGWWRGSYNGQVGWFPSNYVTEEGDSPLGDHVGSLSEKLAAVVNNLNTGQVLHVVQALYPFSSSNDEELNFEKGDVMDVIEKPENDPEWWKCRKINGMVGLVPKNYVTVMQNNPLTSGLEPSPPQCDYIRPSLTGKFAGNPWYYGKVTRHQAEMALNERGHEGDFLIRDSESSPNDFSVSLKAQGKNKHFKVQLKETVYCIGQRKFSTMEELVEHYKKAPIFTSEQGEKLYLVKHLS

SEQ ID NO: 28 - DNA, 21 bp, Artificial Sequence
  siRNA Human PP1
gagacgcuacaacaucaaatt

SEQ ID NO: 29 - DNA, 21 bp, Artificial Sequence
  siRNA Human PP1
uuugauguuguagcgucuctt

SEQ ID NO: 30 - DNA, 21 bp, Artificial Sequence
  siRNA Human PP1
gugcucuuacgguuuauuguu

SEQ ID NO: 31 - DNA, 21 bp, Artificial Sequence
  siRNA Human PP1
guauacaccuagaguuuuatt

SEQ ID NO: 32 - DNA, 21 bp, Artificial Sequence
  siRNA Human PP1
uaaaacucuagguguauactt

SEQ ID NO: 33 - DNA, 21 bp, Artificial Sequence
  siRNA Human PP1
gcuuucuauggacuaauaaau

SEQ ID NO: 34 - DNA, 5916 bp, Homo sapiens
gcccctccctccgcccgcccgccggcccgcccgtcagtctggcaggcaggcaggcaatcggtccgagtggctgtcggctcttcagctctcccgctcggcgtcttccttcctcctcccggtcagcgtcggcggctgcaccggcggcggcgcagtccctgcgggaggggcgacaagagctgagcggcggccgccgagcgtcgagctcagcgcggcggaggcggcggcggcccggcagccaacatggcggcggcggcggcggcgggcgcgggcccggagatggtccgcgggcaggtgttcgacgtggggccgcgctacaccaacctctcgtacatcggcgagggcgcctacggcatggtgtgctctgcttatgataatgtcaacaaagttcgagtagctatcaagaaaatcagcccctttgagcaccagacctactgccagagaaccctgagggagataaaaatcttactgcgcttcagacatgagaacatcattggaatcaatgacattattcgagcaccaaccatcgagcaaatgaaagatgtatatatagtacaggacctcatggaaacagatctttacaagctcttgaagacacaacacctcagcaatgaccatatctgctattttctctaccagatcctcagagggttaaaatatatccattcagctaacgttctgcaccgtgacctcaagccttccaacctgctgctcaacaccacctgtgatctcaagatctgtgactttggcctggcccgtgttgcagatccagaccatgatcacacagggttcctgacagaatatgtggccacacgttggtacagggctccagaaattatgttgaattccaagggctacaccaagtccattgatatttggtctgtaggctgcattctggcagaaatgctttctaacaggcccatctttccagggaagcattatcttgaccagctgaaccacattttgggtattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggatacagatcttaaatttgtcaggacaagggctcagaggactggacgtgctcagacatcggtgttcttcttcccagttcttgacccctggtcctgtctccagcccgtcttggcttatccactttgactcctttgagccgtttggaggggcggtttctggtagttgtggcttttatgctttcaaagaatttcttcagtccagagaattcctcctggcagccctgtgtgtgtcacccattggtgacctgcggcagtatgtacttcagtgcacctactgcttactgttgctttagtcactaattgctttctggtttgaaagatgcagtggttcctccctctcctgaatccttttctacatgatgccctgctgaccatgcagccgcaccagagagagattcttccccaattggctctagtcactggcatctcactttatgatagggaaggctactacctagggcactttaagtcagtgacagccccttatttgcacttcaccttttgaccataactgtttccccagagcaggagcttgtggaaataccttggctgatgttgcagcctgcagcaagtgcttccgtctccggaatccttggggagcacttgtccacgtcttttctcatatcatggtagtcactaacatatataaggtatgtgctattggcccagcttttagaaaatgcagtcatttttctaaataaaaaggaagtactgcacccagcagtgtcactctgtagttactgtggtcacttgtaccatatagaggtgtaacacttgtcaagaagcgttatgtgcagtacttaatgtttgtaagacttacaaaaaaagatttaaagtggcagcttcactcgacatttggtgagagaagtacaaaggttgcagtgctgagctgtgggcggtttctggggatgtcccagggtggaactccacatgctggtgcatatacgcccttgagctacttcaaatgtgggtgtttcagtaaccacgttccatgcctgaggatttagcagagaggaacactgcgtctttaaatgagaaagtatacaattctttttccttctacagcatgtcagcatctcaagttcatttttcaacctacagtataacaatttgtaataaagcctccaggagctcatgacgtgaagcactgttctgtcctcaagtactcaaatatttctgatactgctgagtcagactgtcagaaaaagctagcactaactcgtgtttggagctctatccatattttactgatctctttaagtatttgttcctgccactgtgtactgtggagttgactcggtgttctgtcccagtgcggtgcctcctcttgacttccccactgctctctgtggtgagaaatttgccttgttcaataattactgtaccctcgcatgactgttacagctttctgtgcagagatgactgtccaagtgccacatgcctacgattgaaatgaaaactctattgttacctctgagttgtgttccacggaaaatgctatccagcagatcatttaggaaaaataattctatttttagcttttcatttctcagctgtccttttttcttgtttgatttttgacagcaatggagaatgggttatataaagactgcctgctaatatgaacagaaatgcatttgtaattcatgaaaataaatgtacatcttctatcttcacattcatgttaagattcagtgttgctttcctctggatcagcgtgtctgaatggacagtcaggttcaggttgtgctgaacacagaaatgctcacaggcctcactttgccgcccaggcactggcccagcacttggatttacataagatgagttagaaaggtacttctgtagggtcctttttacctctgctcggcagagaatcgatgctgtcatgttcctttattcacaatcttaggtctcaaatattctgtcaaaccctaacaaagaagccccgacatctcaggttggattccctggttctctctaaagagggcctgcccttgtgccccagaggtgctgctgggcacagccaagagttgggaagggccgccccacagtacgcagtcctcaccacccagcccagggtgctcacgctcaccactcctgtggctgaggaaggatagctggctcatcctcggaaaacagacccacatctctattcttgccctgaaatacgcgcttttcacttgcgtgctcagagctgccgtctgaaggtccacacagcattgacgggacacagaaatgtgactgttaccggataacactgattagtcagttttcatttataaaaaagcattgacagttttattactcttgtttctttttaaatggaaagttactattataaggttaatttggagtcctcttctaaatagaaaaccatatccttggctactaacatctggagactgtgagctccttcccattccccttcctggtactgtggagtcagattggcatgaaaccactaacttcattctagaatcattgtagccataagttgtgtgctttttattaatcatgccaaacataatgtaactgggcagagaatggtcctaaccaaggtacctatgaaaagcgctagctatcatgtgtagtagatgcatcattttggctcttcttacatttgtaaaaatgtacagattaggtcatcttaattcatattagtgacacggaacagcacctccactatttgtatgttcaaataagctttcagactaatagcttttttggtgtctaaaatgtaagcaaaaaattcctgctgaaacattccagtcctttcatttagtataaaagaaatactgaacaagccagtgggatggaattgaaagaactaatcatgaggactctgtcctgacacaggtcctcaaagctagcagagatacgcagacattgtggcatctgggtagaagaatactgtattgtgtgtgcagtgcacagtgtgtggtgtgtgcacactcattccttctgctcttgggcacaggcagtgggtgtagaggtaaccagtagctttgagaagctacatgtagctcaccagtggttttctctaaggaatcacaaaagtaaactacccaaccacatgccacgtaatatttcagccattcagaggaaactgttttctctttatttgcttatatgttaatatggtttttaaattggtaacttttatatagtatggtaacagtatgttaatacacacatacatacgcacacatgctttgggtccttccataatacttttatatttgtaaatcaatgttttggagcaatcccaagtttaagggaaatatttttgtaaatgtaatggttttgaaaatctgagcaatccttttgcttatacatttttaaagcatttgtgctttaaaattgttatgctggtgtttgaaacatgatactcctgtggtgcagatgagaagctataacagtgaatatgtggtttctcttacgtcatccaccttgacatgatgggtcagaaacaaatggaaatccagagcaagtcctccagggttgcaccaggtttacctaaagcttgttgccttttcttgtgctgtttatgcgtgtagagcactcaagaaagttctgaaactgctttgtatctgctttgtactgttggtgccttcttggtattgtaccccaaaattctgcatagattatttagtataatggtaagttaaaaaatgttaaaggaagattttattaagaatctgaatgtttattcattatattgttacaatttaacattaacatttatttgtggtatttgtgatttggttaatctgtataaaaattgtaagtagaaaggtttatatttcatcttaattcttttgatgttgtaaacgtactttttaaaagatggattatttgaatgtttatggcacctgacttgtaaaaaaaaaaaactacaaaaaaatccttagaatcattaaattgtgtccctgtattaccaaaataacacagcaccgtgcatgtatagtttaattgcagtttcatctgtgaaaacgtgaaattgtctagtccttcgttatgttccccagatgtcttccagatttgctctgcatgtggtaacttgtgttagggctgtgagctgttcctcgagttgaatggggatgtcagtgctcctagggttctccaggtggttcttcagaccttcacctgtgggggggggggtaggcggtgcccacgcccatctcctcatcctcctgaacttctgcaaccccactgctgggcagacatcctgggcaaccccttttttcagagcaagaagtcataaagataggatttcttggacatttggttcttatcaatattgggcattatgtaatgacttatttacaaaacaaagatactggaaaatgttttggatgtggtgttatggaaagagcacaggccttggacccatccagctgggttcagaactaccccctgcttataactgcggctggctgtgggccagtcattctgcgtctctgctttcttcctctgcttcagactgtcagctgtaaagtggaagcaatattacttgccttgtatatggtaaagattataaaaatacatttcaactgttcagcatagtacttcaaagcaagtactcagtaaatagcaagtctttttaaa

SEQ ID NO: 35 - DNA, 1417 bp, Homo sapiens
attaattgcttgccatcatgagcagaagcaagcgtgacaacaatttttatagtgtagagattggagattctacattcacagtcctgaaacgatatcagaatttaaaacctataggctcaggagctcaaggaatagtatgcgcagcttatgatgccattcttgaaagaaatgttgcaatcaagaagctaagccgaccatttcagaatcagactcatgccaagcgggcctacagagagctagttcttatgaaatgtgttaatcacaaaaatataattggccttttgaatgttttcacaccacagaaatccctagaagaatttcaagatgtttacatagtcatggagctcatggatgcaaatctttgccaagtgattcagatggagctagatcatgaaagaatgtcctaccttctctatcagatgctgtgtggaatcaagcaccttcattctgctggaattattcatcgggacttaaagcccagtaatatagtagtaaaatctgattgcactttgaagattcttgacttcggtctggccaggactgcaggaacgagttttatgatgacgccttatgtagtgactcgctactacagagcacccgaggtcatccttggcatgggctacaaggaaaacgtggatttatggtctgtggggtgcattatgggagaaatggtttgccacaaaatcctctttccaggaagggactatattgatcagtggaataaagttattgaacagcttggaacaccatgtcctgaattcatgaagaaactgcaaccaacagtaaggacttacgttgaaaacagacctaaatatgctggatatagctttgagaaactcttccctgatgtccttttcccagctgactcagaacacaacaaacttaaagccagtcaggcaagggatttgttatccaaaatgctggtaatagatgcatctaaaaggatctctgtagatgaagctctccaacacccgtacatcaatgtctggtatgatccttctgaagcagaagctccaccaccaaagatccctgacaagcagttagatgaaagggaacacacaatagaagagtggaaagaattgatatataaggaagttatggacttggaggagagaaccaagaatggagttatacgggggcagccctctcctttagcacaggtgcagcagtgatcaatggctctcagcatccatcatcatcgtcgtctgtcaatgatgtgtcttcaatgtcaacagatccgactttggcctctgatacagacagcagtctagaagcagcagctgggcctctgggctgctgtagatgactacttgggccatcggggggtgggagggatggggagtcggttagtcattgatagaactactttgaaaacaattcagtggtcttatttttgggtgatttttcaaaaaatgta

SEQ ID NO: 36 - DNA, 4145 bp, Homo sapiens
caaacaagtgcggccatttcaccagcccaggctggcttctgctgttgactggctgtggcacctcaagcagcccctttcccctctagcctcagtttatcaccgcaagagctaccattcatctagcacaacctgaccatcctcacactggtcagttccaaccttcccaggaatcttctgtggccatgttcactccggttttacagaacagagaacagaagctcagagaagtgaagcaacttgcccagctatgagagacagagccaggatttgaaaccagatgaggacgctgaggcccagagagggaaagccacttgcctagggacacacagcggggagaggtggagcagggcctctatttcgagacccctgactccacacctggtgtttgtgccaagaccccaggctgcctcccaggtcctctgggacagcccctgccttctaccaggaccatgggtagcaacaagagcaagcccaaggatgccagccagcggcgccgcagcctggagcccgccgagaacgtgcacggcgctggcgggggcgctttccccgcctcgcagacccccagcaagccagcctcggccgacggccaccgcggccccagcgcggccttcgcccccgcggccgccgagcccaagctgttcggaggcttcaactcctcggacaccgtcacctccccgcagagggcgggcccgctggccggtggagtgaccacctttgtggccctctatgactatgagtctaggacggagacagacctgtccttcaagaaaggcgagcggctccagattgtcaacaacacagagggagactggtggctggcccactcgctcagcacaggacagacaggctacatccccagcaactacgtggcgccctccgactccatccaggctgaggagtggtattttggcaagatcaccagacgggagtcagagcggttactgctcaatgcagagaacccgagagggaccttcctcgtgcgagaaagtgagaccacgaaaggtgcctactgcctctcagtgtctgacttcgacaacgccaagggcctcaacgtgaagcactacaagatccgcaagctggacagcggcggcttctacatcacctcccgcacccagttcaacagcctgcagcagctggtggcctactactccaaacacgccgatggcctgtgccaccgcctcaccaccgtgtgccccacgtccaagccgcagactcagggcctggccaaggatgcctgggagatccctcgggagtcgctgcggctggaggtcaagctgggccagggctgctttggcgaggtgtggatggggacctggaacggtaccaccagggtggccatcaaaaccctgaagcctggcacgatgtctccagaggccttcctgcaggaggcccaggtcatgaagaagctgaggcatgagaagctggtgcagttgtatgctgtggtttcagaggagcccatttacatcgtcacggagtacatgagcaaggggagtttgctggactttctcaagggggagacaggcaagtacctgcggctgcctcagctggtggacatggctgctcagatcgcctcaggcatggcgtacgtggagcggatgaactacgtccaccgggaccttcgtgcagccaacatcctggtgggagagaacctggtgtgcaaagtggccgactttgggctggctcggctcattgaagacaatgagtacacggcgcggcaaggtgccaaattccccatcaagtggacggctccagaagctgccctctatggccgcttcaccatcaagtcggacgtgtggtccttcgggatcctgctgactgagctcaccacaaagggacgggtgccctaccctgggatggtgaaccgcgaggtgctggaccaggtggagcggggctaccggatgccctgcccgccggagtgtcccgagtccctgcacgacctcatgtgccagtgctggcggaaggagcctgaggagcggcccaccttcgagtacctgcaggccttcctggaggactacttcacgtccaccgagccccagtaccagcccggggagaacctctaggcacaggcgggcccagaccggcttctcggcttggatcctgggctgggtggcccctgtctcggggcttgccccactctgcctgcctgctgttggtcctctctctgtggggctgaattgccaggggcgaggcccttcctctttggtggcatggaaggggcttctggacctagggtggcctgagagggcggtgggtatgcgagaccagcacggtgactctgtccagctcccgctgtggccgcacgcctctccctgcactccctcctggagctctgtgggtctctggaagaggaaccaggagaagggctggggccggggctgagggtgcccttttccagcctcagcctactccgctcactgaactccttccccacttctgtgccacccccggtctatgtcgagagctggccaaagagcctttccaaagaggagcgatgggcccctggccccgcctgcctgccaccctgccccttgccatccattctggaaacacctgtaggcagaggctgccgagacagaccctctgccgctgcttccaggctgggcagcacaaggccttgcctggcctgatgatggtgggtgggtgggatgagtaccccctcaaaccctgccctccttagacctgagggacccttcgagatcatcacttccttgcccccatttcacccatggggagacagttgagagcggggatgtgacatgcccaaggccacggagcagttcagagtggaggcgggcttggaacccggtgctccctctgtcatcctcaggaaccaacaattcgtcggaggcatcatggaaagactgggacagcccaggaaacaaggggtctgaggatgcattcgagatggcagattcccactgccgctgcccgctcagcccagctgttgggaacagcatggaggcagatgtggggctgagctggggaatcagggtaaaaggtgcaggtgtggagagagaggcttcaatcggcttgtgggtgatgtttgaccttcagagccagccggctatgaaagggagcgagcccctcggctctggaggcaatcaagcagacatagaagagccaagagtccaggaggccctggtcctggcctccttccccgtactttgtcccgtggcatttcaattcctggccctgttctcctccccaagtcggcaccctttaactcatgaggagggaaaagagtgcctaagcgggggtgaaagaggacgtgttacccactgccatgcaccaggactggctgtgtaaccttgggtggcccctgctgtctctctgggctgcagagtctgccccacatgtggccatggcctctgcaactgctcagctctggtccaggccctgtggcaggacacacatggtgagcctagccctgggacatcaggagactgggctctggctctgttcggcctttgggtgtgtggtggattctccctgggcctcagtgtgcccatctgtaaaggggcagctgacagtttgtggcatcttgccaagggtccctgtgtgtgtgtatgtgtgtgcatgtgtgcgtgtctccatgtgcgtccatatttaacatgtaaaaatgtcccccccgctccgtcccccaaacatgttgtacatttcaccatggccccctcatcatagcaataacattcccactgccaggggttcttgagccagccaggccctgccagtggggaaggaggccaagcagtgcctgcctatgaaatttcaacttttcctttcatacgtctttattacccaagtcttctcccgtccattccagtcaaatctgggctcactcaccccagcgagctctcaaatccctctccaactgcctaaggccctttgtgtaaggtgtcttaatactgtcctttttttttttttaacagtgttttgtagatttcagatgactatgcagaggcctgggggacccctggctctgggccgggcctggggctccgaaattccaaggcccagacttgcggggggtgggggggtatccagaattggttgtaaatactttgcatattgtctgattaaacacaaacagacctcagaaaaaaaaaaaaaaaaaaaaaaaaaaaaaaaaaaaaaaaaaaaaaaaa

SEQ ID NO: 37 - DNA, 2689 bp, Homo sapiens
acatctcccggcggcgggccgcggaagcagtgcagacgcggctcctagcggatgggtgctattgtgaggcggttgtagaagagtttcgtgagtgctcgcagctcatacctgtggctgtgtatccgtggccacagctggttggcgtcgccttgaaatcccaggccgtgaggagttagcgagccctgctcacactcggcgctctggttttcggtgggtgtgccctgcacctgcctcttcccccattctcattaataaaggtatccatggagaacactgaaaactcagtggattcaaaatccattaaaaatttggaaccaaagatcatacatggaagcgaatcaatggactctggaatatccctggacaacagttataaaatggattatcctgagatgggtttatgtataataattaataataagaattttcataaaagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggcctgccgtggtacagaactggactgtggcattgagacagacagtggtgttgatgatgacatggcgtgtcataaaataccagtggaggccgacttcttgtatgcatactccacagcacctggttattattcttggcgaaattcaaaggatggctcctggttcatccagtcgctttgtgccatgctgaaacagtatgccgacaagcttgaatttatgcacattcttacccgggttaaccgaaaggtggcaacagaatttgagtccttttcctttgacgctacttttcatgcaaagaaacagattccatgtattgtttccatgctcacaaaagaactctatttttatcactaaagaaatggttggttggtggttttttttagtttgtatgccaagtgagaagatggtatatttggtactgtatttccctctcattttgacctactctcatgctgcagagggtactttaagacatactccttccatcaaatagaaccactatgaagctacctcaaacttccagtcaggtagttgcaattgaattaaattaggaataaataaaaatggatactggtgcagtcattatgagaggcaatgattgttaatttacagctttcatgattagcaagttacagtgatgctgtgctatgaattttcaagtaattgtgaaaaagttaaacattgaagtaatgaatttttatgatattccccccacttaagactgtgtattctagttttgtcaaactgtagaaatgatgatgtggaagaacttaggcatctgtgggcatggtcaaaggctcaaacctttattttagaattgatatacacggatgacttaactgcatttttagaccatttatctgggattatggttttgtgatgtttgtcctgaacacttttgttgtaaaaaaataataataatgtttaatattgagaaagaaactaatattttatgtgagagaaagtgtgagcaaactaacttgacttttaaggctaaaacttaacattcatagaggggtggagttttaactgtaaggtgctacaatgcccctggatctaccagcataaatatcttctgatttgtccctatgcatatcagttgagcttcatataccagcaatatatctgaagagctattatataaaaaccccaaactgttgattattagccaggtaatgtgaataaattctataggaacatatgaaaatacaacttaaataataaacagtggaatataaggaaagcaataaatgaatgggctgagctgcctgtaacttgagagtagatggtttgagcctgagcagagacatgactcagcctgttccatgaaggcagagccatggaccacgcaggaagggcctacagcccatttctccatacgcactggtatgtgtggatgatgctgccagggcgccatcgccaagtaagaaagtgaagcaaatcagaaacttgtgaagtggaaatgttctaaaggtggtgaggcaataaaaatcatagtactctttgtagcaaaattcttaagtatgttattttctgttgaagtttacaatcaaaggaaaatagtaatgttttatactgtttactgaaagaaaaagacctatgagcacataggactctagacggcatccagccggaggccagagctgagccctcagcccgggaggcaggctccaggcctcagcaggtgcggagccgtcactgcaccaagtctcactggctgtcagtatgacatttcacgggagatttcttgttgctcaaaaaatgagctcgcatttgtcaatgacagtttcttttttcttactagacctgtaacttttgtaaatacacatagcatgtaatggtatcttaaagtgtgtttctatgtgacaattttgtacaaatttgttattttccatttttatttcaaaatatacattcaaacttaaaattaaaaaaaaaaaaaaaaa

SEQ ID NO: 38 - DNA, 3007 bp, Homo sapiens
aaggagagagggagggcggagggcggaggggcggcgggaggagggcggggaggagcgctcttcctggttgggccctgccctgagctgccaccgggaagccagcctcagggactgcagcgacccccaaacacccctcccccaggatgtcggaggagatcatcacgccggtgtactgcactggggtgtcagcccaagtgcagaagcagcgggccagggagctgggcctgggccgccatgagaatgccatcaagtacctgggccaggattatgagcagctgcgggtgcgatgcctgcagagtgggaccctcttccgtgatgaggccttccccccggtaccccagagcctgggttacaaggacctgggtcccaattcctccaagacctatggcatcaagtggaagcgtcccacggaactgctgtcaaacccccagttcattgtggatggagctacccgcacagacatctgccagggagcactgggggactgctggctcttggcggccattgcctccctcactctcaacgacaccctcctgcaccgagtggttccgcacggccagagcttccagaatggctatgccggcatcttccatttccagctgtggcaatttggggagtgggtggacgtggtcgtggatgacctgctgcccatcaaggacgggaagctagtgttcgtgcactctgccgaaggcaacgagttctggagcgccctgcttgagaaggcctatgccaaggtaaatggcagctacgaggccctgtcagggggcagcacctcagagggctttgaggacttcacaggcggggttaccgagtggtacgagttgcgcaaggctcccagtgacctctaccagatcatcctcaaggcgctggagcggggctccctgctgggctgctccatagacatctccagcgttctagacatggaggccatcactttcaagaagttggtgaagggccatgcctactctgtgaccggggccaagcaggtgaactaccgaggccaggtggtgagcctgatccggatgcggaacccctggggcgaggtggagtggacgggagcctggagcgacagctcctcagagtggaacaacgtggacccatatgaacgggaccagctccgggtcaagatggaggacggggagttctggatgtcattccgagacttcatgcgggagttcacccgcctggagatctgcaacctcacacccgacgccctcaagagccggaccatccgcaaatggaacaccacactctacgaaggcacctggcggcgggggagcaccgcggggggctgccgaaactacccagccaccttctgggtgaaccctcagttcaagatccggctggatgagacggatgacccggacgactacggggaccgcgagtcaggctgcagcttcgtgctcgcccttatgcagaagcaccgtcgccgcgagcgccgcttcggccgcgacatggagactattggcttcgcggtctacgaggtccctccggagctggtgggccagccggccgtacacttgaagcgtgacttcttcctggccaatgcgtctcgggcgcgctcagagcagttcatcaacctgcgagaggtcagcacccgcttccgcctgccacccggggagtatgtggtggtgccctccaccttcgagcccaacaaggagggcgacttcgtgctgcgcttcttctcagagaagagtgctgggactgtggagctggatgaccagatccaggccaatctccccgatgagcaagtgctctcagaagaggagattgacgagaacttcaaggccctcttcaggcagctggcaggggaggacatggagatcagcgtgaaggagttgcggacaatcctcaataggatcatcagcaaacacaaagacctgcggaccaagggcttcagcctagagtcgtgccgcagcatggtgaacctcatggatcgtgatggcaatgggaagctgggcctggtggagttcaacatcctgtggaaccgcatccggaattacctgtccatcttccggaagtttgacctggacaagtcgggcagcatgagtgcctacgagatgcggatggccattgagtcggcaggcttcaagctcaacaagaagctgtacgagctcatcatcacccgctactcggagcccgacctggcggtcgactttgacaatttcgtttgctgcctggtgcggctagagaccatgttccgatttttcaaaactctggacacagatctggatggagttgtgacctttgacttgtttaagtggttgcagctgaccatgtttgcatgaggcagggactcggtcccccttgccgtgctcccctccctcctcgtctgccaagcctcgcctcctaccacaccacaccaggccaccccagctgcaagtgccttccttggagcagagaggcagcctcgtcctcctgtcccctctcctcccagccaccatcgttcatctgctccgggcagaactgtgtggcccctgcctgtgccagccatgggctcgggatggactccctgggccccacccattgccaagccaggaaggcagctttcgcttgttcctgcctcgggacagccccgggtttccccagcatcctgatgtgtcccctctccccacttcagaggccacccactcagcaccaccggcctggccttgcctgcagactataaactataaccactagctcgacacagtctgcagtccaggcgtgtggagccgcctcccggctcggggaggccccggggctgggaacgcctgtgccttcctgcgccgaagccaacgccccctctgtccttccctggccctgctgccgaccaggagctgcccagcctgtgggcggtcggccttccctccttcgctccttttttatattagtgattttaaaggggactcttcagggacttgtgtactggttatgggggtgccagaggcactaggcttggggtggggaggtcccgtgttccatatagaggaaccccaaataataaaaggccccacatctgtctgtg

SEQ ID NO: 39 - DNA, 1492 bp, Homo sapiens
cgggcgacagcagggccgcggtgcagtgtccgacccgagagttgcggcctgagtcaccggccccgccctccggagccggacgctgcgggaggcccgggagcggcagtggaaccgactcccagaactccggacgtgtgcggcgcagtgagtcgcagccatgttcctggttaactcgttcttgaagggcggcggcggcggcggcgggggaggcgggggcctgggtgggggcctgggaaatgtgcttggaggcctgatcagcggggccgggggcggcggcggcggcggcggcggcggcggcggtggtggaggcggcggtggcggtggaacggccatgcgcatcctaggcggagtcatcagcgccatcagcgaggcggctgcgcagtacaacccggagcccccgcccccacgcacacattactccaacattgaggccaacgagagtgaggaggtccggcagttccggagactctttgcccagctggctggagatgacatggaggtcagcgccacagaactcatgaacattctcaataaggttgtgacacgacaccctgatctgaagactgatggttttggcattgacacatgtcgcagcatggtggccgtgatggatagcgacaccacaggcaagctgggctttgaggaattcaagtacttgtggaacaacatcaaaaggtggcaggccatatacaaacagttcgacactgaccgatcagggaccatttgcagtagtgaactcccaggtgcctttgaggcagcagggttccacctgaatgagcatctctataacatgatcatccgacgctactcagatgaaagtgggaacatggattttgacaacttcatcagctgcttggtcaggctggacgccatgttccgtgccttcaaatctcttgacaaagatggcactggacaaatccaggtgaacatccaggagtggctgcagctgactatgtattcctgaactggagccccagacccgccccctcactgccttgctataggagtcacctggagcctcggtctctcccagggccgatcctgtctgcagtcacatctttgtggggcctgctgacccacaagcttttgttctctcagtacttgttacccagcttctcaacatccagggcccaatttgccctgcctggagttccccctggctctaggacactctaacaagctctgtccacgggtctccccattcccaccaggccctgcacacacccactccgtaacctctcccctgtacctgtgccaagcctagcacttgtgatgcctccatgccccgagggccctctctcagttctgggaggatgactccagtccctgcacgccctggcacacccttcacggttgctacccaggcggccaagctccagaccgtgccagacccaggtgccccagtgcctttgtctatattctgctcccagcctgccaggcccaggaggaaataaacatgccccagttgctgatctctaaaaaaaaaaaaaaa

SEQ ID NO: 40 - DNA, 2849 bp, Homo sapiens
cccgcctcctggtaggagggggtttccgcttccggcagcagcggctgcagcctcgctctggtccctgcggctggcggccgagccgtgtgtctcctcctccatcgccgccatattgtctgtgtgagcagaggggagagcggccgccgccgctgccgcttccaccacagctctatcaaggcttgtcaagcagtgtgctcatcacatggtaaatcatgcagcgtggaacctcataaaatctccaagaaacatcattcacccatactgactagtttcacatctctttgtttgaagaaaacaggtctgaaacaaggtcttacccccagctgcttctgaacacagtgactgccagatctccaaacatcaagtccagctttgtccgccaacctgtctgacatgtcgggacccgtgccaagcagggccagagtttacacagatgttaatacacacagacctcgagaatactgggattacgagtcacatgtggtggaatggggaaatcaagatgactaccagctggttcgaaaattaggccgaggtaaatacagtgaagtatttgaagccatcaacatcacaaataatgaaaaagttgttgttaaaattctcaagccagtaaaaaagaagaaaattaagcgtgaaataaagattttggagaatttgagaggaggtcccaacatcatcacactggcagacattgtaaaagaccctgtgtcacgaacccccgccttggtttttgaacacgtaaacaacacagacttcaagcaattgtaccagacgttaacagactatgatattcgattttacatgtatgagattctgaaggccctggattattgtcacagcatgggaattatgcacagagatgtcaagccccataatgtcatgattgatcatgagcacagaaagctacgactaatagactggggtttggctgagttttatcatcctggccaagaatataatgtccgagttgcttcccgatacttcaaaggtcctgagctacttgtagactatcagatgtacgattatagtttggatatgtggagtttgggttgtatgctggcaagtatgatctttcggaaggagccatttttccatggacatgacaattatgatcagttggtgaggatagccaaggttctggggacagaagatttatatgactatattgacaaatacaacattgaattagatccacgtttcaatgatatcttgggcagacactctcgaaagcgatgggaacgctttgtccacagtgaaaatcagcaccttgtcagccctgaggccttggatttcctggacaaactgctgcgatatgaccaccagtcacggcttactgcaagagaggcaatggagcacccctatttctacactgttgtgaaggaccaggctcgaatgggttcatctagcatgccagggggcagtacgcccgtcagcagcgccaatatgatgtcagggatttcttcagtgccaaccccttcaccccttggacctctggcaggctcaccagtgattgctgctgccaacccccttgggatgcctgttccagctgccgctggcgctcagcagtaacggccctatctgtctcctgatgcctgagcagaggtgggggagtccaccctctccttgatgcagcttgcgcctggcggggaggggtgaaacacttcagaagcaccgtgtctgaaccgttgcttgtggatttatagtagttcagtcataaaaaaaaaattataataggctgattttcttttttctttttttttttaactcgaacttttcataactcaggggattccctgaaaaattacctgcaggtggaatatttcatggacaaatttttttttctcccctcccaaatttagttcctcatcacaaaagaacaaagataaaccagcctcaatcccggctgctgcatttaggtggagacttcttcccattcccaccattgttcctccaccgtcccacactttagggggttggtatctcgtgctcttctccagagattacaaaaatgtagcttctcaggggaggcaggaagaaaggaaggaaggaaagaaggaagggaggacccaatctataggagcagtggactgcttgctggtcgcttacatcactttactccataagcgcttcagtggggttatcctagtggctcttgtggaagtgtgtcttagttacatcaagatgttgaaaatctacccaaaatgcagacagatactaaaaacttctgttcagtaagaatcatgtcttactgatctaaccctaaatccaactcatttatacttttatttttagttcagtttaaaatgttgataccttccctcccaggctccttaccttggtcttttccctgttcatctcccaacatgctgtgctccatagctggtaggagagggaaggcaaaatctttcttagttttctttgtcttggccattttgaattcatttagttactgggcataacttactgctttttacaaaagaaacaaacattgtctgtacaggtttcatgctagagctaatgggagatgtggccacactgacttccattttaagctttctaccttcttttcctccgaccgtccccttccctcacatgccatccagtgagaagacctgctcctcagtcttgtaaatgtatcttgagaggtaggagcagagccactatctccattgaagctgaaatggtagacctgtaattgtgggaaaactataaactctcttgttacagccccgccaccccttgctgtgtgtatatatataatactttgtccttcatatgtgaaagatccagtgttggaattctttggtgtaaataaacgtttggttttatttatcaaaaaaaaaaaaaaa

SEQ ID NO: 41 - DNA, 1674 bp, Homo sapiens
gcggccgcccgccgccgcgctcctcctcctcctcctccagcgcccggcggcccgctgcctcctccgcccgacgccccgcgtcccccgccgcgccgccgccgccaccctctgcgccccgcgccgccccccggtcccgcccgccatgcccggcccggccgcgggcagcagggcccgggtctacgccgaggtgaacagtctgaggagccgcgagtactgggactacgaggctcacgtcccgagctggggtaatcaagatgattaccaactggttcgaaaacttggtcggggaaaatatagtgaagtatttgaggccattaatatcaccaacaatgagagagtggttgtaaaaatcctgaagccagtgaagaaaaagaagataaaacgagaggttaagattctggagaaccttcgtggtggaacaaatatcattaagctgattgacactgtaaaggaccccgtgtcaaagacaccagctttggtatttgaatatatcaataatacagattttaagcaactctaccagatcctgacagactttgatatccggttttatatgtatgaactacttaaagctctggattactgccacagcaagggaatcatgcacagggatgtgaaacctcacaatgtcatgatagatcaccaacagaaaaagctgcgactgatagattggggtctggcagaattctatcatcctgctcaggagtacaatgttcgtgtagcctcaaggtacttcaagggaccagagctcctcgtggactatcagatgtatgattatagcttggacatgtggagtttgggctgtatgttagcaagcatgatctttcgaagggaaccattcttccatggacaggacaactatgaccagcttgttcgcattgccaaggttctgggtacagaagaactgtatgggtatctgaagaagtatcacatagacctagatccacacttcaacgatatcctgggacaacattcacggaaacgctgggaaaactttatccatagtgagaacagacaccttgtcagccctgaggccctagatcttctggacaaacttctgcgatacgaccatcaacagagactgactgccaaagaggccatggagcacccatacttctaccctgtggtgaaggagcagtcccagccttgtgcagacaatgctgtgctttccagtggtctcacggcagcacgatgaagactggaaagcgacgggtctgttgcggttctcccacttttccataagcagaacaagaaccaaatcaaacgtcttaacgcgtatagagagatcacgttccgtgagcagacacaaaacggtggcaggtttggcgagcacgaactagaccaagcgaagggcagcccaccaccgtatatcaaacctcacttccgaatgtaaaaggctcacttgcctttggcttcctgttgacttcttcccgacccagaaagcatggggaatgtgaagggtatgcagaatgttgttggttactgttgctccccgagcccctcaactcgtcccgtggccgcctgtttttccagcaaaccacgctaactagctgaccacagactccacagtggggggacgggcgcagtatgtggcatggcggcagttacatattattattttaaaagtatatattattgaataaaaggttttaaaagaaaaaaaaaaaaaaaaaa

SEQ ID NO: 42 - DNA, 1128 bp, Homo sapiens
gcttctcgttgtgccccgcccgcaagcgccctcctccgggccttcgtgacagccaggtcgtgcgcgggtcatcctgggattggtagttcgctttctctcatttagccagtttctttctctaccggggactccgtgtcccggcatccaccgcggcacctgacccttggcgcttgcgtgttgccctcttccccaccctccctaatttccactccccccaccccacttcgcctgccgcggtcgggtccgcggcctgcgctgtagcggtcgccgccgttccctggaagtagcaacttccctaccccaccccagtcctggtccccgtccagccgctgacgtgaagatgagcagctcagaggaggtgtcctggatttcctggttctgtgggctccgtggcaatgaattcttctgtgaagtggatgaagactacatccaggacaaatttaatcttactggactcaatgagcaggtccctcactaccgacaagctctagacatgatcttggacctggagcctgatgaagaactggaagacaaccccaaccagagtgacctgattgagcaggcagccgagatgctttatggattgatccacgcccgctacatccttaccaaccgtggcatcgcccagatgttggaaaagtaccagcaaggagactttggttactgtcctcgtgtgtactgtgagaaccagccaatgcttcccattggcctttcagacatcccaggtgaagccatggtgaagctctactgccccaagtgcatggatgtgtacacacccaagtcatcaagacaccatcacacggatggcgcctacttcggcactggtttccctcacatgctcttcatggtgcatcccgagtaccggcccaagagacctgccaaccagtttgtgcccaggctctacggtttcaagatccatccgatggcctaccagctgcagctccaagccgccagcaacttcaagagcccagtcaagacgattcgctgattccctcccccacctgtcctgcagtctttgacttttcctttcttttttgccaccctttcaggaaccctgtatggtttttagtttaaattaaaggagtcgttattgtggtgggaatatgaaataaagtagaagaaaaggcc

SEQ ID NO: 43 - DNA, 2643 bp, Homo sapiens
gcttcagttaccagccggctacgtcgcgcctgcgctttgacccccagtttgcgccccaactccggtcgtgcggccgcccggggagggctctgcagttgcgcagcttgctccccggcccttttcccctccgctccccgccgcctcctgacgccgggcgtgacgtcaccacgcccggcggccgccattacagagagccgagctctggagcctcagcgagcggaggaggaggcgcagcggccgacggccgagtactgcggtgagagccagcgggccagcgccagcctcaacagccgccagaagtacacgaggaaccggcggcggcgtgtgcgtgtaggcccgtgtgcgggcggcggcgcgggagcagcgcggagcggcagccggctggggcgggtggcatcatggacgagaaggtgttcaccaaggagctggaccagtggatcgagcagctgaacgagtgcaagcagctgtccgagtcccaggtcaagagcctctgcgagaaggctaaagaaatcctgacaaaagaatccaacgtgcaagaggttcgatgtccagttactgtctgtggagatgtgcatgggcaatttcatgatctcatggaactgtttagaattggtggcaaatcaccagatacaaattacttgtttatgggagattatgttgacagaggatattattcagttgaaacagttacactgcttgtagctcttaaggttcgttaccgtgaacgcatcaccattcttcgagggaatcatgagagcagacagatcacacaagtttatggtttctatgatgaatgtttaagaaaatatggaaatgcaaatgtttggaaatattttacagatctttttgactatcttcctctcactgccttggtggatgggcagatcttctgtctacatggtggtctctcgccatctatagatacactggatcatatcagagcacttgatcgcctacaagaagttccccatgagggtccaatgtgtgacttgctgtggtcagatccagatgaccgtggtggttggggtatatctcctcgaggagctggttacacctttgggcaagatatttctgagacatttaatcatgccaatggcctcacgttggtgtctagagctcaccagctagtgatggagggatataactggtgccatgaccggaatgtagtaacgattttcagtgctccaaactattgttatcgttgtggtaaccaagctgcaatcatggaacttgacgatactctaaaatactctttcttgcagtttgacccagcacctcgtagaggcgagccacatgttactcgtcgtaccccagactacttcctgtaatgaaattttaaacttgtacagtattgccatgaaccatatatcgacctaatggaaatgggaagagcaacagtaactccaaagtgtcagaaaatagttaacattcaaaaaacttgttttcacatggaccaaaagatgtgccatataaaaatacaaagcctcttgtcatcaacagccgtgaccactttagaatgaaccagttcattgcatgctgaagcgacattgttggtcaagaaaccagtttctggcatagcgctatttgtagttacttttgctttctctgagagactgcagataataagatgtaaacattaacacctcgtgaatacaatttaacttccatttagctatagctttactcagcatgactgtagataaggatagcagcaaacaatcattggagcttaatgaacatttttaaaaataattaccaaggcctcccttctacttgtgagttttgaaattgttctttttattttcagggataccgtttaatttaattatatgatttgtctgcactcagtttattccctactcaaatctcagccccatgttgttctttgttattgtcagaacctggtgagttgttttgaacagaactgttttttccccttcctgtaagacgatgtgactgcacaagagcactgcagtgtttttcataataaacttgtgaactaagaactgagaaggtcaaattttaattgtatcaatgggcaagactggtgctgtttattaaaaaagttaaatcaattgagtaaattttagaatttgtagacttgtaggtaaaataaaaatcaagggcactacataacctctctggtaactccttgacattcttcagattaacttcaggatttatttgtatttcacatattacaatttgtcacattgttggtgtgcactttgtgggttcttcctgcatattaacttgtttgtaagaaaggaaatctgtgctgcttcagtaagacttaattgtaaaaccatataacttgagatttaagtctttgggttttgttttaataaaacagcatgttttcaggtagagcttaaactaaatgatgtgtttacttagtgcagtttctggttatgaatattatattgctatgtgtatattatatggactctttaaaatgattgacagattggcaaattcttaaatctttgtacattgttgagtcatatgttcttagagttaaatttgtctcagataagaaagtgttaaagcattagcctgtgtcaagttctttgagtgatactagtgaaaccaaatagaaaactattgttggatcatgatttagtcttatgtacattcacccga

SEQ ID NO: 44 - DNA, 3945 bp, Homo sapiens
aggtgacgtcactggccaggccagccggcgccattttgaaagtggagtcgcctgcccctgccgctgccgccgccgccgtcgctgtcgtagtcgccgccgccgctgccggagaaagagcacgagcggggaagccccagagtgaaatctagcatcctgccggctggtctgcccgcccctccttccttttccccccggcccccgtcccctccccccgcaggtgccatccgccgccatccgccctctctacccccccatccccaggtgaggggggtgagttcaggaagcggagaccccgaggaacccagcagggtcaccatttgcagcgcaacatggcaggagctggaggagggaatgatattcagtggtgtttttctcaggtgaaaggagcagtagatgatgatgtagcagaagcagatataatttctacagtagaatttaatcattctggagaattactagcaacaggagataaaggtggtagagttgtcatctttcaacaggagcaggagaacaaaatccagtctcatagcagaggagaatataatgtttacagcaccttccagagccatgaaccagagtttgactacttgaaaagtttagaaatagaagaaaagatcaacaaaattaggtggttaccccagaaaaatgctgctcagtttttattgtctaccaatgataaaacaataaaattatggaaaatcagtgaaagggacaaaagaccagaagggtataacttgaaagaggaggatggaaggtatagagatcctactacagttactacactacgagtgccagtctttaggcctatggatctaatggttgaggccagtccacgaagaatatttgccaatgctcatacatatcacatcaactcaatttctattaatagtgattatgaaacatatttatctgcagatgatttgcggattaatctttggcatctggaaattacagacaggagttttaacattgtggatatcaagcctgccaatatggaagagctaacagaggtgattacagcagcagaatttcatccaaacagctgtaacacatttgtatacagcagcagtaaaggaactattcggctatgtgacatgagggcatctgccctctgtgatagacattctaaattgtttgaagaacctgaagatcccagtaacaggtcatttttttccgaaatcatctcctctatttcggatgtaaaattcagccatagtggtcgatatatgatgactagagactatttgtcagtcaaaatttgggacttaaatatggaaaacaggcctgtggaaacataccaggtgcatgaatacctcagaagtaaactctgttcactgtatgaaaatgactgcatatttgacaaatttgaatgttgttggaatggatctgacagtgttgtcatgactggatcttacaataatttcttcagaatgtttgacagaaacacaaagcgagacataaccctagaagcatcgcgggaaaacaataagcctcgcacagttctgaagcctcgcaaagtctgtgcaagtggcaagcgaaagaaagatgaaataagtgttgacagcctagacttcaataagaaaatccttcacacagcctggcaccccaaggaaaatatcattgccgtagctactacaaacaatctgtatatatttcaagacaaagtgaattagggttggcattcctagcagaagaacccacttcctgcttagttgagatagttgaatctagcattcgttcctataaaagagagaggtccattgtggcgcccctttccagtgtttgacagtgtgccattcgacaacacattgttatagctacatggagaaagctctgtggattcatcactgtggtgttctccatgtctgctagccatttaggtaagggtagggcacttttaatttaaatgacttcttgcaccatcttgcctaatggactagattggactgtatcaacattgatttactccactttttatgccttccattgtgatgacgtcaaacacagtgaaagccttcagtcatgctatgggatttaattgtgtatcctcattactgtatcatttgtggggtacaccccttccccctttttttaaattaaatacagctcattcttactgtggcttgtagcattcctcctcttctggcctcctggactgctccccttcatctcttacccttgccccctccacccggtcttggtggtggtatattaaaaaaagaaagaatgaaagcacacaaaatgagtcagtttggggtcagtggtataaagggggtatatgttgcaaacaaatgttttagtaacagttggctgtaatcactcctcgccgtgtctggcactgaaaataaggaaaaaaaacctactactgaataaaagtgacaaagaatggagaatctggttttctttttctttttaacctacctcttgtagccaatattttgtgtcatacctttgggcacagtgaaacaaaatgggttttcattgtttattggtatttttgttaattatttttaacaagtgttcttttacatgcaggaggagaggtattggttctctatgaacatattttgaatataggttttattaaggatttcacaatctataaatgctactagtttttttttttttttttaccatcatgagggtattggatacattgtgtctctatttaactcattatgtgttaatgaaattgttgtaaatgggaaccaaatttgtagaacttaatttctactttttagagtgcttaatttcatttttgccttactaaatagtcaaagacttataaaacatttttaacaagttagaacttttttgttattcagtcatataaaatagcagaaaactaacatcaagtgacactgcactaaatactttttttgtattttactgctatcaaatcagaatgaaatatactttaccatagatatttttcttctatttttggttttccaaagctatatgaaagacaaatttttaaaggtacagcgttcaaaaagtgcttaatgaactccaacagctgcctcaaataaaaatctgtatatgaatacattttccctaagcggtgatacattatccaaagatgaagagtgctcctatttttaataggtagaatgtacctttgtcagtcttgcagaaactttaatggagaagaaatggactttatttttgaaaggtgaaatgaacaggcatttatattattagagaatggtagtcttattttggtggaacataatgtaacaaccttgaatttcagaggactctgagtgcttctatgtccactacctattgttctattcttcaataatgaaaaagaattcaacgagccgaccgtgattccctctactacaaatattatgtcttgtaagttagcatttttagcacacaggagaaattttatgtaataaaattactgtatcttttggatttaacaaatttgtatttgaaacacattctatgtctgataattcttaatggcacttttactaatttatttggggatcttgggtacattcttaatttgtgtttattcttcacgcttgacttgcaagtgggatattcccctgccacaagtgtcaaacagtgatattcttcctgtgttgtgactggacagttttccagatcttttttgggagattttcctacagcttggttgtatgtcttgagataacaccaccaaacagctctcagaaattcttttttgattgatcagtagctatgatgattctcctccatgacactaaggattagtttatatatttaagagaaataattgctaaaattaaaatgcctctatcaaggaatgctattataaattattgttaacattctcaagtattaattttttaatttcattggtgtagcaaactctaagcccagccactcattttacatggccatggttaatctttttattaataaaaattatacttagaataaaaaaaa

SEQ ID NO: 45 - DNA, 2519 bp, Homo sapiens
aatcttggtcgctaggacacggctaacttccgctttcttccccctctcctaggctcaaactagtcaaatcttgttcactcgaccaatggcaaatcggaagtgggcgggacttcacaagtccggaccaaagaaacgcgagcttagccctgggtagcgcggccaatggccgtggagcagcccctgtaaactggctcgggcgcccccacgcccgcccttccttcttctcccagcattgccccccccacgtttcagcacagcgctggccgcagtctgacaggaaagggacggagccaagatggcggcggccgacggcgacgactcgctgtaccccatcgcggtgctcatagacgaactccgcaatgaggacgttcagcttcgcctcaacagcatcaagaagctgtccaccatcgccttggcccttggggttgaaaggacccgaagtgagcttctgcctttccttacagataccatctatgatgaagatgaggtcctcctggccctggcagaacagctgggaaccttcactaccctggtgggaggcccagagtacgtgcactgcctgctgccaccgctggagtcgctggccacagtggaggagacagtggtgcgggacaaggcagtggagtccttacgggccatctcacacgagcactcgccctctgacctggaggcgcactttgtgccgctagtgaagcggctggcgggcggcgactggttcacctcccgcacctcggcctgcggcctcttctccgtctgctacccccgagtgtccagtgctgtgaaggcggaacttcgacagtacttccggaacctgtgctcagatgacacccccatggtgcggcgggccgcagcctccaagctgggggagtttgccaaggtgctggagctggacaacgtcaagagtgagatcatccccatgttctccaacctggcctctgacgagcaggactcggtgcggctgctggcggtggaggcgtgcgtgaacatcgcccagcttctgccccaggaggatctggaggccctggtgatgcccactctgcgccaggccgctgaagacaagtcctggcgcgtccgctacatggtggctgacaagttcacagagctccagaaagcagtggggcctgagatcaccaagacagacctggtccctgccttccagaacctgatgaaagactgtgaggccgaggtgagggccgcagcctcccacaaggtcaaagagttctgtgaaaacctctcagctgactgtcgggagaatgtgatcatgtcccagatcttgccctgcatcaaggagctggtgtccgatgccaaccaacatgtcaagtctgccctggcctcagtcatcatgggtctctctcccatcttgggcaaagacaacaccatcgagcacctcttgcccctcttcctggctcagctgaaggatgagtgccctgaggtacggctgaacatcatctctaacctggactgtgtgaacgaggtgattggcatccggcagctgtcccagtccctgctccctgccattgtggagctggctgaggacgccaagtggcgggtgcggctggccatcattgagtacatgcccctcctggctggacagctgggagtggagttctttgatgagaaacttaactccttgtgcatggcctggcttgtggatcatgtatatgccatccgcgaggcagccaccagcaacctgaagaagctagtggaaaagtttgggaaggagtgggcccatgccacaatcatccccaaggtcttggccatgtccggagaccccaactacctgcaccgcatgactacgctcttctgcatcaatgtgctgtctgaggtctgtgggcaggacatcaccaccaagcacatgctacccacggttctgcgcatggctggggacccggttgccaatgtccgcttcaatgtggccaagtctctgcagaagatagggcccatcctggacaacagcaccttgcagagtgaagtcaagcccatcctagagaagctgacccaggaccaggatgtggacgtcaaatactttgcccaggaggctctgactgttctgtctctcgcctgatgctggaagaggagcaaacactggcctctggtgtccaccctccaacccccacaagtccctctttggggagacactggggggcctttggctgtcactccctgtgcatggtctgaccccaggccccttcccccagcacggttcctcctctccccagcctgggaagatgtctcactgtccacctcccaacgggctaggggagcacggggttggacaggacagtgaccttgggaggaaggggctactccgcccacgtcagggagagatgtgagcatcccgggtcactggatcctgctgctgtaatgggaacccctcccccatttacttctccacctcccgtcctccccatcattggtttttttttgtgtgtcaactgtgccgtttttattttattccttttattttcccccttttcacagagaaataaaggtctagaagtagttggtcaaaaaaaaaa

SEQ ID NO: 46 - protein, 360 aa, Homo sapiens [sequence omitted]

SEQ ID NO: 47 - protein, 384 aa, Homo sapiens
MSRSKRDNNFYSVEIGDSTFTVLKRYQNLKPIGSGAQGIVCAAYDAILERNVAIKKLSRPFQNQTHAKRAYRELVLMKCVNHKNIIGLLNVFTPQKSLEEFQDVYIVMELMDANLCQVIQMELDHERMSYLLYQMLCGIKHLHSAGIIHRDLKPSNIVVKSDCTLKILDFGLARTAGTSFMMTPYVVTRYYRAPEVILGMGYKENVDLWSVGCIMGEMVCHKILFPGRDYIDQWNKVIEQLGTPCPEFMKKLQPTVRTYVENRPKYAGYSFEKLFPDVLFPADSEHNKLKASQARDLLSKMLVIDASKRISVDEALQHPYINVWYDPSEAEAPPPKIPDKQLDEREHTIEEWKELIYKEVMDLEERTKNGVIRGQPSPLAQVQQ

SEQ ID NO: 48 - protein, 536 aa, Homo sapiens
MGSNKSKPKDASQRRRSLEPAENVHGAGGGAFPASQTPSKPASADGHRGPSAAFAPAAAEPKLFGGFNSSDTVTSPQRAGPLAGGVTTFVALYDYESRTETDLSFKKGERLQIVNNTEGDWWLAHSLSTGQTGYIPSNYVAPSDSIQAEEWYFGKITRRESERLLLNAENPRGTFLVRESETTKGAYCLSVSDFDNAKGLNVKHYKIRKLDSGGFYITSRTQFNSLQQLVAYYSKHADGLCHRLTTVCPTSKPQTQGLAKDAWEIPRESLRLEVKLGQGCFGEVWMGTWNGTTRVAIKTLKPGTMSPEAFLQEAQVMKKLRHEKLVQLYAVVSEEPIYIVTEYMSKGSLLDFLKGETGKYLRLPQLVDMAAQIASGMAYVERMNYVHRDLRAANILVGENLVCKVADFGLARLIEDNEYTARQGAKFPIKWTAPEAALYGRFTIKSDVWSFGILLTELTTKGRVPYPGMVNREVLDQVERGYRMPCPPECPESLHDLMCQCWRKEPEERPTFEYLQAFLEDYFTSTEPQYQPGENL

SEQ ID NO: 49 - protein, 277 aa, Homo sapiens
MENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETDSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYH

SEQ ID NO: 50 - protein, 714 aa, Homo sapiens
MSEEIITPVYCTGVSAQVQKQRARELGLGRHENAIKYLGQDYEQLRVRCLQSGTLFRDEAFPPVPQSLGYKDLGPNSSKTYGIKWKRPTELLSNPQFIVDGATRTDICQGALGDCWLLAAIASLTLNDTLLHRVVPHGQSFQNGYAGIFHFQLWQFGEWVDVVVDDLLPIKDGKLVFVHSAEGNEFWSALLEKAYAKVNGSYEALSGGSTSEGFEDFTGGVTEWYELRKAPSDLYQIILKALERGSLLGCSIDISSVLDMEAITFKKLVKGHAYSVTGAKQVNYRGQVVSLIRMRNPWGEVEWTGAWSDSSSEWNNVDPYERDQLRVKMEDGEFWMSFRDFMREFTRLEICNLTPDALKSRTIRKWNTTLYEGTWRRGSTAGGCRNYPATFWVNPQFKIRLDETDDPDDYGDRESGCSFVLALMQKHRRRERRFGRDMETIGFAVYEVPPELVGQPAVHLKRDFFLANASRARSEQFINLREVSTRFRLPPGEYVVVPSTFEPNKEGDFVLRFFSEKSAGTVELDDQIQANLPDEQVLSEEEIDENFKALFRQLAGEDMEISVKELRTILNRIISKHKDLRTKGFSLESCRSMVNLMDRDGNGKLGLVEFNILWNRIRNYLSIFRKFDLDKSGSMSAYEMRMAIESAGFKLNKKLYELIITRYSEPDLAVDFDNFVCCLVRLETMFRFFKTLDTDLDGVVTFDLFKWLQLTMFA

SEQ ID NO: 51 - protein, 268 aa, Homo sapiens
MFLVNSFLKGGGGGGGGGGGLGGGLGNVLGGLISGAGGGGGGGGGGGGGGGGGGGGTAMRILGGVISAISEAAAQYNPEPPPPRTHYSNIEANESEEVRQFRRLFAQLAGDDMEVSATELMNILNKVVTRHPDLKTDGFGIDTCRSMVAVMDSDTTGKLGFEEFKYLWNNIKRWQAIYKQFDTDRSGTICSSELPGAFEAAGFHLNEHLYNMIIRRYSDESGNMDFDNFISCLVRLDAMFRAFKSLDKDGTGQIQVNIQEWLQLTMYS

SEQ ID NO: 52 - protein, 391 aa, Homo sapiens
MSGPVPSRARVYTDVNTHRPREYWDYESHVVEWGNQDDYQLVRKLGRGKYSEVFEAINITNNEKVVVKILKPVKKKKIKREIKILENLRGGPNIITLADIVKDPVSRTPALVFEHVNNTDFKQLYQTLTDYDIRFYMYEILKALDYCHSMGIMHRDVKPHNVMIDHEHRKLRLIDWGLAEFYHPGQEYNVRVASRYFKGPELLVDYQMYDYSLDMWSLGCMLASMIFRKEPFFHGHDNYDQLVRIAKVLGTEDLYDYIDKYNIELDPRFNDILGRHSRKRWERFVHSENQHLVSPEALDFLDKLLRYDHQSRLTAREAMEHPYFYTVVKDQARMGSSSMPGGSTPVSSANMMSGISSVPTPSPLGPLAGSPVIAAANPLGMPVPAAAGAQQ

SEQ ID NO: 53 - protein, 350 aa, Homo sapiens
MPGPAAGSRARVYAEVNSLRSREYWDYEAHVPSWGNQDDYQLVRKLGRGKYSEVFEAINITNNERVVVKILKPVKKKKIKREVKILENLRGGTNIIKLIDTVKDPVSKTPALVFEYINNTDFKQLYQILTDFDIRFYMYELLKALDYCHSKGIMHRDVKPHNVMIDHQQKKLRLIDWGLAEFYHPAQEYNVRVASRYFKGPELLVDYQMYDYSLDMWSLGCMLASMIFRREPFFHGQDNYDQLVRIAKVLGTEELYGYLKKYHIDLDPHFNDILGQHSRKRWENFIHSENRHLVSPEALDLLDKLLRYDHQQRLTAKEAMEHPYFYPVVKEQSQPCADNAVLSSGLTAAR

SEQ ID NO: 54 - protein, 215 aa, Homo sapiens
MSSSEEVSWISWFCGLRGNEFFCEVDEDYIQDKFNLTGLNEQVPHYRQALDMILDLEPDEELEDNPNQSDLIEQAAEMLYGLIHARYILTNRGIAQMLEKYQQGDFGYCPRVYCENQPMLPIGLSDIPGEAMVKLYCPKCMDVYTPKSSRHHHTDGAYFGTGFPHMLFMVHPEYRPKRPANQFVPRLYGFKIHPMAYQLQLQAASNFKSPVKTIR

SEQ ID NO: 55 - protein, 309 aa, Homo sapiens
MDEKVFTKELDQWIEQLNECKQLSESQVKSLCEKAKEILTKESNVQEVRCPVTVCGDVHGQFHDLMELFRIGGKSPDTNYLFMGDYVDRGYYSVETVTLLVALKVRYRERITILRGNHESRQITQVYGFYDECLRKYGNANVWKYFTDLFDYLPLTALVDGQIFCLHGGLSPSIDTLDHIRALDRLQEVPHEGPMCDLLWSDPDDRGGWGISPRGAGYTFGQDISETFNHANGLTLVSRAHQLVMEGYNWCHDRNVVTIFSAPNYCYRCGNQAAIMELDDTLKYSFLQFDPAPRRGEPHVTRRTPDYFL

SEQ ID NO: 56 - protein, 447 aa, Homo sapiens
MAGAGGGNDIQWCFSQVKGAVDDDVAEADIISTVEFNHSGELLATGDKGGRVVIFQQEQENKIQSHSRGEYNVYSTFQSHEPEFDYLKSLEIEEKINKIRWLPQKNAAQFLLSTNDKTIKLWKISERDKRPEGYNLKEEDGRYRDPTTVTTLRVPVFRPMDLMVEASPRRIFANAHTYHINSISINSDYETYLSADDLRINLWHLEITDRSFNIVDIKPANMEELTEVITAAEFHPNSCNTFVYSSSKGTIRLCDMRASALCDRHSKLFEEPEDPSNRSFFSEIISSISDVKFSHSGRYMMTRDYLSVKIWDLNMENRPVETYQVHEYLRSKLCSLYENDCIFDKFECCWNGSDSVVMTGSYNNFFRMFDRNTKRDITLEASRENNKPRTVLKPRKVCASGKRKKDEISVDSLDFNKKILHTAWHPKENIIAVATTNNLYIFQDKVN

SEQ ID NO: 57 - protein, 589 aa, Homo sapiens
MAAADGDDSLYPIAVLIDELRNEDVQLRLNSIKKLSTIALALGVERTRSELLPFLTDTIYDEDEVLLALAEQLGTFTTLVGGPEYVHCLLPPLESLATVEETVVRDKAVESLRAISHEHSPSDLEAHFVPLVKRLAGGDWFTSRTSACGLFSVCYPRVSSAVKAELRQYFRNLCSDDTPMVRRAAASKLGEFAKVLELDNVKSEIIPMFSNLASDEQDSVRLLAVEACVNIAQLLPQEDLEALVMPTLRQAAEDKSWRVRYMVADKFTELQKAVGPEITKTDLVPAFQNLMKDCEAEVRAAASHKVKEFCENLSADCRENVIMSQILPCIKELVSDANQHVKSALASVIMGLSPILGKDNTIEHLLPLFLAQLKDECPEVRLNIISNLDCVNEVIGIRQLSQSLLPAIVELAEDAKWRVRLAIIEYMPLLAGQLGVEFFDEKLNSLCMAWLVDHVYAIREAATSNLKKLVEKFGKEWAHATIIPKVLAMSGDPNYLHRMTTLFCINVLSEVCGQDITTKHMLPTVLRMAGDPVANVRFNVAKSLQKIGPILDNSTLQSEVKPILEKLTQDQDVDVKYFAQEALTVLSLA

SEQ ID NO: 58 - DNA, 1987 bp, Homo sapiens
ggaggggagagaaagagcgagagaaggggaaagacaagtcgggagaggccggtaggcgtgaggcgggcctgaagcggcagcgggcggccttcgtccggcgagagctaggccgaggacccgcgccgcgctccccggcacctcaccgcgtccttcaccgactcccgcggcgcgcggccgggcggggaagggcgggcgggggtctcctccaggctgcgcgctcggagccgcctgctgggcttgggcggggcgcggggcccgcggccgccctacccggctcagtcctccccctgtgggacctggcgacggcggcggagggagaggggagcggcgcccgggccggggccgggggcgggtggggaggggggagggcggcggccgggctggggctcgggatccgcatcgggatcgggccgccatggacgacaaggcgttcaccaaggagctggaccagtgggtcgagcagctgaacgagtgtaagcagctgaacgagaaccaagtgcggacgctgtgcgagaaggcaaaggaaattttaacaaaagaatcaaatgtgcaagaggttcgttgccctgttactgtctgtggagatgtgcatggtcaatttcatgatcttatggaactctttagaattggtggaaaatcaccggatacaaactacttattcatgggtgactatgtagacagaggatattattcagtggagactgtgactcttcttgtagcattaaaggtgcgttatccagaacgcattacaatattgagaggaaatcacgaaagccgacaaattacccaagtatatggcttttatgatgaatgtctgcgaaagtatgggaatgccaacgtttggaaatattttacagatctctttgattatcttccacttacagctttagtagatggacagatattctgcctccatggtggcctctctccatccatagacacactggatcatataagagccctggatcgtttacaggaagttccacatgagggcccaatgtgtgatctgttatggtcagatccagatgatcgtggtggatggggtatttcaccacgtggtgctggctacacatttggacaagacatttctgaaacctttaaccatgccaatggtctcacactggtttctcgtgcccaccagcttgtaatggagggatacaattggtgtcatgatcggaatgtggttaccattttcagtgcacccaattactgttatcgttgtgggaaccaggctgctatcatggaattagatgacactttaaaatattccttccttcaatttgacccagcgcctcgtcgtggtgagcctcatgttacacggcgcaccccagactacttcctataaatttctcctgggaaacctgcctttgtatgtggaagtatacctggctttttaaaatatatgtatttaaaaacaaaaagcaacagtaatctatgtgtttctgtaacaaattgggatctgtcttggcattaaaccacatcatggaccaaatgtgccatactaatgatgagcatttagcacaatttgagactgaaatttagtacactatgttctaggtcagtctaacagtttgcctgctgtatttatagtaaccattttcctttggactgttcaagcaaaaaaggtaactaactgcttcatctccttttgcgcttatttggaaattttagttatagtgtttaactggcatggattaatagagttggagttttatttttaagaaaaattcacaagctaacttccactaatccattatcctttattttattgaaatgtataattaacttaactgaagaaaaggttcttcttgggagtatgttgtcataacatttaaagagatttcccttcatttaaactaaattactgttttatgttgatctgcatatttctgtatatttgtcatgacagtgcttgcatcctatttggtgtactcagcaaataaacttttcattttaaacaaaaacattcaaaaaaaaaaaaaaaaaa

SEQ ID NO: 59 - protein, 309 aa, Homo sapiens
MDDKAFTKELDQWVEQLNECKQLNENQVRTLCEKAKEILTKESNVQEVRCPVTVCGDVHGQFHDLMELFRIGGKSPDTNYLFMGDYVDRGYYSVETVTLLVALKVRYPERITILRGNHESRQITQVYGFYDECLRKYGNANVWKYFTDLFDYLPLTALVDGQIFCLHGGLSPSIDTLDHIRALDRLQEVPHEGPMCDLLWSDPDDRGGWGISPRGAGYTFGQDISETFNHANGLTLVSRAHQLVMEGYNWCHDRNVVTIFSAPNYCYRCGNQAAIMELDDTLKYSFLQFDPAPRRGEPHVTRRTPDYFL

SEQ ID NO: 60 - protein, 11 aa, Artificial Sequence
  L803 (Tocris Bioscience) phosphopeptide derived from GSK-3 recognition motif
KEAPPAPPQSP

(19) United States
(12) Patent Application Publication
(10) Pub. No.: US 2012/0156180 A1
(43) Pub. Date: Jun. 21, 2012

(54) COMPOSITIONS AND METHODS FOR THE TREATMENT OF KRABBE AND OTHER NEURODEGENERATIVE DISEASES

(75) Inventor: Ernesto Bongarzone, Chicago, IL (US)

(73) Assignee: The Board of Trustees of the University of Illinois, Urbana, IL (US)

(21) Appl. No.: 13/390,073

(22) PCT Filed: Aug. 10, 2010
(86) PCT No.: PCT/US2010/045087

§ 371 Date: Feb. 10, 2012

Related U.S. Application Data
(60) Provisional application No. 61/232,607, filed on Aug. 10, 2009.
(60) Provisional application No. 61/294,607, filed on Jan. 13, 2010.

(51) Int. Cl. A61K 48/00; C07K 14/145; C07D 211/32; A61P 25/28; A61K 38/08; A61K 31/445; A61K 35/12; C07H 21/02; C07K 7/06

(52) U.S. Cl. 424/93.7; 536/24.5; 530/322; 546/233; 530/327; 514/17.7; 514/44 R; 514/331

(57) ABSTRACT

Provided are compositions and methods for the treatment of Krabbe and other neurodegenerative diseases, including storage diseases such as GM1 gangliosidosis, Niemann-Pick disease, Tay-Sachs disease, Sandhoff disease, metachromatic leukodystrophy, Canavan disease, Pelizaeus-Merzbacher disease, and storage conditions facilitated by aging of lysosomal functions, which are associated with psychosine (and/or other storage material)-mediated axonal degeneration. Compositions and methods employ (1) one or more inhibitor of a phos-photransferase activity of one or more kinase(s) such as, for example, CDK5, P38, jnk, src, CK2, PKC, GSK3α and β; (2) one or more inhibitor of a phosphotransferase activity of one or more phosphatase(s) such as, for example, the Ser/Thr protein phosphatase PP1 and Tyr protein phosphatase PP2; one or more inhibitor of a caspase/calpain activity of one or more caspases such as caspase 3 and calpains such as calpain 1 and 2; and (4) one or more inhibitor of a sodium/calcium exchange protein such as, for example, NCX1. Inhibitors include small molecules, including the GSK3β inhibitor L803 and the NCX1 inhibitor flecainide, and siRNA molecules that downmodulate cellular levels of one or more mRNA, including siRNA that are capable of downmodulating the cellular expression of PP1. Inhibitors disclosed can cross the blood-brain barrier and, thus, are available to the central nervous system (CNS) and effective in reducing psychosine-mediated axonal degeneration.

### CROSS REFERENCE TO RELATED APPLICATIONS

This application claims the benefit of U.S. Provisional Patent Application No. 61/232,607, filed Aug. 10, 2009, and U.S. Provisional Patent Application No. 61/294,607, filed Jan. 13, 2010, the entire disclosures of these provisional patent applications are hereby incorporated by reference in their entirety.

### GOVERNMENT SPONSORED RESEARCH OR DEVELOPMENT

This invention was made with government support under 1R01NS065808-01A1 awarded by the National Institutes of Health. The government has certain rights in the invention.

### SEQUENCE LISTING

The present application includes a Sequence Listing in electronic format as a text file entitled “Sequence_Listing_{—}10Aug2010.txt” which was created on Aug. 10, 2010, and which has a size of 261 bytes. The contents of txt file “Sequence_Listing_{—}10Aug2010.txt” are incorporated by reference herein.

### BACKGROUND OF THE DISCLOSURE

1. Technical Field

The present disclosure is directed, generally, to the treatment of Krabbe and other neurodegenerative diseases, including storage diseases such as GM1 gangliosidosis, Niemann-Pick disease, Tay-Sachs disease, Sandhoff disease, metachromatic leukodystrophy, Canavan disease, Pelizaeus-Merzbacher disease, and storage conditions facilitated by aging of lysosomal functions, which are associated with psychosine (and/or other storage material)-mediated axonal degeneration. More specifically, provided herein are compositions and methods for the treatment of neurodegenerative diseases that comprise (1) one or more inhibitor(s) of a phosphotransferase activity of one or more kinase(s) such as, for example, CDK5, P38, jnk src, CK2, PKC, GSK3α, and GSK3β; (2) one or more inhibitor(s) of a phosphotransferase activity of a phosphatase such as, for example, phosphatases such as the Ser/Thr protein phosphatase PP1 and tyrosine protein phosphatases PP2; and/or (3) one or more inhibitor(s) of a sodium/calcium exchange protein such as, for example, NCX1. Inhibitors include small molecules, as exemplified herein by the NCX1 inhibitor flecainide; peptides, as exemplified herein by the GSK3β inhibitor L803; and siRNA molecules that downmodulate cellular levels of one or more mRNA, as exemplified herein by siRNA that are capable of downmodulating the cellular expression of PP1. Each of the inhibitors provided herein, when administered to a patient having a neurodegenerative disease such as Krabbe disease and involving abnormal activities of PP1, CDK5, GSK3β, and/or PKC is capable of reducing the extent of psychosine-mediated axonal degeneration. To achieve therapeutic benefit, the inhibitors presented herein are capable of crossing the blood-brain barrier such that they are available to the central nervous system (CNS) and, consequently, are effective in the treatment of a wide variety of neurodegenerative diseases, including neuropathies, which are associated with elevated psychosine levels, in particular such pediatric leukodystrophies as Krabbe disease.

2. Description of the Related Art

Krabbe disease (KD) is an autosomal recessive neurodegenerative disease that is caused by the toxic accumulation of galactosylsphingosine (psychosine) in the myelin-forming cells of the nervous system. A deficiency of the lysosomal enzyme galactosylceramidase (GALC, E.C. 3.2.1.46; an enzyme that hydrolyzes galactosylceramide (GalCer), psychosine, monogalactosylceramide, and lactosylcerannide) leads to the accumulation of psychosine in myelin-forming cells, which causes demyelination of the brain and nerves in affected individuals. Wenger et al., In “The Metabolic and Molecular Bases of Inherited Disease” (Scriver et al. (eds), McGraw-Hill: New York, 3669, 3670, 3687 (2001)); Aicardi, J. Inherit. Metab. Dis. 16:733-743 (1993); Igisu and Suzuki, Science 224:753-755 (1984); Suzuki, Neurochem. Res. 23:251-259 (1998); Wenger et al., Mol. Genet. Metab. 70:1-9 (2000); and Suzuki and Suzuki, Neurochem. Pathol. 3:53-68 (1985). Accumulation of undigested psychosine in oligodendrocytes and Schwann cells is believed to cause the death of myelinating glia and demyelination throughout the white matter. As disease progresses, oligodendrocytes die due to the toxic accumulation of psychosine.

KD is not the only example of a disease where undigested substrates become progressively toxic. There are more than 60 different forms of lysosomal storage diseases and most are affected with neurological impairments. In most cases, the mechanisms that mediate neuronal and axonal damage are unknown. Particularly, metachromatic leukodystrophy, GM1 gangliosidosis, Niemann-Pick, Sandoff and Tay-Sachs diseases are all caused by the toxic accumulation of specific lipids in the brain and affected of severe neurological deficits, fitting the model of axonal transport deficiency. Of further relevance, neuropathatic defects seen in elderly remain mostly uncharacterized. Aging is a process that may diminish the functionality of the lysosomal comparment, causing abnormal—albeit at low levels—digestion of various cellular components. Progressive accumulation of small amounts of undigested compounds may gestate the conditions for axonal and neuronal defects late in life.

KD patients are also affected with astrogliosis and the formation of multinuclear globoid cells derived from infiltrating monocyte-macrophages. Igisu and Suzuki, Science 224:753-755 (1984) and Suzuki, Neurochem. Res. 23:251-259 (1998). The disappearance of myelinating cells induces further myelin breakdown, stalling myelin production and leading to further infiltration of macrophages. During the early stages of disease, the local resident microglia (i.e. the CNS macrophages) phagocytize myelin debris. The infiltration of blood-derived hematogenous cells appears to reflect the need for additional phagocytic activity, which resident microglia can no longer adequately provide.

Activated microglia and astrocytes secrete numerous signalling molecules such as the proinflammatory cytokines IL-6, TNF-α, and monocyte chemoattractive protein (MCP-1). Wu et al., J. Neuropathol. Exp. Neurol. 59:628-639 (2000) and LeVine and Brown, J. Neuroimmunol. 73:47-56 (1997). In particular, MCP-1 regulates the transendothelial migration of monocytes into the brain and appears to play a fundamental role in attracting and promoting waves of infiltrating monocytic cells, which worsen the myelin microenvironment.

A large array of genetic mutations affects the metabolism of myelin components in pediatric leukodystrophies. Boespflug-Tanguy et al., Curr. Neurol. Neurosci. Rep. 8:217-229 (2008) and Costello et al., Neurologist 15:319-328 (2009). In light of this, the majority of attention has been put on the mechanisms of demyelination in these diseases, leaving a significant void regarding the contribution of neuronal stress to their neurological phenotypes. Dickerman et al., J. Neurol. Sci. 50:181-190 (1981); Ida et al., Mol. Chem. Neuropathol. 13:195-204 (1990); Igisu and Suzuki, Science 224:753-755 (1984); Jatana et al., Neurosci. Lett. 330:183-187 (2002); Nagara et al., Brain Res. 391:79-84 (1986); Suzuki, Neurochem. Res. 23:251-259 (1998); and Tanaka and Webster, J. Neuropathol. Exp. Neurol. 52:490-498 (1993).

GALC deficiency affects globally both neural and non-neural cells, posing a formidable challenge to efficiently delivering sufficient and timely amounts of GALC before irreversible degeneration occurs. To reduce demyelination, current therapies for Krabbe disease, such as hematogenous replacement through bone marrow transplantation (BMT), seek to provide the missing GALC enzyme to myelinating glia via infiltrating macrophages that are present in bone marrow cells transplanted from a healthy donor into an affected patient. The replacement of the bone marrow in KD with that from healthy donors provides the recipient with a constant and self-renewable source of monocytic cells able to replenish the pool of microglia in the nervous system and, consequently, to infiltrate with cells that produce GALC in situ. Eglitis and Mezey, Proc. Natl. Acad. Sci. USA 94:4080-4085 (1997) and Krivit et al., Cell Transplant 4:385-392 (1995). To date, hematopoietic replacement constitutes the only available therapy to reduce disease severity in some clinical cases of KD. Krivit et al., Curr. Opin. Neurol. 12:167-176 (1999).

Transplantation of human cord blood cells in presymptomatic Krabbe infants has proven useful in limiting disease progression but does not appear to completely cure the disease since treated babies develop neurological sequelae. Escolar et al., N. Engl. J. Med. 352:2069-2081 (2005). In experiments using the Twitcher mouse, a model of KD that includes a mutation in the gene encoding the GALC, hematopoietic replacement by BMT increases the life span of mutant mice by up to 150 days. While BMT-treated mice have improved myelination and ameliorated motor defects (Yeager et al., Science 225:1052-1054 (1984)), they invariably die with severe neurological deficits. Bambach et al., Bone Marrow Transplant 19:399-402 (1997). Thus, notwithstanding the benefits attributable to the use of BMT, KD patients continue to suffer from ongoing axonopathy and neurological deterioration. This suggests that the pathogenic mechanisms in KD are more complex than previously thought and that new therapeutic strategies are needed to further reduce the severity of and, ultimately, to achieve a cure for KD.

One interpretation for the limited therapeutic efficacy of BMT rests in the dynamics of accumulation of donor-derived enzyme in the nervous system. In KD, disease progresses by first activating local microglia in the central nervous system (CNS) and by later stimulating the recruitment of macrophages from the blood stream, which become globoid cells. Kobayashi et al., Brain Res. 352:49-54 (1985). None of these cellular responses are instantaneous, however. In fact, 1-2 months are needed to turn over about one third of the residing microglia. Thus, even when BMT is performed very early after birth, a significant amount of time elapses before donor-derived macrophages reach the CNS and contribute significantly with corrective GALC enzyme. Using the Twitcher mouse model, Wu et al. detected donor-derived cells in the central white matter about 1-2 months after BMT. Am. J. Pathol. 156:1849-1854 (2000). Consequently, the slow rate of entry of donor-derived cells and the delayed correction of the metabolic defect might account for a failure to prevent some neurodegenerative processes.

The role of neuronal loss in Krabbe disease is not well understood, but a consensus is emerging that dysfunction of axons and neurons leads to permanent neurological deficits in several neurodegenerative disorders, including multiple sclerosis, Alzheimer disease, Parkinson disease, and others. Preliminary studies provide evidence that Krabbe disease is also compounded by axonal defects. Thus, in addition to the loss of myelin, neurodegeneration is likely a limiting factor in reducing the efficiency of traditional therapies.

Lysosomal enzymes, such as GALC, have the common property of following the secretory vesicular pathway. Secretion to the extracellular milieu appears to play a fundamental role in correcting lysosomal deficiencies. A normal cell secretes the corrective enzyme, which can then be taken up by enzyme-deficient cells. This physiological process, called cross-correction, can occur by cell surface mannose-6-phosphate receptor-mediated endocytosis and also by direct cell-to-cell transfer. Marzella and Glaumann, Int. Rev. Exp. Pathol. 25:239-278 (1983); Jourdian, Prog. Clin. Biol. Res. 97:85-93 (1982); and Sly et al., Methods Cell Biol. 23:191-214 (1981). Existing strategies for treating lysosomal storage diseases are based on cross-correction, which can be initiated after enzyme delivery by transduction with viral vectors (Lin et al., Mol. Ther. 12:422-430 (2005); Meng et al., Mol. Genet. Metab. 84:332-343 (2005); and Dolcetta et al., J. Gene Med. 8:962-971 (2006)), enzyme supplementation (Kobayashi and Suzuki, J. Biol. Chem. 256:1133-1137 (1981)), and cell replacement such as through BMT (Malatack et al., Pediatr. Neurol. 29:391-403 (2003) and Pastores and Barnett, Expert Opin. Emerg. Drugs 10:891-902 (2005)).

The delay in metabolic correction in the weeks following BMT, when the nervous system of KD patients is exposed to very low, if any, therapeutic GALC enzyme levels, leaves psychosine accumulation and degenerative processes essentially untreated. Once enzyme cross-correction begins, quiescent oligodendrocyte progenitors in the CNS might be engaged for re-myelination. Nait-Oumesmar, Eur. J. Neurosci. 11:4357-4366 (1999). Even with the benefit of enzyme cross-correction, however, neurological sequelae (motor deficits) arise and handicap Krabbe patients permanently. Thus, even though myelin degeneration is the hallmark in the pathology of KD, the presence of different degrees of neurodegeneration, including axonal degeneration with selective loss of large-diameter axons, suggests that some neural pathways are damaged or rendered dysfunctional during the time when insufficient enzyme is available. Sourander and Olsson, Acta Neuropathol. 11:69-81 (1968); Jacobs et al., J. Neurol. Sci. 55:285-304 (1982); Schlaepfer and Prensky, Acta Neuropathol. 20:55-66 (1972); Kurtz and Fletcher, Acta Neuropathol. 16:226-232 (1970); Duchen et al., Brain 103:695-710 (1980); Sakai et al., J. Neurochem. 66:1118-1124 (1996); Kobayashi et al., Brain Res. 202:479-483 (1980); Galbiati et al., J. Neurosci. 27:13730-13738 (2007); Nagara et al., Brain Res. 244:289-294 (1982); Taniike et al., J. Neuropathol. Exp. Neurol. 58:644-653 (1999); Ohno et al., Brain Res. 602:268-274 (1993); and Matsushima et al., Cell 78:645-656 (1994).

The accumulation of a neurotoxin such as psychosine could affect neuronal functions at various levels. A few reports of selective absence of large-diameter axons in KD raise the possibility that axonal stability is compromised in this disease. The axon is a very vulnerable structure of the neuron. Most neurons extend a single long axon that mediates communication between the neuronal body and an effector cell. Because the axon lacks genetic material and the protein synthesis machinery to produce its protein components, neurons have developed mechanisms to transport lipids, proteins, and vesicles from the perikaryon to the terminal end of the axon. Hirokawa and Takemura, Curr. Opin. Neurobiol. 14:564-573 (2004). This refined mechanism of axonal transport is tightly regulated by phosphotransferase activity of kinases (e.g., CDK5, GSK3β, and PKC) and phosphatases (e.g., Ser/Thr protein phosphatase PP1) (Morfini et al., Embo J. 23:2235-2245 (2004); Morfini et al., Proc. Natl. Acad. Sci. USA 104:2442-2447 (2007); Hooper et al., J. Neurochem. 104:1433-1439 (2008)), which provide adequate levels of phospho-modifications to molecular motors (kinesins and dyneins) and other cytoskeletal proteins (Brady et al., Proc. Natl. Acad. Sci. USA 87:1061-1065 (1990); Hirokawa et al., J. Cell Biol. 114:295-302 (1991)).

The dependence on phosphotransferase activities renders axonal transport highly vulnerable to pathological conditions that affect the activities of those enzymes. Lee and Hollenbeck, J. Biol. Chem. 270:5600-5605 (1995) and Morfini et al., Neuromolecular Med. 2:89-99 (2002). For example, CDK5 regulates GSK3β-phosphorylation of kinesin, releasing cargoes from motors, in particular, neuronal domains. Morfini et al., Neuromolecular Med. 2:89-99 (2002). Alterations in the CDK5-GSK3β pathways can block axonal transport, leading to axonal dysfunction and degeneration. Morfini et al., Methods Mol. Biol. 392:51-69 (2007); Pigino et al., J. Neurosci. 23:4499-4508 (2003); and Lazarov et al., J. Neurosci. 25:2386-2395 (2005).

Axonal dysfunction might precede the death of the neuronal body by long periods of time (several months or even years in humans). This process seems to start at the synaptic end of the axons, where structural and functional defects begin to impact synaptic efficiency. Axons that have been “primed” by a “degenerative stimulus” (e.g., injury, toxins, and inflammation) can then “die back” very slowly towards the body of the neuron. Coleman and Perry, Trends Neurosci. 25:532-537 (2002). Thus, any given neuron may be anatomically intact while its axon is already dysfunctional and slowly dying back.

While the effects of psychosine on myelinating glia have been described, the molecular mechanism of psychosine pathogenesis mediated in axonal/neuronal degeneration in KD remains unknown. Psychosine rapidly accumulates up to 100-fold in white matter of KD (Ida et al., Mol. Chem. Neuropathol. 13:195-204 (1990) and Svennerholm et al., J. Lipid Res. 21:53-64 (1980)) and is toxic to a wide variety of cell types (Komiyama and Suzuki, Brain Res. 637:106-113 (1994) and Dickerman et al., J. Neurol. Sci. 50:181-190 (1981)). Some of the known downstream effects of psychosine include altered mitochondrial activity and induction of caspase-mediated apoptotic cell death. Strasberg, Biochem. Cell Biol. 64:485-489(1986); Tapasi et al., Indian J. Biochem. Biophys. 35:161-165 (1998); Jatana et al., Neurosci. Lett. 330:183-187 (2002); Zaka and Wenger, Neurosci. Lett. 358:205-209 (2004); and Haq et al., J. Neurochem. 86:1428-1440 (2003).

The relevance of neurodegeneration to classical demyelinating disorders such as KD and other leukodystrophies is starting to be appreciated. This may be highlighted by the intimate interaction between axons and myelin sheaths. For example, the formation of a functional node of Ranvier not only depends on the coordinated synthesis, apposition and compaction of internodal myelin sheaths (Simons and Trajkovic, J. Cell Sci. 119:4381-4389 (2006) and Susuki and Rasband, Curr. Opin. Cell Biol. 20:616-623 (2008)), but also on the transport of nodal ion channels and accessory proteins by the axon (de Waegh et al., Cell 68:451-463 (1992)). The transport of these components from the soma to the cellular process is a fundamental mechanism ensuring that proteins and lipids are found in the appropriate microdomain of the cell in a coordinated manner. Since more than 99% of axonal proteins are produced in the neuronal soma and delivered by axonal transport, neurons are likely the best example of dependence on cellular transport mechanisms being vital for survival and function. De Vos et al., Annu. Rev. Neurosci. 31:151-173 (2008); Hafezparast et al., Science 300:808-812 (2003); Puls et al., Nat. Genet. 33:455-456 (2003); Reid et al., Am. J. Hum. Genet. 71:1189-1194 (2002); and Zhao et al., Cell 105:587-597 (2001).

Fast axonal transport (FAT) is used for the rapid translocation of cargoes to and from the axonal terminus. Brady and Sperry, Curr. Opin. Neurobiol. 5:551-558 (1995); Hirokawa, Science 279:519-526 (1998); and Hirokawa et al., J. Cell Biol. 114:295-302 (1991). Because neurons are highly dependent on this process, it is believed that defects in FAT may contribute to neurodegeneration. De Vos et al., Annu. Rev. Neurosci. 31:151-173 (2008); Lazarov et al., J. Neurosci. 27:7011-7020 (2007); Morfini et al., Nat. Neurosci. 9:907-916 (2006); Pigino et al., J. Neurosci. 23:4499-4508 (2003); and Szebenyi et al., Neuron 40:41-52 (2003). Moreover, mutations in the molecular motors kinesin and dynein, which regulate antero and retrograde FAT, respectively, cause specific forms of axonal degeneration. Brady, Trends Cell Biol. 5:159-164 (1995); Hirokawa et al., J. Cell Biol. 114:295-302 (1991); Hafezparast et al., Science 300:808-812 (2003); Puls et al., Nat. Genet. 33:455-456 (2003); Reid et al., Am. J. Hum. Genet. 71:1189-1194 (2002); and Zhao et al., Cell 105:587-597 (2001). One major example of this is the progressive dying-back neuropathology, where stress and damage of axons largely precedes neuronal death. Coleman and Perry, Trends Neurosci. 25:532-537 (2002)). It is, however, unknown whether FAT is affected in leukodystrophies such as KD. FAT in KD has been investigated using the Twitcher mouse. Cantuti & Bongarzone, In review. This work demonstrates that FAT is defective in this myelin mutant and contributes to the establishment of a dying-back type of neuronal damage.

It was recently found that psychosine preferentially accumulates in lipid rafts in the nervous system of Twitcher mice and KD patients (White et al., J. Neurosci. 29(19):6068-6077 (2009)), suggesting that psychosine accumulation in these membrane microdomains exerts architectural and functional changes in rafts, modifying raft-associated signaling. Mounting evidence suggests that rafts are particularly important during axon formation, pre-synaptic assembly, and targeting of ion channels to the axolemma, serving as mobile structural scaffolding platforms to assemble membranous components in the axon. Ahmari et al., Nat. Neurosci. 3:445-451 (2000); Lai and Jan, Nat. Rev. Neurosci. 7:548-562 (2006); Ziv and Garner, Nat. Rev. Neurosci. 5:385-399 (2004); and Bresler et al., J. Neurosci. 24:1507-1520 (2004).

In view of this evidence and because (1) GALC-deficiency increases endogenous storage of psychosine in neurons, (2) psychosine preferentially accumulates in lipid rafts, and (3) defective axonal transport and axonal injury are simultaneous in the Twitcher mouse, it is believed that psychosine accumulation leads to the inhibition of axonal transport. Psychosine can produce a progressive and sustainable blockage to both antero and retrograde modes of axonal transport, further underscoring its toxicity. Overall, psychosine accumulation in KD appears to have at least two effects: (1) triggering the death of myelinating glia and demyelination and (2) blocking axonal transport in neurons, setting the stage for axonal degeneration and neuronal dysfunction.

Establishing the conditions to prevent axonal degeneration in KD (and hence, to ameliorate neurological sequelae) requires the identification of molecular targets for preventive and protective therapy. Unfortunately, previous studies have failed to identify the downstream effectors in psychosine-mediated axonal degeneration. Moreover, those effectors involved in glial degeneration do not appear to exert the same fundamental roles in axonal transport and/or axonal dynamics. Strasberg, Biochem. Cell Biol. 64:485-489 (1986); Tapasi et al., Indian J. Biochem. Biophys. 35:161-165 (1998); Jatana et al., Neurosci. Lett. 330:183-187 (2002); Zaka and Wenger (2004) Neurosci. Lett. 358:205-209 (2002); and Haq et al., J. Neurochem. 86:1428-1440 (2003).

Despite the benefits of bone marrow transplantation in the treatment of Krabbe disease as well as other related neurodegenerative diseases, the delayed CNS response to donor-derived macrophages, which results in a delayed contribution of the corrective enzyme GALC, compromises the ultimate therapeutic efficacy of this treatment regimen as a result of the accumulation of psychosine in axons and the corresponding irreversible psychosine-mediated axonal degeneration. What is critically needed in the art are compositions and methods for the treatment of neurodegenerative diseases, such as Krabbe disease, which, when employed alone or in combination with existing BMT regimens, enhance axonal stability by blocking or substantially reducing psychosine-induced axonopathy.

### SUMMARY OF THE DISCLOSURE

The present disclosure achieves these and other related needs by providing compositions and methods for the treatment of Krabbe and other neurodegenerative diseases, including metachromatic leukodystrophy, GM1 gangliosidosis, Niemann-Pick disease, Sandhoff disease and Tay-Sachs disease as well as neurodegeneration in aging, which compositions and methods employ one or more inhibitor(s) of one or more downstream effector(s) of psychosine-mediated axonal degeneration. The inhibitors presented herein are capable of accessing the central nervous system (CNS) via the blood-brain barrier (BBB) and, hence, are effective in reducing psychosine-induced axonopathy. These inhibitors may, optionally, be employed in conjunction with existing bone marrow transplantation (BMT) regimens for the treatment of Krabbe and other neurodegenerative diseases. By administering an inhibitor of a downstream effector of psychosine-mediated axonal degeneration, the toxicity of psychosine is reduced or eliminated in an acute manner. This pharmacological intervention allows sufficient time for the accumulation of infiltrating bone marrow-derived GALC-expressing cells, such as GALC-expressing macrophages, which ultimately reverse psychosine-mediated toxicity through the conversion of psychosine to a non-toxic reaction product.

Thus, it was found, as part of the present disclosure, that compounds that are capable of downregulating the expression and/or antagonizing the activity of a broad range of effector molecules are effective in reducing the axonal degeneration resulting from psychosine accumulation.

Within certain embodiments, the present disclosure provides inhibitory nucleic acids, including siRNA molecules, and small-molecule and peptide antagonists of kinases such as CDK5, P38, jnk src, caspase 3, calpains, CK2, PKC, GSK3α, and GSK3β; phosphatases such as the Ser/Thr protein phosphatase PP1 and tyrosine protein phosphatases PP2; and sodium/calcium exchange proteins such as NCX1, each of which is effective in reducing psychosine-mediated neurotoxicity, in particular psychosine-mediated axonopathy.

Within certain aspects of these embodiments are provided siRNA molecules that are targeted against, and lead to the downregulation of, mRNA that encode an effector of psychosine-mediated axonal degeneration. For example, provided are siRNA that are targeted against mRNA that encode PP1. siRNA of the present disclosure comprise an antisense strand of between 15 nucleotides and 50 nucleotides, or between 18 and 40 nucleotides, or between 20 and 35 nucleotides, or between 21 and 30 nucleotides, which is capable of specifically binding to a target mRNA encoding a psychosine effector selected from CDK5, P38, jnk, src, caspase 3, calpains, CK2, PKC, GSK3α, GSK3β, PP1, PP2, and NCX1.

Exemplified herein are siRNA that bind to the α- and β-isoforms of the Ser/Thr protein phosphatase PP1 and that comprise between 15 and 50 nucleotides of an antisense sequence that is capable of specifically binding to an α- or β-isoform of PP1 mRNA encoded by the cDNA presented in SEQ ID NO: 13 (murine PP1, α-isoform), SEQ ID NO: 12 (human PP1, α-isoform), SEQ ID NO: 15 (murine PP1, β-isoform); and/or SEQ ID NO: 14 (human PP1, β-isoform). Within certain aspects, the siRNA may between 15 and 50 contiguous nucleotides of the following sequences: (a) 5′-CCAGAUCGUU UGUACAGAAA UCUCGAGAUU UCUGUACAAA CGAUCUGG-3′ (SEQ ID NO: 7), which binds to the mRNA encoding the catalytic subunit of mouse protein phosphatase 1, alpha isoform (NM_{—}031868, FIG. 29, SEQ ID NO: 13); (b) 5′-UUUGAUGUUG UAGCGUCUCt t-3′ (SEQ ID NO: 29), which binds to the mRNA encoding the catalytic subunit of human protein phosphatase 1, alpha isoform (NM_{—}206873.1, FIG. 28, SEQ ID NO: 12); (c) 5′-GGCGUCCUUG AAAGUGUUAA AUCUCGAGAU UUAACACUUU CAAGGACGC-3′ (SEQ ID NO: 9), which binds to the mRNA encoding the catalytic subunit of mouse protein phosphatase 1, beta isoform (NM_{—}172707; SEQ ID NO: 15); and (d) 5′-UAAAACUCUA GGUGUAUACt t-3′ (SEQ ID NO: 32), which binds to the mRNA encoding the catalytic subunit of human protein phosphatase 1, beta isoform (NM_{—}002709.2; SEQ ID NO: 14). Within certain aspects, siRNA of the present disclosure may include one or more modification to confer in vivo stability such as, for example, a “tt” 3′-overhang as is exemplified in the human PP1 antisense siRNA sequences presented in SEQ ID NOs: 28 and 29.

Within other aspects are provided siRNA that bind to mRNA that encode CDK5, P38, jnk, src, caspase 3, calpains, CK2, PKC, GSK3α and β, PP2; and NCX1 and that comprise between 15 and 50, or between 18 and 40, or between 20 and 35, or between 21 and 30 consecutive nucleotides of the antisense sequence of SEQ ID NO: 16 (NM_{—}004935; CDK5); SEQ ID NO: 17 (NM_{—}001146156.1; GSK3β); SEQ ID NO: 18 (NM_{—}002737.2; PKC); SEQ ID NO: 19 (NM_{—}006153.4; NCK1);); SEQ ID NO: 34 (NM_{—}002745.4; p38); SEQ ID NO: 35 (NM_{—}002750.2; INK); SEQ ID NO: 36 (NM_{—}005417.3; SRC); SEQ ID NO: 37 (NM_{—}004346.3; caspase 3); SEQ ID NO: 38 (NM_{—}005186.2; calpain 1, large subunit); SEQ ID NO: 39 (NM_{—}001749.2; calpain, small subunit); SEQ ID NO: 40 (NM_{—}177559.2; CK2, alpha subunit); SEQ ID NO: 41 (NM_{—}001896.2; CK2, alpha prime subunit); SEQ ID NO: 42 (NM_{—}001320.5; CK2, beta subunit); SEQ ID NO: 43 (NM_{—}002715.2; PP2, catalytic subunit, α isoform); SEQ ID NO: 44 (NM_{—}002717.3; PP2, regulatory subunit B); SEQ ID NO: 45 (NM_{—}014225.5; PP2, regulatory subunit A); and SEQ ID NO: 58 (NM_{—}001009552.1; PP2, catalytic subunit, β isoform).

Within still further aspects, siRNA of the present disclosure are modified and/or conjugated to a component that permits the transfer of the siRNA across the blood-brain barrier of a patient. Exemplified herein are siRNA that are conjugated to chimeric rabies virus glycoprotein fragment RVG-9R NH₂-YTIWMPEBPR PGTPCDIFTN SRGKRASNGG GGRRRRRRRR R—COOH (SEQ ID NO: 11).

Within other embodiments, the present disclosure provides compositions comprising small-molecule and peptide antagonists of kinases such as CDK5, P38, jnk, src, caspase 3, calpains, CK2, PKC, GSK3α and β, PP1, PP2; and NCX1, each of which is effective in reducing psychosine-mediated neurotoxicity, in particular psychosine-mediated axonopathy. Exemplified herein are proteins and compositions comprising the peptide GSK3β antagonist L803 (Tocris Bioscience, Ellisville, Mo.), which comprises the amino acid sequence Lys-Glu-Ala-Pro-Pro-Ala-Pro-Pro-Gln-pSer-Pro (SEQ ID NO: 28). Also exemplified herein are compositions comprising the small-molecule NCX1 antagonist flecainide.

Compositions according to the present disclosure may comprise one or more siRNA molecule(s) that are targeted against, and lead to the downregulation of, mRNA that encode an effector of psychosine-mediated axonal degeneration and/or one or more small-molecule and/or peptide antagonist of an effector of psychosine-mediated axonal degeneration. For example, compositions of the present disclosure may comprise two or more siRNA molecules each of which is targeted against one or more mRNA that encodes a kinase such as CDK5, P38, jnk, src, CK2, PKC, GSK3α and β; caspases such as caspase 3; calpains such as calpain 1 and 2; a phosphatase such as the Ser/Thr protein phosphatase PP1 and tyr protein phosphatase PP2; and/or a sodium/calcium exchange proteins such as NCX1. Alternatively, compositions of the present disclosure comprise two or more antagonists of a kinase such as CDK5, P38, jnk, src, CK2, PKC, GSK3α and β; caspases such as caspase 3; calpains such as calpain 1 and 2; a phosphatase such as the Ser/Thr protein phosphatase PP1 and tyr protein phosphatase PP2; and/or a sodium/calcium exchange proteins such as NCX1.

Typically, each siRNA is modified or conjugated to a second component such that the siRNA and/or antagonist is capable of crossing the blood-brain barrier and, thereby, gaining access to the axons of the central nervous system. For example, each siRNA may be conjugated to chimeric rabies virus glycoprotein fragment RVG-9R NH₂-YTIWMPEBPR PGTPCDIFTN SRGKRASNGG GGRRRRRRRR R—COOH (SEQ ID NO: 11).

Within still further embodiments, the present disclosure provides methods for the treatment of a neurodegenerative disease in a patient suffering from a psychosine-mediated neurological disorder, which methods comprise the step of administering to the patient a composition comprising one or more siRNA molecule(s) each of which is targeted against, and leads to the downregulation of, mRNA that encode an effector of psychosine-mediated axonal degeneration. Within certain aspects, these methods comprise the step of administering to the patient a composition comprising one or more siRNA molecule(s) each of which is targeted against mRNA that encode CDK5, P38, jnk, src, caspase 3, calpains, CK2, PKC, GSK3α and β, PP1, PP2; and NCX1. Optionally, these methods may further comprise the step of administering to the patient a composition comprising GALC-expressing cell, such as a macrophage within a bone marrow sample from a suitable donor.

Within related embodiments, the present disclosure provides methods for the treatment of a neurodegenerative disease in a patient suffering from a psychosine-mediated neurological disorder, which methods comprise the step of administering to the patient a composition comprising one or more small molecule and/or peptide antagonist of an effector of psychosine-mediated axonal degeneration. Within certain aspects, these methods comprise the step of administering to the patient a composition comprising one or more small molecule and/or peptide antagonist of CDK5, P38, jnk, src, caspase 3, calpains, CK2, PKC, GSK3αand β, PP1, PP2; and/or NCX1. Optionally, these methods may further comprise the step of administering to the patient a composition comprising a GALC-expressing cell, such as a macrophage within a bone marrow sample from a suitable donor.

Depending upon the particular treatment regimen employed, the methods of the present disclosure comprise the step of administering a composition comprising one or more siRNA(s) and/or one or more antagonist(s) between 0 days and 60 days following the birth of the patient. More typically, the composition comprising one or more siRNA(s) and/or one or more antagonist(s) is administered to the patient between 0 days and 30 days following the birth of the patient, or between 0 days and 15 days following the birth of the patient or between 0 days and 7 days following the birth of the patient.

In those aspects of the present methods that further comprise the step of administering to the patient a composition comprising a GALC-expressing cell, the composition comprising a GALC-expressing cell is administered between 0 days and 120 days following the birth of the patient, or between 14 days and 90 days following the birth of the patient, or between 30 days and 60 days following the birth of the patient.

### BRIEF DESCRIPTION OF THE FIGURES

FIG. 1 is a bar graph depicting levels of psychosine in blood and serum isolated from the Twitcher mouse, which carries a somatic mutation in the gene encoding the lysosomal enzyme galactosylceramidase (GALC).

FIG. 2 demonstrates that bone marrow transplantation (BMT) improves survival and myelin of Twitcher mice. (A) Newborn Twitcher (Twi) pups received a combined treatment (CT) with total congenic (GALC+/+) bone marrow (3×10⁷cells/animal) and with a single injection of lentiviral vector carrying GALC (10⁷ particles/animal). Some mice received only BMT. Each group includes 12 mice. (B) Brains collected at P7, P45, and at maximal survival (75-125 days) were used for determination of GALC activity expressed as reconstituted activity with respect to wild-type brain and psychosine concentration, expressed as fold increase with respect to wild type levels. Results are mean±SD from 3-5 samples per group. (C-E) Myelination was studied by electron microscopy of transverse sections from sciatic nerves. G-ratio was calculated from at least 200 axons per nerve from wild type (WT), untreated (NT), and combined treated (CT) Twitcher nerves. Data are mean±SD from 4 nerves per group, p<0.05. D and E show electron micrographs of a treated and non-treated Twitcher nerve, respectively, at 10,000-fold magnification.

FIG. 3 demonstrates that GALC deficiency activity in Twitcher neurons leads to the accumulation of psychosine. (A) Granule neurons (GN) were purified from wild-type pups and analyzed by immunoblot for their expression of GALC. A single 75 kDa band was detected. Blots of total brain proteins contained various immunoreactive bands ranging from 70 to 85 kDa. (B) Graph showing the concentration of psychosine in extracts from wild type (WT) and Twitcher (Twi) granule neurons (GN). Data are expressed as mean±SD in pmol per mg of protein. (C and D) LC-MS-MS chromatograms identifying the peak of psychosine (arrows) in extracts from WT and Twitcher neurons.

FIG. 4 demonstrates reduced axonal transport in Twitchers. The transport of syntaxin and SNAP25 in the sciatic nerve was examined by immunoblot of P15 nerves. Expression of both synaptic-associated proteins was reduced in the Twitcher (TW1) sciatic nerve. Actin was used as housekeeping gene.

FIG. 5 demonstrates chromatolysis in the Twitcher mouse. (A) Coronal sections of WT (left) and TWI (right) lumbar spinal cord at P7, P15, and P30 stained with Nissl show a decrease in the number of Nissl+ neurons in the TWI. (B) Counting of the Nissl+ motoneurons in the ventral horns of the WT and TWI spinal cord at P7, P15, and P30. The counting is expressed as number of cells per square millimeter. (C) Western blot analysis of lysates of brain, spinal cord, and sciatic nerve for myelin basic protein (MBP) and protein zero (P0) at P7, P15, and P30. Loss of these myelin specific proteins is evident at P15 and P30.

FIG. 6 demonstrates loss of Nissl in Twitcher spinal motor. Nissl staining of the lumbar region of Twitcher spinal cord (A,C) shows loss of Nissl in ventral horn motor neurons as compared to WT (B,D). Numerous Twitcher neurons appear as ghost profiles (arrows in C) with little Nissl. (E) Quantitation of Nissl+ cells per area revealed significant (˜50%) reduction in P40 but not in P7 TWI spinal cords.

FIG. 7 demonstrates that apoptosis is a late event in the Twitcher neuropathology. (A-I) WT and TWI spinal cord stained for TUNEL, NeuN, and DAPI, magnification 40-fold. Several TUNEL+/NeuN+ neurons were detected in the TWI gray matter at P40 (A-C). Tunel+ glia in the white matter (D-F) were also detected. No TUNEL+cells were detected in the WT tissue (G-I). (J) Counting of the NeuN+ motoneurons in the ventral horns of the lumbar spinal cord. The counting is expressed as cells per square millimeter. No significant changes were detected at any time point indicating that the activation of the death pathway in the neuronal soma was a late event. (K) Counting of TUNEL+/NeuN+ cells in the ventral horns of the lumbar spinal cord. The counting is expressed as cells per square millimeter. (L,M) Representative Western blot of sciatic nerve lysate at P7 and P30 (L) and relative quantification (M, comprehensive of the P15 nerves) showing the increase in Bad and Bax in the young animal. The data are expressed as fold changes respect the age matched WT samples.

FIG. 8 presents evidence of early axonopathy in the Twitcher nervous system. FIGS. 8A-8I shows confocal microscopy of coronal and longitudinal sections of P7, P15, and P30. TWI-Thyl.1 shows axonal dystrophy along the TWI axons, while WT axons did not show any abnormalities (FIGS. 8G-8I). FIGS. 8D and 8G and coronal sections of cords while FIGS. 8A-8C, 8E, 8F, 8H, and 8I are longitudinal sections. FIGS. 8E and 8H are 5-fold magnification of sections of P30 WT and TWI-Thyl.1 spinal cord longitudinal sections, which indicate that axonal dystrophy widely affected the axons of the TWI white matter. FIGS. 8J-8L are confocal imaging of P15 (8J) and P30 (8K) TWI-Thyl.1 sciatic nerves, which shows that the peripheral nerves are also affected by axonal dystrophy, while the P15 WT axons (8L) are unaltered.

FIG. 9 demonstrates exacerbated abundance of membranous vesicles in Twitcher axons. Optic nerves (FIGS. 9A and 9C) and sciatic nerves (FIGS. 9B and 9D) from P40 Twitchers were processed for electron microscopy observation. Arrows point to membranous vesicles accumulated in central and peripheral axons in the mutant animal. All micrographs are at 10,000-fold magnification.

FIG. 10 demonstrates that kinesin levels are decreased in the Twitcher sciatic nerves. FIGS. 10A-10B are the results of an immunoblot analysis of KHC, KLC, and actin in spinal cord and sciatic nerve at P7, P15, and P30. No significant changes were detected in the Twitcher spinal cord at any time point (FIG. 10A, and FIGS. 10C and 10E for the quantification), while the sciatic nerve showed the decrease of KHC and KLC at P15 and P30 (FIG. 10B, and FIGS. 10D and 10F for the quantification). The results are averages of 4 animals per condition.

FIG. 11 presents evidence of defective axonal transport in the Twitcher mouse. FIG. 11A is a Western blot analysis of the non-ligated control (NL) and of the proximal (PS) and distal (DS) stumps of the ligated WT (left panel) and Twitcher (TWI) (right panel) nerves. While the WT accumulated mitochondria (represented by the mitochondrial protein HSP60), synaptic vesicles (represented by the synaptic vesicle SNAP25) and KHC (antibody H2), ligated Twitcher showed little or no accumulation of any of the transported molecules. The experiment was run in triplicate and the bands of the immunoblot were quantified. Values were averaged and normalized to the loading control (actin). FIGS. 11B-11C show quantification of the ligation experiment performed on the P7 (FIG. 11C) and P30 (FIG. 11D) WT and Twitcher animals. The decrease in the accumulation of transported cargoes was evident at P7, when demyelination was not present. FIGS. 11D-I show TEM pictures of non-ligated (FIGS. 11D and 11G) and ligated (FIGS. 11E, 11F, 11H, and 11I) wild type (FIGS. 11D-11F) and Twitcher (FIGS. 11G-11I) sciatic nerves. The WT axons displayed abundant accumulation of vesicular material towards the site of ligation (FIGS. 11E and 11F), while several Twitcher axons was significantly less (FIGS. 11H and 11I).

FIG. 12 presents a model for dysfunctional fast axonal transport as a pathogenic mechanism in leukodystrophies. As disclosed herein, axonal transport of cargoes can be targeted and disrupted by an abnormal level of psychosine, a substrate that fails to be degraded in Krabbe disease. Other lysosomal deficiencies also lead to the accumulation of various lipids and other metabolites whose effect on fast axonal transport is yet to be determined. Many of these deficiencies are affected by demyelination and neurodegeneration of the nervous system. By this model, consequent to the loss of myelin, accumulation of substrates in axonal compartments led to deficiencies in the transport rates of cargoes along the axon, establishing the conditions for axonal dysfunction and degeneration. The two pathogenic pathways may converge at a certain point in disease and synergize into a compounding phenotype.

FIG. 13 demonstrates that axons degenerate in Twitcher mice. Longitudinal sections of the spinal cord of TWI-YFPax mice were examined by confocal microscopy at P7 (FIG. 13A), P15 (FIG. 13B), and P30 (FIG. 13C). Arrows point to varicosities and swellings in motor axons that occurred only in the mutants (FIGS. 13A-13C) but not in the wild-type (FIG. 13D). Similarly, axonopathic figures were detected in TWI-YFPax cerebellar peduncles (FIG. 13E), sciatic nerves (FIG. 13G), and striated mossy fibers (not shown) but not in the corresponding WT-sections (FIGS. 13F and 13H).

FIG. 14 demonstrates that Twitcher neurons produce psychosine. FIGS. 14A and 14B show the determination of psychosine concentration by HPLC-MS-MS of spinal cord (FIG. 14A) and sciatic nerve (FIG. 14B) at P7, P15, P30, and P40. The quantification shows that psychosine, which accumulates exponentially during the disease, is significantly higher than the WT controls even at P3 (enlargement in FIGS. 14A and 14B). The difference was more evident at P3 in the sciatic nerve. FIGS. 14C and 14D show HPLC-MS-MS determination of psychosine concentration in WT and Twitcher primary neurons after 8 days of culture. Although the Twitcher neurons accumulated less psychosine than Twitcher oligodendrocytes, they accumulated significantly more than the WT cells (FIG. 14D). FIG. 14E shows HPLC-MS-MS determination of psychosine in NSC34 cells that have been incubated with 5 μM psychosine.

FIG. 15 demonstrates that galactosyl-psychosine but not glucosyl-psychosine is accumulated in Twitcher brain. FIG. 15A shows that HPLC-mass spectrometry (LC-MS-MS) using a C18 HPLC column (Waters) was unable to distinguish galactosyl from glucosyl-psychosines, which appeared with the same m/z value. FIG. 15B shows derivatization of psychosines using NBD-F. FIG. 15C shows chromatograms of NBD-galactosyl-psychosine as a function of the retention time (RT in min., left chart) and of m/z ion mass (right chart) using a polar aklyamide HPLC column (Supelco, Supelcosil™ ABZ+ column, cat #57917; SigmaAldrich; St. Louis, Mo.). FIG. 15D shows chromatograms of NBD-glucosyl-psychosine as function of the retention time (RT in min., left chart) and of m/z ion mass (right chart). FIG. 15E shows a protocol using the alkylamide-HPLC discriminated both NBD-psychosines in a mixture (50:50) with RT of 9.45 min (NBD-galactosyl-psychosine) and 10 min (NBD-gluco-psychosine) (left chart). Both peaks showed the same m/z ion mass of 625 (right chart). FIG. 15F shows P40 and FIG. 15G shows Twitcher brain lipid extracts analyzed by either C18-LC-MS-MS or by NBD-F derivatization/alkylamide-LC-MS-MS. NBD-galactosyl-psychosine (m/z 625) was detected in the mutant brain with a RT of 9.45 min. NBD-gluco-psychosine was not detected.

FIG. 16 demonstrates that galactosyl-psychosine but not glucosyl-psychosine is accumulated in the Twitcher mouse brain. FIG. 16A shows that HPLC-mass spectrometry (LC-MS-MS) using a C18 HPLC column (Waters) was unable to distinguish galactosyl- from glucosyl-psychosines, which appeared with the same m/z value. FIG. 16B shows derivatization of psychosines using NBD-F. FIG. 16C shows chromatograms of NBD-galactosyl-psychosine as a function of the retention time (RT in min., left chart) and of m/z ion mass (right chart) using a polar alkylamide HPLC column (Supelco, supelcosil ABZ column, Cat. No. 57917). FIG. 16D shows chromatograms of NBD-glucosyl-psychosine as a function of the retention time (RT in min., left chart) and m/z ion mass (right chart). FIG. 16E shows that the new protocol using the alkylamide-HPLC discriminated both NBD-psychosines in a mixture (50:50) with RT of 9.45 min (NBD-galactosyl-psychosine) and 10 min (NBD-glucosyl-psychosine) (left chart). Both peaks showed the same m/z ion mass of 635 (right chart). FIG. 16F shows P40(g) Twitcher brain lipid extracts analyzed by either C18-LC-MS-MS or by NBD-F derivatization/alkylamide-LC-MS-MS. NBD-galactosyl-psychosine (m/z 625) was detected in the mutant brain with a RT of 9.45 min; NBD-glucosyl-psychosine was not detected.

FIG. 17 demonstrates neuronal expression of enzymes involved in the metabolism of psychosine. FIG. 17A shows real-time PCR analysis of mRNA expression of GALC and CGT in acutely purified cultures of GN maintained for 3 and 8 days in vitro. FIG. 17B shows that CGT was immunodetected in extracts of NSC34 motoneuronal cells and protein extracts from P7 wild type (WT) and Twitcher (TWI) spinal cords. FIG. 17C shows immunodetection of CGT in large ventral horn motor neurons. FIG. 17D shows background staining in the absence of a primary antibody. Magnification in FIGS. 17C and 17D is 100-fold.

FIG. 18 demonstrates that psychosine accumulates in Twitcher lipid rafts. Psychosine accumulations were analyzed by mass spectrometry in lipid raft fractions prepared from wild-type (WT) and Twitcher (TWI) mice at P3 and P40. FIG. 18A shows that total psychosine concentrations were much greater in TWI brains as compared to WT brains. Data are means±SD from 2-4 mice per time point. FIG. 18B presents representative data from mass spectrometric analysis of psychosine in raft fractions, which shows a significantly larger peak in P3 TWI vs P3 WT. FIG. 18C shows preferential distribution of psychosine in raft fractions (3-5) in all samples with much greater accumulations in raft fractions of TWI mice.

FIG. 19 demonstrates that psychosine blocks fast axonal transport. FIG. 19A shows that psychosine exhibited a strong inhibitory effect on both antero and retrograde transport in whole-mount preparations of giant squid axons. FIG. 19B shows that vehicle controls exhibited no defective transport rates.

FIG. 20 demonstrates that psychosine is a pathogenic lipid that inhibits fast axonal transport. FIGS. 20A-20D show primary cultures of Twitcher granular neurons cultured for 1 (FIG. 20A), 5 (FIG. 20B), and 8 (FIG. 20C) days in vitro. Mutant cells degenerated faster than in sister WT cultures (FIG. 20D). FIGS. 20E-20J show primary cortical neurons incubated with 0.1, 1, and 10 μM psychosine (FIGS. 20E-20G), D-Sphingosine (negative control, FIG. 20H), C6-ceramide (positive control, FIG. 20I) and vehicle (0.1% ethanol, FIG. 20J). FIG. 20K shows NSC34 cells treated with 10 μM psychosine and the number cells with processes longer than 2 cells diameters were counted. FIG. 20L shows primary cortical neurons cultured as with psychosine and control sphingolipids and neuronal survival was the MTT assay. The results are shown as percentage of the control and are means±SEM of three independent experiments. FIGS. 20M-20O show extruded preparations of squid axoplasms incubated with psychosine or control lipids. Upon perfusion, the transport rate of vesicles was recorded by videomicroscopy. Psychosine strongly inhibited both modes of FAT. Data represent 3-6 axoplasms per condition.

FIG. 21 demonstrates that psychosine inhibits axonal transport by activating PP1. FIG. 21A shows PP1 activity that was fluorometrically determined in brain and sciatic nerve extracts from wild-type (WT) and Twitcher (TWI) (n=2 per time point per genotype). FIG. 21B shows PP1 activity increased in cortical neurons after incubation with psychosine for 1 hour (n=3). FIGS. 21C-21E show Axoplasm preparations infused with 5 FM psychosine alone (FIG. 21E) or co-infused with 200 nM of okadaic acid (FIG. 21C) or 50 nM of inhibitor (FIG. 21D). PP1 inhibitor significantly ameliorated inhibition of fast axonal transport by psychosine. FIG. 21F shows immunoblots of total brain protein extracts with antibodies against total neurofilaments (NF) or phosphorylated neurofilaments (SMI 31) revealed a lower abundance of phosphorylated neurofilaments in Twitcher brains. Actin was used as housekeeping gene for protein loading control.

FIG. 22 demonstrates abnormal NCXI and Ca⁺⁺ levels in Twitcher CNS. FIG. 22A shows relative changes in intraneuronal Ca⁺⁺ measured by patch-clamping of hippocampal CA2 neurons with Fura2. Data represent net changes in Fura2 fluorescence from neurons of P20 Twitcher (n=10) and age-matched wild-types over 4 seconds after a train of 15 action potentials (AP train, arrow). FIGS. 22B and 22C show confocal images from transverse sections of the spinal cord of Twitcher and wild-type mice, respectively, after immunostaining with anti-NCX1.

FIG. 23 demonstrates that early treatment with flecainide is neuroprotective in Twitcher mice. Twitcher-YEPax mice were treated with flecainide (30 mg/kg body weight/day) or vehicle starting at postnatal day P5 (early group) or P9 (fate group) and continued until P30. FIG. 23A shows delayed onset of twitching by calculating the percentage of mice twitching at 15, 20, 25, and 30 days of age (n=4 mice per group). FIGS. 23B and 23D-23G show longitudinal sections of spinal cords from mice sacrificed at P30 (lumbar region) observed by YFP confocal microscopy. The frequency of axononathic figures (swellings, varicosities, breaks; arrowheads in FIGS. 23D-23G) per area was assessed and plotted in FIG. 23B. FIG. 23C is an immunoblot of protein extracts from lumbar spinal cord, which shows that early flecainide treatment reduced the expression of NCX1. Late flecainide treatment showed no differences in NCX1 expression, compared with vehicle-treated Twitchers.

FIG. 24 demonstrates that the RVG peptide binds to neurons and crosses the blood-brain barrier (BBB). FIGS. 24A-24F show N2A cells exposed to 100 pmol of RVG-FITC per ml (FIGS. 24A and 24D) or to vehicle (FIGS. 24C and 24F) for 4 h before fixation and counterstaining with a whole cell fluorescent stain. HeLa cells were also incubated with RVG-FITC under identical experimental conditions (FIGS. 24B and 24E). Green fluorescent particles of RVG-FITC were only detected in N2A cells but not in HELA cells or in mock-N2A cells. FIGS. 24G-24I show two-day-old wild type pups intravenously injected with 20 μl of RVP-FITC containing 50 pmol of peptide (FIGS. 24G and 24H) or 5% glucose saline (vehicle, FIG. 24I). Brain cryosections were observed by confocal microscopy. Neurons in the cortex (FIGS. 24G and 24I) contained green fluorescent deposits of RVG-FITC peptide. Brain tissue from mock (vehicle) treated mice showed background fluorescence without any specific pattern (FIG. 24I).

FIG. 25 demonstrates siRNA-mediated reduction of catalytic α- and β-PP1 subunit expression in N24, N2A (FIGS. 25A and 25B), and HeLa (FIG. 25C) cells exposed to 10 pmol of siRNA or scrambled (scr) primers for catalytic α- and β-PP1 subunits. Primers were mixed with 100 pmol of RVG-FLIC and incubated for 4 hours. Cells were then incubated in siRNA-free fresh medium for 48 hours before real time (RT) (FIGS. 25A and 25C) or immunoblot (FIG. 25B) analyses for catalytic α- and β-PP1 subunit expression. RT-PCR, normalized using RLPO as the internal housekeeping gene, showed significant reduction in mRNA levels for either subunit in N2A cells (FIG. 25A) but not in HeLa cells (FIG. 25C). Immunoblotting analysis showed reduced abundance of each protein subunit in siRNA-treated N2A cells (FIG. 25B), but not in HeLa cells (not shown). Expression of each subunit was normalized against kinesin as the housekeeping protein and expressed as fold changes.

FIG. 26 demonstrates that PP1 mediates psychosine-inhibition of FAT. FIGS. 26A-26B show experiments using extruded axoplasm from the giant axon of squid Loligo pealei, which permitted the identification of PP1 as a mediator in the inhibition of FAT induced by psychosine. Okadaic acaid and inhibitor I2 were used to block phosphatase activities. Co-perfusion of 200 nM okadaic acid (FIG. 26A) or 50 nM 12 (FIG. 26B) with 5 μM psychosine prevented FAT inhibition induced by psychosine. FIG. 26C shows that psychosine induced a dose-dependent increase in PP1 activity in acutely purified embryonic cortical neurons. Data is expressed as fluorescence units/mg prot/h originating from 3 independent experiments. FIG. 26D shows that PP1 activity increased in nerve tissues from the Twitcher mouse. PP1 activity was measured in freshly prepared extracts from brain, spinal cord, and sciatic nerves from Twitcher (TWI) and age-matched wild type (WT) at P15. Data is expressed as fluorescence units/mg prot/h; n=3 animals per condition per genotype. FIG. 26E shows that spinal cord and sciatic nerve protein extracts immunoblotted for each of the three catalytic PP1 subunits. Sciatic nerves showed a substantial accumulation of PP1β and γ. Actin and neurofilament M (NFM) were used as loading controls.

FIG. 27 demonstrates that psychosine induces the activation of GSK3β which ultimately inhibits FAT. FIG. 27A shows that the activation of GSK3β occurs after PP1-mediated removal of phosphate at Ser9 and can be visualized in this blot by the decrease in binding of anti-phospho-Ser9 antibody. P6 and P30 Twitcher (TW1) and wild type (WT) spinal cord protein extracts were blotted with anti-phospho-Ser9. Twitcher spinal cords contained significantly more active (less immunoreactive) GSK3β than the wild type controls. The abnormal GSK3β activity led to increased phosphorylation of KLC motors, which was detected by a reduced binding of the phosphodependent mAb 63.90. Actin was used as a loading control. FIG. 27B shows that extruded axoplasms exhibited abnormal activation of GSK3β for the inhibition of FAT induced by psychosine. Co-perfusion of 100 nM of GSK3β inhibitor ING35 significantly prevented FAT inhibition by psychosine. FIG. 27C presents a model showing that psychosine inhibition of fast axonal transport (FAT) involves the activation of PP1, which dephosphorylates GSK3β. Increased GSK3β activity led to the abnormal phosphorylation of KLCs (pKLC) and release of cargoes from motors and FAT inhibition. Reduction of FAT triggered the aberrant translocation of axonal components and led to degeneration.

FIG. 28 is the nucleotide sequence of Homo sapiens protein phosphatase 1, catalytic subunit, α-isoform (NM_{—}206873.1; SEQ ID NO: 12).

FIG. 29 is the nucleotide sequence of Mus musculus protein phosphatase 1, catalytic subunit, α-isoform (NM_{—}031868.2; SEQ ID NO: 13).

FIG. 30 is the nucleotide sequence of Homo sapiens protein phosphatase 1, catalytic subunit, β-isoform (NM_{—}002709.2; SEQ ID NO: 14).

FIG. 31 is the nucleotide sequence of Mus musculus protein phosphatase 1, catalytic subunit, β-isoform (NM_{—}172707.3; SEQ ID NO: 15).

FIG. 32 is the nucleotide sequence of Homo sapiens cyclin-dependent kinase 5 (CDK5) (NM_{—}004935.3; SEQ ID NO: 16).

FIG. 33 is the nucleotide sequence of Homo sapiens glycogen synthase kinase 3β (GSK3β) (NM_{—}001146156.1; SEQ ID NO: 17).

FIG. 34 is the nucleotide sequence of Homo Sapiens PKC (NM_{—}002737.2; SEQ ID NO: 18).

FIG. 35 is the nucleotide sequence of Homo sapiens NCK adaptor protein 1 (NCK1) (NM_{—}006153.4; SEQ ID NO: 19).

FIG. 36 is the amino acid sequence of Homo sapiens protein phosphatase 1, catalytic subunit, α-isoform (NM_{—}206873.1; SEQ ID NO: 20) encoded by the nucleotide sequence of SEQ ID NO: 12.

FIG. 37 is the amino acid sequence of Mus musculus protein phosphatase 1, catalytic subunit, α-isoform (NM_{—}031868.2; SEQ ID NO: 21) encoded by the nucleotide sequence of SEQ ID NO: 13.

FIG. 38 is the amino acid sequence of Homo sapiens protein phosphatase 1, catalytic subunit, β-isoform (NM_{—}002709.2; SEQ ID NO: 22) encoded by the nucleotide sequence of SEQ ID NO: 14.

FIG. 39 is the amino acid sequence of Mus musculus protein phosphatase 1, catalytic subunit, β-isoform (NM_{—}172707.3; SEQ ID NO: 23) encoded by the nucleotide sequence of SEQ ID NO: 15.

FIG. 40 is the amino acid sequence of Homo sapiens cyclin-dependent kinase 5 (CDK5) (NM_{—}004935.3; SEQ ID NO: 24) encoded by the nucleotide sequence of SEQ ID NO: 16.

FIG. 41 is the amino acid sequence of Homo sapiens glycogen synthase kinase 3 β (GSK3β) (NM_{—}001146156.1; SEQ ID NO: 25) encoded by the nucleotide sequence of SEQ ID NO: 17.

FIG. 42 is the amino acid sequence of Homo Sapiens PKC (NM_{—}002737.2; SEQ ID NO: 26) encoded by the nucleotide sequence of SEQ ID NO: 18.

FIG. 43 is the amino acid sequence of Homo sapiens NCK adaptor protein 1 (NCK1) (NM_{—}006153.4; SEQ ID NO: 27) encoded by the nucleotide sequence of SEQ ID NO: 19.

FIG. 44 is the nucleotide sequence of Homo sapiens P38 (NM_{—}002745.4; SEQ ID NO: 34).

FIG. 45 is the nucleotide sequence of Homo sapiens jnk (NM_{—}002750.2; SEQ ID NO: 35).

FIG. 46 is the nucleotide sequence of Homo sapiens src (NM_{—}005417.3; SEQ ID NO: 36).

FIG. 47 is the nucleotide sequence of Homo sapiens caspase 3 (NM_{—}004346.3; SEQ ID NO: 37).

FIG. 48 is the nucleotide sequence of Homo sapiens calpain 1, large subunit (NM_{—}005186.2; SEQ ID NO: 38).

FIG. 49 is the nucleotide sequence of Homo sapiens calpain, small subunit (NM_{—}001749.2; SEQ ID NO: 39).

FIG. 50 is the nucleotide sequence of Homo sapiens calcium kinase 2, alpha subunit (NM_{—}177559.2; SEQ ID NO: 40).

FIG. 51 is the nucleotide sequence of Homo sapiens calcium kinase 2, alpha prime subunit (NM_{—}001896.2; SEQ ID NO: 41).

FIG. 52 is the nucleotide sequence of Homo sapiens calcium kinase 2, beta subunit (NM_{—}001320.5; SEQ ID NO: 42).

FIG. 53 is the nucleotide sequence of Homo sapiens protein phosphatase 2, catalytic subunit, alpha isozyme (NM_{—}002715.2; SEQ ID NO: 43).

FIG. 54 is the nucleotide sequence of Homo sapiens protein phosphatase 2, regulatory subunit B, alpha (NM_{—}002717.3; SEQ ID NO: 44).

FIG. 55 is the nucleotide sequence of Homo sapiens protein phosphatase 2, regulatory subunit A, alpha (NM_{—}014225.5; SEQ ID NO: 45).

FIG. 56 is the amino acid sequence of Homo sapiens P38 (NM NM_{—}002745.4; SEQ ID NO: 46) encoded by the nucleotide sequence of SEQ ID NO: 34.

FIG. 57 is the amino acid sequence of Homo sapiens jnk (NM_{—}002750.2; SEQ ID NO: 47) encoded by the nucleotide sequence of SEQ ID NO: 35.

FIG. 58 is the amino acid sequence of Homo sapiens src (NM_{—}005417.3; SEQ ID NO: 48) encoded by the nucleotide sequence of SEQ ID NO: 36.

FIG. 59 is the amino acid sequence of Homo sapiens caspase 3 (NM NM_{—}004346.3; SEQ ID NO: 49) encoded by the nucleotide sequence of SEQ ID NO: 37.

FIG. 60 is the amino acid sequence of Homo sapiens calpain 1, large subunit (NM_{—}005186.2; SEQ ID NO: 50) encoded by the nucleotide sequence of SEQ ID NO: 38.

FIG. 61 is the amino acid sequence of Homo sapiens calpain, small subunit (NM_{—}001749.2; SEQ ID NO: 51) encoded by the nucleotide sequence of SEQ ID NO: 39.

FIG. 62 is the amino acid sequence of Homo sapiens CK2, alpha subunit (NM_{—}177559.2; SEQ ID NO: 52) encoded by the nucleotide sequence of SEQ ID NO: 40.

FIG. 63 is the amino acid sequence of Homo sapiens CK2, alpha prime subunit (NM_{—}001896.2; SEQ ID NO: 53) encoded by the nucleotide sequence of SEQ ID NO: 41.

FIG. 64 is the amino acid sequence of Homo sapiens CK2, beta subunit (NM_{—}001320.5; SEQ ID NO: 54) encoded by the nucleotide sequence of SEQ ID NO: 42.

FIG. 65 is the amino acid sequence of Homo sapiens PP2, catalytic subunit, alpha isozyme (NM_{—}002715.2; SEQ ID NO: 55) encoded by the nucleotide sequence of SEQ ID NO: 43.

FIG. 66 is the amino acid sequence of Homo sapiens protein phosphatase 2, regulatory subunit B, alpha (NM_{—}002717.3; SEQ ID NO: 56) encoded by the nucleotide sequence of SEQ ID NO: 44.

FIG. 67 is the amino acid sequence of Homo sapiens protein phosphatase 2, regulatory subunit A, alpha (NM_{—}014225.5; SEQ ID NO: 57) encoded by the nucleotide sequence of SEQ ID NO: 45

FIG. 68 is the nucleotide sequence of Homo sapiens protein phosphatase 2, catalytic subunit, beta isozyme (NM_{—}001009552.1; SEQ ID NO: 58).

FIG. 69 is the amino acid sequence of Homo sapiens protein phosphatase 2, catalytic subunit, beta isozyme (NM_{—}001009552.1; SEQ ID NO: 58) encoded by the nucleotide sequence of SEQ ID NO: 59.

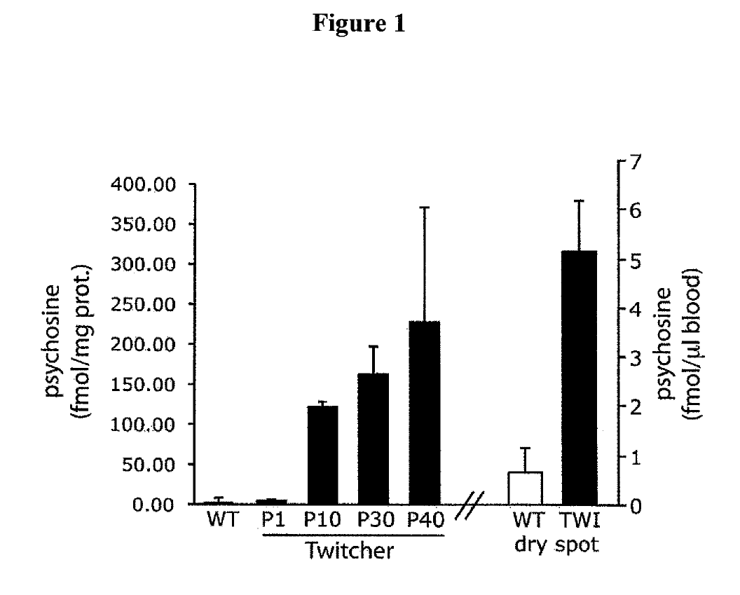

### DETAILED DESCRIPTION OF THE DISCLOSURE

The present disclosure is based upon the unexpected discovery that the administration of compositions comprising one or more inhibitor(s) and/or one or more antagonist(s) of one or more downstream effector(s) of psychosine-mediated axonal degeneration, especially when used in combination with existing treatment modalities such as, for example, bone marrow transplantation (BMT), are effective in reducing and/or eliminating the axonopathy that is associated with Krabbe and other neurodegenerative diseases.

The survival of neurons depends significantly on proper communication with their targets, communication that depends largely on a functional axonal transport and an adequate balance of ions. Axons can be very long (up to one meter in the case of some motor neurons) accounting for most of the neuronal volume, making the maintenance of this structure an important and highly vulnerable aspect of the normal neuronal physiology. Insults affecting axonal structure and function generate the risk of degeneration and neuronal death. Defective axonal transport is reflected in altered trafficking and distribution of on channels, synaptic components, and associated organelles rendering the axon dysfunctional.

As disclosed herein, wild-type neurons from healthy individuals normally express the ubiquitous lysosomal enzyme GALC. Neurons from individuals carrying one or more autosomal recessive mutation(s) in the gene encoding GALC accumulate significant concentrations of the neurotoxin psychosine. Without being limited by mechanistic theory, this finding that GALC-deficient neurons accumulate the same neurotoxin that causes the death of myelinating cells suggests that KD neurons are dysfunctional due to an intrinsic metabolic defect in their lysosomes. It is presently disclosed that the deficiency of GALC in KD not only affects myelination but also triggers intrinsic and contemporaneous defects in neurons and axons. Thus, the presently disclosed treatment modalities for KD and related neurodegenerative diseases are directed at the reduction of axonal degeneration while complementing existing treatment regimens that seek to prevent demyelination through GALC reconstitution.

The present disclosure demonstrates that the pathogenic mechanism of GALC deficiency in KD involves the psychosine-mediated increases in the activity of PP1 in neurons, which leads to the deregulation of the basic components of the axonal transport machinery. PP1 enzymatic activity blocks fast axonal transport and inhibition of this phosphatase significantly protects both antero and retrograde transport modes. Phosphatases are widely distributed in mammalian cells, with PP1 (˜38 kDa) as one of the most conserved phosphatases in eukaryotes. The specificity and activity of PP1 is controlled by about 50 different interacting proteins, which, depending upon the cell type, modulate the catalytic and PP1 subunits or act by scaffolding PP1 to specialized subcellular compartments. Ceulemans and Bollen, Physiol Rev 84:1-39 (2004). In neurons, the role of PP1 in axonal transport depends on PP1 activity associated with the transport machinery, where it appears to regulate various kinases such as GSK3 in the axon.

The progressive accumulation of psychosine in neurons facilitates the abnormal activity of PP1, which impairs fast axonal transport (FAT) and thus alters the homeostasis of vital functional domains in the axon, such as those controlling the intracellular concentration of Ca⁺⁺. Because neurons are generated and mature long before myelinating glia, neurons are exposed to toxic psychosine at an earlier time in development, which likely undermines the possibility of recovery by the time BMT is administered. Thus, the compositions and methods disclosed herein are aimed at treating KD by reducing stress load to neurons as early as possible during postnatal development.

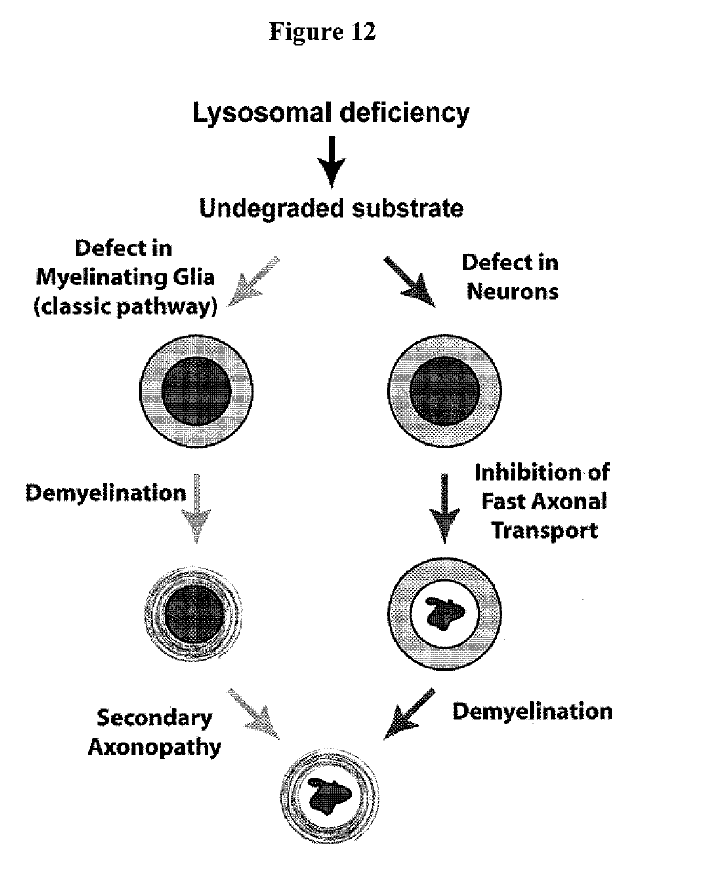

The data presented herein demonstrate that while neuronal loss occurs during brain formation, it is an abnormal occurrence in early infancy and adulthood where it leads to irreversible and devastating neurological consequences. Deregulation of FAT in KD reduces the motility of membrane cargoes between neuronal cell bodies and the synaptic terminals thereby establishing the conditions for a dying-back axonopathy (FIG. 12), which results in abnormal neuronal loss and a pre-demyelination neurological defect. This mechanism underscores the role of dysfunctional axonal transport in KD as well as other similar leukodystrophies.

The present disclosure further demonstrates that FAT is inhibited in the Twitcher mouse model of KD. This finding is consistent with the dying-back mode of neurodegeneration that starts with very early reductions in the antero and retrograde transport of axonal cargoes before any sign of major neuronal dysfunction. It is demonstrated herein that psychosine accumulates in mutant neurons and that this sphingolipid is sufficient to block FAT.

It is disclosed herein that: (1) BMT-treated Twitcher mice show neuronal and axonal damage by the time sufficient therapeutic GALC enzyme accumulates in the nervous system; (2) psychosine is produced and accumulates in neurons in the absence of mutant glia, causing the blockage of fast axonal transport via the activity of protein phosphatase 1 (PP1); (3) mutant neurons show abnormal intracellular levels of Ca⁺⁺ linked to deregulated expression of the Ca⁺⁺ exchanger (NCX1); (4) pharmacological intervention to inhibit PP1 protects axonal transport, while administration of the drug flecainide to normalize NCX1 activities reduces axonopathy in Twitcher mice; and (5) administration of the drug L803, an inhibitor of GSK3β, decreased psychosine-mediated neurotoxicity.

These observations suggest that GALC-deficient neurons mount a stress response that contributes to pathology and that PP1 and NCX1 are two key mediators of the axonal defects of KD that result from the accumulation of toxic levels of psychosine. The fact that long-lived treated Twitcher mice had a significant metabolic correction and ameliorated myelination but still died of neurological phenotype suggests that delaying correction of the metabolic defect does not fully address a more complex disease mechanism. GALC deficiency causes demyelination with a progressive neuronal stress response leading to axonal transport defects via PP1 activity, increased accumulation of Ca⁺⁺ via increased expression of the NCX1 exchanger, and degeneration of axons. Based upon these observations, the present disclosure provides that the activity of PP1 and the NCX1 exchanger may be modulated to enhance neuroprotection in KD and in related neurodegenerative diseases.

Traditional therapies such as BMT, which are based on the reconstitution of the missing enzymatic activity in the nervous system after infiltration of donor-derived macrophages, exhibit a lag time during which correction of CNS deficiency of GALC is low because of low numbers of donor infiltrating cells. By administering neuroprotective agents to reduce axonal stress during this lag of time, the beneficial effects of BMT may be enhanced once GALC correction starts in the CNS. Moreover, once GALC activity increases and begins to clear accumulated psychosine, the need for further neuroprotective therapies may be avoided.

While traditional BMT does not address these neuronal defects, the timely delivery of neuroprotection to mutant neurons prior to or contemporaneously with BMT, is effective in overcoming the deficiencies in BMT that result from a delayed accumulation of GALC within the neurons of the central nervous system. Thus, the presently disclosed compositions and methods complement and/or synergize with existing BMT therapeutic regiments for the treatment of Krabbe and other neurodegenerative diseases.

Neurodegeneration involves defects in axonal transport via PP1 activity and abnormal exposure of axons to calcium via NCX1 activity. Thus, the reduction of neuronal and axonal stress provides a meaningful approach to improve neurological functions in GALC deficiency and to enhance the therapeutic outcome of traditional enzyme replacement by BMT. Within certain embodiments, the present disclosure provides neuroprotective strategies that can enhance the therapeutic benefits of traditional BMT-based treatments.

Specifically, provided herein are compositions and methods that are effective in: (1) achieving the controlled and specific reduction of neuronal PP1 activity using siRNA specific silencing protects axonal transport in mutant neurons; (2) improving NCX1-mediated influx of calcium in axons by administering flecainide, a small molecule antiarrhythmic drug with a proven ability to reduce sodium channel firing and NCX1 activity; and (3) decreasing psychosine-mediated neurotoxicity by administering L803, a peptide antagonist of GSK3β. It is further provided that these neuroprotective strategies when combined with metabolic correction after BMT substantially and unexpectedly improves clinical outcome for patients with Krabbe and other neurodegenerative diseases.

Improving the communication between the soma and the periphery occurs by silencing neuronal PP1 activity through PP1 siRNA treatment and ameliorating both anterograde and retrograde axonal transport rates, which reduces axonal stress and, hence, NCX1 accumulation. Similarly, flecainide treatment reduces the entry of sodium and, hence, counteracts the reverse activity of NCX1 exchanger, leading to reduced calcium-related stress.

The presently disclosed role of PP1, NCX1, and GSK3β activity in mediating neuronal dysfunction in KD provides a unique opportunity to improve the BMT-based metabolic corrective strategies that are currently used to treat this and other related leukodystrophies. It will be understood that the insight disclosed herein may be extrapolated to other lysosomal storage disorders and neurodegenerative diseases, such as metachromatic leukodystrophy, GM1 gangliosidosis, Niemann-Pick disease, Tay-Sachs disease and aging-related neuropathy, which, like KD, are associated with axonal transport deficiences alike those produced by psychosine for which there are no available treatment modalities.

Compositions Comprising Inhibitors and Antagonists of Psychosine-Mediated Neurotoxicity

As described above, the present disclosure provides inhibitory nucleic acids, including siRNA molecules, and small-molecule and peptide antagonists of kinases such as CDK5, P38, jnk, src, CK2, PKC, GSK3α and β; caspases such as caspase 3, phosphatases such as the Ser/Thr protein phosphatase PP1 and Tyr protein phosphatase PP2; and sodium/calcium exchange proteins such as NCX1, each of which is effective in reducing psychosine-mediated neurotoxicity, in particular psychosine-mediated axonopathy.

(a) siRNA Inhibitors

Within certain embodiments are provided siRNA molecules that are targeted against, and lead to the downregulation of, mRNA that encode an effector of psychosine-mediated axonal degeneration. For example, provided are siRNA that are targeted against mRNA that encode CDK5, P38, jnk, src, caspase 3, calpains, CK2, PKC, GSK3α and β, PP1, PP2; and NCX1.

siRNA of the present disclosure comprise an antisense strand of between 15 nucleotides and 50 nucleotides, or between 18 and 40 nucleotides, or between 20 and 35 nucleotides, or between 21 and 30 nucleotides, each of which is capable of specifically binding to a target mRNA encoding a psychosine effector selected from CDK5, P38, jnk, src, caspase 3, calpains, CK2, PKC, GSK3α and β, PP1, PP2; and NCX1.

Exemplified herein are siRNA that bind to the α- and β-isoforms of the Ser/Thr protein phosphatase PP1 and that comprise between 15 and 50 nucleotides of an antisense sequence that is capable of specifically binding to an α- or β-isoform of PP1 mRNA encoded by the cDNA presented in SEQ ID NO: 13 (murine PP1, α-isoform), SEQ ID NO: 12 (human PP1, α-isoform), SEQ ID NO: 15 (murine PP1, β-isoform); and/or SEQ ID NO: 14 (human PP1,13-isoform).

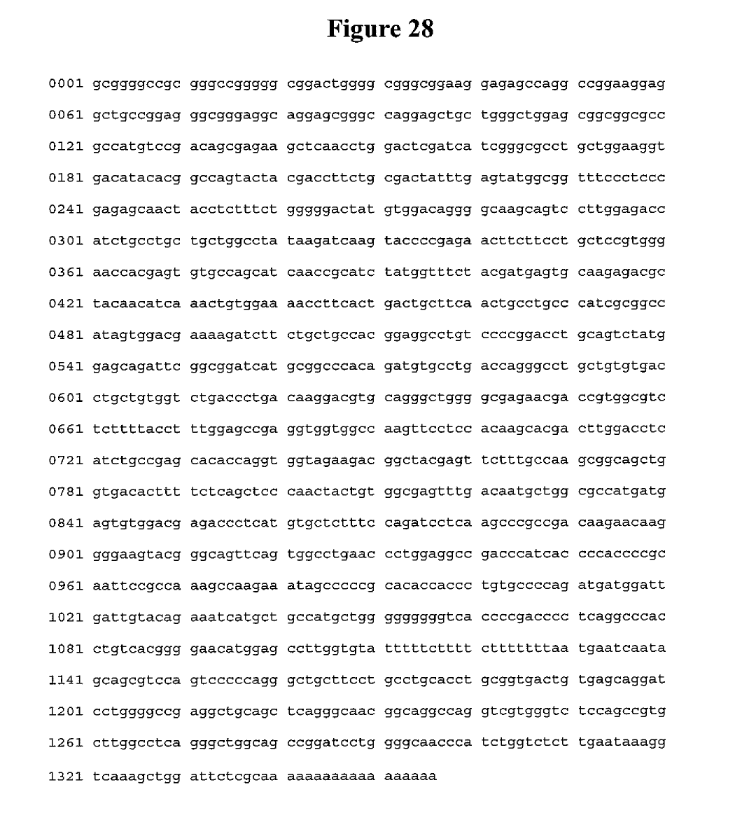

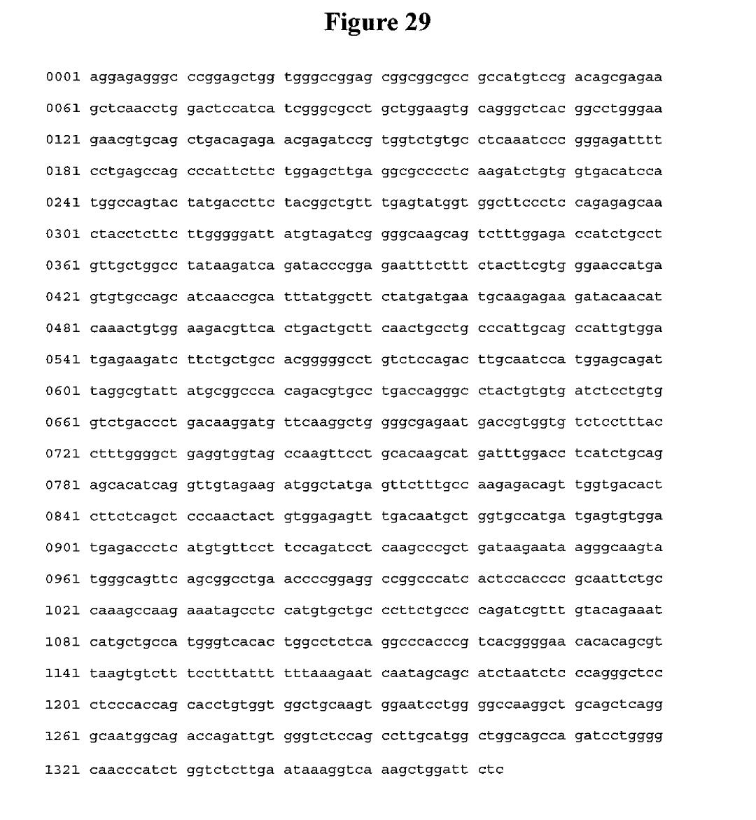

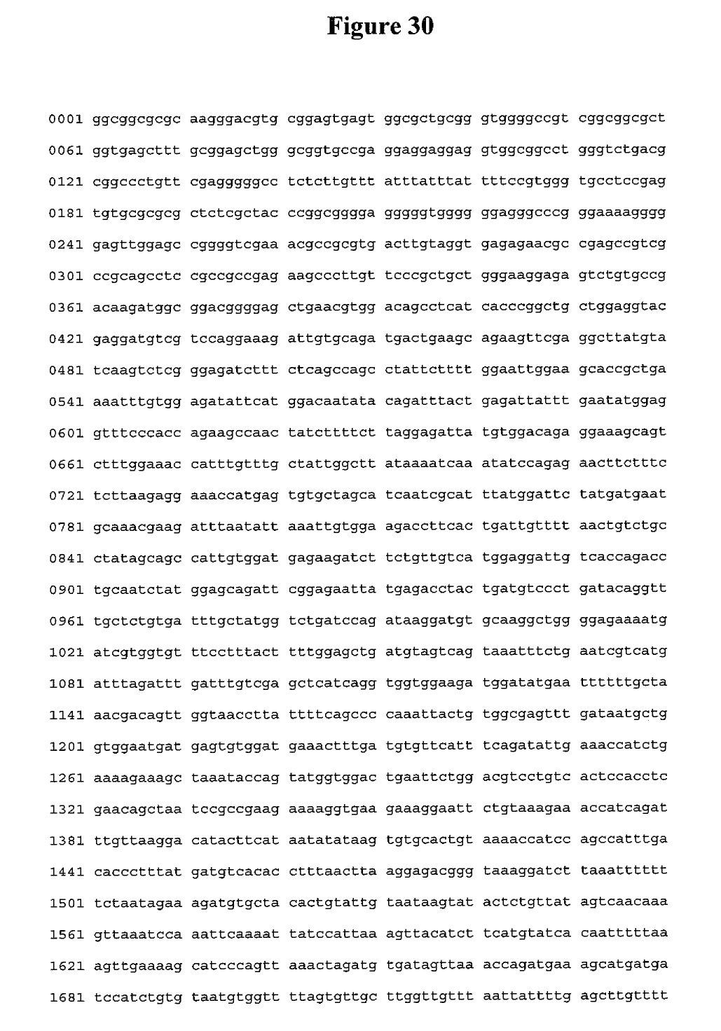

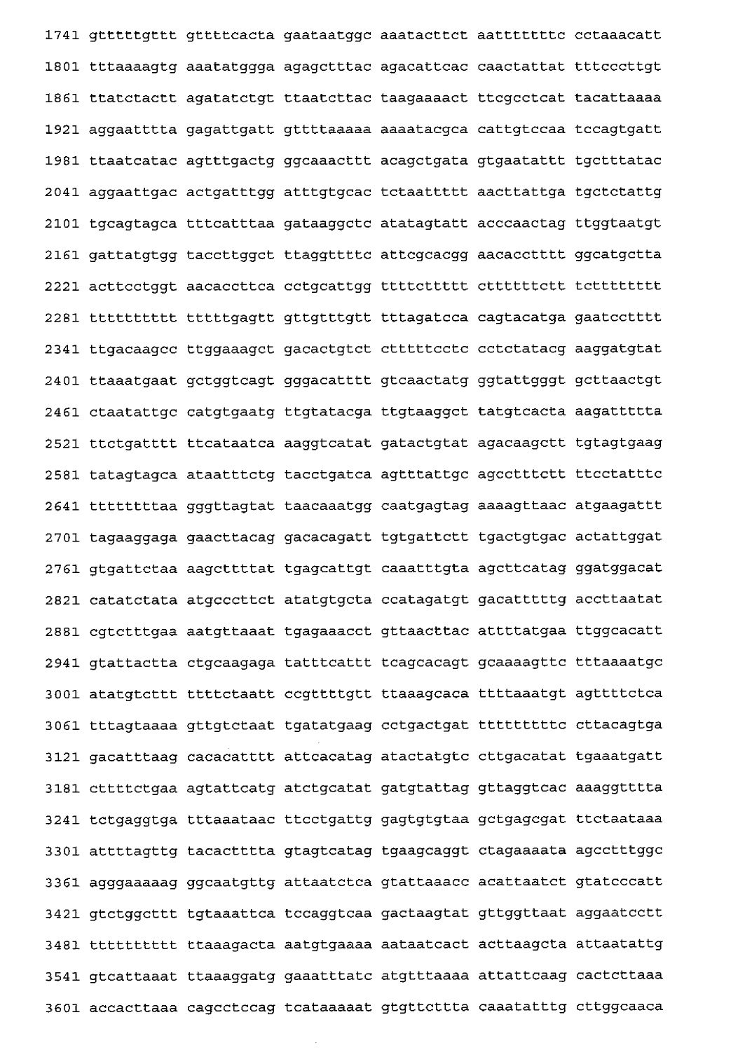

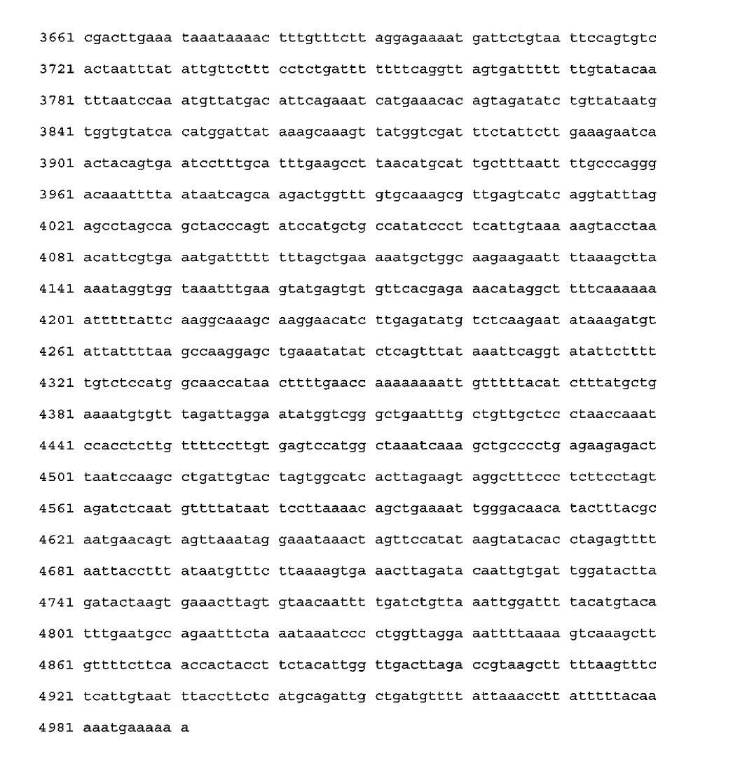

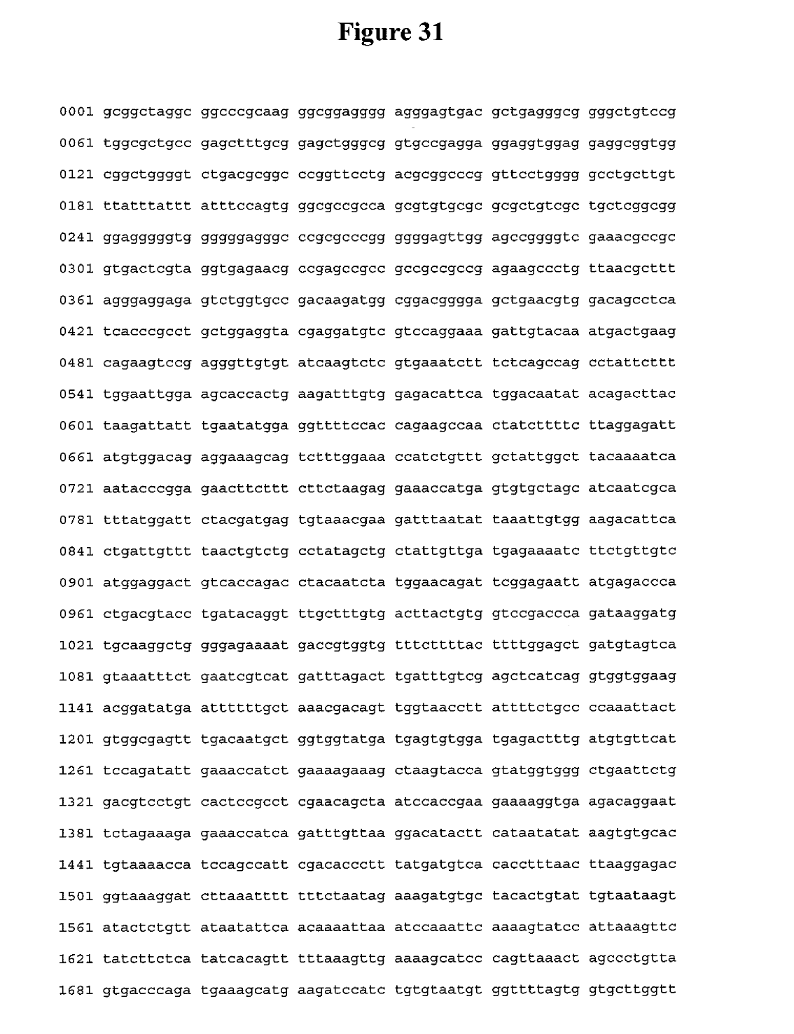

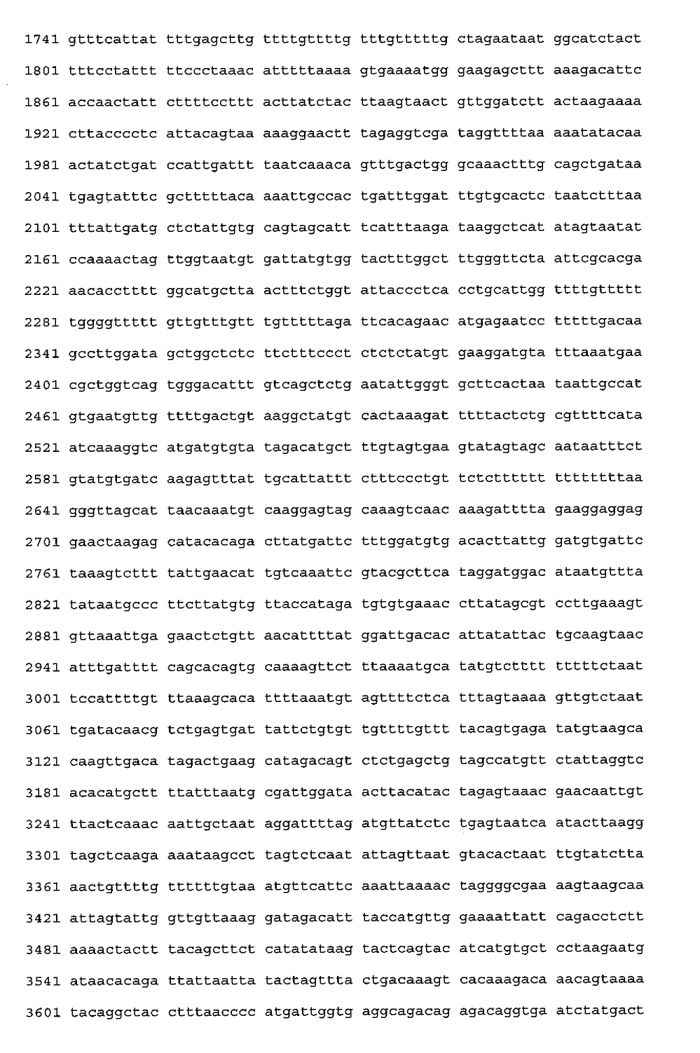

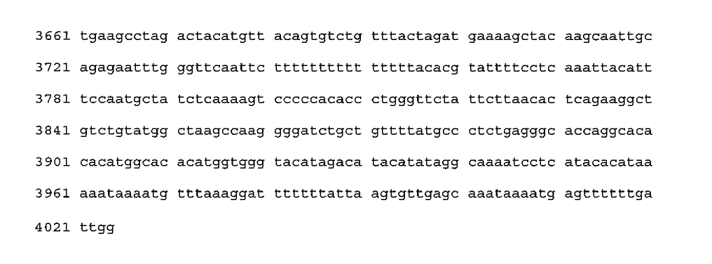

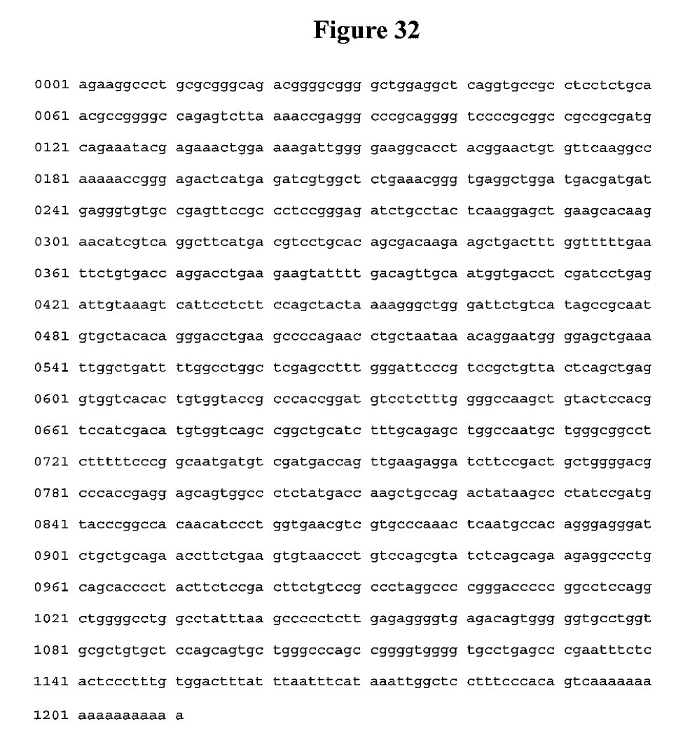

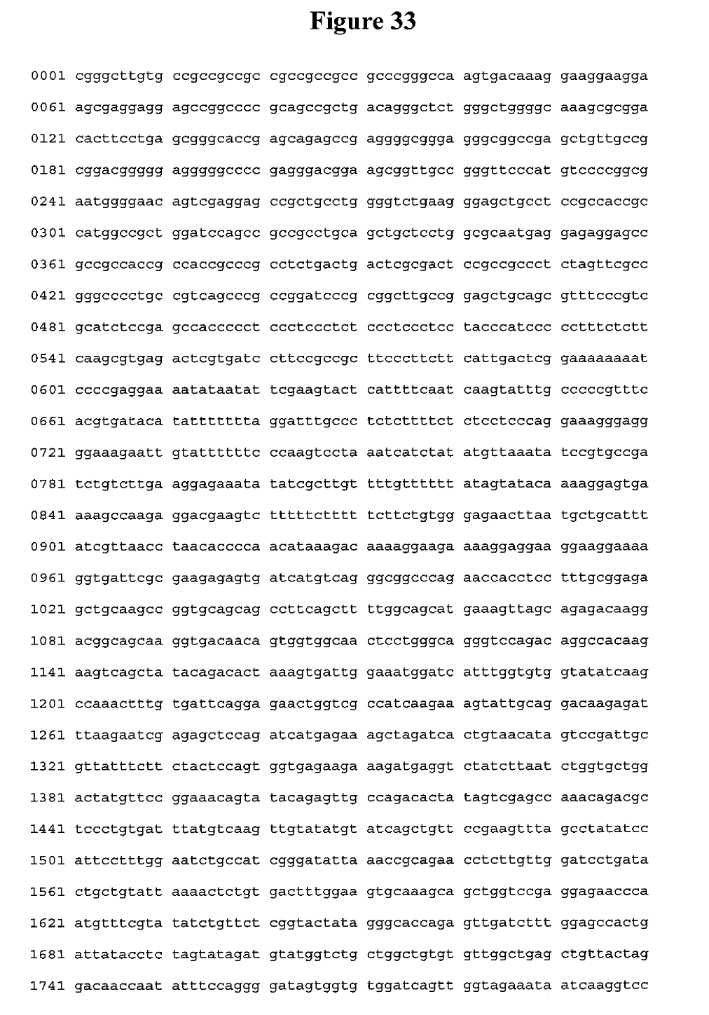

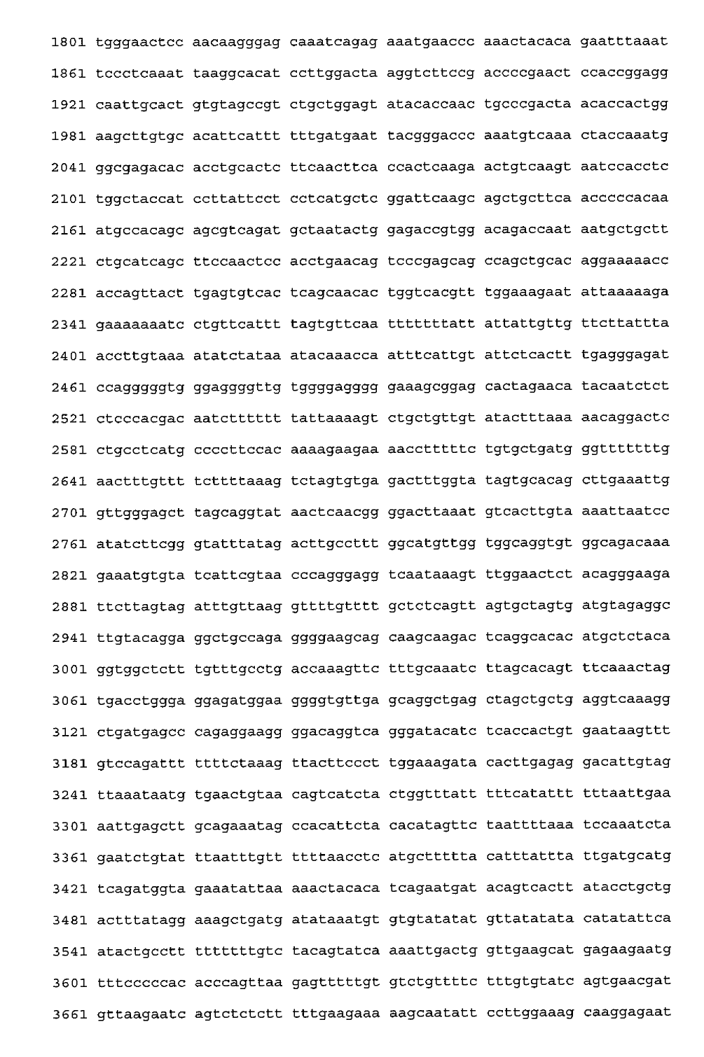

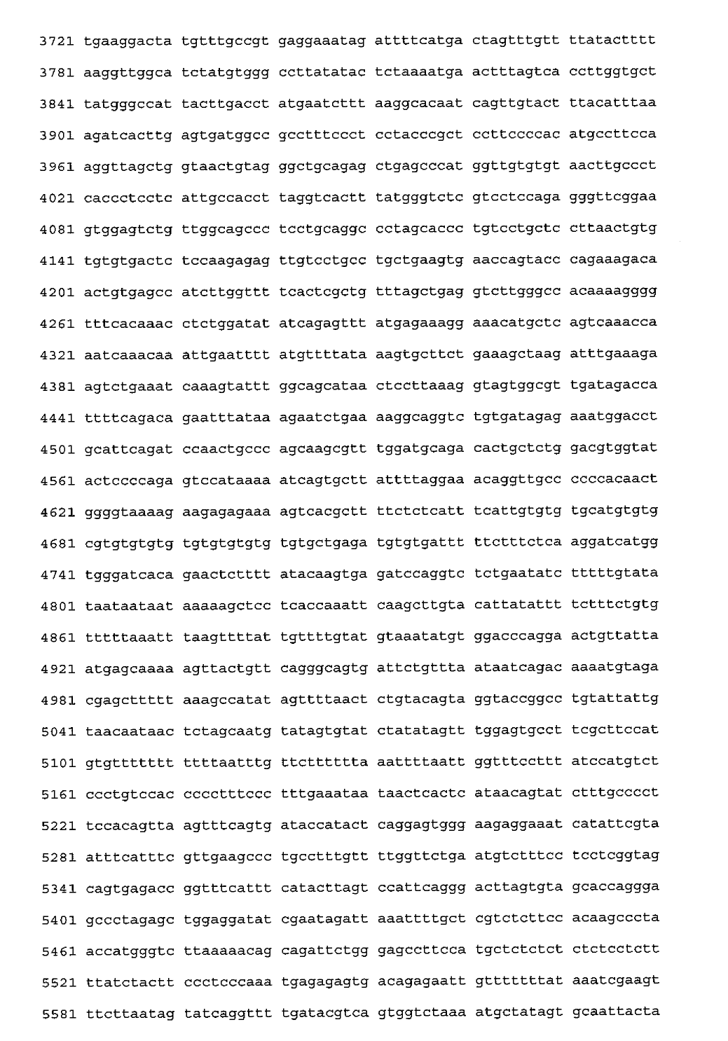

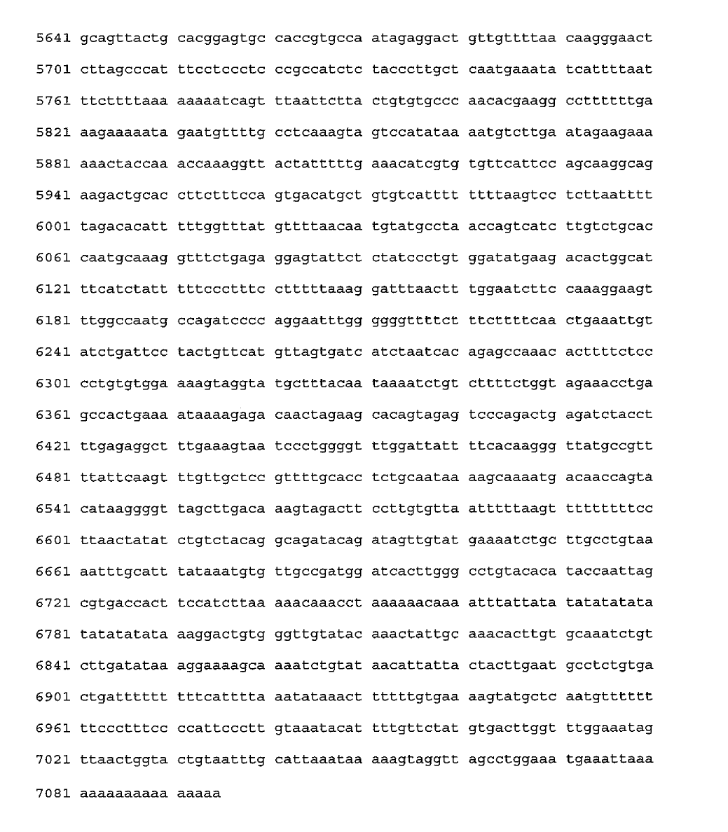

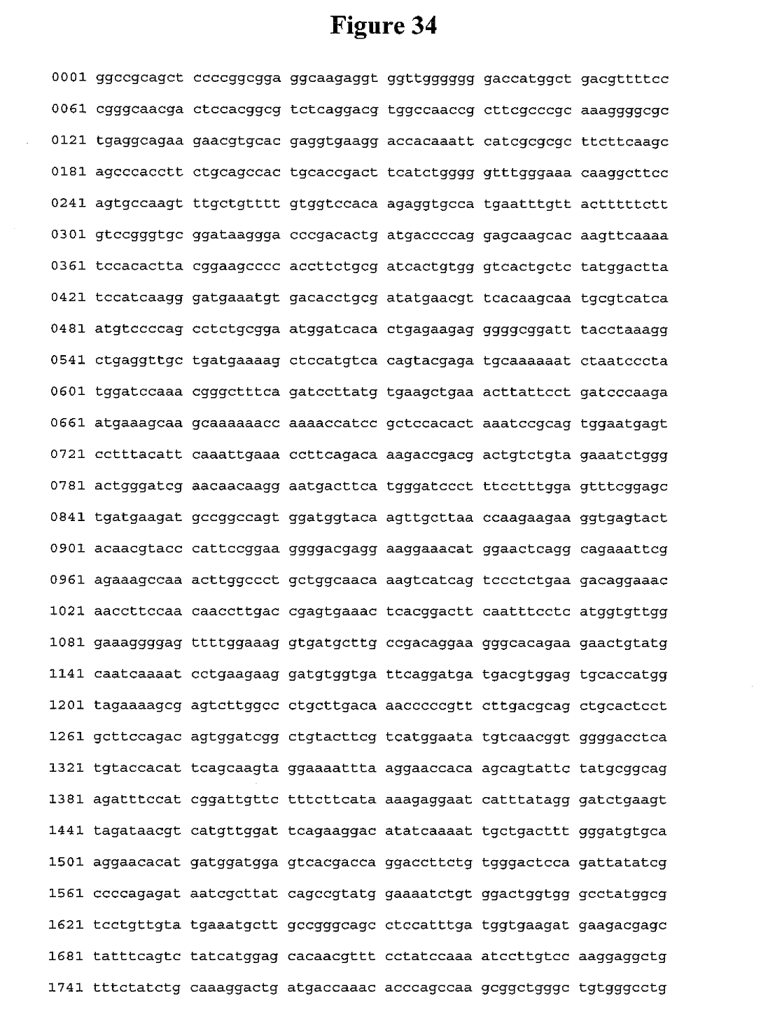

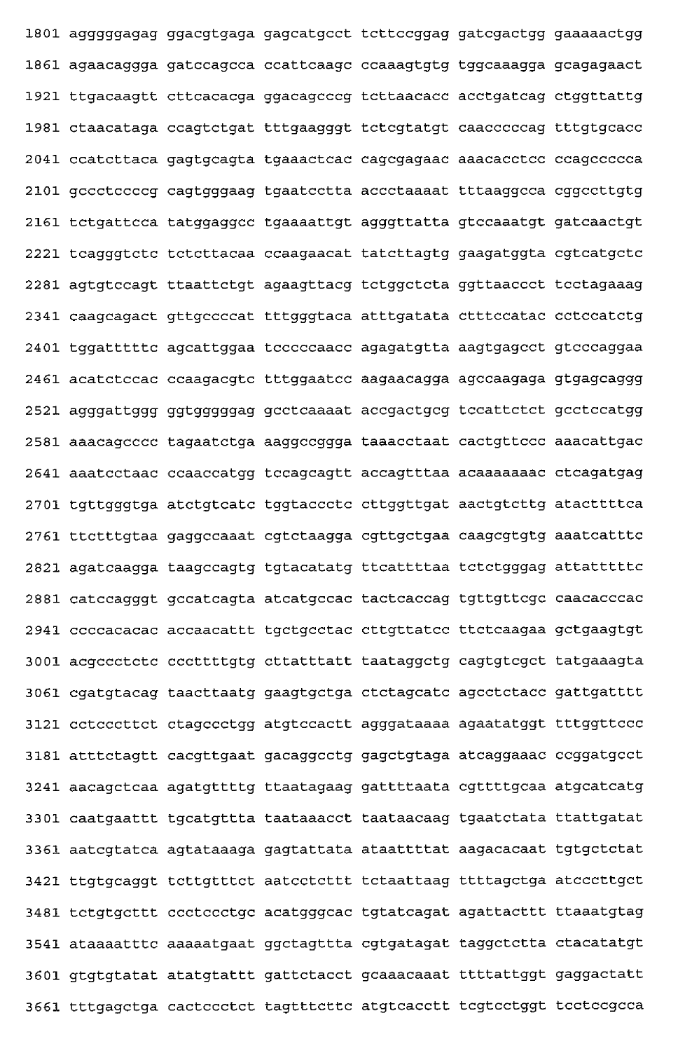

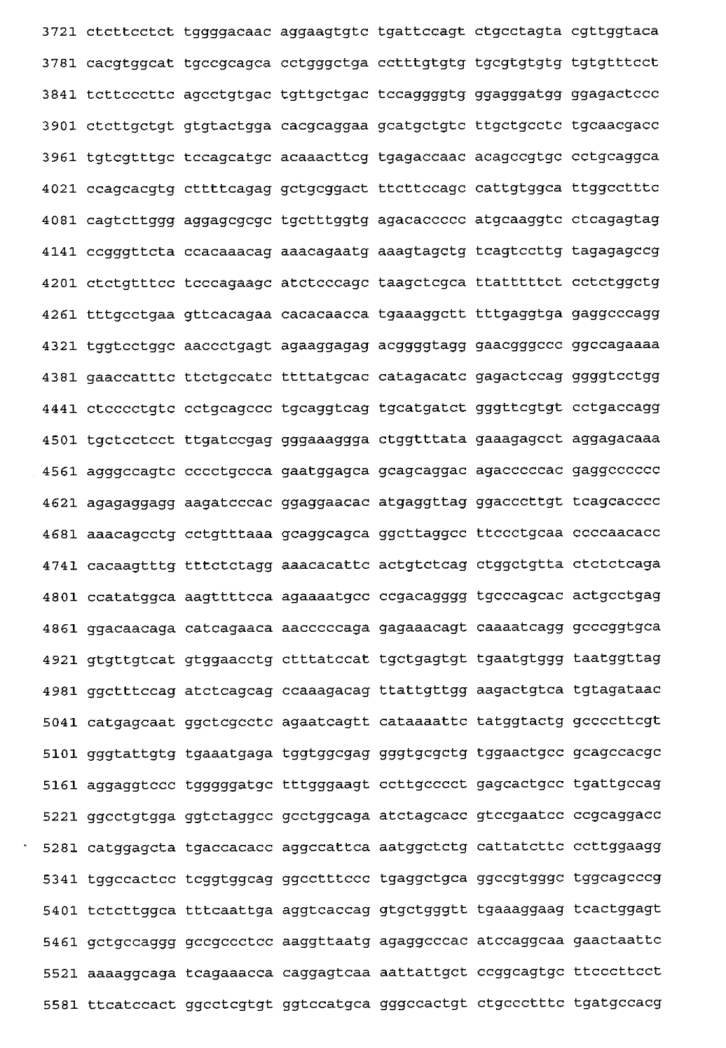

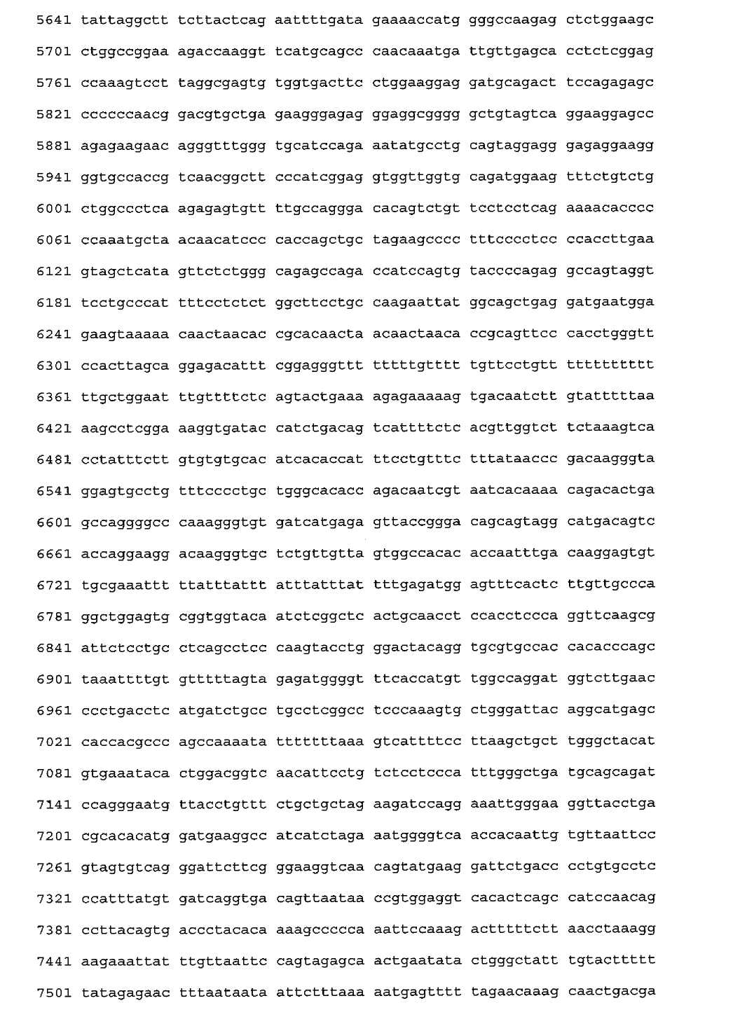

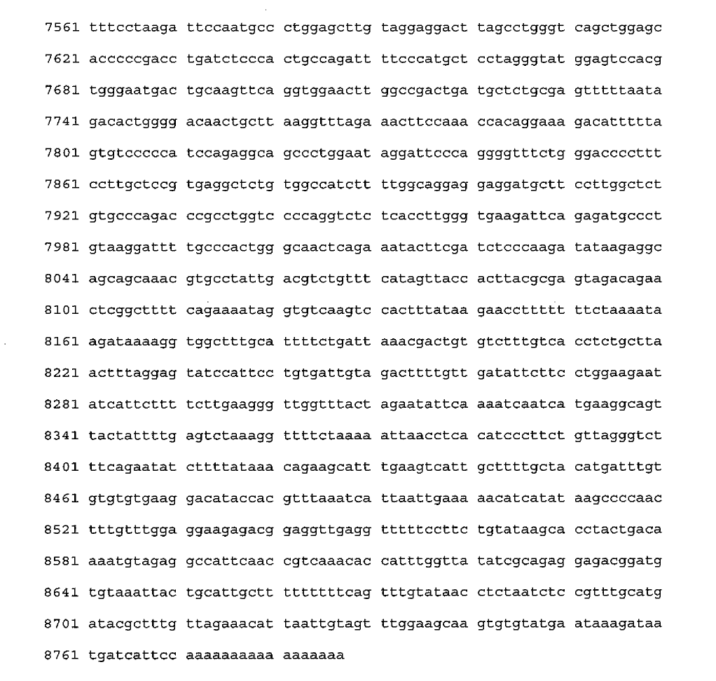

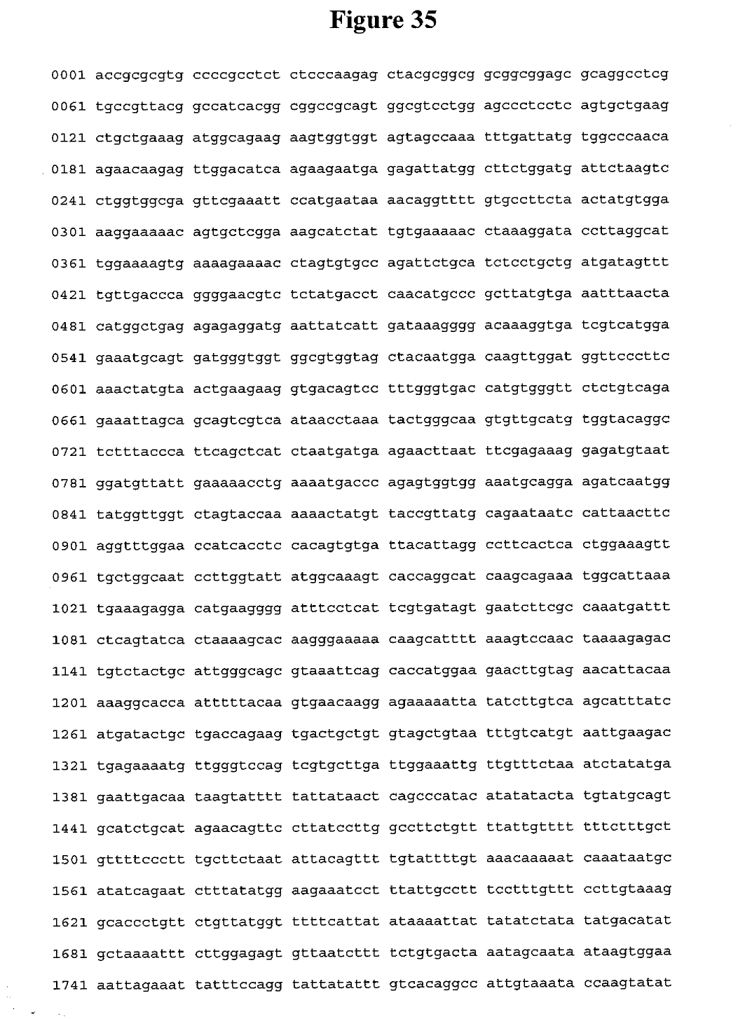

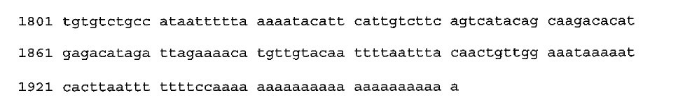

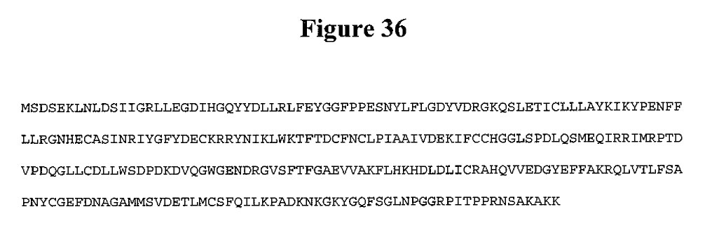

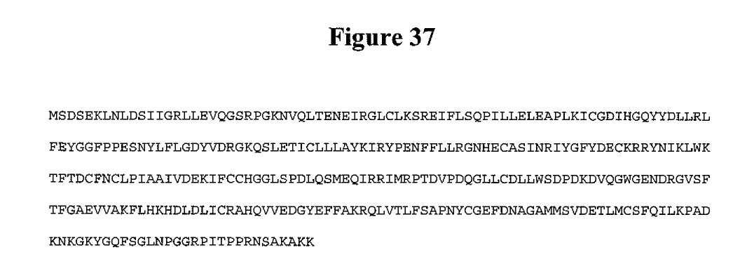

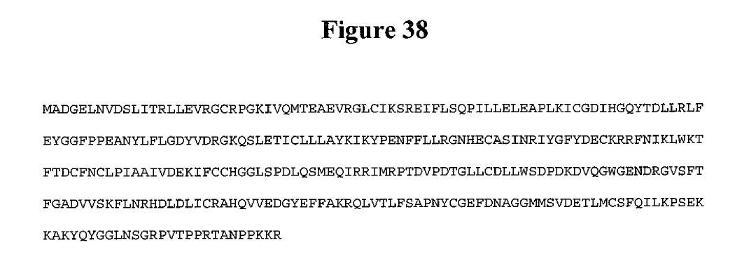

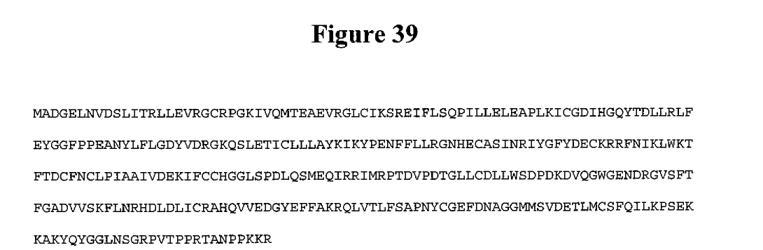

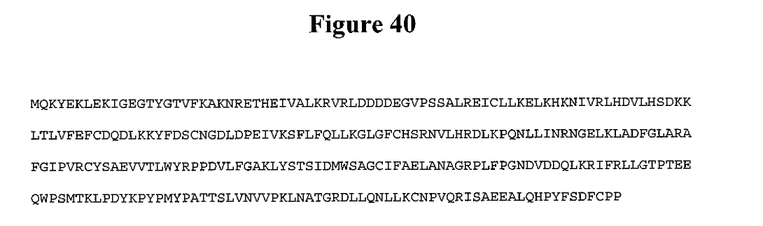

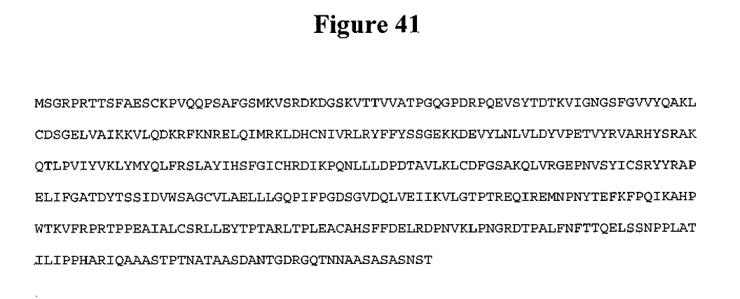

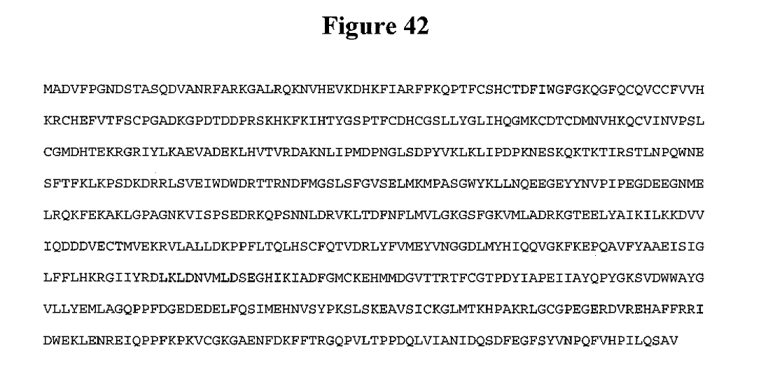

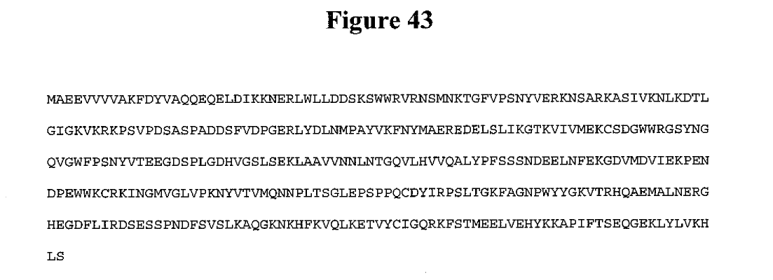

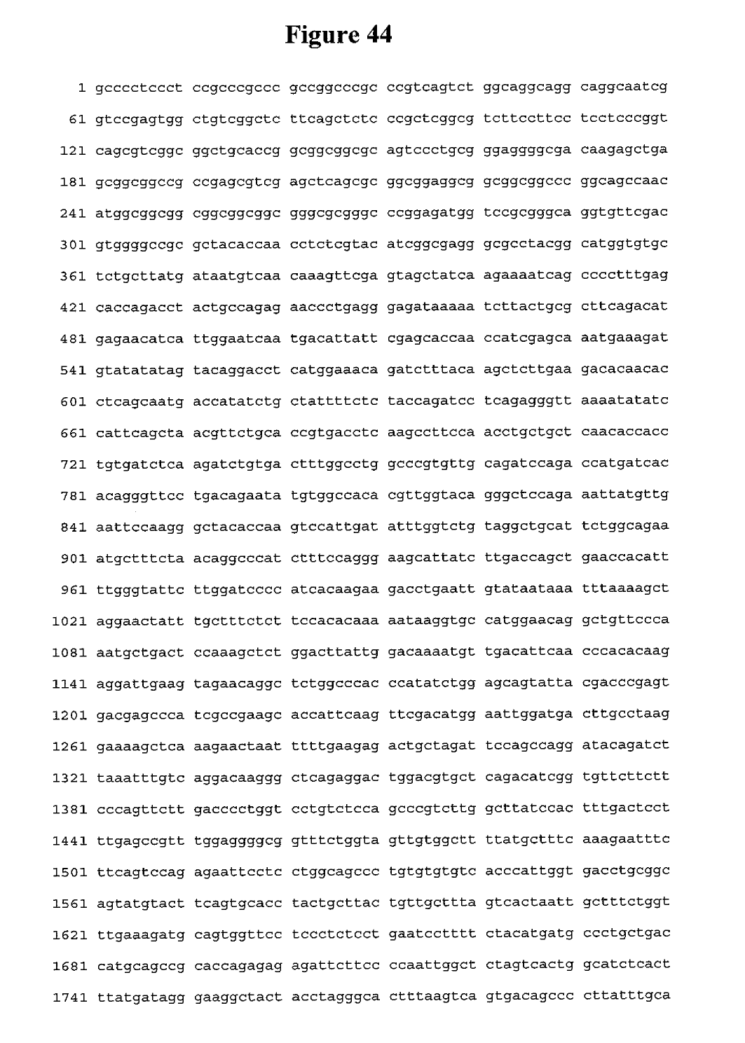

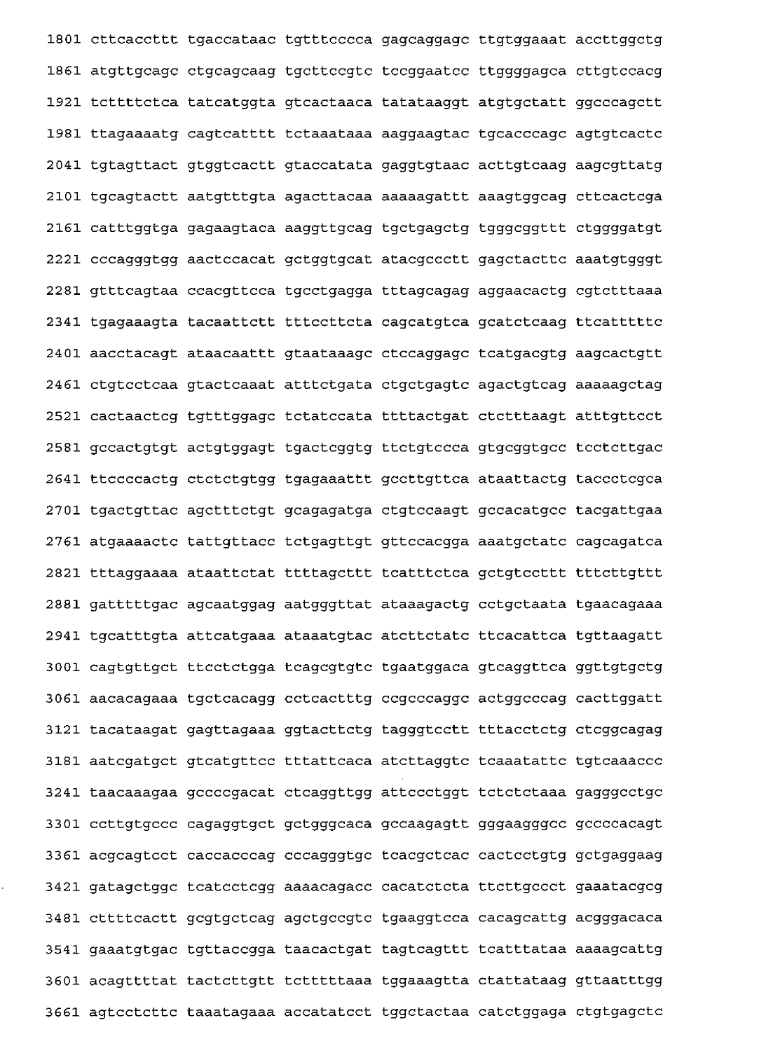

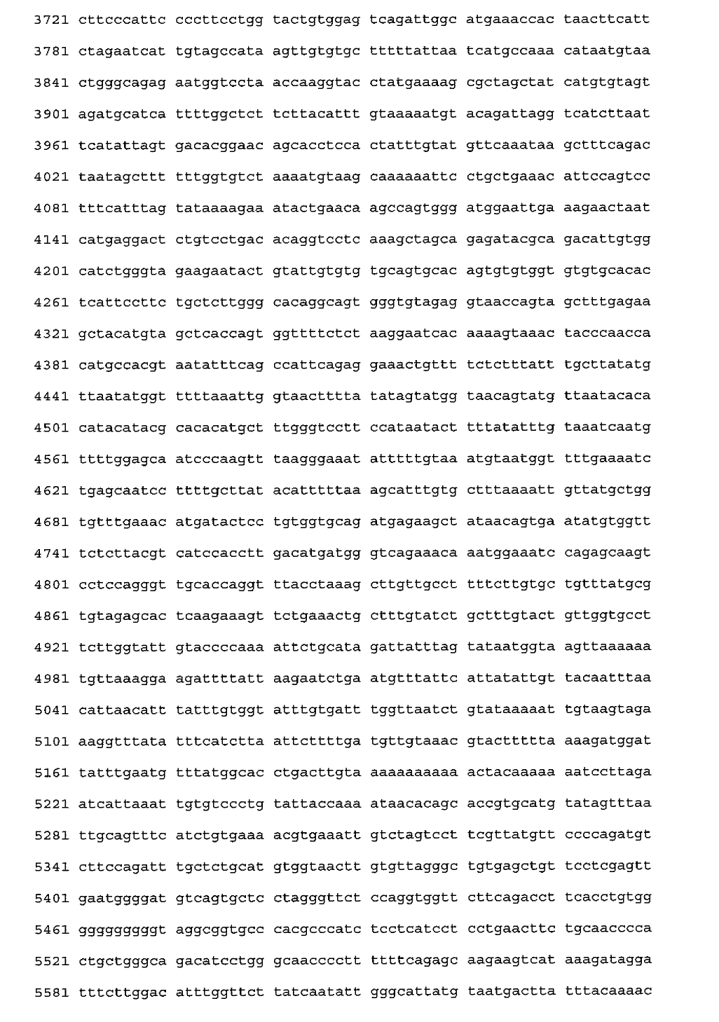

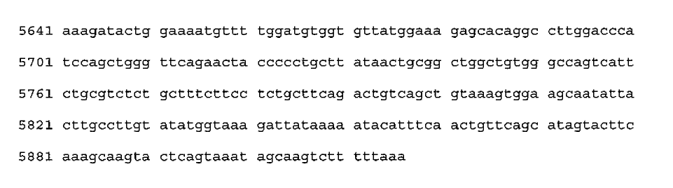

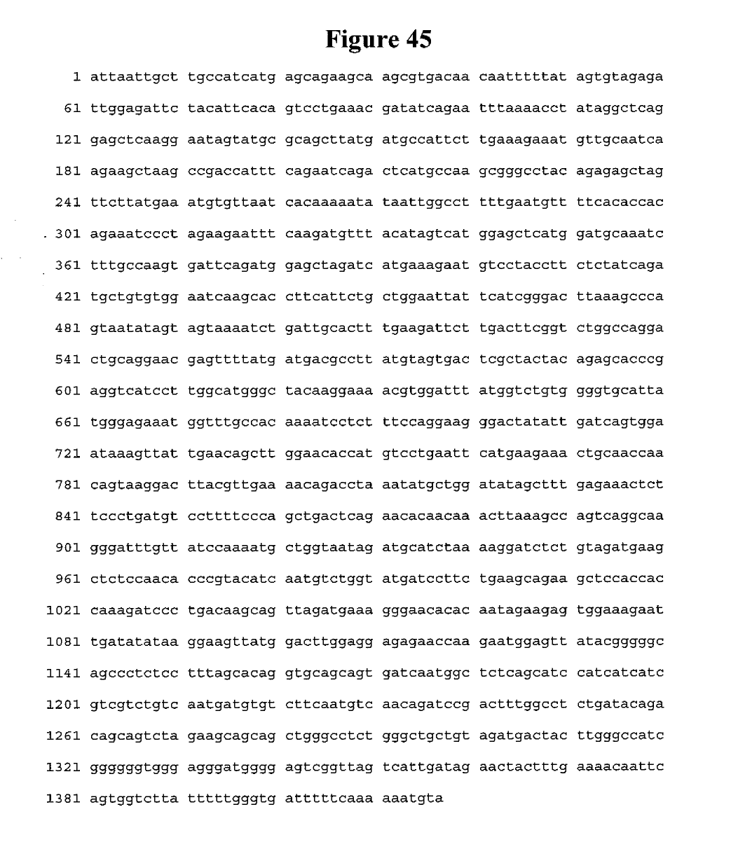

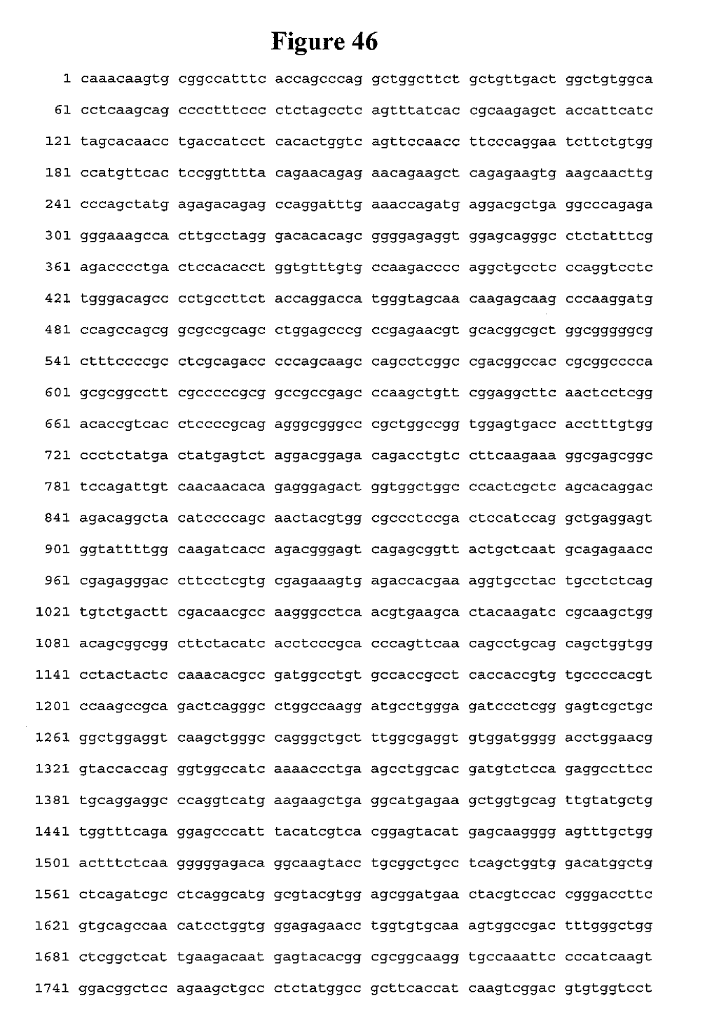

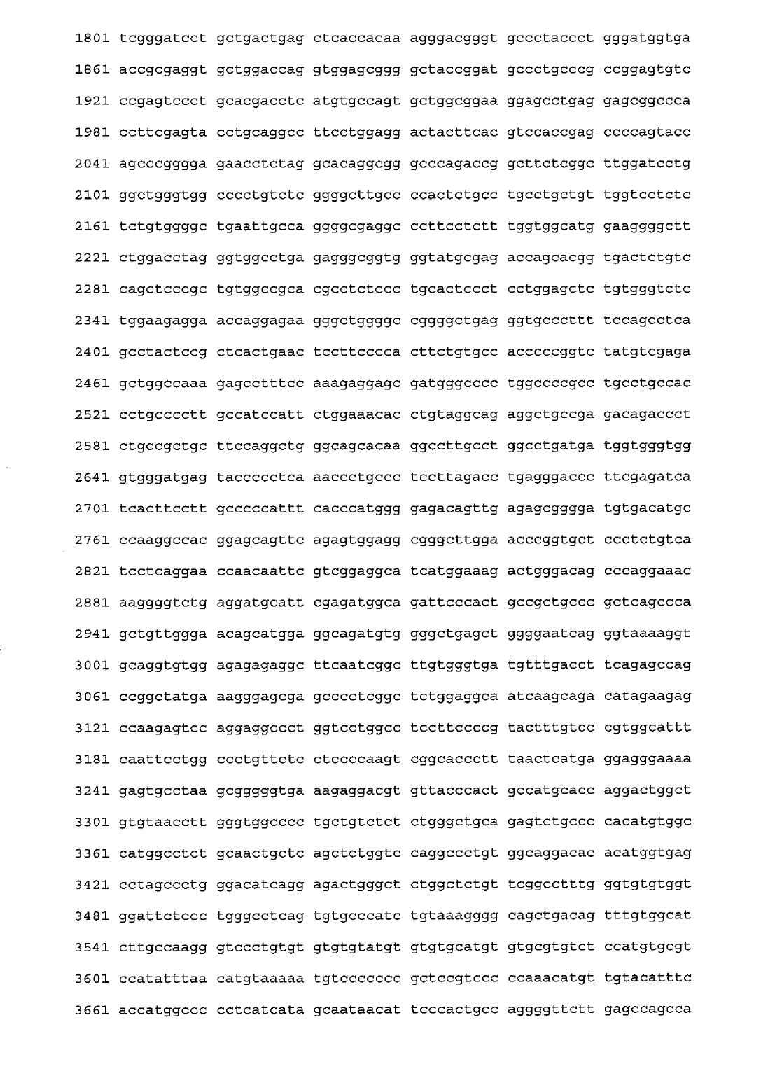

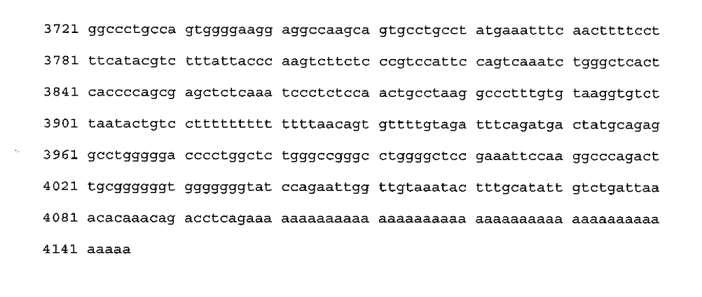

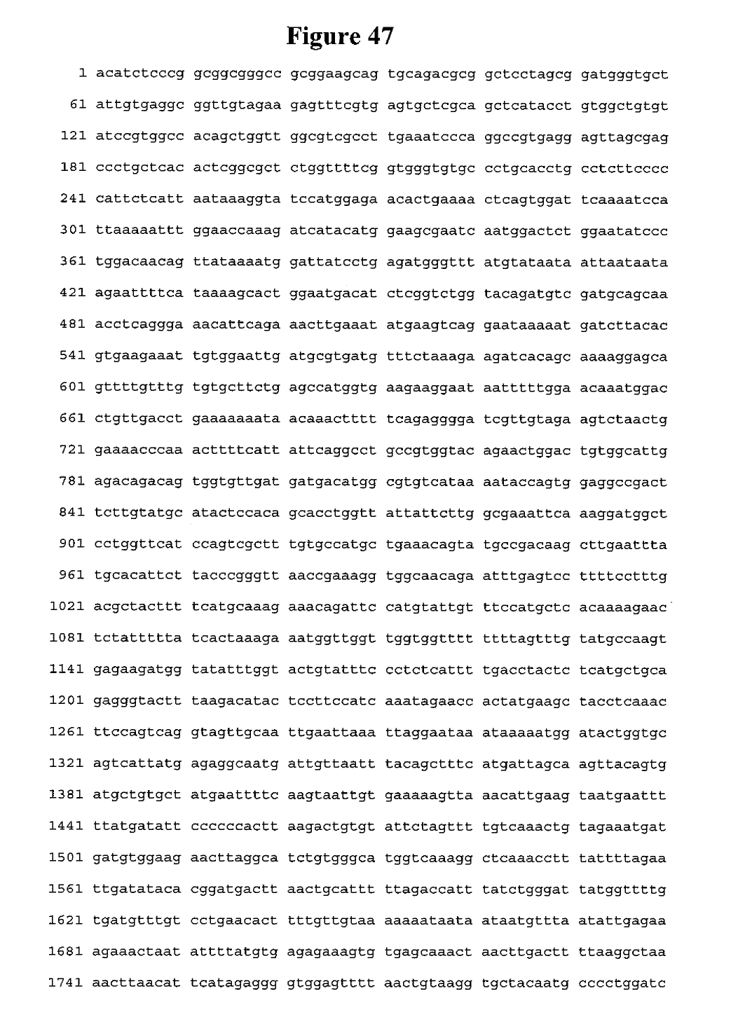

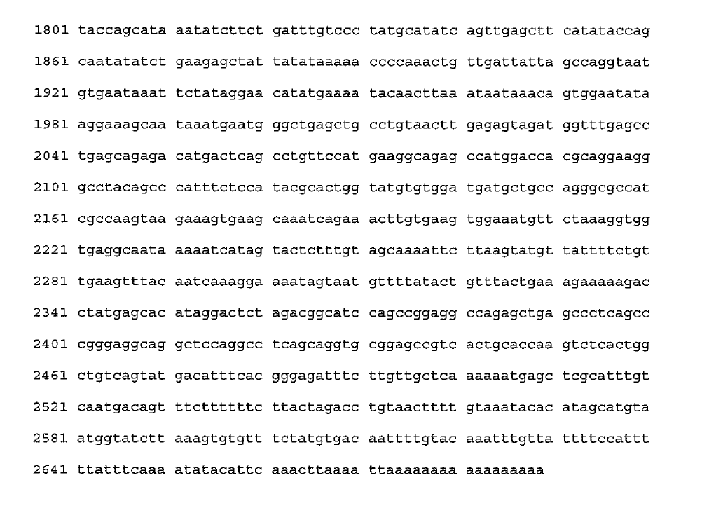

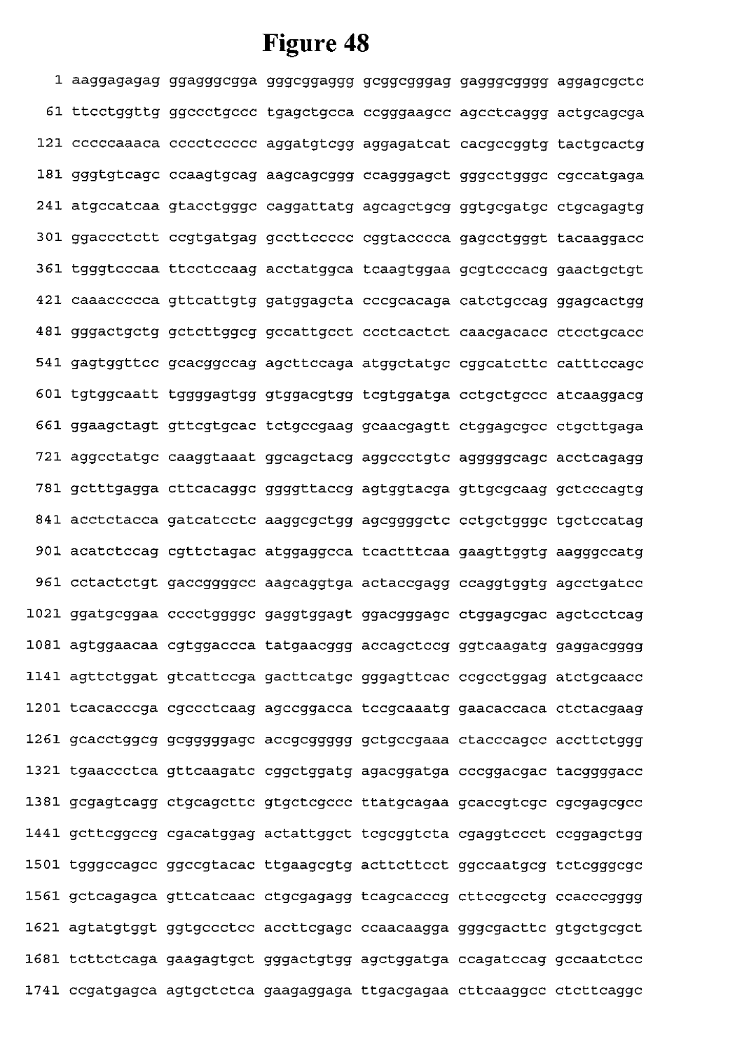

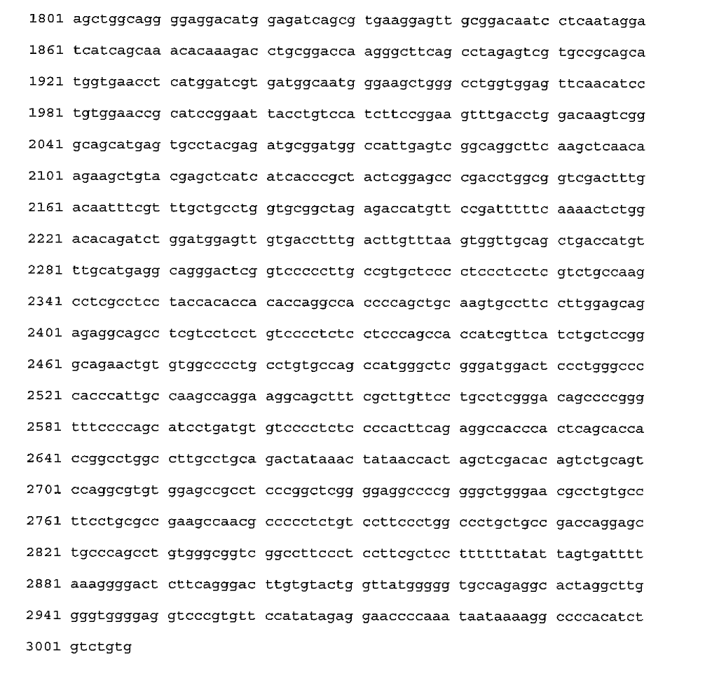

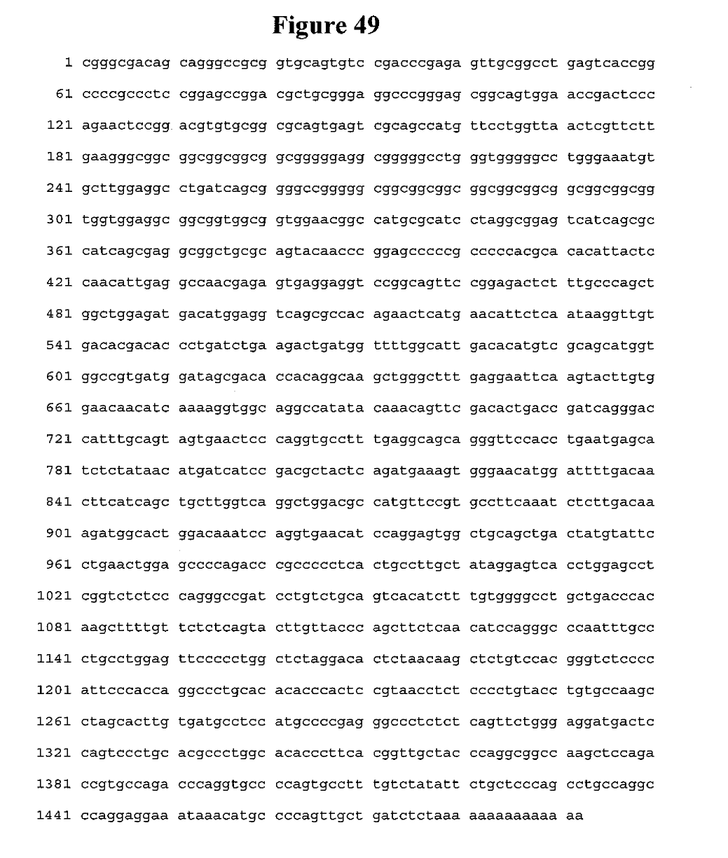

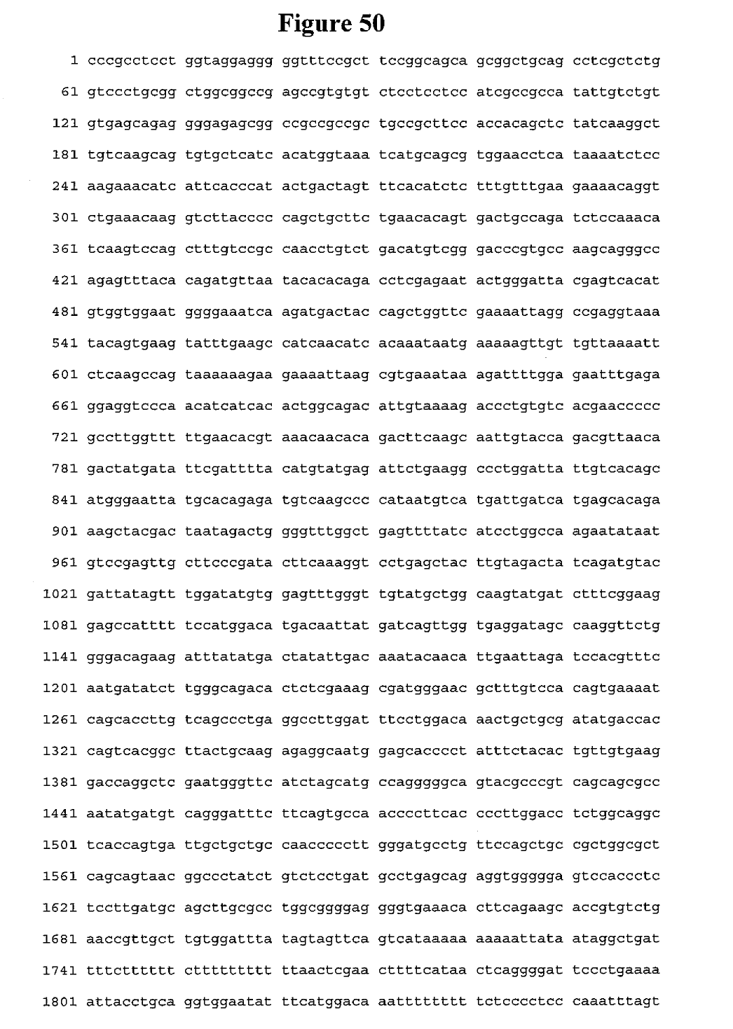

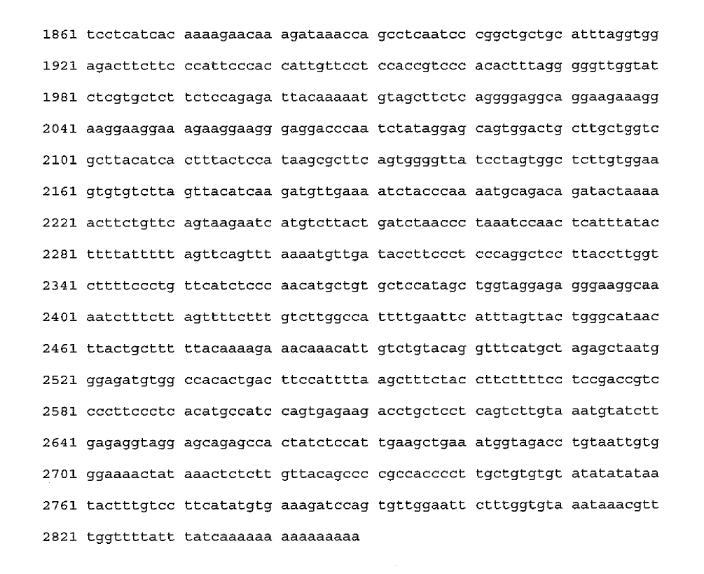

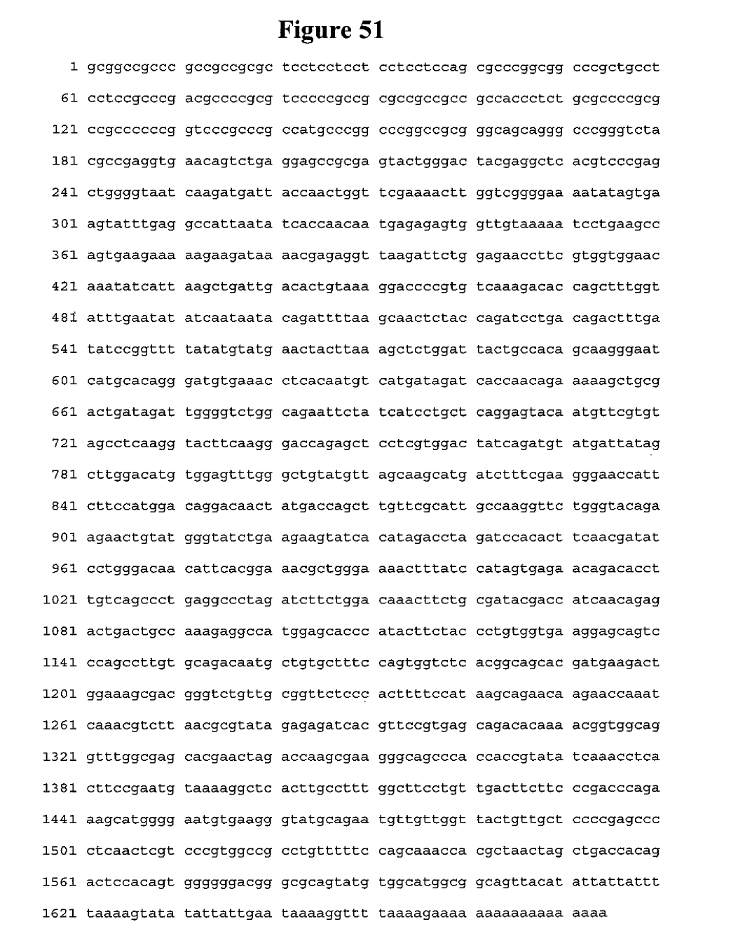

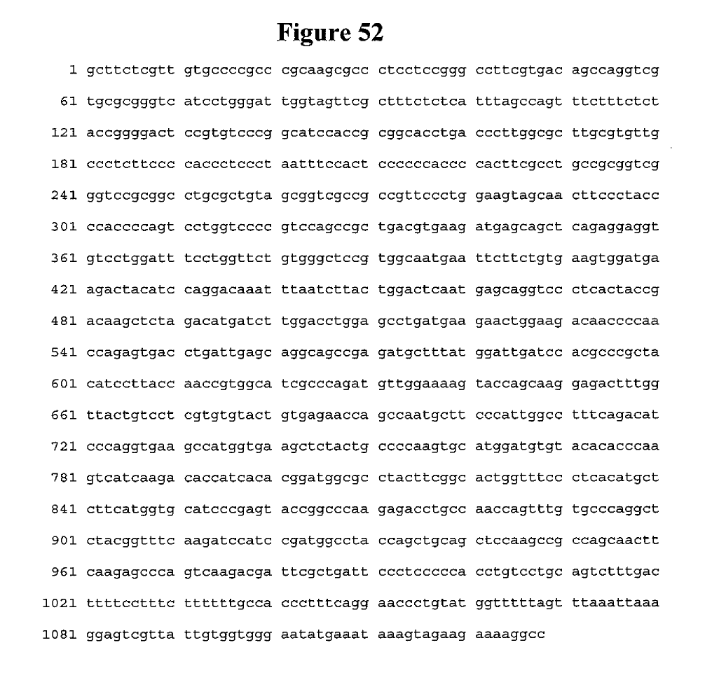

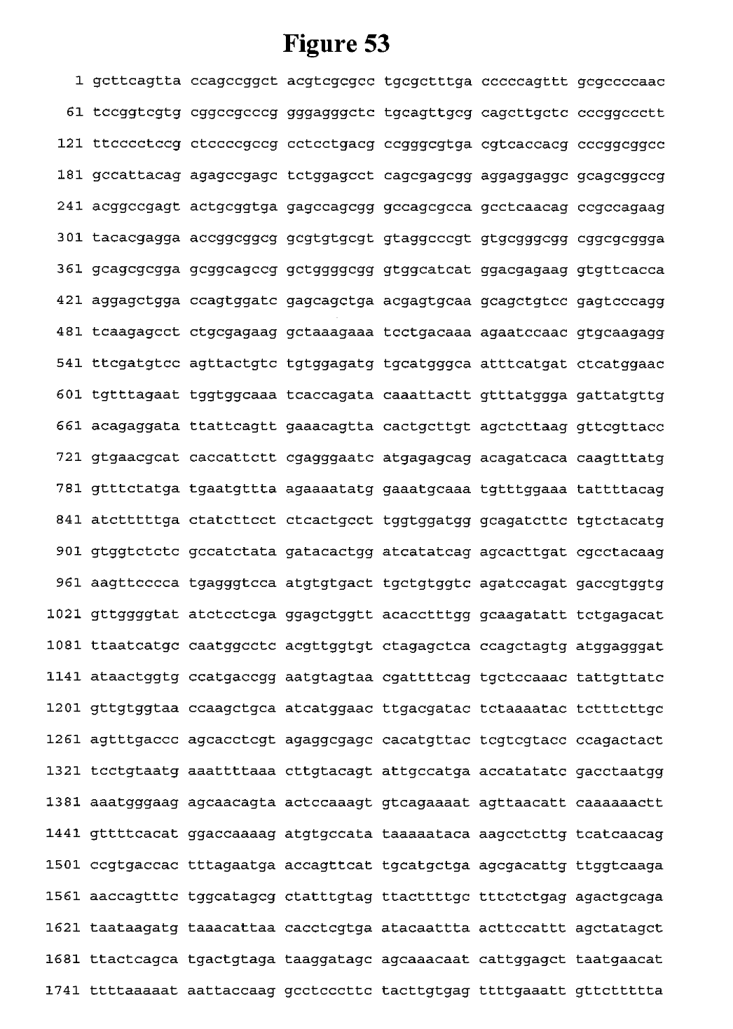

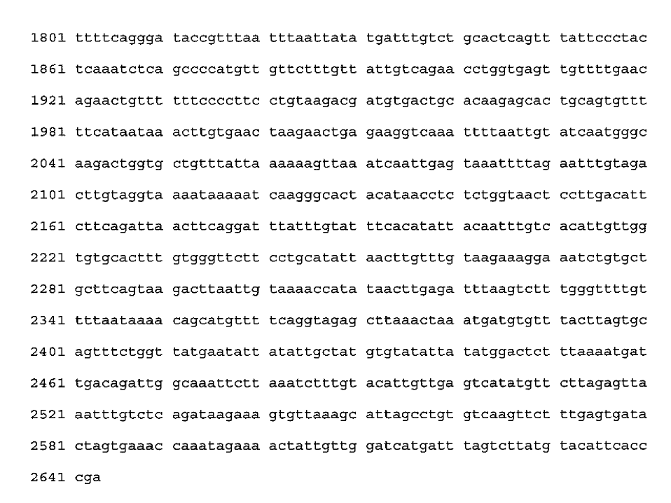

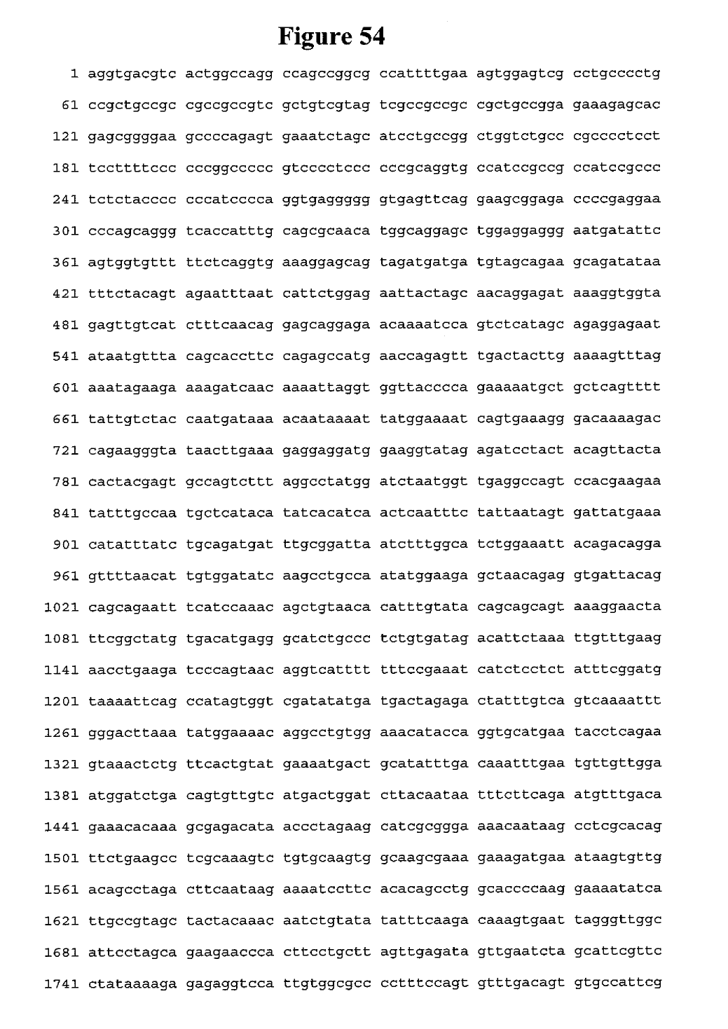

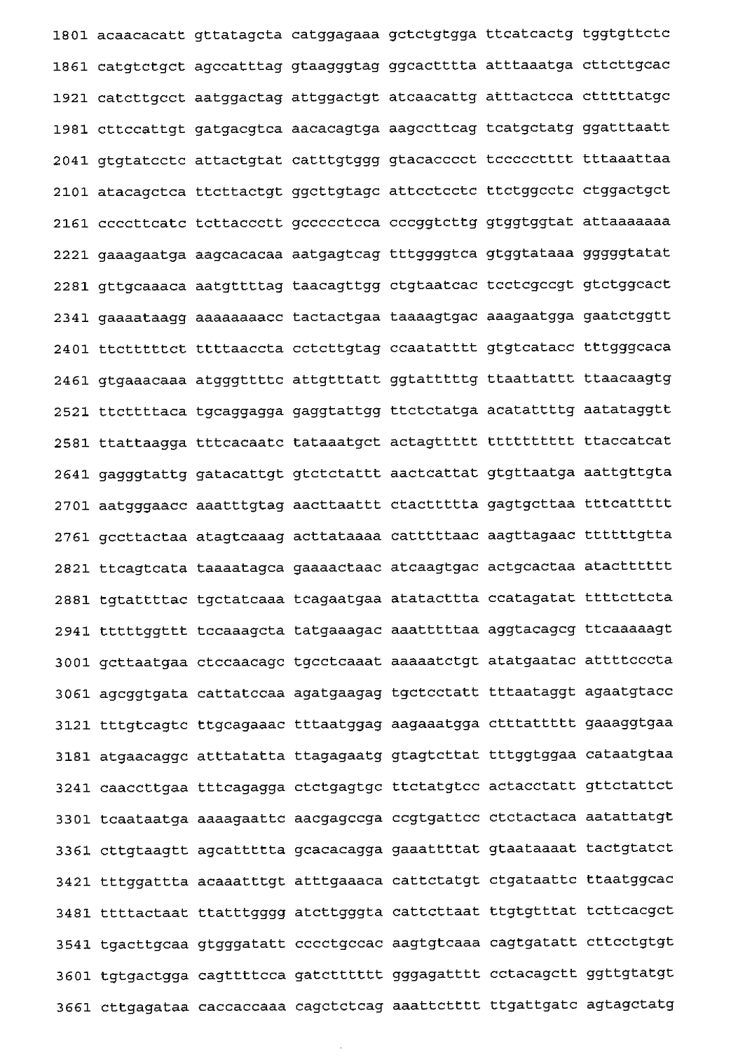

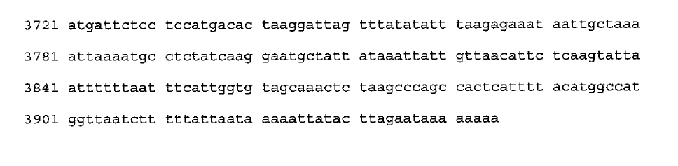

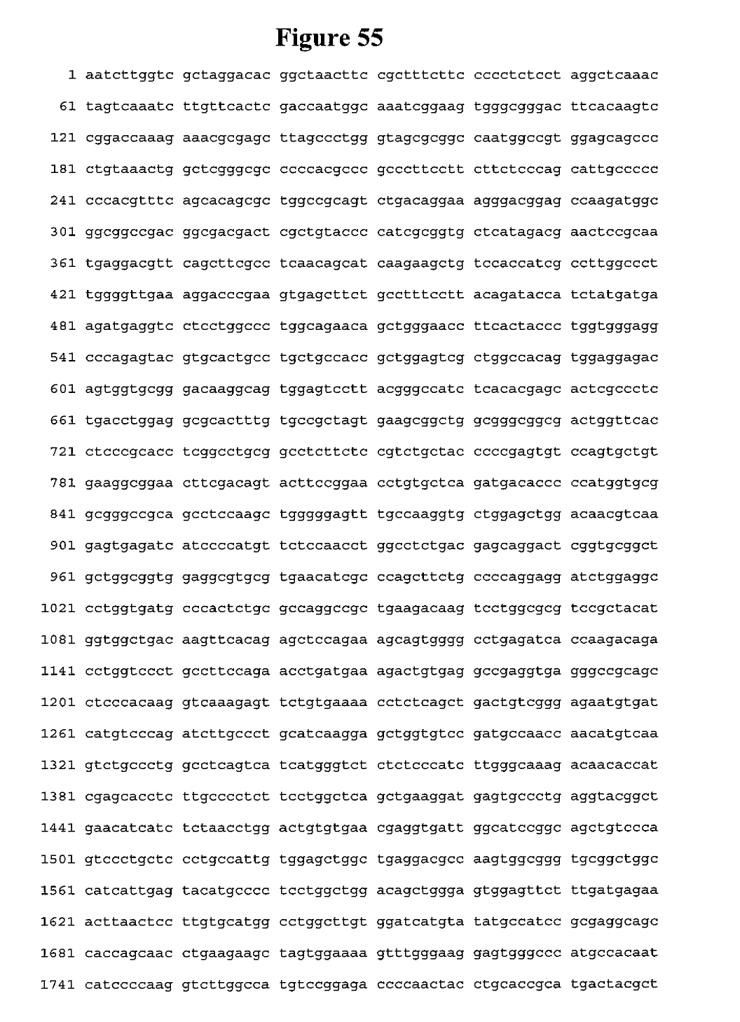

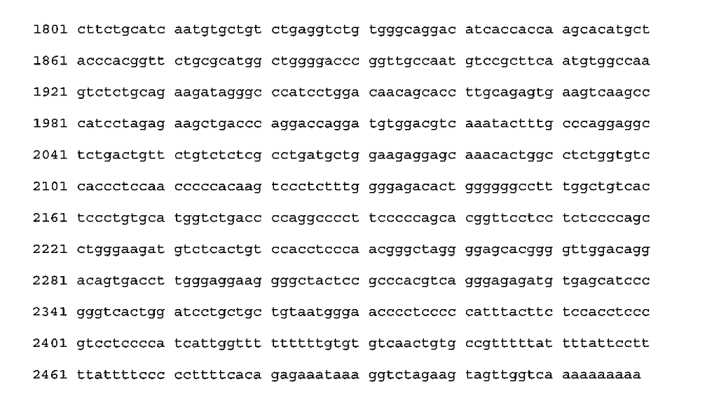

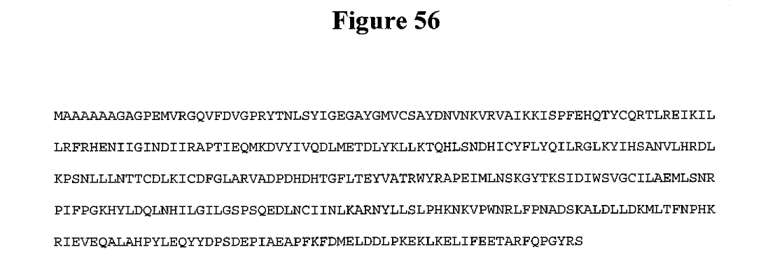

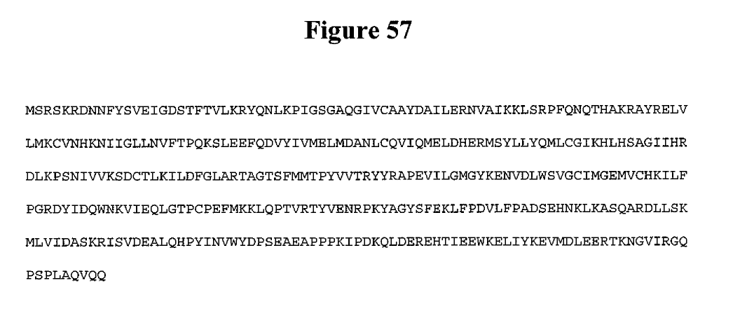

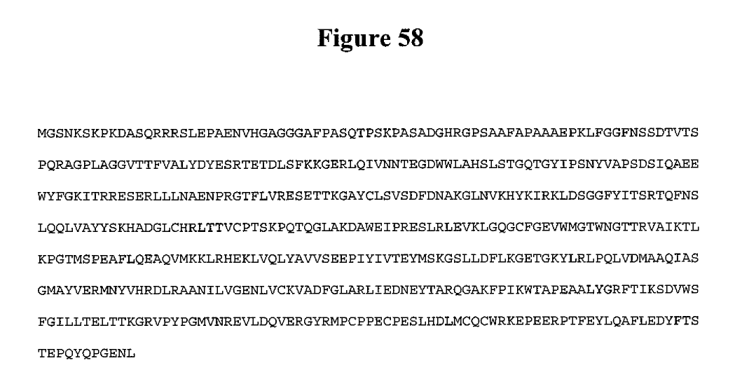

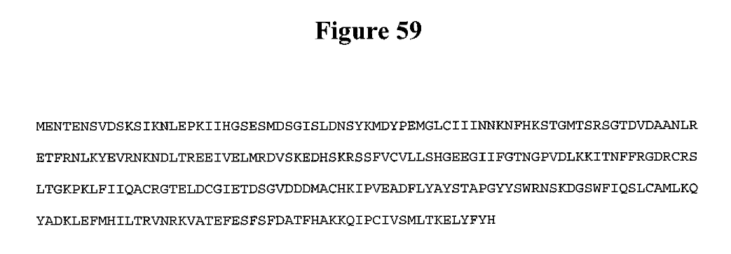

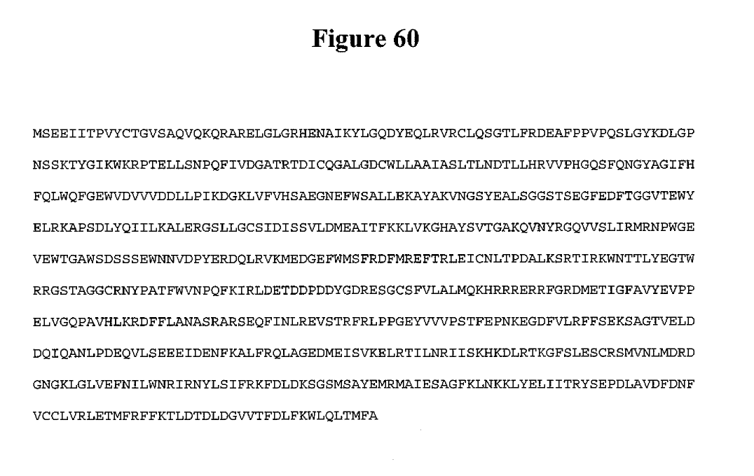

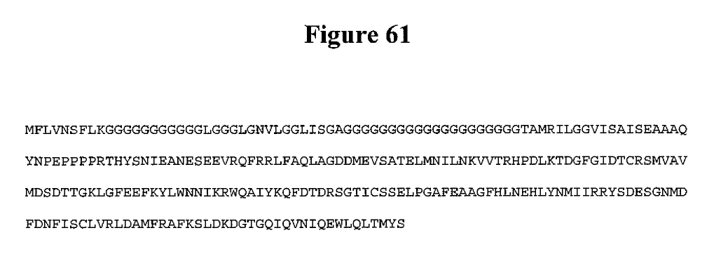

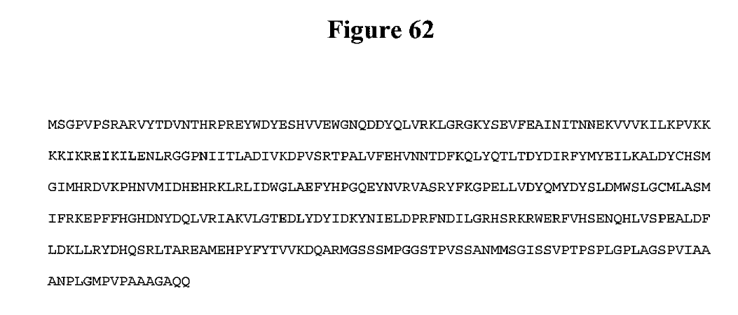

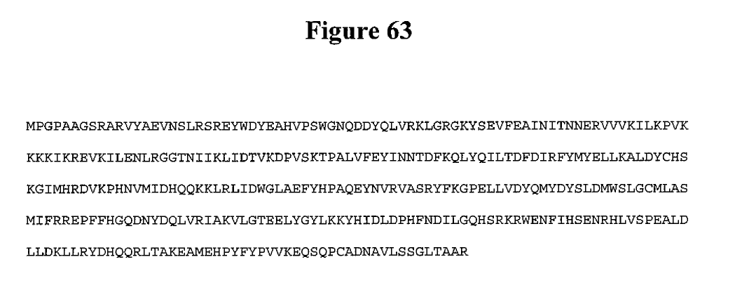

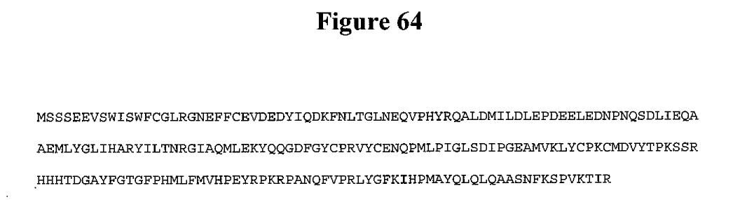

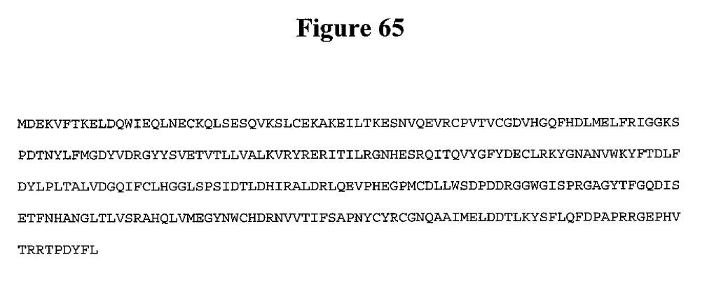

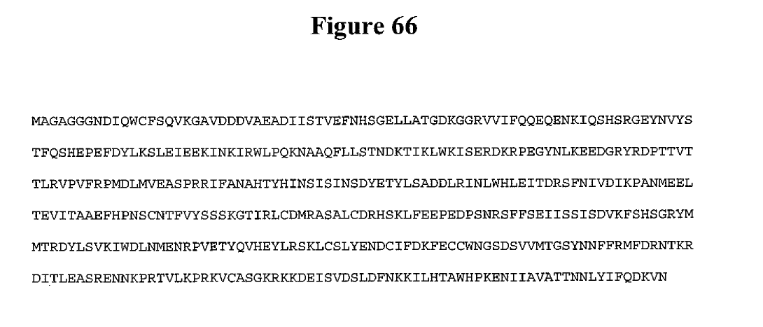

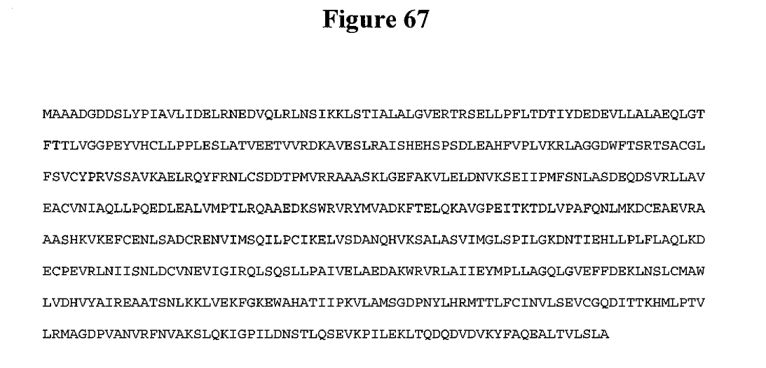

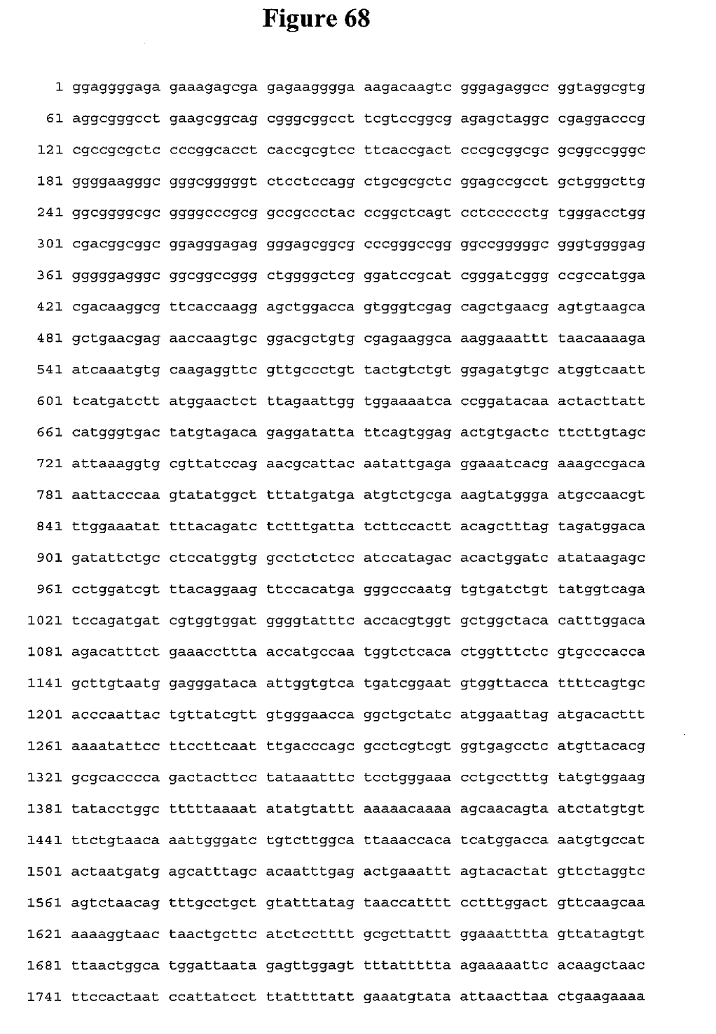

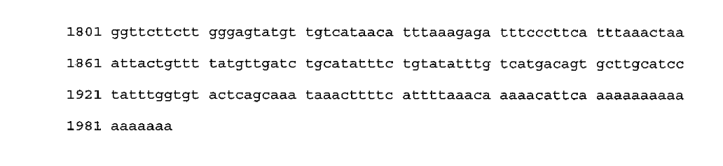

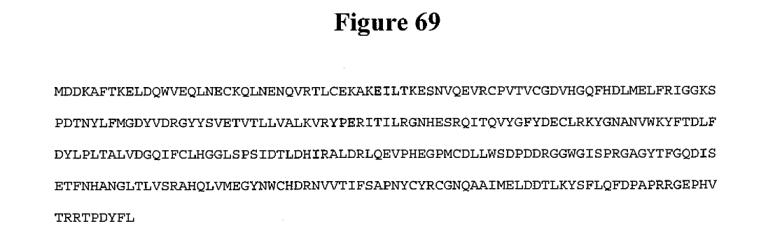

Within certain aspects, the siRNA may be between 15 and 50 contiguous nucleotides of the following sequences: (a) 5′-CCAGAUCGUU UGUACAGAAA UCUCGAGAUU UCUGUACAAA CGAUCUGG-3′ (SEQ ID NO: 7), which binds to the mRNA encoding the catalytic subunit of mouse protein phosphatase 1, alpha isoform (NM_{—}031868, FIG. 29, SEQ ID NO: 13); (b) 5′-UUUGAUGUUG UAGCGUCUCt t-3′ (SEQ ID NO: 29), which binds to the mRNA encoding the catalytic subunit of human protein phosphatase 1, alpha isoform (NM_{—}206873.1, FIG. 28, SEQ ID NO: 12); (c) 5′-GGCGUCCUUG AAAGUGUUAA AUCUCGAGAU UUAACACUUU CAAGGACGC-3′ (SEQ ID NO: 9), which binds to the mRNA encoding the catalytic subunit of mouse protein phosphatase 1, beta isoform (NM_{—}172707; SEQ ID NO: 15); and (d) 5′-UAAAACUCUA GGUGUAUACt t-3′ (SEQ ID NO: 32), which binds to the mRNA encoding the catalytic subunit of human protein phosphatase 1, beta isoform (NM_{—}002709.2; SEQ ID NO: 14). Within certain aspects, siRNA of the present disclosure may include one or more modification to confer in vivo stability such as, for example, a “tt” 3′-overhang as is exemplified in the human PP1 antisense siRNA sequences presented in SEQ ID NOs: 28 and 29.

Within other aspects, the present disclosure provides siRNA that bind to mRNA that encode CDK5, GSK3β, PKC, NCX1, P38, jnk, src, caspase 3, calpains, calcium kinase 2 (CK2), and protein phospatase 2 (PP2), and that comprise between 15 and 50, or between 18 and 40, or between 20 and 35, or between 21 and 30 consecutive nucleotides of the antisense sequence of SEQ ID NO: 16 (NM_{—}004935; CDK5); SEQ ID NO: 17 (NM_{—}001146156.1; GSK3β); SEQ ID NO: 18 (NM_{—}002737.2; PKC); SEQ ID NO: 19 (NM_{—}006153.4; NCK1); SEQ ID NO: 34 (NM_{—}002745.4; p38); SEQ ID NO: 35 (NM_{—}002750.2; JNK); SEQ ID NO: 36 (NM_{—}005417.3; SRC); SEQ ID NO: 37 (NM_{—}004346.3; caspase 3); SEQ ID NO: 38 (NM_{—}005186.2; calpain 1, large subunit); SEQ ID NO: 39 (NM_{—}001749.2; calpain, small subunit); SEQ ID NO: 40 (NM_{—}177559.2; CK2, alpha subunit); SEQ ID NO: 41 (NM_{—}001896.2; CK2, alpha prime subunit); SEQ ID NO: 42 (NM_{—}001320.5; CK2, beta subunit); SEQ ID NO: 43 (NM_{—}002715.2; PP2, catalytic subunit, α isoform); SEQ ID NO: 44 (NM_{—}002717.3; PP2, regulatory subunit B); SEQ ID NO: 45 (NM_{—}014225.5; PP2, regulatory subunit A); and SEQ ID NO: 58 (NM_{—}001009552.1; PP2, catalytic subunit, β isoform).

The extent of inactivation of CDK5, P38, jnk, src, caspase 3, calpains, CK2, PKC, GSK3α, GSK3β, PP1, PP2; and/or NCX1 correlates with axonal protection, which can be confirmed by (1) microscope assessment of axonal swellings, fragmentations, and structure of the node of Ranvier; (2) biochemical measurement of the transport of axonal components; and (3) electrophysiological assays such as calcium homeostasis. Each of these assays is well known in the art and is described in further detail within the presently disclosed Examples.

Because of the neural degeneration associated with Krabbe and related diseases is associated with psychosine accumulation within the central nervous system, siRNA of the present disclosure may be modified and/or conjugated to a component that permits the transfer of the siRNA across the blood-brain barrier of a patient. The reduction of CDK5, P38, jnk, src, caspase 3, calpains, CK2, PKC, GSK3α, GSK3β, PP1, PP2, and/or NCX1 activity of neurons may be achieved using intravenous delivery of small interfering RNA (siRNA) complexed with, for example, the chimeric rabies virus glycoprotein fragment RVG9R, which can cross the blood-brain barrier (BBB) and specifically binds to nicotinic acetylcholine receptors in neurons, to reduce the expression of CDK5, GSK3β, PKC, NCX1, and/or PP1. Thus, provided herein are siRNA that are conjugated to RVG-9R (NH₂-YTIWMPEBPR PGTPCDIFTN SRGKRASNGG GGRRRRRRRR R—COOH; SEQ ID NO: 11). Alternative peptides that may be suitably employed for achieveing transport of siRNA across the blood-brain barrier are well know in the art and are exemplified by those described in Banks and Kastin, Brain Res. Bull. 15(3):287-92 (1985) and Egleton and Davis, NeuroRx 2(1):44-53 (2005), which are incorporated by reference herein.

It is further contemplated that additional and/or synergistic activity may be achieved by the administration of two or more siRNA each of which is targeted against one or more effector of psychosine-mediated neurodegeneration, each of which leads to the downregulation of the mRNA encoding the effector. For example, compositions of the present disclosure may comprise two or more siRNA molecules each of which is targeted against one or more mRNA that encodes a kinase such as CDK5, P38, jnk, src, CK2, PKC, GSK3α and β, a phosphatase such as the Ser/Thr protein phosphatase PP1 and Tyr protein phosphatase PP2; and/or a sodium/calcium exchange proteins such as NCX1.

(b) Compositions Comprising Antagonists of Psychosine-Mediated Neuronal Degeneration

Within other embodiments, the present disclosure provides compositions comprising small-molecule and/or peptide antagonists of kinases such as CDK5 (SEQ ID NO: 24), GSK3β (SEQ ID NO: 25), P38 (SEQ ID NO: 46), jnk (SEQ ID NO: 47), CK2 (alpha subunit, SEQ ID NO: 52; alpha prime subunit, SEQ ID NO: 53; and/or beta subunit, SEQ ID NO: 54), src (SEQ ID NO: 48), and PKC (SEQ ID NO: 26); phosphatases such as the Ser/Thr protein phosphatase PP1 (α-isoform, SEQ ID NO: 20; β-isoform, SEQ ID NO: 22) and/or PP2 (α-isoform, catalytic subunit, SEQ ID NO: 55; α-isoform, regulatory subunit B, SEQ ID NO: 56; α-isoform, regulatory subunit A, SEQ ID NO: 57; β-isoform, catalytic subunit, SEQ ID NO: 59); proteases such as caspase 3 (SEQ ID NO: 49) and calpains (e.g., calpain 1, large subunit, SEQ ID NO: 50; calpain, small subunit, SEQ ID NO: 51); and sodium/calcium exchange proteins such as NCX1 (SEQ ID NO: 27), each of which is effective in reducing psychosine-mediated neurotoxicity, in particular psychosine-mediated axonopathy. Exemplified herein are compositions comprising the peptide GSK3β antagonist L803 (Tocris Bioscience, Ellisville, Mo.), which comprises the amino acid sequence Lys-Glu-Ala-Pro-Pro-Ala-Pro-Pro-Gln-pSer-Pro (SEQ ID NO: 28).

Another target to block psychosine induced axonopathy involves ion channels, including Nav1.2, Nav1.6, calcium channels and potassium channels since these are likely perturbed when axonal transport is defective. Twitcher neurons, upon electrical stimulation, exhibit longer latency times to remove intracellular Ca⁺⁺. This appears to be related to abnormal accumulation of the Na⁺/Ca⁺⁺ exchanger (NCX1). NCX1 is a known mediator of neuronal retention of Ca⁺⁺, which responds to exacerbated Na⁺ channel activity by reversing activity and increasing the influx of Ca⁺⁺ into the neuron. Stys et al., J. Neurosci. 12:430-439 (1992).

Ca⁺⁺ accumulation in the axons can also be reduced by blocking, or partially blocking, the activity of NCX1 by administering an inhibitor of NCX1, such as the blood-brain permeable antiarrythmic drug flecainide that decreases the exacerbated firing of Na⁺ channels and normalizes the exchange of Ca⁺⁺-mediated by NCX1. Flecainide as well as the anti-epilepsy drugs lamotrigine, topiramate, and carbamazepine were tested as part of the present disclosure for their potential to reduce axonal degeneration. Flecainide, in particular, has been successful in reducing excessive firing of sodium channels, decreasing sodium influx, and protecting axons in models of acute and chronic demyelination. Stys et al., Neuroreport 9:447-453 (1998); Leppanen and Stys, J. Neurophysiol. 78:2095-2107 (1997); Waxman et al., Brain Res. 644:197-204 (1994); Mueller and Baur, Clin. Cardiol. 9:1-5 (1986); Ransom and Brown, Neuron 40:2-4 (2003); Fern et al., J. Pharmacol. Exp. Ther. 266:1549-1555 (1993); and Black et al., Brain 129:3196-3208 (2006).

The extent of neuroprotection conferred by small-molecule and/or peptide antagonists disclosed herein may be assessed, as described within the Examples, with a transgenic Twitcher mouse that carries a fluorescent tag to allow direct visualization of axonopathy by confocal microscopy. The efficacy of compositions of the present disclosure may be tested by analysis of motor horn neurons in the lumbar/sacral spinal cord of the Twitcher mouse by measuring the number of healthy neurons following administration of the composition. Using the reporter transgenic Twitcher line (Twitcher-YFPax), which allows axonal marking by expression of fluorescent YFP, reversal of axonal pathology can be detected as early as P7, and at later time-points, which indicates progressive axonal generation.

Methods for the Treatment of Neurodegenerative Disorders

Within still further embodiments, the present disclosure provides methods for the treatment of a neurodegenerative disease in a patient suffering from a psychosine-mediated neurological disorder, which methods comprise the step of administering to the patient a composition comprising one or more siRNA molecule(s) each of which is targeted against, and leads to the downregulation of, mRNA that encode an effector of psychosine-mediated axonal degeneration. Within certain aspects, these methods comprise the step of administering to the patient a composition comprising one or more siRNA molecule(s) each of which is targeted against mRNA that encode CDK5, P38, jnk, src, caspase 3, calpains, CK2, PKC, GSK3α and β, PP1, PP2; and NCX1. Optionally, these methods may further comprise the step of administering to the patient a composition comprising GALC-expressing cell, such as a macrophage within a bone marrow sample from a suitable donor.

Within related embodiments, the present disclosure provides methods for the treatment of a neurodegenerative disease in a patient suffering from a psychosine-mediated neurological disorder, which methods comprise the step of administering to the patient a composition comprising one or more small molecule and/or peptide antagonist of an effector of psychosine-mediated axonal degeneration. Within certain aspects, these methods comprise the step of administering to the patient a composition comprising one or more small molecule and/or peptide antagonist of CDK5, P38, jnk, src, caspase 3, calpains, CK2, PKC, GSK3α and β, PP1, PP2; and NCX1. Optionally, these methods may further comprise the step of administering to the patient a composition comprising a GALC-expressing cell, such as a macrophage within a bone marrow sample from a suitable donor.

Typically, neuroprotective treatments targeting CDK5, P38, jnk, src, caspase 3, calpains, CK2, PKC, GSK3α and β, PP1, PP2; and/or NCX1, may be started at birth and continued into postnatal life, when neurons are most vulnerable and before the accumulation of GALC, and the corresponding metabolic correction of the enzyme deficiency, following BMT. Improvement of neuroprotection combined with BMT may be assessed based on axonal integrity, biochemical correction of the metabolic error, effect on nerve conduction, and in vivo non-invasive diffusion tensor MRI evaluation of myelination and demyelination.

The GALC deficiency associated with Krabbe disease leads to a defect in axonal transport and contributes to neurodegeneration and a significant reduction in synaptic-associated proteins in nerves distal to the spinal cord. This reduction, which is suggestive of defective vesicle transport, is observed as early as 15 days after birth, when demyelination has not yet begun and before the onset of clinical symptoms, further supports the early deficiencies in axonal transport that are associated with the deficiency in wild-type GALC expression.

Accordingly, depending upon the particular treatment regimen employed, the methods of the present disclosure comprise the step of administering a composition comprising one or more siRNA(s) and/or one or more antagonist(s) between 0 days and 60 days following the birth of the patient. More typically, the composition comprising one or more siRNA(s) and/or one or more antagonist(s) is administered to the patient between 0 days and 30 days following the birth of the patient, or between 0 days and 15 days following the birth of the patient or between 0 days and 7 days following the birth of the patient.

In those aspects of the present methods that further comprise the step of administering to the patient a composition comprising a GALC-expressing cell, the composition comprising a GALC-expressing cell is administered between 0 days and 120 days following the birth of the patient, or between 14 days and 90 days following the birth of the patient, or between 30 days and 60 days following the birth of the patient.

It will be understood that the methods disclosed herein may be advantageously applied to other demyelinating lysosomal storage disorders that are associated with psychosine accumulation and/or mediated by biological mechanisms identical or similar in molecular events to those observed in psychosine storage. Thus, in addition to their efficacy in the treatment of Krabbe disease, the methods disclosed herein are effective in the treatment of axonal degeneration in other lysosomal storage diseases and leukodystrophies such as metachromatic leukodystrophy, Canavan, Tay-Sachs, Niemann-Pick, Gaucher, Muccopolysacharidoses, Sandhoff, Morquio, Pelizaeus-Merzbacher and other diseases, which differ in genetic etiologies, that share with KD both myelin and axonal defects as well as the neurodegenerative process associated with aging. Because neurotrophic factors must be translocated to the cell body of the neuron by axonal transport to induce specific gene expression needed for neuronal survival and because this is a universal event for all neurons, impaired axonal transport results in inefficient trophic support of neuronal cells, progressive damage, and eventual death of the neurons. For example, it is belived that the muscle wasting seen in almost all myelin diseases is the consequence of defective axonal transport, loss of proper function of the associated motor neurons and muscle denervation.

All patents, patent application publications, and patent applications, whether U.S. or foreign, and all non-patent publications referred to in this specification are expressly incorporated herein by reference in their entirety.

### EXAMPLES

### Example 1

### General Methods

Animals

Breeder Twitcher heterozygous mice (C57BL/6J, twi/+, CD45.2 allele) and C57B16J mice carrying the CD45.1 allele were purchased from the Jackson Laboratory (Bar Harbor, Me.) and maintained under standard housing conditions. Analysis of the Twitcher mutation was performed as described in Dolcetta et al., J. Gene Med. 8:962-971 (2006). Twitcher mice were crossed with the Thyl.1:YFP line H+/+ Tg mice to produce TWI+/− thyl.1:YFP+/−. Mutant Twitchers expressing YFP (TWI-YFPax) were identified by PCR as described in Feng et al., Neuron 28:41-51 (2000) and Dolcetta et al., (2006). TWI and TWI-YFP genotypes were identified by PCR from tail DNA as described in Sakai et al., J. Neurochem. 66:1118-1124 (1996) and Feng et al., (2000).

Tissue Collection, Histology, and Immunohistochemistry

After performing all proper in vivo determinations, tissue was collected from mice deeply anesthetized and killed by perfusion with saline. Tissue dedicated for biochemistry was rapidly frozen on dry ice, while that dedicated to histology is postfixed in 4% paraformaldehyde. Additionally, ˜1 mm-thick pieces of sciatic, optic nerves, and spinal cord are cut in cross-sections and postfixed in 2% paraformaldehyde, 2% glutaraldehyde, 0.1 M cacodylate for electron microscopy.

Cryosections were prepared (20 μm) and mounted onto lysine-coated slides. For immunofluorescence staining, sections were dried for 15 minutes at 37° C., and washed in PBS to remove the OCT. The sections were then blocked/permeabilized in 5% bovine serum albumin (BSA), 0.5% Triton X-100/PBS for one hour at room temperature. The sections were then incubated with the primary antibody NeuN (Abcam; 1:100) or CGT (Abcam; 1:100) diluted in 2% BSA, 0.5% Triton X-100/PBE buffer overnight at 4° C., with mild agitation. After washing with PBS, slides were incubated with fluorescent secondary antibodies (Alexa 555) for 1 hour at room temperature, washed in PBS and counterstained with propidium iodide. Mouting was performed with Vectashield (Vector, Burlingame, Calif.). Confocal microscopy was performed using a confocal laser Meta Leica scanning microscope. In some experiments, counterstaining with dapi or propidium iodine was carried out before mounting. For the TUNEL staining, the assay was performed according to the manufacturer instructions (Roche). Briefly, the sections were dried at 37° C. for 15 minutes and washed in PBS to remove the OCT. The slides were then permeabilized in a solution of 0.1% Triton X-100, 0.1% Na Citrate in PBS for 2 minutes on ice. After two rinses in PBS, the slides were incubated with the mix of enzyme and label for 60 minutes at 37° C. in a humidified chamber. After two rinses in PBS, the slides were mounted with permount or the NeuN staining was performed.

After dissection and postfixation in 4% paraformaldehyde for 12 h, samples were saturated in 20% sucrose, mounted in OCT, and cryosectioned following well-established laboratory procedures. Galbiati et al., J. Neurosci. 27:13730-13738 (2007); Givogri et al., J. Neurosci. Res. 66:679-690 (2001); and Bongarzone et al., Methods 10:489-500 (1996). Briefly, appropriate samples were permeabilized with 0.1% Triton X-100, blocked with 5% BSA in PBS, and incubated overnight at 4° C. with primary antibodies (PP1, NF-160, Nav1.2 channel, Nav1.6 channel, Kv Channel, CASPR, GFAP, APP, NCX1, synaptophysin, α-synuclein, anti-α-tubulin, and glutamate receptor 2/3). After washes, slides were incubated for 2 h with secondary Alexa-labeled antibodies, counterstained with DAPI, and mounted. Donor-derived cells were recognized by CFP-fluorescence in slides examined by confocal microscopy.

Nissl Staining

Sections from the isolated tissues were prepared and stained with cresyl violet. 30 micron-thick sections were treated with 100% ethanol to remove the water and xylene to remove the fats. The sections were then re-hydrated in increasing dilutions of ethanol and in distilled water. The staining was performed for 5 min in 0.1% cresyl violet (prepared in distilled water and 3% acetic acid). Destaining was performed by dipping the slides in 1% acetic acid, 70% ethanol and in 1% acetic acid, 100% ethanol. The slides were then rinsed in 100% ethanol and mounted with permount. For the cell counting, only deeply stained motoneurons of the spinal cord ventral horn were counted as viable.

Hematopoietic Reconstitution and Chimerism

Infiltration of donor cells was evaluated by CFP fluorescence microscopy. FACS was employed to determine engraftment on blood withdrawn at P30 and at maximal survival time. Galbiati et al., J. Neurosci. 27:13730-13738 (2007) and Galbiati et al., J. Neurosci. (2008). Fifty μl of heparinized whole blood was obtained from the tail vein and incubated for 10 min at 4° C. with lysing buffer (155 mM ammonium chloride, 10 mM potassium bicarbonate, 0.1 mM EDTA, pH 8) to eliminate red blood cells. After washing, cells were centrifuged and fixed with 1% of paraformaldehyde in PBS. Reconstitution of myeloid, B-lymphoid, and T-lymphoid lineages were verified with appropriate PE-FITC labeled antibodies for Mac-1, B220, CD4, and CD8. Hsu et al., Blood 96:3757-3762 (2000). Similarly, engraftment of CFP+ donor cells in bone marrow preparations was done from material obtained from flushed femurs collected from killed mice. Analysis was conducted on a FACscan instrument after passing a total of 10⁴ events and analyzed with Cell Quest software. Galbiati et al., J. Neurosci. 27:13730-13738 (2007).

Globoid Cell Counting

Globoid cells, a hallmark of KD, were identified in cryosections from spinal cord, brain, and optic and sciatic nerves with peroxidase-BS-I-B4 lectin (Bandeirae simplicifolia, Sigma). Slides were rinsed with PBS, quenched with 10% methanol and 3% oxygen peroxide, and incubated with peroxidase-conjugated lectin overnight at 4° C. Color development was carried out by incubation with diaminobenzidine and oxygen peroxide. After sequential dehydration, clearing and mounting on Permount, samples were observed and lectin⁺ cell density (number of lectin⁺ cells per area) was assessed by counting in an upright Zeiss microscope. Galbiati et al., J. Neurosci. Res. (2009).

Cell Cultures

The procedure for primary cell culture of glial cells has been described in detail in Bongarzone et al., Methods 10:489-500 (1996). Cell cultures of cortical neurons were prepared as previously described. Kaech and Banker, Nat. Protoc. 1:2406-2415 (2006). E16 pregnant females were sacrificed, the brains of the litter were collected, and the cortex was isolated. The brain was chopped, treated with 0.25% trypsin and then passed through a fire polished pipette. The cells were then plated in DMEM (Mediatech) supplemented with 10% fetal bovine serum (FBS) and, after 2 hours, the medium was changed to Neurobasal medium supplemented with B27. For cell survival, the MTT assay (Chemicon) was performed as indicated by the supplier. Briefly, 5000 cells/well were plated in a 96 well plate, and the stimuli were administered for 24 hours. At the end of the incubation time, the MTT reagent was added and, after 4 hours, the reaction was stopped and the absorbance was read at 570 nm. NSC34 cells were grown in DMEM supplemented with 5% FBS, L-glutamine (Gibco) and penicillin/streptomycin (Gibco). For the experiments, the cells were serum deprived for 12 hours before the addition of the different treatments. Psychosine, D-Sphingosine, and C6-Ceramide were purchased from Sigma and resuspended in ethanol to the desired concentration.

Inflammation Analysis

To study the long-tem effect of the treatments on neuroinflammation, protein extracts from spinal cord, brain, and optic and sciatic nerves were prepared at a concentration of 100 μg/ml in the recommended lysis buffer and processed using the RayBio Mouse Cytokine Antibody Array G series 1000 according to RayBiotech protocols. IFNγ, IL-1α, IL-1β, IL-2, IL-4, IL-6, RANTES, SDF-1, and TNFα and other cytokines were quantitatively analyzed by an Elisa-capture-based method. Results were acquired by laser scanning and measurement of fluorescence intensity in the array using a Confocal dual-laser scanner Scan Array Lite (Perkin Elmer). Continuous monitoring of body weight and signs of alopecia also helped to evaluate development of graft-vs-host disease.

GALC Correction and Psychosine Accumulation in Treated Twitchers

Both GALC activity and psychosine accumulation were measured in extracts from brain, spinal cord, and optic and sciatic nerves of treated mice at P30 and at maximal survival. Tissues were homogenized in deionized water with proteinase inhibitors (Roche) and GALC activity measured using LRh-6-GalCer (N-lissamine rhodaminyl-6-aminohexanoylgalactosyl ceramide) as described (Dolcetta et al., J. Gene Med. 8:962-971 (2006) and Marchesini et al., Chem. Phys. Lipids 53:165-175 (1990)) with results expressed as mean nmol/mg protein/h from at least 5-7 animals per group.

Psychosine was determined by liquid chromatography tandem mass spectrometry (LC-MS/MS) of methanol/chloroform extracts then partially purified on a strong cation exchanger column. After evaporation to dryness, each residue was dissolved in 200 μl of methanol containing 5 mM ammonium formate, and 10 μl aliquots were analyzed using LC-MS/MS. The HPLC system included Shimadzu (Columbia, Md.) LC-10Advp pumps with a Leap (Carrboro, N.C.) HIS PAL autosampler. Psychosine was measured using a Waters XTerra 3.5 μm, MS C18, 2.1×100 mm analytical column. Positive ion electrospray tandem mass spectrometry was performed using an Applied Biosystems (Foster City, Calif.) API 4000 triple quadrupole mass spectrometer with a collision energy of 29 eV for psychosine and 37 eV for the internal standard, lyso-lactosylceramide. The dwell time was 1.0 s/ion during multiple reaction monitoring. Results were expressed as mean pmol psychosine/mg protein from at least 5-7 animals per group. Galbiati et al., J. Neurosci. 27:13730-13738 (2007).

Electron Microscopy

Tissue for EM was rapidly collected after dissection, immersion-fixed in 2% paraformaldehyde:2% glutaraldehyde for 2-4 hours, postfixed in osmium tetroxide, and ultrathin sections counterstained with uranile/lead. Givogri et al., J. Neurosci. 67:309-320 (2002). Tissue was embedded in epoxi resin and 5 to 10 one-p.m semithin sections from the lumbar spinal cord and from sciatic nerves were stained with toluidine blue and analyzed by light microscopy under a 100× objective. Myelinated and non-myelinated axons in the ventral and dorsal columns and in the sciatic nerve were counted. Ultrathin sections (60 nm-thick) were were cut with a Diatome diamond knife on a Leica Ultracut UCT microtome, collected on Formvar-coated one-hole grids, and counterstained with uranile/lead. Samples were observed at 10,000× or 50,000× magnification in a Leo 850 electron microscope. Calibers of at least 500 axons and the corresponding myelinated caliber were determined for each sample. G-ratio, a well-characterized parameter to quantify myelination, was calculated as the ratio of the axonal to the myelinated diameter. Axonal pathology (swellings, accumulation of membranous organelles, etc.) was studied from the same samples at the EM level.

Expression Analysis by Quantitative PCR (QPCR)

Samples of RNA were prepared using Trizol as recommended by the manufacturer (Invitrogen). cDNA derived from approximately 100 ng of starting RNA was used for real-time QPCR on a Bio-Rad iCycler4 with the Bio-Rad Sybr Green Supermix. The following target genes were tested: NCX1, Nav1.2, Nav1.6, and GAPDH in 25 μl reactions. Relative quantification was obtained as described. Hirokawa et al., J. Cell Biol. 114:295-302 (1991).

The RNA from cultured cortical neurons was purified with Trizol (Invitrogen), according to the manufacturers instructions. The cells were left in trizol for 5 minutes, then the trizol was collected and mixed with chloroform. The samples were shaken and then spun down. The aqueous phase was collected and the RNA was precipitated with isopropanol overnight at −20° C. The RNA was collected and washed with 75% ethanol. The quality of the RNA was determined by measuring the absorbance at 260 nm and 280 nm. Retrotranscription was performed with the Superscript III (Invitrogen) according to the manufacturer instructions. Real Time PCR analysis was performed with primers specific for GALC, CGT, and the 60S acidic ribosomal protein P0 (RPLP0), that was used as the internal control. The primers were tested on a standard curve and the efficiency and the correlation coefficient were higher than 90% and 0.990, respectively. The results of the PCR were calculated with the Delta-delta Ct method. PCR primers are presented in Table 1.

TABLE 1

| Sequence | | |
|---|---|---|
| Identi- | Primer | |
| fier | Name | Primer Sequence |
| SEQ ID | GALC | 5′-CTGGATACTCTATGGCTCCTTGAC-3′ |
| NO: 1 | Forward | |
| SEQ ID | GALC | 5′-AGTGGTGA GCG TAAATATCTCGTC-3′ |
| NO: 2 | Reverse | |
| SEQ ID | CGT | 5′-CAATAATCCCAGTTATCGGCAGAG-3′ |
| NO: 3 | Forward | |
| SEQ ID | CGT | 5′-TCCAATAGGTAGTCCGATTGACAG-3′ |
| NO: 4 | Reverse | |
| SEQ ID | RPLP0 | 5′-CACGAAGCTA ACGACTATCGC-3′ |
| NO: 5 | Forward | |
| SEQ ID | RPLP0 | 5′-CTCTAGGGACTCGTTCGTGC-3′ |
| NO: 6 | Reverse | |

PP1 Enzyme Activity Assay

Samples were processed for quantitation of PP1 with the Molecular Probes RediPlate™ 96 EnzChek® Serine/Threonine Phosphatase Assay Kit (Molecular Probe), as described by the manufacturer. Samples were homogenized in buffer (50 mM Tris-HCl pH 7.0, containing 0.1 mM CaCl₂, 125 μg/ml BSA, 0.05% Tween 20) using a IKA Ultra-Turrax T8 homogenizer. An equal amount of protein was loaded in each well of the 96-well plate and fluorescence was read at an excitation of 370 nm and an emission of 460 nm.

Expression Analysis by Immunoblotting

Tissues were isolated and either frozen for long term storage or directly homogenized in lysis buffer (1 mM PMSF, 2 mM Sodium Orthovanadate, 1 mM NaF, 20 mM Tris HCl pH 7.4, 1% Triton X100, 150 mM NaCl, 5 mM MgCl₂, 300 nM Okadaic acid). Samples were then briefly sonicated on ice and spun down at 5000 rpm for 5 min to remove the debris. The amount of protein of the supernatant was then quantified with the Bradford assay (Biorad) and equal amount of proteins were loaded on a 4-12% Bis-Tris gel (Invitrogen). After protein determination, samples were diluted to the same concentration and 10-20 μg of total protein electrophoresed on 4-12% Tris-glycine Nupage (Invitrogen) gels at 80 V in MOPS-SDS running buffer. After at 80 mV the gels were transferred for 2 hour at 120 PVDF on a PVDF membrane (Biorad). The membrane was blocked in 5% milk, 1% BSA, 0.05% Tween 20 in Tris Glycine buffer (blocking solution), then probed with primary antibodies overnight at 4° C. and with the secondary horse radish peroxidase conjugated antibodies for 1 hour at room temperature. Antibodies were prepared in blocking solution. The primary antibodies were: anti-actin (Sigma), anti-CGT (Abnova), anti-GALC (Santa Cruz), anti-HSP60 (Santa Cruz), anti-SNAP25 (Abcam), anti-active Bax (Santa Cruz), anti-Bad (Santa Cruz), anti-MBP (Chemicon), anti-P0 (Chemicon), anti-KHC H2, anti-KLC L2, anti-APP, anti-NCX1, anti-synaptophysin, anti-synaptotagmin, anti-GAPDH, and anti-PP1 catalytic subunit antibodies. The membrane was washed for at least one hour after the primary and secondary antibody incubations and developed in the Enhanced Luminescence kit (Thermo Scientific). After exposure, the bands were quantified with the software imageJ and the genes of interest were normalized to the relative loading control.

Membrane Action Potential and Calcium Electrophysiology

Coronal slices covering the hippocampal formation were incubated for 1 h at 34° C. in oxygenated artificial cerebrospinal fluid (ACSF) composed of 125 mM NaCl, 26 mM NaHCO₃, 25 mM glucose, 2.5 mM KCl, 1.25 mM NaH₂PO₄, 2 mM MgCl₂, 2 mM CaCl₂ and then moved to X-Y translational stage mounted on an air table. Cells were visualized using a 60× water-immersion lens in an Olympus BX50WI microscope. Whole-cell recordings were obtained from hippocampal and cortical pyramidal cells (5-10 cell/slice) using an Axon Instruments Multiclamp 7008 amplifier, Digidata 1322A, and pClamp 9 software and borosilicate recording pipettes filled with solution containing 140 mM potassium gluconate, 4 mM NaCl, 10 mM Hepes, 4 mM ATP, and 0.3 mM GTP at 290-295 mOsm and pH 7.25-7.3.

Voltage responses to current were measured using current step injections (from −250 pA to 200 pA in intervals of 50 pA). Action potentials were produced by short-current injections. Calcium responses to action potentials were measured using fluo-4 (Kd 345 nM, a calcium-sensitive dye, Invitrogen) and a Cooke Sensicam CCD camera (Imaging Workbench 6.0).

Stereology

For unbiased stereological studies, 30-μm-thick spinal cord cross-sections were selected (one every 10 sections) and stained accordingly. Quantification of positive cell markers was performed with design-based stereology system (StereoInvestigator version 8, MBF Bioscience, Williston, Vt., USA). Briefly, the spinal cord ventral horns were traced under 5× objective and all cell markers were counted under 63× objective (Zeiss AX10 microscope, Carl Zeiss Ltd., Hertfordshire, England). The sampling parameters were set up according to the software guide to achieve the coefficient of error range between 0.09 and 0.12 using the Gundersen test, normally a counting frame size 100×100 μm, optical dissector height 20 μm, and an average of 10 sampling sites per section were chosen.

Sciatic Nerve Ligation

Animals were anesthetized by intraperitoneal injection of avertina. The sciatic nerve of the right leg was then exposed and a surgical thread was used to ligate the nerve. The wound was then closed and, 6 hours after the surgery, the tissue was collected. The proximal and distal stumps were collected from the ligated nerve, and the controlateral, unligated nerve was used as control of unaltered transport. The tissue was processed for immunoblot analysis or TEM.

Vesicle Motility Assays in Isolated Axoplasm

Axoplasm was extruded from giant axons of the squid Loligo pealii (Wood Hole Marine Biological Laboratory) as described previously. Szebenyi et al., Neuron 40:41-52 (2003) and Morfini et al., Nat. Neurosci. 9:907-916 (2006). Sphingolipids were diluted into X/2 buffer (175 mM potassium aspartate, 65 mM taurine, 35 mM betaine, 25 mM glycine, 10 mM HEPES, 6.5 mM MgCl2, 5 mM EGTA, 1.5 mM CaCl₂ and 0.5 mM glucose, pH 7.2) supplemented with 2-5 mM ATP and 20 μl was added to perfusion chambers. Preparations were analyzed on a Zeiss Axiomat with a 100×, 1.3 n.a. objective, and DIC optics. Hamamatsu Argus 20 and Model 2400 CCD cameras were used for image processing and analysis. Organelle velocities were measured with a Photonics Microscopy C2117 video manipulator (Hamamatsu).

Statistical Analysis

Results were the average from 3-4 different experiments and are expressed as mean±SE. Data were analyzed by the Student's t test and p values <0.05 were considered statistically significant.

Example 2

### Significant Reconstitution of GALC Activity and Myelin Preservation in Twitcher Mice after Bone Marrow Transplantation

This Example demonstrates that BMT (alone or in combination with gene therapy) is a meaningful approach to prevent some, but not all, of the pathologies associated with KD.

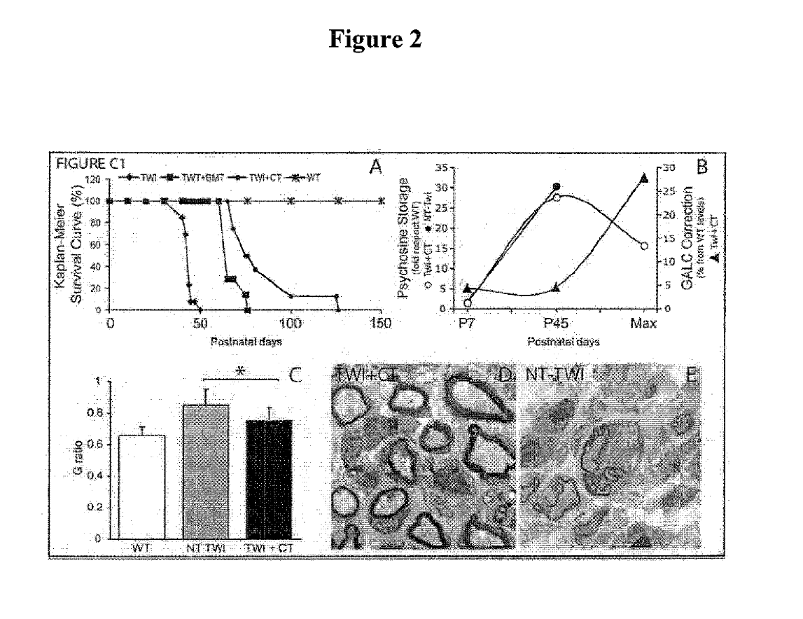

Healthy bone marrow was transplanted to newborn Twitcher mice, a model for KD, in combination with lentiviral gene therapy. These mice had longer survival (FIG. 2A), improved myelination (FIGS. 2C-E), fewer globoid cells, and amelioration of motor defects (not shown) as compared to untreated Twitcher mice. Cerebral GALC activity remained <5% of the normal value during the first 2 months after treatment but was increased to ˜30% with respect to normal levels in long-lived mutants (FIG. 2B). This paralleled the kinetics of brain infiltration by donor-derived macrophages (not shown). During the first weeks after treatment, brain psychosine accumulated similarly in both treated and non-treated Twitcher mice, but it was significantly reduced in the brain of long-lived treated mice (FIG. 2B). In long-lived treated Twitcher mice, myelination was significantly protected, with G-ratio in axons from the sciatic nerve indicating significant preservation of myelinated axons in nerves from the treated mutant (FIG. 2C). Myelinated axons were seen in the sciatic nerve of long-lived Twitcher-CT mice (FIG. 2D) in contrast to the abundance of nude axons and poor-quality myelin seen in untreated mice (FIG. 2E).

### Example 3

### Psychosine is Accumulated in Twitcher Neurons

The expression of GALC was examined in granule neurons (GN) of wild type mice. Granule neurons represent the most abundant neuron type in the CNS and their axons are generally not myelinated. Thus, axonal/neuronal defects are dissociated from demyelination.

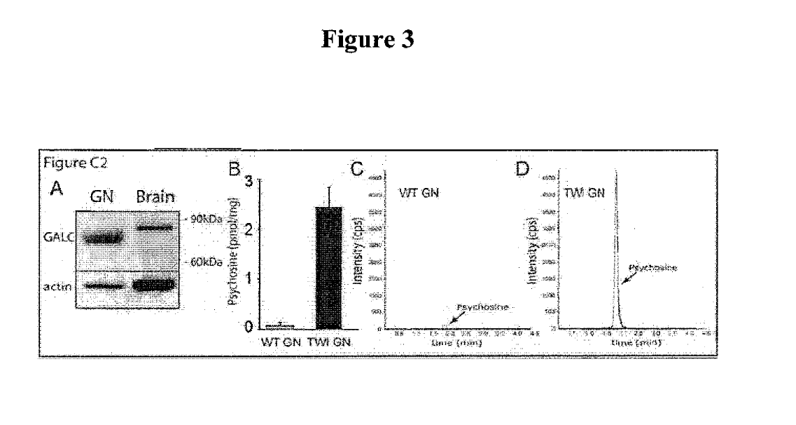

GN were isolated from early postnatal cerebellum of wild type pups and cultured up to 8 days in vitro. GN were >95% enriched in neurons as determined by triple immunohistology for NeuN (neuron), GFAP (astrocytes), and 04 (oligodendrocytes). Immunoblotting using anti-GALC antibodies revealed a single band of −75 kDa in protein extracts from GN while extracts from brain showed a band of slightly higher size (FIG. 3A). Various sizes ranging from 50 to 80 kDa have been reported. Wenger et al., Mol. Genet. Metab. 70:1-9 (2000).

Twitcher GN accumulation of psychosine was measured using mass spectrometry analysis. During an 8-day incubation, mutant neurons significantly accumulated psychosine (˜2.5 pmol/mg, FIG. 3B). The LC-MS-MS chromatograms (presented in FIGS. 3C and 3D) show the detected peak of psychosine in wild-type and Twitcher neurons, respectively.

### Example 4

### Defective Axonal Transport in Twitcher Neurons

This Example demonstrates that neurons of GALC deficient Twitcher mutants develop defective axonal transport.

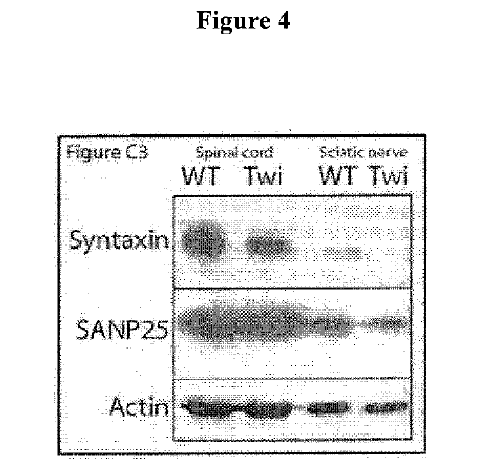

Because granules accumulate the potent toxin psychosine and because axonal transport is integral to neurons, Twitcher mice were evaluated for impaired axonal transport. Assuming that perturbed axonal transport would be reflected in an altered distribution of proteins associated with synaptic vesicles, the abundance of two such proteins, syntaxin and SNAP25, were measured in extracts isolated from the spinal cord and from distal sciatic nerves of Twitchers at P15 (a week before demyelination is detectable in the mutant). lmmunoblot analysis using specific antibodies revealed about 50% less SNAP25 in Twitcher sciatic nerves compared with WT nerves at P15 and almost complete absence of syntaxin in the mutant nerves (FIG. 4).

### Example 5

### Degeneration of Twitcher Neurons During Postnatal Development

This Example demonstrates a progressive degeneration in mutant neurons in Twitcher mice.

To evaluate the relevance of neurodegeneration in the Twitcher mouse, the beginning signs of Twitcher neuron distress were determined. Nissl staining was performed in coronal sections of the spinal cord of WT and Twitcher at 7, 15, and 30 postnatal days (P7, P15 and P30, respectively). Nissl staining specifically labels the rough endoplasmic reticulum (rER) in the cell body, and is frequently used to distinguish between viable neurons, which are strongly stained, and dying neurons, with little or no Nissl staining. Cragg, Brain Res. 23:1-21 (1970). The loss of Nissl staining, also called chromatolysis, marks the dissolution of the Nissl bodies (large stacks of rER) and indicates that the cell is losing its cytoplasmic architecture.

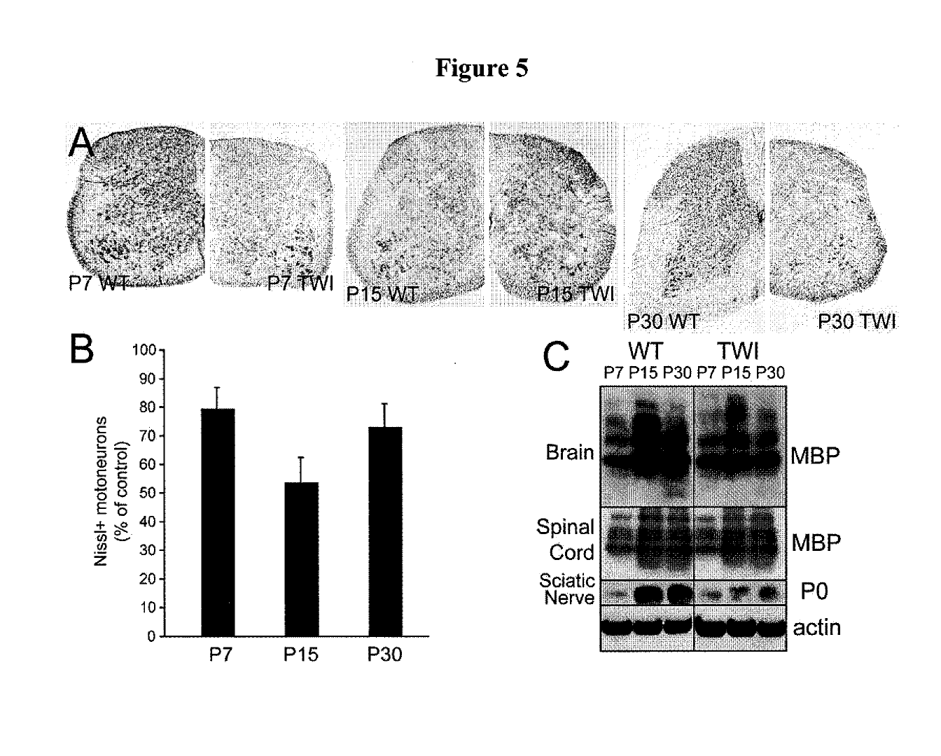

At all time points, the Twitcher spinal cord showed a decrease in the number of Nissl motor neurons in the ventral horns of the gray matter suggesting ongoing chromatolysis in the Twitcher neurons (FIG. 5A and its quantification in FIG. 5B). At P30, the number of Nissl+ SMN appeared to recover (FIG. 5B). The apparent recovery was, however, the result of a reduction of the width of the Twitcher spinal cord at this stage.

The decrease in the number of Nissl+ SMNs at later stages of the disease indicated secondary damage caused by demyelination in the Twitcher mouse. Loss of myelin affected the P30 Twitcher central and peripheral nervous systems, as shown by the decrease in the amount of the myelin components myelin basic protein (MBP) and Protein Zero (P0) in brain, spinal cord, and sciatic nerve (FIG. 5C). Twitcher demyelination starts around P15-P20, while the decrease in the number of Nissl+ SMN started at P7, suggesting that demyelination may not be the initial trigger of the Twitcher chromatolysis.

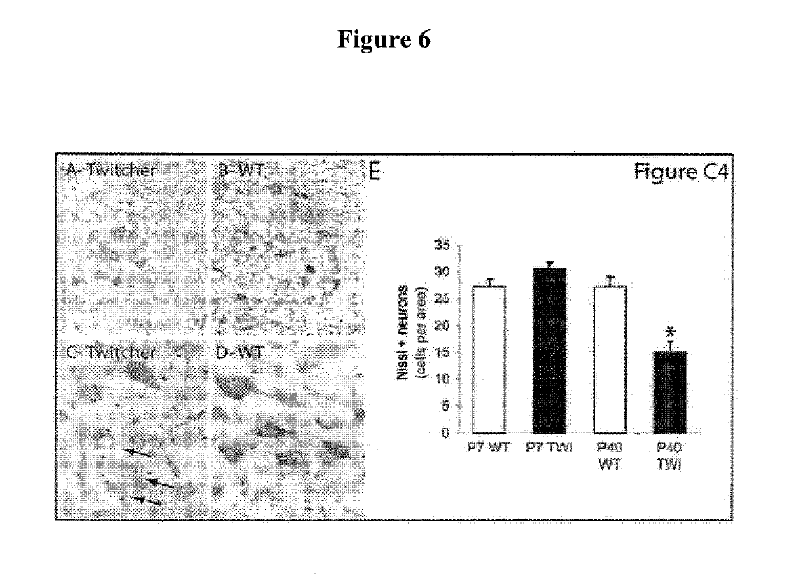

Nissl staining of Twitcher spinal cords at P7 and P40, two developmental time points characterized, respectively, by the absence and presence of demyelination, revealed reduced numbers of Nissl motor neurons in the ventral horns of the P40 Twitcher spinal cord (lumbar/sacral area) as compared to tissue from wild-type age-matched mice (FIG. 6B). Many neurons were seen as ghost profiles with little or no Nissl (arrows in 6C). Countliss1⁺ neurons in serial sections of the lumbar spinal cord showed that ˜50% of mutant motor neurons became dysfunctional in the lumbar spinal cord of aging Twitcher mice, while no decline was detected at younger ages (P7) (FIG. 6E).

Myelin degeneration has generally been considered to be the main pathological hallmark in studies of KD. Suzuki, Neurochem. Res. 23:251-259 (1998) and Takahashi et al., Acta Neuropathol. 59:159-166 (1983). Sporadic case reports have, however, also detected signs of axonal and neuronal degeneration in autopsy material. Duchen et al., Brain 103:695-710 (1980); Galbiati et al., J. Neurosci. 27:13730-13738 (2007); Jacobs et al., J. Neurol. Sci. 55:285-304 (1982); Kobayashi et al., Brain Res. 202:479-483 (1980); Kurtz and Fletcher, Acta Neuropathol. 16:226-232 (1970); Matsushima et al., Cell 78:645-656 (1994); Nagara and Suzuki, Lab. Invest. 47:51-59 (1982); Ohno et al., Brain Res. 625:186-196 (1993); Sakai et al., J. Neurochem. 66:1118-1124 (1996); Schlaepfer and Prensky, Acta Neuropathol. 20:55-66 (1972); Sourander and Olsson, Acta Neuropathol. 11:69-81 (1968); Taniike et al., J. Neuropathol. Exp. Neurol. 58:644-653 (1999); and Wu et al., Am. J. Pathol. 156:1849-1854 (2000)). In the Twitcher mouse model, Jacobs et al. found reduced numbers of large diameter axons, an observation that suggests deregulated mechanisms of cytoskeletal growth. Jacobs et al., J. Neurol. Sci. 55:285-304 (1982). Other studies have also shown abnormal postural reflexes, grasp, limb strength, and some motor deficiencies in young Twitcher mice. Olmstead, Behav. Brain Res. 25:143-153 (1987). Although the general consensus is that axonal degeneration is likely a side effect of myelin loss, the cause for these early neurological deficiencies has remained unresolved.

Neurodegeneration was studied in the lower spinal cord motorneurons and their long axons, which target the lower limbs as well as axons in the ventral columns of the spinal cord. A dying-back mode of neuronal stress occurs in these cells in the twitcher mouse was identified. Neuronal death (tunel staining) was only detected when the mutant animal was sick (e.g., after 30 days of age) but not in neurons of younger animals. This suggests that neuronal involvement is a late event in the pathophysiology of this disease. DNA fragmentation in late stages coincides with demyelination, astrogliosis and inflammation, events that may combine and compound neuronal dysfunction. de la Monte et al., Lab. Invest. 80:1323-1335 (2000); Karnes et al., Neuroscience 159:804-818 (2009); and Martin et al., Biol. Blood Marrow Transplant 12:184-194 (2006). Indeed, the early reduction of Nissl staining in motorneurons and the higher abundance of pro-apoptotic proteins in nerves from P7 mutants also pointed to the development of neuronal distress in this mutant in the absence of classical neuronal apoptosis. By using a double transgenic Twitcher line (Twi-YFPax), in which axons are labeled by the Thyl.1-driven expression of YFP in spinal cord motorneurons, it was demonstrated that axonal dystrophism (e.g., swelling, breaks and varicosities) was already present at very early stages of postnatal development (P7) and long before demyelination and neuronal damage occured. These axonopathological features rapidly progressed in numbers and distribution as the mutants aged. The presence of early axonal problems strongly suggested that axonal dysfunction appeared before neuronal cell bodies were affected in this disease, supporting the hypothesis of a dying-back pathology.

The loss of synapses and axonal injury occur before apoptosis is activated in the neuronal soma and even if apoptosis is prevented. Sagot et al., J. Neurosci. 15:7727-7733 (1995). The results presented herein provide a structural basis to understand some of the observed changes in neurological abilities in KD. Neuronal apoptosis may not be a major player in early stages of neurodegeneration but may combine with demyelination at later more affected stages.

### Example 6

### Apoptosis is a Late Event in the Twitcher Neurons

Example 5 demonstrated the decrease of Nissl⁺ SMN at all time points but does not elucidate its causative mechanism. Indeed, chromatolysis can be the result of several conditions and might not provide a clear indication of the nature of the neuronal insult. Cragg, Brain Res. 23:1-21 (1970). One possibility is that the Twitcher mutation induces apoptosis in the SMN, as occurs in myelinating glia. Jatana et al., Neurosci. Lett. 330:183-187 (2002); Tanaka and Webster, J. Neuropathol. Exp. Neurol. 52:490-498 (1993); and Zaka and Wenger, Neurosci. Lett. 358:205-209 (2004)).

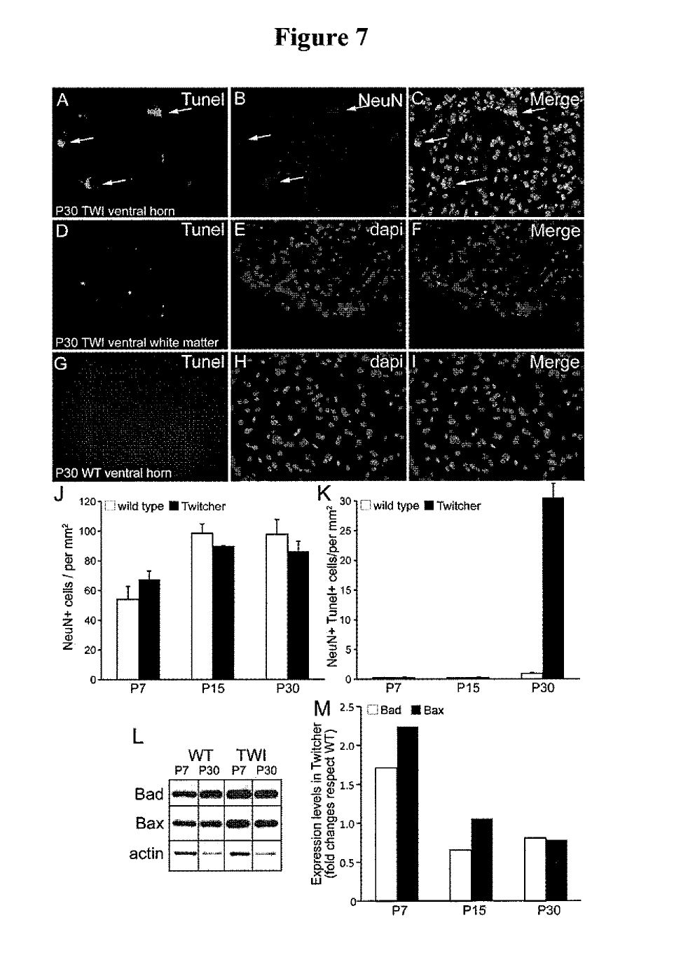

To understand whether the disappearance of Nissl⁺ neurons in the Twitcher mouse was caused by apoptosis, the terminal deoxynucleotidyl transferase dUTP nick end labeling (TUNEL) assay was performed on coronal sections of the spinal cord of WT and Twitcher animals. The TUNEL assay detects cleavages in DNA, a classic feature of apoptosis. Gavrieli et al., J. Cell Biol. 119:493-501 (1992) and Wijsman et al., J. Histochem. Cytochem. 41:7-12 (1993). In the Twitcher mouse, several TUNEL+ cells were detected at P30 in both the Twitcher gray and white matter (FIG. 7A and FIG. 7D, and counting in FIG. 7K), but not in the WT (FIG. 7G). This result agrees with the previous studies showing apoptotic death in the Twitcher animals. Wenger et al., in “The Metabolic and Molecular Bases of Inherited Disease” (Scriver et al., (eds) McGraw-Hill: New York, 3669, 3670, and 3687 (2001)). Notably, several large TUNEL⁺ motor neurons were found in the gray matter (FIGS. 7A-C). These cells were positive for the neuron specific marker neuronal Nuclei (NeuN), indicating that these cells were dying neurons. Interestingly, the neurons in the ventral horns showed cytoplasmic rather than nuclear localization of the TUNEL staining (FIG. 7A). Although the reason for cytoplasmic localization of the TUNEL staining has not yet been explained, it has previously been reported for neurons undergoing chromatolysis. Karnes et al., Neuroscience 159:804-818 (2009). Motor neuron TUNEL⁺ cells at time points earlier than P30 could not be detected, suggesting that apoptosis in the SMN was a late event.

When expression of pro-apoptotic effectors (Bad and Bax) was examined, both pro-apoptotic proteins were found to be higher in sciatic nerves from P7 Twitchers, (FIG. 7L and relative quantification in FIG. 7M). Oltvai et al., Cell 74:609-619 (1993) and Roy et al., Mol. Cell 33:377-388 (2009). Both proteins were not significantly increased in mutant spinal cords as compared to wild type controls (data not shown). The increase in these two pro-apoptotic proteins in the nerves at early postnatal times suggested an early stress on the nerves. At this stage, there was neither demyelination nor inflammation, for which Twitcher neurons may not fully activate death mechanism.

### Example 7

### Axonal Dystrophy in the Twitcher Mouse

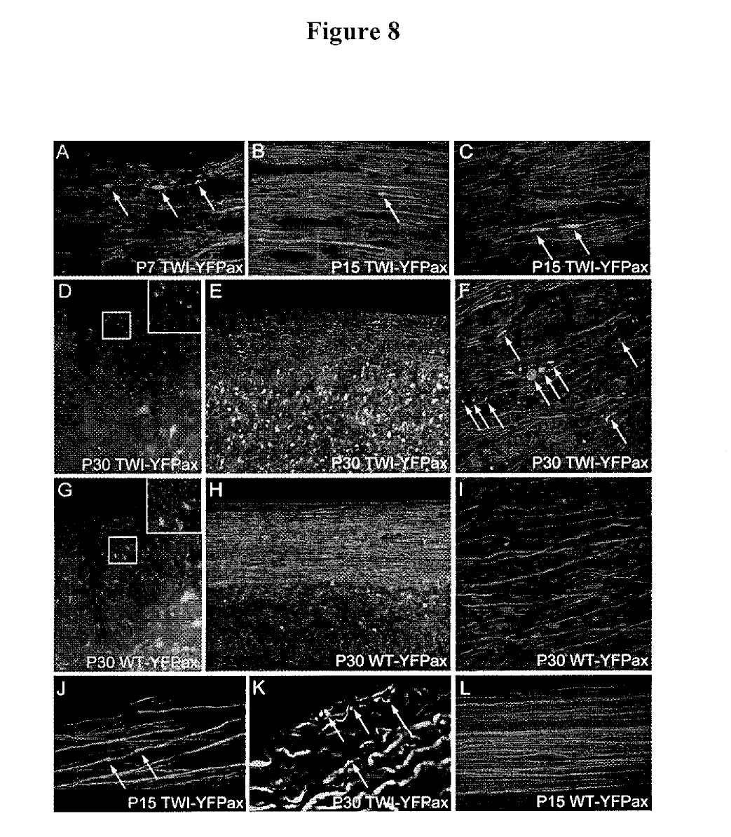

The late appearance of apoptotic markers in the neuronal soma often indicates that insults begin in the axon and eventually lead to dramatic changes in the cell body. Coleman, Nat. Rev. Neurosci. 6:889-898 (2005). The possibility that the site of injury in the Twitcher neurons was along the axonal processes was investigated. To determine if neuronal processes were affected in the disease, the Twitcher mouse was crossed with the Thyl.1-YFP transgenic mouse line, in which the yellow fluorescent protein (YFP) specifically labels some neurons and permits clear axonal marking. Feng et al., Neuron 28:41-51 (2000). FIG. 8 shows the results of the investigation of TWI-YFPax spinal cord at P7, P15 and P30 (FIGS. 8A-8F). It was found that the Twitcher mouse had fewer intact YFP axons in the white matter, as compared to the WT (compare FIG. 8E with FIG. 8H). Mutant axons showed varicosities and swellings, as well as breaks, along the axons as early as P7 (arrows in FIG. 8A), while the WT axons did not show any sign of morphological changes (FIGS. 8G-8I). These axonal profiles often appeared as tandemly repeated enlargements along the axon, suggesting a multifocal insult to that particular axon (arrows in FIGS. 8A, 8B, 8C, and 8F).

Axonal dystrophy has been reported in several neurodegenerative disorders and animal models as a sign of early axonal stress and are often observed before cell death occurs. Coleman, Nat. Rev. Neurosci. 6:889-898 (2005); Kornek et al., Brain 124:1114-1124 (2001); Stokin et al., Science 307:1282-1288 (2005); and Tsai et al., Nat. Neurosci. 7:1181-1183 (2004). Importantly, the axonal varicosities that were present at P7 in the Twitcher spinal cord were found at a time when demyelination was not yet detectable. Dystrophic axons were evident also in the TWI-YFPax sciatic nerve (FIGS. 8J and 8K), indicating that the Twitcher neuropathology can affect both the central and the peripheral processes. Since affected axons were found to display multiple varicosities in both the central and peripheral nervous system, these experiments suggested that axonal dystrophy is a generalized problem along the neuronal processes of the Twitcher mouse.

### Example 8

### Trafficking of Kinesin is Altered in the Twitcher Axons

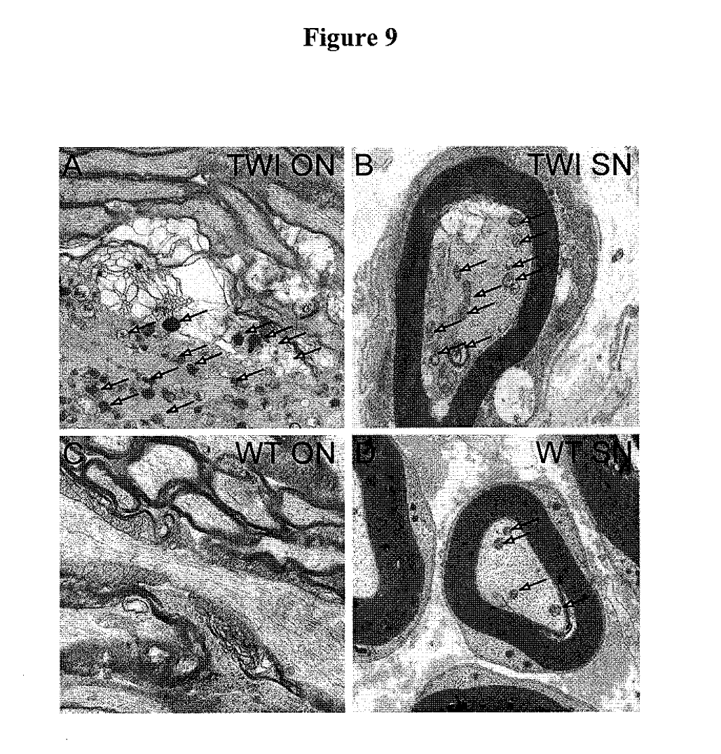

Conclusive data regarding the molecular mechanism that causes axonal swelling in neuropathologies have not been described. Several studies have, however, suggested that a local defect in axonal transport might cause the focal accumulation of untransported material, like membrane bound organelles (MBOs), and as a result, the enlargement of the axon. Coleman, Nat. Rev. Neurosci. 6:889-898 (2005). Interestingly, transmission electron microscopy (TEM) of the Twitcher sciatic and optic nerves showed the presence of abundant vesicles in the Twitcher axons (FIG. 9). Accumulation of vesicles suggests that the axonal swelling observed in TWI-YFPax mice was caused by deregulated transport along axons.

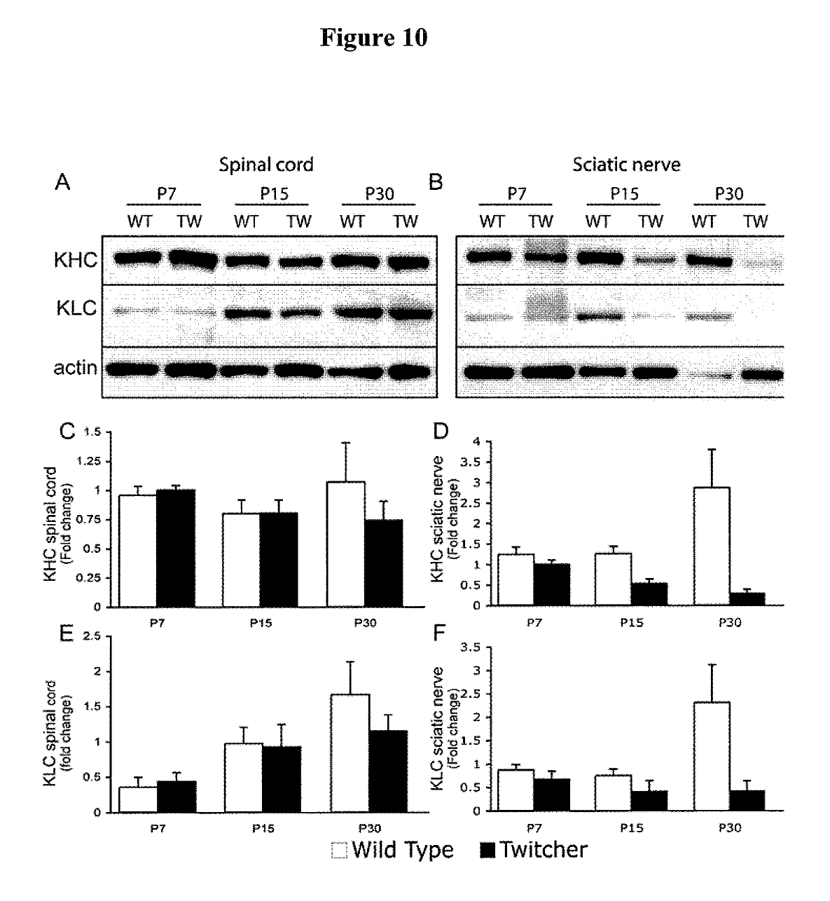

To determine if the transport machinery of the Twitcher neurons was compromised, the amounts of kinesin heavy and light chains (KHC and KLC, respectively), the enzyme responsible for fast anterograde axonal transport, were quantified in the spinal cord and sciatic nerve of the Twitcher animals (FIG. 10). FIG. 10A showed that there was no significant difference in the amounts of KHC and KLC of the WT and Twitcher spinal cords (quantification in FIGS. 10C and 10E). A strong reduction in the amount of both chains was, however, detected in the sciatic nerve (FIG. 10B and quantification in FIGS. 10D and 10F), suggesting a defect in the trafficking of kinesin. Since the levels of kinesin did not change in the spinal cord, where the neuronal cell bodies are located, these data suggest that the observed decrease in kinesin in the sciatic nerve was caused by a defect in the activity of the motor, rather than by a change in its gene expression.

### Example 9

### The Efficiency of the Twitcher Axonal Transport is Reduced

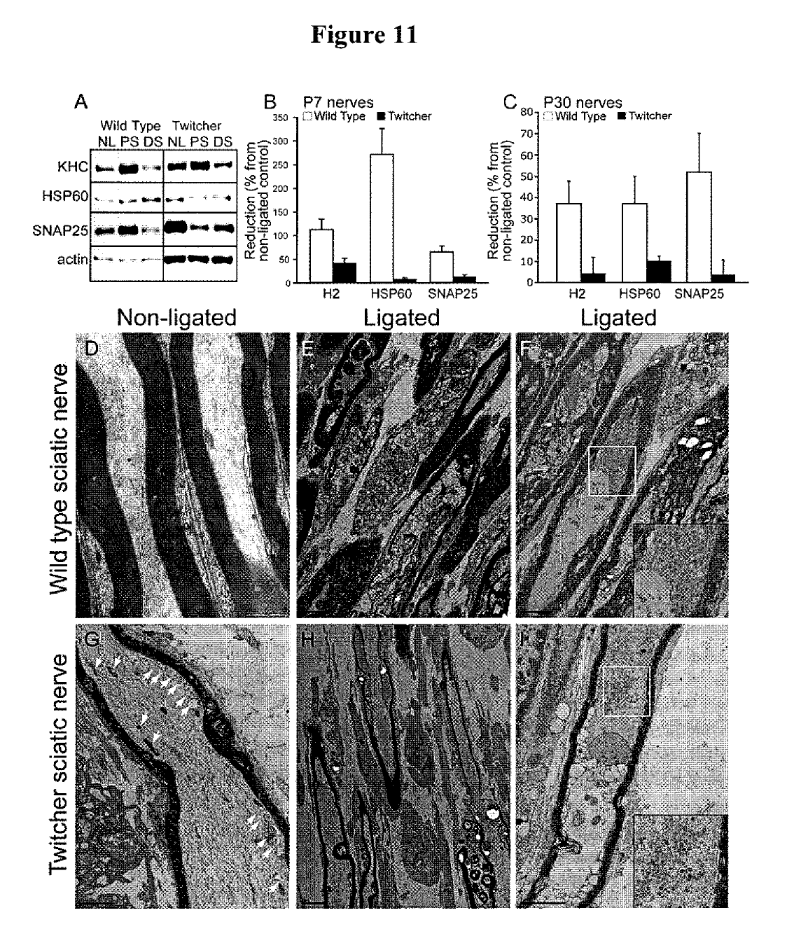

To determine if axonal transport was indeed affected by KD disease, a ligation of the sciatic nerve of Twitcher mice was performed. WT and Twitcher mice at P30 were unilaterally ligated for 6 hours and the proximal and distal halves of the nerve, relative to the ligature, were collected and processed for immunoblot analysis and transmission electron microscopy (TEM) (FIG. 11). In this model, transported cargoes accumulate at the site of the ligature and the extent of the accumulation provides an indication of the transport efficiency.

While the ligated WT axons accumulated KHC, the synaptic marker SNAP25, and the mitochondrial marker Heat Shock Protein 60 (HSP60), the Twitcher mouse showed reduced accumulation of those proteins (FIG. 11A and quantification in FIG. 11C). The decrease in all of these markers suggested that the defect in Twitcher axonal transport was not limited to a specific type of cargo but was rather a generalized problem of trafficking. TEM further confirmed these results. While most of the WT axons contained accumulated MBOs (FIGS. 11E and 11F), fewer Twitcher axons showed a similar accumulation, even in the axons that were myelinated (FIGS. 11H and 11I). Moreover, vesicular structures were observed beneath the plasma membrane in the unligated Twitcher control (arrows in FIG. 11G). The presence of these vesicular accumulations suggested a defect in the sorting of the transported MBOs, a process that is tightly regulated by various enzymatic activities. Hooper et al., J. Neurochem. 104:1433-1439 (2008); Morfini et al., Proc. Natl. Acad. Sci. USA 104:2442-2447 (2007); Morfini et al., Embo 1 23:2235-2245 (2004); and Runnegar et al., Biochem. I 342(Pt 1):1-6 (1999).

Axonal transport defects are observed in several pathologies and their role as causative agents or pathological consequences is often a subject of debate. To understand whether the Twitcher axonal transport defect is responsible for the observed neurodegeneration, and to eliminate the possibility that it was secondary to demyelination, the ligation experiment was repeated on P7 animals. Even at this young age, a reduction in the amount of accumulated organelles was observed in mutant nerves (FIG. 11B), further suggesting that defective axonal transport was at least partially responsible for the observed axonal and neuronal stress.

A fundamental step in understanding the role of neurodegeneration in KD is finding the mechanism that leads to axonopathy. The results presented herein indicate that Twitcher neurons were affected by slowed axonal transport, a condition that can easily lead to synaptic dysfunction and axonal retraction. Coleman, Nat. Rev. Neurosci. 6:889-898 (2005). The relevance of fast axonal transport (FAT) to neuronal survival and function is best exemplified by the discovery that mutations in the function of kinesin or dynein lead to neurodegeneration. For example, mutations in Kinesin-1A cause a partial inhibition of FAT and lead to one form of hereditary spastic paraplegia (Reid et al., Am. J. Hum. Genet. 71:1189-1194 (2002)) while mutations in Kinesin-1B lead to a form of Charcot-Marie Tooth disease (Zhao et al., Cell 105:587-597 (2001)). In addition, it has been shown that mutations in the dynein complex are found in some forms of motor neuron disease. Puls et al., Nat. Genet. 33:455-456 (2003). These results exemplify the sensitivity of neurons to defects in axonal transport. The consensus is that these mutations trigger a dying-back pathology in axons and eventually, death of affected neurons, even if the mutations affect all somatic cells in the organism.

Studies have indicated that a decrease in axonal transport efficiency is a common degenerative mechanism for neurons in several unrelated diseases including Hungtington's disease, Alzheimer's disease, and amyotrophic lateral sclerosis. As a result, there have been efforts to determine the role of altered transport in the pathogenesis of these diseases. Morfini et al., J. Neurosci. 29:12776-12786 (2009). A crucial question in these studies was whether a deficit in transport is causative of pathology or simply a consequence of neuronal dysfunction. Interestingly, in most cases it has been demonstrated that defects in axonal transport can be detected before the onset of the symptoms (Ferguson et al., Brain 120(Pt 3):393-399 (1997)), suggesting that transport deficiency is likely a causative event and not necessarily a consequence of a related dysfunction. The role of deficient FAT in leukodystrophies, other forms of lysosomal storage diseases, and aging have has not been determined.

The data presented herein suggest that deregulated FAT is causative for axonal dysfunction and demonstrates that deficits of FAT appear as early as P7, when Twticher mice do not show any clinical sign of neuropathology and when demyelination is not yet involved (demyelination starts after the second week of age). Myelin regulates the rate of axonal transport (de Waegh et al., Cell 68:451-463 (1992)) and the loss of myelin may compound transport deficiencies.

In the case of KD, the presently disclosed data suggests that late stages of neuropathology (e.g., demyelination and axonal dysfunction) may involve at least two pathways: (1) the classical pathway in which defects in myelinating glia lead to demyelination and subsequently to axonal dysfunction as a secondary event and (2) the defective metabolism of galactosyl-sphingolipids may also autonomously affect mutant neurons, which may activate mechanisms that deregulate axonal transport in some neuronal tracts at earlier stages, before demyelination (FIG. 12). In both cases, the endpoint is a compounding myelin and axonal dysfunction. This model suggests a more complicated disease process than was previously assumed.

### Example 10

### Degeneration of Twitcher Axons During Postnatal Development

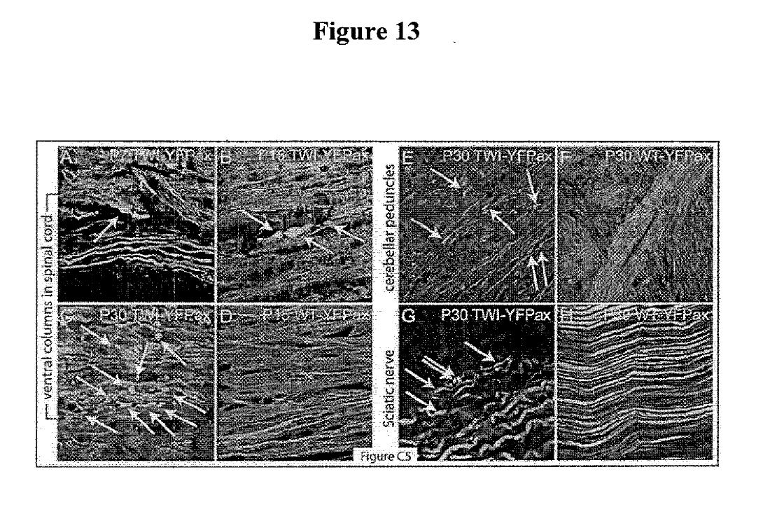

The presence of damaged axons was detected in Twitcher mice crossed with Thyl.1-YFP transgenic mice (Twitcher-YFPax) in which the Thyl.1-YFP drives expression of fluorescent YFP specifically to neurons and permits axonal marking. Feng et al., Neuron 28:41-51 (2000). FIG. 13 shows images from confocal hemisections of ventral columns of the spinal cord (FIGS. 13A-13D), cerebellar peduncles (FIGS. 13E and 13F), and longitudinal sections from the sciatic nerve (FIGS. 13G and 13H). In all samples from mutant mice, pathological figures (swellings, varicosities, and breaks) were detected along some axons (arrows). Furthermore, axonopathic figures were observed as early as P7 (FIG. 13A) and were present at all levels of the neuroaxis, with higher frequency in spinal cord and sciatic nerves.

### Example 11

### Psychosine Preferentially Accumulates in Lipid Rafts in Twitcher Brains

The above Examples demonstrated axonopathy and axonal transport defects in the Twitcher mouse, which is a classic model of demyelination. Since loss of myelin was not present at P7, when the first signs of stalled axonal transport occur, the observed effects could not, however, be explained solely by the presence of demyelination. In addition, accumulation of vesicles in myelinated Twitcher axons indicated that demyelination did not account for the decrease in axonal trafficking.

One explanation for the observed neuropathology is that psychosine, the potent neurotoxin that induces demyelination in the Twitcher mouse, also targets neurons. Psychosine may accumulate in the Twitcher neurons independently of myelin, affecting neuronal stability even in the absence of the myelin-related pathology.

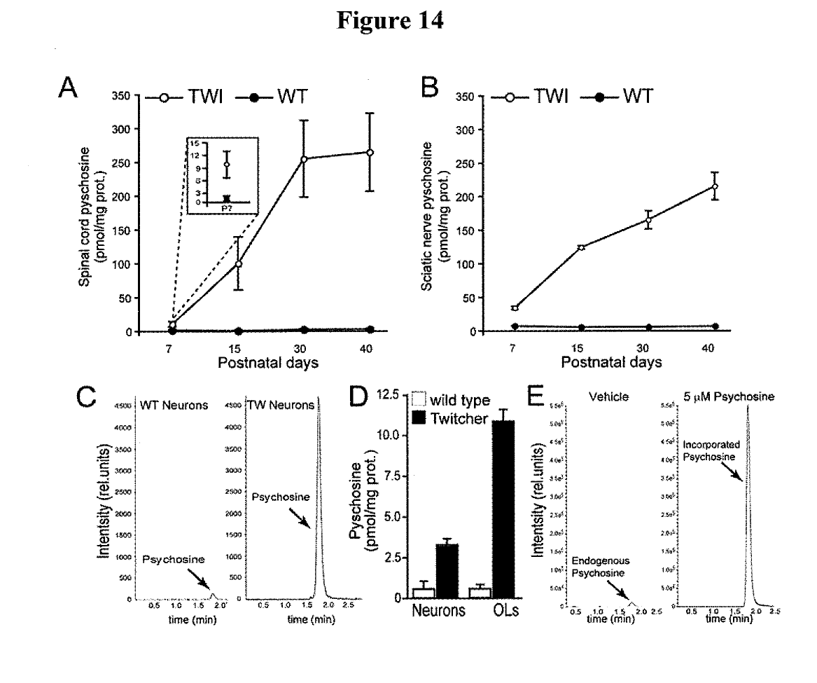

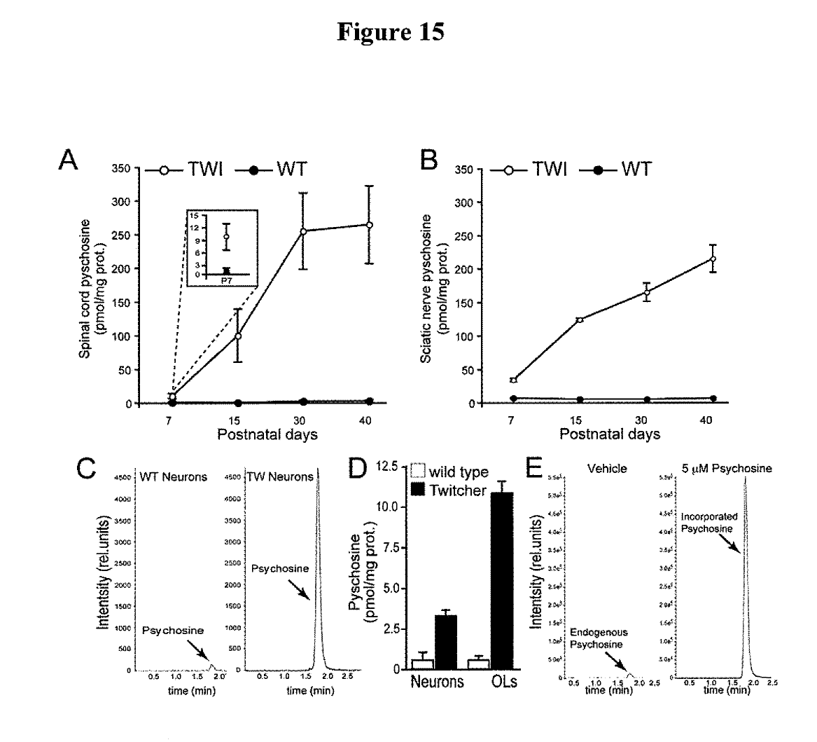

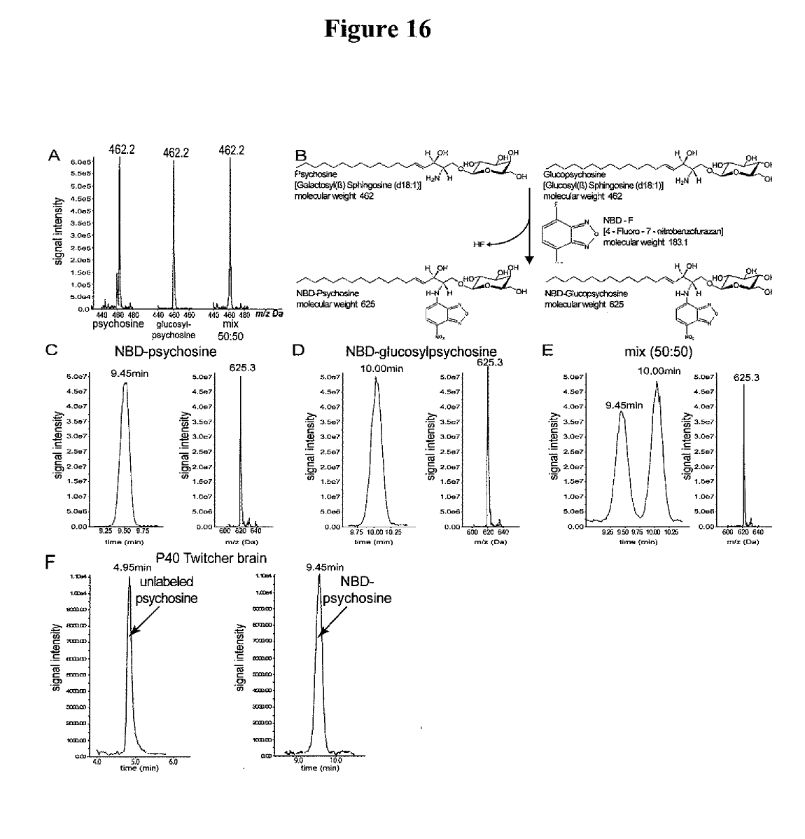

To prove this hypothesis, high performance liquid chromatography mass spectrometry (HPLC-MS-MS) was performed to quantify the amount of psychosine accumulated in the Twitcher spinal cord and sciatic nerves at P3, P15, P30, and P40 (FIGS. 14A and 14B). By using HPLC-MS-MS, galactosyl-psychosine was quantified and distinguished from glucosyl-psychosine, another brain glycosyl-sphingolipid with an ion mass identical to that of psychosine (FIG. 15). Although in low amounts, psychosine was already significantly higher in the Twitcher tissues at P3, demonstrating that the accumulation of psychosine starts prior to and independently of myelination/demyelination in the Twitcher mouse. These data do not, however, rule out the possibility that immature glia, and not neurons, might still be responsible for a portion of psychosine synthesis at early ages.

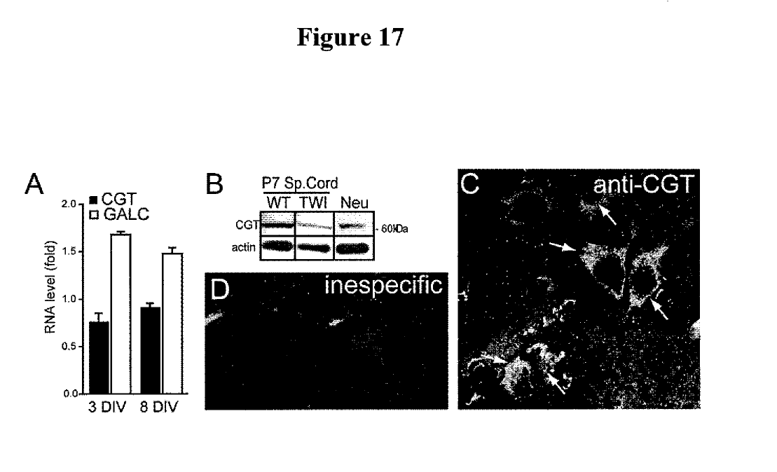

Since neurons express ceramide galactosyltransferase (CGT), the enzyme responsible for psychosine synthesis both in vitro (FIGS. 17A and 17B) and in vivo (FIGS. 17C and 17D), it is reasonable to assume that neurons might also produce psychosine. To determine if neuronal synthesis of psychosine was observable, HPLC-MS-MS was performed on WT and Twitcher cultured neurons to quantify the amount of accumulated psychosine. FIGS. 14C and 14D show that, although the neuronal psychosine was not as abundant as was seen in purified Twitcher oligodendrocytes, Twitcher neurons accumulate significantly more than control WT neurons. The combination of these results strongly supports the idea of neuronal synthesis of psychosine. Since it was also demonstrated that neurons can take up psychosine upon exogenous exposure (FIG. 14E), the possibility of the transfer of this lipid from glia to neurons was not ruled out.

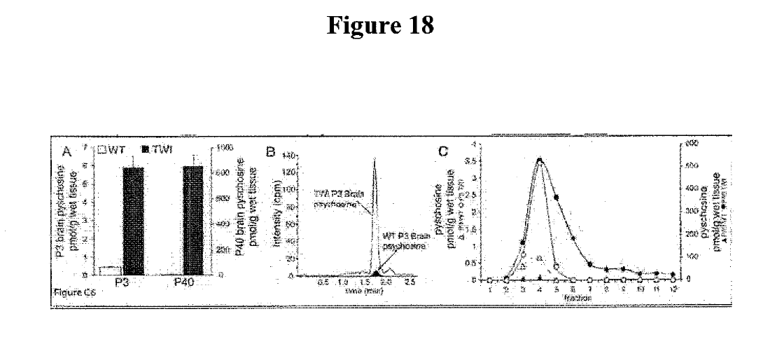

To examine the effects of psychosine on cell membranes from the Twitcher CNS, lipid rafts were isolated from brains at P3 and P40, analyzed by mass spectrometry for psychosine concentration in raft and non-raft fractions at each time point, and compared to the wild-type. Total concentrations of psychosine were significantly higher (p<0.05) in the Twitcher brain at both time points (FIG. 18A). FIG. 18B shows representative data from mass spectrometric analyses of raft fractions prepared from P3 mouse brains. Psychosine was detected at much higher levels in samples prepared from Twitcher mice. Psychosine concentrations in the brain rafts (fractions 4-6) at P3 were about 5 pmol/g of wet tissue in the mutant, representing a 6-fold increase over that in the WT, while psychosine concentration in Twitcher brain rafts at P40 was about 1000 pmol/g of wet tissue as compared to less than 3 pmol/g in the wild-type, representing an increase of over 300-fold in Twitcher vs. wild-type mice (FIG. 18C). Importantly, comparison of the total psychosine to the psychosine contained in lipid rafts from these samples showed that over 50% of psychosine in Twitcher brains was present in the rafts.

### Example 12

### Psychosine can Block Fast Axonal Transport

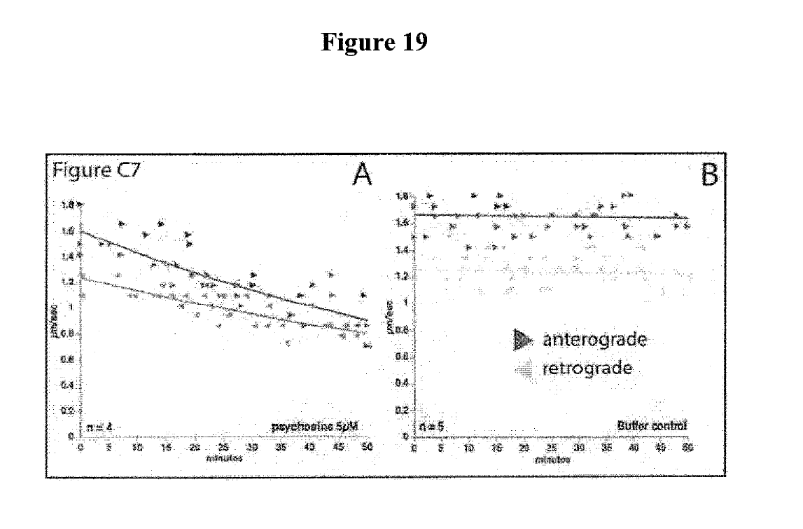

To test whether psychosine exerts a role in neurodegeneration by affecting axonal transport, an experiment was performed using axoplasms isolated from giant squid axons, an approach used to examine the effects on antero and retrograde transport rates of a variety of molecules. Morfini et al., Neuromolecular Med. 2:89-99 (2002). Axoplasms extruded from their plasma membrane and infused with 5 μM of psychosine showed a rapid reduction of both antero and retrograde axonal transport rates (FIG. 19A). These date demonstrated that axonal transport is sensitive to this sphingolipid. No reduction in transport rates was seen in vehicle (10% ethanol-saline) infused axoplasms (FIG. 19B).

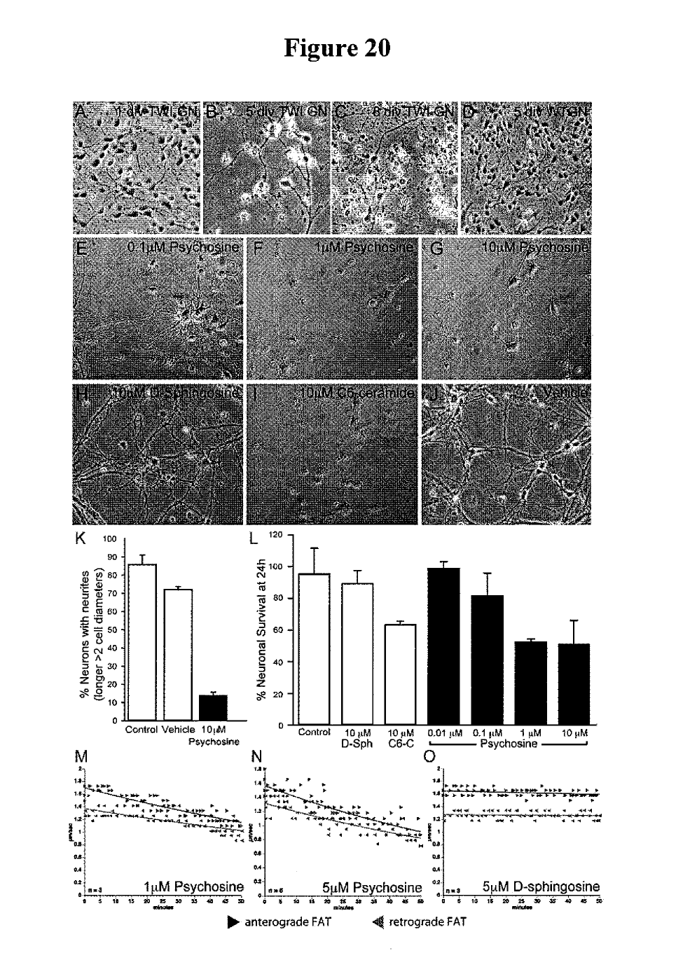

The hypothesis that Twitcher neurons are affected in a cell autonomous manner was tested. Twitcher neurons were isolated and cultured for up to 8 days. Mutant neurons rapidly manifested less neurite outgrowth and most were dead by the end of the experiment (FIGS. 20A-20C). To test the hypothesis that the presence of psychosine was detrimental to the survival of these neurons, the effect of psychosine treatment on embryonic primary cortical neurons was tested. Psychosine-treated cortical neurons showed a decrease in the number of neurites (FIGS. 20E, 20F, and 20G). This effect was comparable to the positive control C6 ceramide (FIG. 20I), a well-known apoptotic inducer, and specific for psychosine, because the sphingolipid D-sphingosine did not exert any effect (FIG. 20H). The cytotoxicity of psychosine was determined with the MTT assay, which directly measures mitochondrial activity (FIG. 20L). Psychosine was toxic even at low concentrations (1 μM), at which neurite retraction was not evident, suggesting that psychosine has a toxic effect even in the young animals when its concentration is not high and does not result in severe axonal impairment.

Whether psychosine is a pathogenic effector capable of triggering axonal defects in the Twitcher mouse was assessed. To test psychosine effect on FAT, a model of vesicular transport based on the squid Loligo Pelai was employed. This approach has been extensively characterized to examine the effects of different pathogenic proteins. Morfini et al., Neuromolecular. Med. 2:89-99 (2002). Candidate molecules were perfused in a microchamber containing the axoplasm preparations and the average motility of MBO was measured over a period of time. This model has played a fundamental role in the discovery of kinesin-1S (Brady, Nature 317:73-75 (1985)) and the regulatory mechanisms of FAT (Morfini et al., Neuromolecular. Med. 2:89-99 (2002); Morfini et al., Embo J. 23:2235-2245 (2004); and Ratner et al., J. Neurosci. 18:7717-7726 (1998)) as well as the pathogenic mechanism of various proteins and neurotoxins (Morfini et al., Proc. Natl. Acad. Sci. USA 104:2442-2447 (2007); Morfini et al., Nat. Neurosci. 9:907-916 (2006); and Pigino et al., Proc. Natl. Acad. Sci. USA 106:5907-5912 (2009)). Furthermore, antero and retrograde modes of transport in squid axoplasm are identical to those of intact axons (Lasek and Brady, Nature 316:645-647 (1985)) and all regulatory mechanisms discovered in the squid axoplasm are identical to the mammalian neuron.

Pure preparations of extruded axoplasm isolated from the squid were perfused with different concentrations of psychosine (or related controls) and the speed of MBO was recorded over time. FIG. 20O shows that perfusion of squid axoplasm with control D-Sphingosine resulted in typical transport rates of 1.5-2 mm/sec (anterograde FAT) and 1-1.4 mm/sec (retrograde transport). In contrast, 1 μM and 5 μM psychosine resulted in a strong inhibition of both modes of axonal transport. These data demonstrated that axonal transport can be specifically regulated by psychosine because D-Sphingosine did not affect the speed of anterograde or retrograde transport. These data not only demonstrated that psychosine is the likely trigger of the Twitcher axonopathy and that alteration in the metabolism of a sphingolipid can induce measurable reductions of the efficiency of axonal transport.

Without being limited by mechanistic theory, it is believed that the progression of KD is compounded with a dying back pathology because of a deficiency of GALC that is related to a mechanism of pathogenesis that interrupts FAT and thus axonal function. Because psychosine is a lipid raft-associated neurotoxin that accumulates in KD (Galbiati et al., Neurochem. Res. 32:377-388 (2007); Galbiati et al., J. Neurosci. Res. 87:1748-1759 (2009); and White et al., J. Neurosci. 29:6068-6077 (2009)), it is likely that psychosine may interfere with FAT. This was supported by quantifying psychosine in spinal cord and sciatic nerve extracts. Significant levels of psychosine were detected at P3, a much earlier developmental time than previously suggested. Suzuki, Neurochem. Res. 23:251-259 (1998). The presence of psychosine at P3 (before major myelination) suggested that psychosine may be synthesized by neural cells other than myelinating glia, such as neurons and that premature exposure of axons to psychosine are relevant to the disease process. Studies using cultures of acutely isolated neurons confirmed this by demonstrating that psychosine accumulates to significant levels in these cells and that mutant neurons degenerate faster than wild type controls, indicating that Twitcher neurons are affected by an intrinsic mechanism of degeneration.

The observed in vitro effects of psychosine on neurons suggests that psychosine is a pathogenic effector of FAT inhibition. This was confirmed by using extruded axoplasm preparations isolated from the giant axons of the squid Loligo pealei. These data demonstrated that psychosine was sufficient to inhibit both antero and retrograde modes of FAT. FAT depends on regulated activity of molecular motors, which rely on the activity of numerous enzymes. Hooper et al., J. Neurochem. 104:1433-1439 (2008); Morfini et al., Proc. Natl. Acad. Sci. USA 104:2442-2447 (2007); Morfini et al., Embo J 23:2235-2245 (2004); and Runnegar et al., Biochem. J. 342(Pt 1):1-6 (1999). Psychosine is known to inhibit kinases such as PKC (Hannun and Bell Science 235:670-674 (1987)) and because it is a lipid raft associated component (White et al., J. Neurosci. 29:6068-6077 (2009)), it is believed that psychosine acts at the level of membrane microdomains of transported cargoes and the associated signaling cascades.

For psychosine to be sufficient to block FAT, it must reach the axonal compartment via the transport machinery. Psychosine may reach the axon from at least three sources: (i) in situ synthesis in the axonal compartment; (ii) neuronal synthesis and transport via membrane-bound cargoes; and (iii) lipid transfer from myelin sheaths/surrounding glia. The synthesis of lipids such as sphingomyelin and phosphatodylcholine has been demonstrated in axons (Krijnse-Locker et al., Mol. Biol. Cell 6:1315-1332 (1995)) and several studies have shown the transport of various lipids and cholesterol along the axon prior to insertion into the axolemma. Vance et al., Biochim. Biophys. Acta 1486:84-96 (2000); Vance et al., J. Neurochem. 62:329-337 (1994). Because psychosine is a lipid raft component, it may translocate in association with cholesterol in the microdomains of axonal cargoes. Lipid transfer between axons and myelin has also been shown for certain species of lipids (Vance et al., Biochim. Biophys. Acta 1486:84-96 (2000)), suggesting that psychosine may be transferred from myelin and surrounding glia.

### Example 13

### Psychosine-mediated Block of Fast Axonal Transport Involves PP1 Dephosphorylating Activity

This Example demonstrates that PP1 mediates psychosine inhibition of axonal transport and that reduction of PP1 activity in GALC-deficient neurons can help to improve axonal transport.

Axonal transport is regulated mainly by phosphorylation/dephosphorylation of motors and other components of the axonal cytoskeleton. This phosphotransferase activity is mediated by a wide array of kinases such as some members of the PKC family and phosphatases such as PP1 and PP2. To examine the potential role of deregulated phosohotransferase activity in the blockage of fast axonal transport by psychosine, specific inhibitors of kinases (Go76, Go83, and PP2) and of phosphatases (okadaic acid and inhibitor 2) were employed.

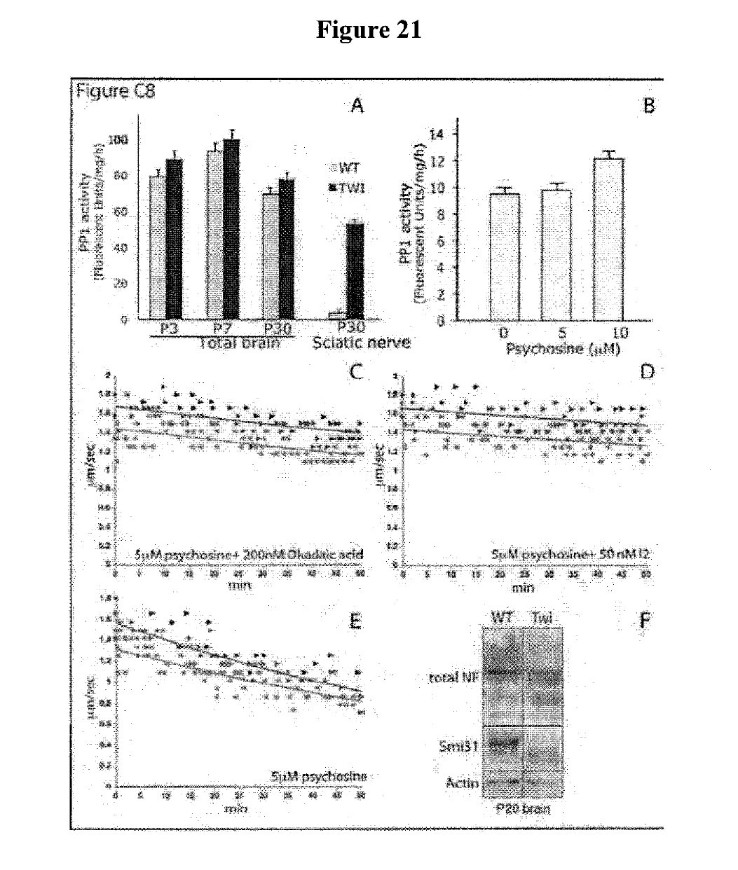

Kinase inhibitors provided no significant protection from psychosine-mediated axonal defects (not shown), whereas axoplasm preparations infused with psychosine and co-infused with okadaic acid (a pan inhibitor of protein phosphatases) or inhibitor 2 (to specifically inhibit PP1) prevented much of the blockage of axonal transport (FIG. 21D).

Measurement of PP1 enzymatic activity in the brain of Twitcher mice at P3, P7, and P30 using a fluorometric phosphatase assay indicated a 10-14% increased PP1 activity as compared with PP1 levels in brains from age-matched wild-type mice. The increase was even higher in the sciatic nerve (FIG. 21A). PP1 activity was induced in enriched cultures of cortical neurons incubated in the presence of psychosine (FIG. 21). Because neurofilaments are some of the downstream targets of PP1 activity (Strack et al., Brain Res Mol Brain Res 49:15-28 (1997)), whether the higher activity of PP1 in the Twitcher brain leads to the decreased abundance of phosphorylated neurofilaments was tested by immunoblotting protein extracts with Smi31, a monoclonal antibody that recognizes a set of epitopes in phosphorylated neurofilaments. FIG. 21F shows that neurofilaments from the mutant brain were less phosphorylated.

### Example 14

### Abnormal Clearance of Intracellular Ca⁺⁺ and Expression of the NXC1 Exchanger in Twitchers

This Example demonstrates that Twitcher neurons are exposed to higher than normal concentrations of calcium over long periods of time, which may trigger calcium-mediated downstream events that destabilize axonal cytoarchitecture and transport, thereby contributing to neuronal demise.

Increased influx of Na⁺ leads to a rapid accumulation of intracellular Ca⁺⁺, due to the reverse activity of the Na⁺/Ca⁺⁺ exchanger NCX1. Stys et al., J. Neurosci. 12:430-439 (1992). This irregular concentration of Ca⁺⁺ triggers the activation of calpains, caspases, and mitochondrial dysfunction leading to ultrastructural alterations in the axon and eventual axonal degeneration. Buki and Povlishock, Acta Neurochir (Wien) 148:181-193 (2006) and Whiteman et al., Faseb J. 18:1395-1397 (2004).

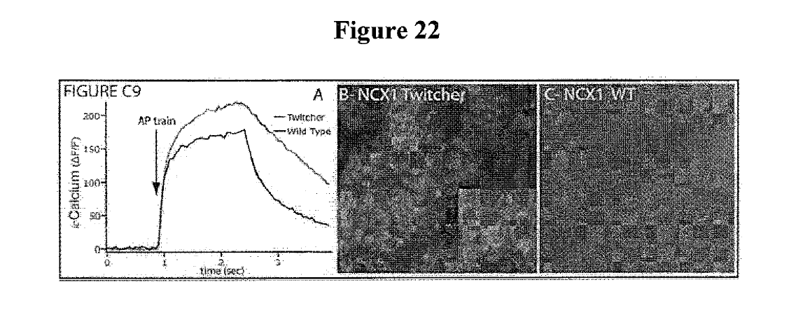

Initial analysis of intracellular calcium levels by patch-clamp using Fura2 dye in hippocampal CA2 pyramidal neurons showed that Twitcher neurons, upon stimulation with an action potential train, exhibited a higher latency in removing intracellular calcium as compared to wild-type neurons (FIG. 22A). Other analyses to examine NCX1 expression in the spinal cord of mutants and age-matched wild-types during development (FIGS. 22B and 22C) showed results at P30. NCX1 was upregulated in the ventral columns of the Twitcher spinal cord (FIG. 22B) but not in the wild-type (FIG. 22C).

### Example 15

### Flecainide Ameliorates some Clinical Signs and Neurodegeneration in Twitchers

This Example demonstrates the therapeutic efficacy of the NCX1 inhibitor flecainide as a neuroprotective agent for leukodystrophies such as KD.

Various drugs that block sustained sodium currents and thereby decrease the reverse activity of NCXI have been used to prevent calcium-mediated axonal damage. Sodium blockers, such as flecainide or phenytoin, have successfully prevented major axonal loss in EAE, spinal cord injury, and hypoxic injury. Bechtold et al., Ann. Neurol. 55:607-616 (2004); Lo et al., J. Neurophysiol. 90:3566-3571 (2003); and Bechtold et al., Brain 128:18-28 (2005). Sodium blockers are increasingly being considered as a pharmacological alternative to prevent axonal loss in myelin disease and three clinical trials are currently under development. Waxman, Nat. Clin. Pract. Neurol. 4:159-169 (2008).

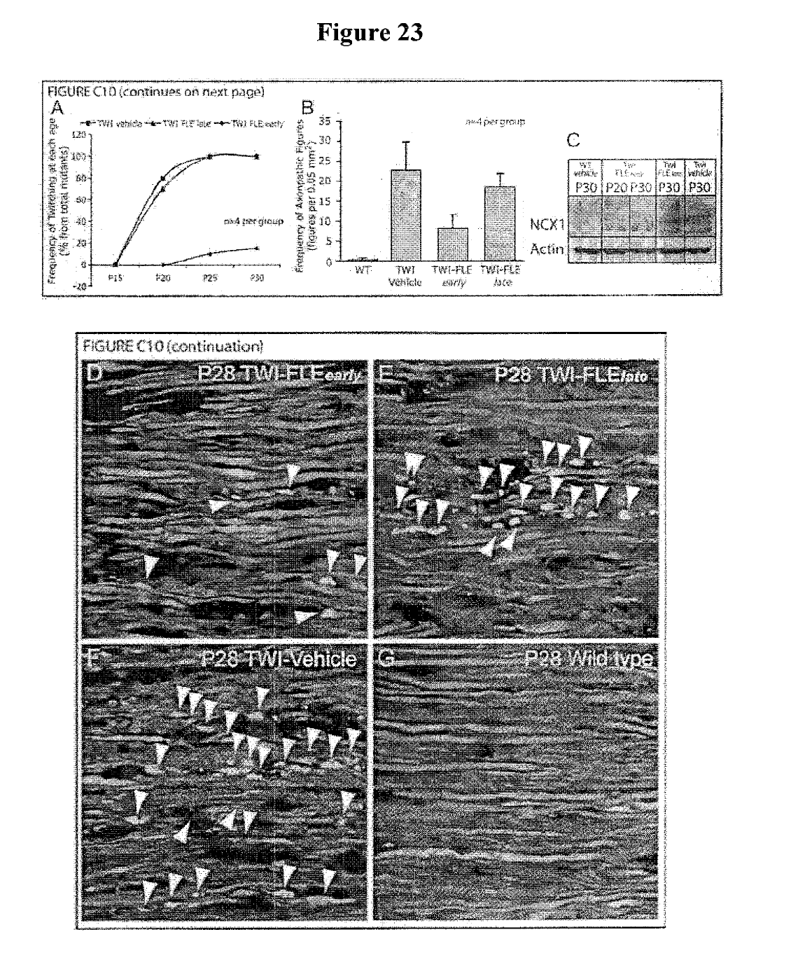

Use of flecainide in Twitcher mice revealed a significant effect of this drug in ameliorating axonal stress during the first weeks of postnatal life, underscoring the potential benefit of its use in KD. Twitcher-YFPax transgenic animals received daily subcutaneous injections of flecainide acetate (30 mg/kg/day of Tambocor (Sigma) in vehicle 2.5% glucose 20 mM HEPES, pH 7.4) or vehicle alone starting from P2 until tissue collection. Bechtold et al., Brain 128:18-28 (2005). This dose was sufficient to reduce axonal degeneration in models of demyelinating EAE (Bechtold et al., Ann Neurol 55:607-616 (2004)) and significantly protected axons in the spinal cord of the Twitchers mouse. Because early P5 administration, as opposed to later administration, of flecainide was suggested by these data, treatment starting at P2 provided an even stronger protective effect. FIG. 23.

To examine whether protection of axons accompanied the flecainide-mediated amelioration of twitching, spinal cord tissue was collected at P30, and longitudinal sections of the ventral white matter were observed by confocal microscopy for axonal integrity, using the YFP expression as reporter. FIG. 23D shows that axonopathic figures (breaks, swellings, and varicosities) were considerably less frequent in Twitcher-YFPax mice treated with flecainide beginning at P5 (arrowheads indicate various axonopathic profiles). Quantitation of these pathologic figures per area showed that early treatment reduced the number of structural pathologies to motor axons by about 50% (FIG. 23B), whereas late treatment with flecainide was not as protective, with a frequency of axonopathic figures in the ventral spinal cord not significantly different from that in vehicle-treated animals (FIGS. 23B, 23E, and 23F).

The protective effect of flecainide was shown to be accompanied by changes in NCX1 expression, by immunoblotting protein extracts from spinal cord with anti-NCX1 antibodies. FIG. 23C shows that the spinal cord of mutants subjected to the early treatment with flecainide had reduced NCX1 expression at early (P20) and late (P30) ages. Reduction of NXC1 was not detected in mutants treated with flecainide late in their life.

### Example 16

### The RVG Peptide Binds to Neurons Exclusively and Crosses the Blood Brain Barrier

This Example demonstrates that the RVG-peptide is capable of crossing the blood-brain barrier to enter the nervous system and bind to neurons.

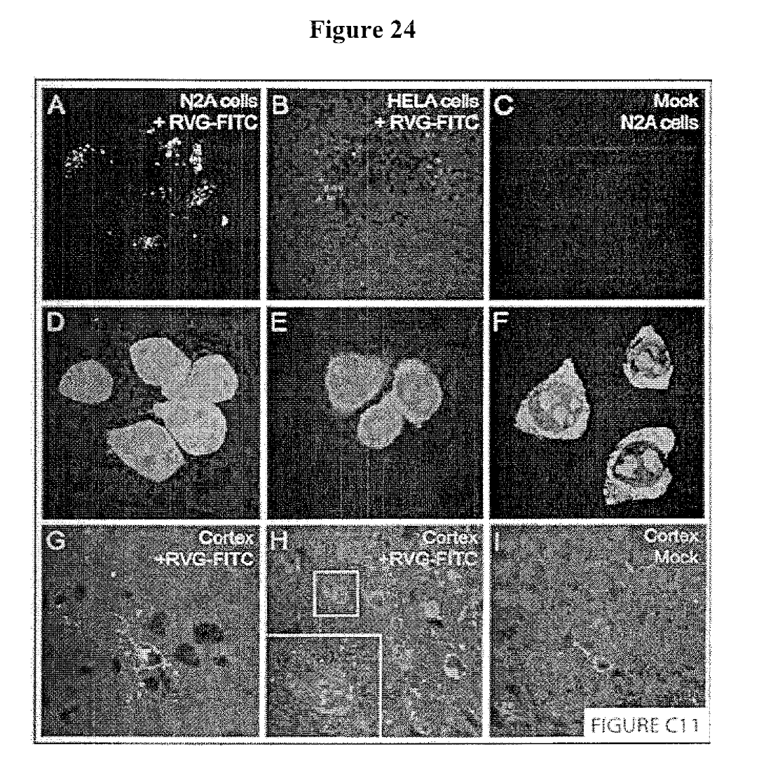

The RVG-peptide binds specifically to neurons and facilitate the delivery of siRNA sequences to the CNS. The RVG-peptide was synthesized and labeled with a fluorescent tag to allow fluorescent microscope visualization of cells that incorporate the peptide. Neuronal 2A (N2A) and non-neuronal HeLa cells were exposed to the peptide before confocal visualization. Numerous intracellular green-fluorescent particles of RVG-FITC were revealed only in N2A cells (FIG. 24A) but not in HeLa cells (FIG. 24B) indicating the specificity of binding of the RVG-peptide to neurons. Cells incubated with the RVG-peptide showed no signs of cell death.

To assess whether the RVG-peptide crosses the blood-brain barrier after intravenous infection, a cohort of 3 newborn pups was infected with RVP-FITC. The peptide was delivered intravenously through the supraorbital vein in 2 day-old pups. Pups had no signs of distress and survived the injection. Animals were killed 6 hours later and brains were cryosectioned and photographed using a confocal microscope. Numerous neurons in the cortex (identified with anti-NeuN antibodies) were found containing intracellular deposits of green fluorescent particles (FIG. 24G and 24H). FIG. 24I shows the absence of neurons from the mock-treated mice.

### Example 17

### Delivery of siRNA-RVG Peptide Decreases the Expression of Catalytic α- and β-PP1 Subunits in N2A Cells but not in HeLa Cells

This Example discloses the controlled reduction of PP1 activity through the siRNA silencing of mRNA encoding the catalytic α- and β-PP1 subunits and demonstrates the reduction of catalytic PPI subunits in neurons using specific siRNA sequences coupled to the RVG peptide.

The successful delivery of siRNA to knock down the catalytic subunits of PP1 in widely distributed cells such as neurons requires that certain functional parameters be met. While viral-based gene transfer is an extremely efficient method to express therapeutic genes in neurons (Dolcetta et al., J. Gene. Med. 8:962-971 (2006); Hughes et al., Mol. Ther. 5:16-24 (2002); Alisky and Davidson, Methods Mol. Biol. 246:91-120 (2004); Martin-Rendon et al., Curr. Opin. Mol. Ther. 3:476-481 (2001); Deglon and Hantraye J. Gene. Med. 7:530-539 (2005); de Boer and Gaillard, Annu. Rev. Pharmacol. Toxicol. 47:323-355 (2007)), it involves intracranial infections, which have limited efficiency in allowing profuse distribution of the therapeutic agent. Also, delivery of vectors in the brain carries other risks such as potential inflammation, cytotoxicity, and the difficulty in regulating how much and how long the gene of interest will be active.

A recently optimized method using a small peptide of the rabies virus glycoprotein (RVG) has successfully delivered silencing siRNAs in a safe. non-invasive, and regulatable manner to CNS neurons. Kumar et al., Nature 448:39-43 (2007). RVG peptide is blood-brain barrier permeable and binds only to the nicotinic acetylcholine receptor present in neurons (Mazarakis et al., Hum Mol Genet 10:2109-2121 (2001)) providing the required cell specificity to deliver siRNA sequences to knock down the expression of a gene only in neurons. Importantly, a single infection provides silencing only for a few days (7-10 days) because of the half-life of the siRNA and the recovery of expression in the absence of a further siRNA sequence, allowing control of the duration of the treatment. Kumar et al., Nature 448:39-43 (2007). The simplicity of this method and the possibility of administering RVG-siRNA complexes repeatedly, without toxicity or immune responses, permits the delivery of siRNA sequences to knock down the expression of both catalytic α- and β-PP1 subunits transiently and specifically in Twitcher neurons. The RVG peptide was successfully delivered to neurons, but not to non-neuronal cells, in vitro and the siRNA strategy disclosed herein led to decreased PP1 expression in neurons.

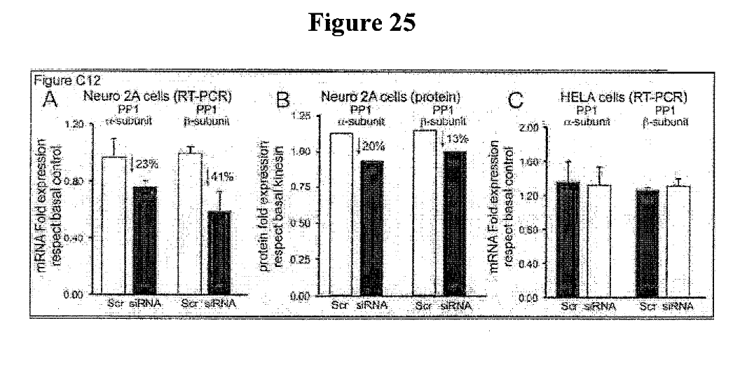

siRNA primers containing sequences specific to the catalytic α- and β-PP1 subunits or scrambled primers were synthesized and coupled to RVG peptide. siRNA-RVG peptide mix was incubated with N2A and HeLa cells for 4 hours. After incubation, cells were replenished with fresh medium and incubated without siRNA-RVG peptide for 48 hours. Cells incubated with the siRNA-RVG mix showed no signs of cell death. Expression of the catalytic α- and β-PP1 subunits was assessed by real time (RT) PCR (FIGS. 25A and 25C) and immunoblotting (FIG. 25B). siRNA sequences led to a partial reduction of both catalytic α- and β-PP1 subunits in N2A cells (shown as % of reduction in FIGS. 25A and 25B). Scrambled primers showed no significant reduction with respect to vehicle-treated N2A cells (FIGS. 25A and 25B). siRNA-RVG-treated HeLa cells showed no silencing, indicating absence of peptide uptake.

To demonstrate the therapeutic efficacy of interfering with PP1 for the treatment of neurodegeneration associated with KD, Twitcher mice were treated with RVG-PP1-siRNA, RVG-siRNA-control scrambled groups, flecainide, and placebos. (Summarized in Table 2). Analyses were performed at 15 days of postnatal (P) age when axonal defects are detected but limited or no demyelination is observed. These experiments employed the reporter Twitcher line expressing axonal YFP (Twi-YFPax) and regulated by the Thyl.1 promoter. This specific axonal label permits the detection of axonal fragmentation, axonal swellings, and axonal varicosities by confocal microscopy as early as P7. Twitcher newborn pups carrying the expression of axonal YFP (Twit-YFPax) were genotyped at P1 (see Example 1).

TABLE 2

| Experimental Groups | Number of Animals |
|---|---|
| Twi-YFPax + single dose RVG-siRNA-PP1 | 6 |
| Twi-YFPax + double dose RVG-siRNA-PP1 | 6 |
| Twi-YFPax + RVG-siRNA control scrambled | 6 |
| Twi-YFPax + Flecainide | 6 |
| Twi-YFPax + placebo | 6 |
| Wild type-YPFax + placebo | 6 |
| Total | 36 |

The transient knock down of PP1 expression in neurons was performed using siRNA targeting the catalytic α- and β-PP1 subunits. A specific siRNA sequence was used for each subunit in combination at a 50:50 molar ratio. A negative control included a mix of scrambled siRNA of each siRNA, also at 50:50 molar ratio. The siRNA presented in Table 3 are exemplified herein without limitation.

TABLE 3

| | | | Genbank |
|---|---|---|---|
| Sequence | | | Accession |
| Identifiers | siRNA Name | siRNA Sequence | Number |
| SEQ ID NO: | Murine PP1α- | 5′-CCAGAUCGUUUGUACAGAAAUCU | NM_031868 |
| 7 | siRNA (antisense | CGAGAUUUCUGUACAAACGAUCUGG-3′ | (SEQ ID NO: 13) |
| | strand) | | |
| SEQ ID NO: | Murine PP1α- | 5′-GUCGUCGAGUCAUCCGCAUUGAUA | |
| 8 | scrambled siRNA | UCUAGCUGAAAUUACCGAGUUAAGA-3′ | |
| SEQ ID NO: | Murine PP1β- | 5′-GGCGUCCUUGAAAGUGUUAAAUCU | NM_172707 |
| 9 | siRNA (antisense | CGAGAUUUAACACUUUCAAGGACGC-3′ | (SEQ ID NO: 15) |
| | strand) | | |
| SEQ ID NO: | Murine PP1β- | 5′-GAAUCUGACCCUCGGUAGCAAUGA | |
| 10 | scrambled siRNA | UAGAGUUUAACACGCUUUCUGUAGA-3′ | |
| SEQ ID NO: | Human PP1α- | 5′-GAGACGCUACAACAUCAAAtt-3′ | NM_206873.1 |
| 28 | siRNA (sense | | (SEQ ID NO: 12) |
| | strand) | | |
| SEQ ID NO: | Human PP1α- | 5′-UUUGAUGUUGUAGCGUCUCtt-3′ | NM_206873.1 |
| 29 | siRNA (antisense | | (SEQ ID NO: 12) |
| | strand) | | |
| SEQ ID NO: | Human PP1α- | 5′-GUGCUCUUACGGUUUAUUGUU-3′ | |
| 30 | siRNA | | |
| | (scrambled) | | |
| SEQ ID NO: | Human PP1β- | 5′-GUAUACACCUAGAGUUUUAtt-3′ | NM_002709.2 |
| 31 | siRNA (sense | | (SEQ ID NO: 14) |
| | strand) | | |
| SEQ ID NO: | Human PP1β- | 5′-UAAAACUCUAGGUGUAUACtt-3′ | NM_002709.2 |
| 32 | scrambled siRNA | | (SEQ ID NO: 14) |
| | (antisense strand) | | |
| SEQ ID NO: | Human PP1β- | 5′-GCUUUCUAUGGACUAAUAAAU-3′ | |
| 33 | scrambled siRNA | | |
| | (scrambled) | | |

Hairpin and loop sequences were generated using available web-based siRNA Wizards. Peptide for RVG-9R was synthesized at the Research Resource Center at the College of Medicine, University of Illinois at Chicago. The sequence for this peptide (SEQ ID NO: 11) is: YTIWMPEBPRPGTPCDIFTNSRGKRASNGGGGRRRRRRRRR. This peptide was conjugated, at its C-terminal end, to the fluorescent tag, Fluorescein. Fluorescence allowed the in situ cellular identification of entry sites for the peptide-siRNA complex.

The mixture of RVG-9R and siRNAs was prepared as described. Kumar et al., Nature 448:39-43 (2007). Peptide was dissolved in physiologic solution at a concentration of 20 μg of RVG-9R per μl. Separately, a mix of both siRNA for the catalytic α- and β-PP1 subunits or their respective scrambled siRNAs at a 50:50 molar ratio was dissolved in physiologic solution at a final concentration of 2 μg of siRNA per μl. Before in vivo injection, a final mixture of RVG-9R-biotin peptide and siRNA was prepared by mixing the stock solutions at a peptide-to-siRNA molar ratio of 10:1 for 15 min, at room temperature. The final peptide-siRNA mix was injected into the temporal vein of recipient mice at P2 or in the tail vein.

Kumar et al. showed that a single injection produced gene silencing in neurons for about 7-10 days. Nature 448:39-43 (2007). To test the effects of a single vs. multiple injections of peptide-siRNA complexes on PP1 silencing and neurodegeneration, one group of Twi-YFPax was injected only at P2 and a second group received one additional injection at P10. Injections at P10 were delivered to the tail vein. Quality controls of efficiency of silencing were done by immunoblotting for catalytic α- and β-PP1 subunit levels in protein extracts from optic and sciatic nerves as examples of anatomical areas with prevalence of axons. Additionally, PP1 activity assays were done on these extracts to quantify phosphatase activity.

### Example 18

### Structural Analysis of the Effects of Neuroprotection on Axonal Degeneration

This example discloses the quantification of the effect of PP1-knock down or flecainide treatment on axonal pathology.

Spinal cord and sciatic nerves were removed from treated and non-treated mice, providing tissue for regular confocal microscopy. Paraformaldehyde-fixed longitudinal 50-μm thick cryosections of spinal cord were used. Whole mount preparations of sciatic nerves were used for confocal analysis. Nerve samples were thoroughly Z-imaged for YEP excitation on a Zeiss Meta-confocal microscope. The number of fragmented or discontinued axons per area in samples from Twi-YFPax mice (treated and non-treated) were counted and the mean values compared to those from WT-YFPax controls. Plotting these numbers against postnatal days allowed a determination of when axonal damage starts and the extent of the effect of each neuroprotective treatment at any given time. Axonal integrity was determined by visualizing continuous YFP fluorescence along a single axon over several hundreds of microns, while visualization of axonal fragments, varicosities, and/or swellings were considered a sign of axonal damage.

### Example 19

### Expression of Channels Involved in the Action Potential and Calcium Flux

During postnatal life, Twitcher mice have deregulated expression of NXC1, Na(v)1.2, and Na(v)1.6 channels (data not shown). Thus, expression of these channels was used as an endpoint to study the effect of protective treatment. For this, tissue samples from spinal cord were processed for RNA isolation and real time PCR of NXC1, Na(v)1.2, and Na(v)1.6 channels as described (Galbiati et al., J. Neurosci. 27:13730-13738 (2007); see, Example 1). After normalization for GAPDH as a housekeeping gene, expression is quantitated (n=3-5 samples per group) and plotted at each developmental age. This was complemented with immunoblotting analysis for each protein and comparison among the various groups.

### Example 24

### Structure of the Node of Ranvier

Maintenance of the node of Ranvier is fundamental for saltatory conduction and its formation is evidently regulated and dependent on a proper axonal transport of the various nodal components. Some of these components, such as sodium channels, appear to be abnormally distributed in Twitcher axons. Kagitani-Shimono et al., Acta Neuropathol. 115:577-587 (2008). The effect of siRNA and flecainide treatments on the stability of the node is studied using sciatic and optic nerves as sources of tissue.

### Example 25

### Psychosine-Induced Inhibition of Fast Axonal Transport by Increasing PP1 Activity

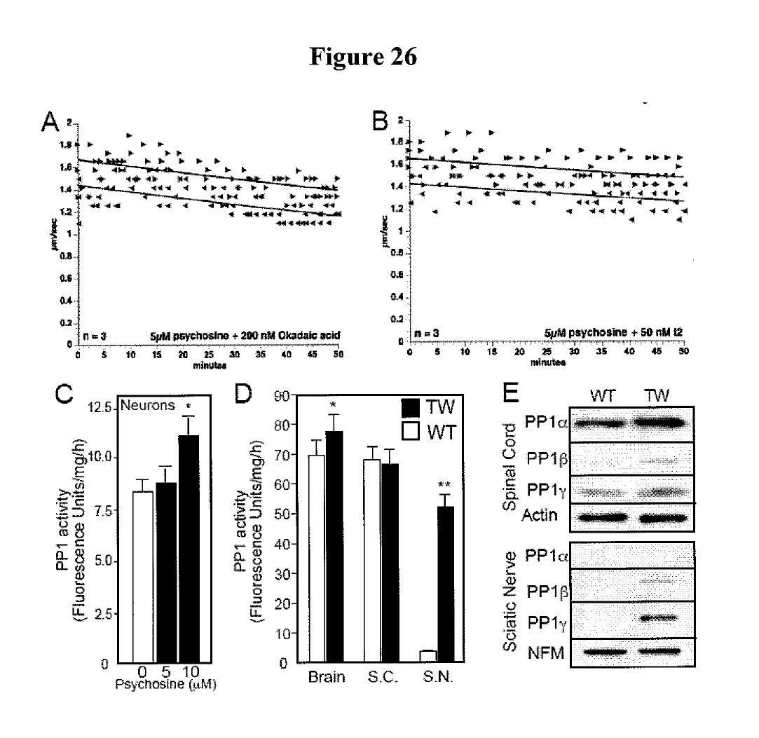

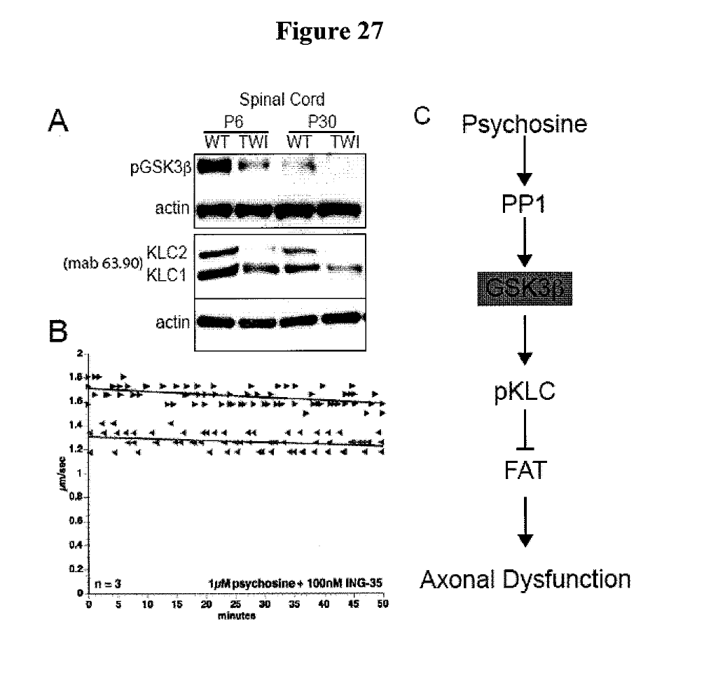

This Example demonstrates that psychosine induces inhibition of fast axonal transport by increasing the phosphatase activity of PP1 (FIG. 26). PP1 is a key enzyme in the regulation of axonal transport, because it controls other phosphotransferase activities that participate in different steps of axonal transport. Among these, GSK3β plays a fundamental role because its kinase activity leads to the phosphorylation of the light chain subunits of kinesin (KLCs). GSK3β is activated by dephosphorylation of ser-9 by PP1. Abnormal phosphorylation of KLCs by GSK3β facilitates the detachment of cargoes from motors and, hence, inhibition of transport. With this in mind, whether FAT inhibition in Krabbe disease was mediated by abnormal kinasing activity of GSK3β. FIG. 27 demonstrates that psychosine-inhibition of FAT is mediated by GSK3β, leading to abnormal phosphorylation of KLCs.

### Example 26

### Sphingomyelin, GM1, GM2, and Sulfatides are Inhibitors of Fast Axonal Transport

This Example demonstrates, through experiments using axoplasm preparation from Loligo Pealei, that substrates that accumulate in other lysosomal storage diseases, which are not related to Krabbe disease, also impair fast axonal transport.

Tested was the effect of perfusing 5 μM of sphingomyelin, GM1, GM2, chondroitin sulfate, and sulfatides, substrates that accumulate in neurological variants of Niemann-Pick disease, GM1 gangliosidosis, Tay-Sachs/Sandhoff diseases, various muccopolysaccharysodes and metachromatic leukodystrophy, respectively. Sphingomyelin, which accumulates in Niemann-Pick disease type A and B, inhibited the anterograde mode of fast axonal transport only. Sphingomyelin did not show any effect on the retrograde mode of transport. Sphingomyelin inhibition was prevented when sphingomyelin was perfused together with 5 μM SB203580, a chemical, cell-permeable, selective, reversible, and ATP-competitive inhibitor of p38 MAP kinase, which also inhibits JNK1 and 2.

Similar results were obtained when axoplasms were perfused with GM1, a ganglioside that accumulates in GM1 gangliosidosis. SB203580 inhibitor also prevented GM1-mediated inhibition of anterograde fast axonal transport. This, and the previous result, demonstrates the involvement of p38/JNK kinases as pathogenic effectors in sphingomyelin and GM1-mediated inhibition of fast axonal transport.

GM2, a ganglioside that accumulates in Tay-Sachs and Sandhoff diseases, also showed specific inhibition of the anterograde but not the retrograde mode of fast axonal transport. Sulfatides, sphingolipids that accumulate in metachromatic leukodystrophy, inhibited both anterograde and retrograde modes of fast axonal transport. In contrast, chondroitin sulfate, which accumulates in muccopolysaccharydosis VII, showed no detectable effect upon perfusion in axoplasm preparations.

The results presented herein demonstrate that: (1) Twitcher mice develop axonopathy; (2) psychosine can block axonal transport; and (3) PP1 and NCX1 are important modulators of neurodegeneration in KD. Moreover, these data further demonstrate that therapeutic compounds and methods based that are effective in decreasing axonal accumulation of psychosine, when used in combination with conventional bone marrow transplantation, may be effectively employed for the treatment of KD. Exemplified herein are siRNA molecules that are capable of downmodulating PP1 expression, flecainaide that is capable of inhibiting the activity of NCX1, and L803 that is capable of inhibiting GSKβ. Each of these exemplary molecules are effective in reducing the axonal accumulation of psychosine and, hence, when used in combination with BMT, are effective in reducing and/or ameliorating the neurodegeneration that is associated with KD and other neurodegenerative diseases.

1. A composition comprising an inhibitory nucleic acid of an effector of psychosine-mediated axonopathy, wherein said effector of psychosine-mediated axonopathy is selected from the group consisting of a kinase, a phosphatase, caspase/calpain, and a sodium/calcium exchange protein.

2. (canceled)

3. The composition of claim 1 wherein said effector of psychosine-mediated axonopathy is selected from the group consisting of CDK5, P38, jnk, src, CK2, PKC, GSK3α, GSK3β, Ser/Thr protein phosphatase PP1, Tyr protein phosphatase PP2, caspase 3, calpain1, calpain 2, and NCX1.

4-6. (canceled)

7. The composition of claim 1 wherein said inhibitory nucleic acid is an siRNA.

8. The composition of claim 7 wherein said siRNA is targeted against an mRNA that encodes the effector, and the effector is selected from the group consisting of CDK5 (SEQ ID NO: 16), GSK3β (SEQ ID NO: 17), PKC (SEQ ID NO: 18), PP1 (SEQ ID NO: 12 and SEQ ID NO: 14), and NCX1 (SEQ ID NO: 19), P38 (SEQ ID NO: 34), jnk (SEQ ID NO: 35), src (SEQ ID NO: 36), caspase 3 (SEQ ID NO: 37); calpain (SEQ ID NO: 38 and SEQ ID NO: 39), CK2 (SEQ ID NO: 40; SEQ ID NO: 41, and SEQ ID NO: 42), and PP2 (SEQ ID NO: 43, SEQ ID NO: 44, SEQ ID NO: 45, and SEQ ID NO: 68).

9-12. (canceled)

13. The composition of claim 7 wherein said siRNA binds to an mRNA encoding the α-isoform or the β-isoform of the Ser/Thr protein phosphatase PP1.

14-20. (canceled)

21. The composition of claim 9 wherein said siRNA is conjugated to a component that permits the transfer of the siRNA across the blood-brain barrier of a patient.

22. The composition of claim 21 wherein said siRNA is conjugated to chimeric rabies virus glycoprotein fragment RVG-9R, which comprises the amino acid sequence NH₂-YTIWMPEBPRPGTPCDIFTN SRGKRASNGGGGRRRRRRRRR-COOH (SEQ ID NO: 11).

23. A composition comprising an antagonist of an effector of psychosine-mediated axonopathy, wherein the antagonist is a small-molecule antagonist or a peptide antagonist.

24. The composition of claim 23 wherein said effector of psychosine-mediated axonopathy is selected from the group consisting of a kinase, a phosphatase, and a sodium/calcium exchange protein.

25. The composition of claim 24 wherein said effector of psychosine-mediated axonopathy is a kinase that comprises an amino acid sequence selected from the group consisting of CDK5 (SEQ ID NO: 24), GSK3β (SEQ ID NO: 25), PKC (SEQ ID NO: 26), P38 (SEQ ID NO: 46), jnk (SEQ ID NO: 47), CK2 (SEQ ID NO: 52, SEQ ID NO: 53, and SEQ ID NO: 54), and src (SEQ ID NO: 48).

26. The composition of claim 24 wherein said effector of psychosine-mediated axonopathy is a phosphatase selected from the group consisting of the α-isoform of the Ser/Thr protein phosphatase PP1 (SEQ ID NO: 20), the β-isoform of the Ser/Thr protein phosphatase PP1 (SEQ ID NO: 22), the α-isoform of the Ser/Thr protein phosphatase PP2 (SEQ ID NO: 55), and the β-isoform of the Ser/Thr protein phosphatase PP2 (SEQ ID NO: 69).

27. The composition of claim 24 wherein said effector of psychosine-mediated axonpathy is NCX1 (SEQ ID NO: 27).

28. The composition of claim 27 wherein said antagonist is flecainide.

29-31. (canceled)

32. The composition of claim 25 wherein said effector of psychosine-mediated axonopathy is GSK3β (SEQ ID NO: 25), and wherein said antagonist is a peptide antagonist that comprises the amino acid sequence Lys-Glu-Ala-Pro-Pro-Ala-Pro-Pro-Gln-pSer-Pro (SEQ ID NO: 60).

33-36. (canceled)

37. A method for the treatment of a neurodegenerative disease in a patient suffering from a psychosine-mediated neurological disorder, storage disease, and/or aging-related neuropathy, said method comprising the step of:
(a) administering to said patient a composition comprising an inhibitor of an effector of psychosine-mediated axonal degeneration, wherein the inhibitor is selected from the group consisting of a small-molecule antagonist of said effector, a peptide antagonist of said effector, or a siRNA molecule(s) that is targeted against, and leads to the downregulation of, a mRNA that encodes said effector.

38. The method of claim 37 wherein said inhibitor is the siRNA molecule(s), and wherein the siRNA molecule(s) is administered to said patient between 0 days and 60 days following the birth of said patient.

39. (canceled)

40. The method of claim 37 wherein said inhibitor is the siRNA molecule(s), and wherein the siRNA molecule(s) is targeted against an mRNA that encodes CDK5 (SEQ ID NO: 16), GSK3β (SEQ ID NO: 17), PKC (SEQ ID NO: 18), PP1 (SEQ ID NO: 12 or SEQ ID NO: 14), NCX1 (SEQ ID NO: 19), P38 (SEQ ID NO: 34), jnk (SEQ ID NO: 35), src (SEQ ID NO: 36), caspase 3 (SEQ ID NO: 37); calpain (SEQ ID NO: 38 and SEQ ID NO: 39), CK2 (SEQ ID NO: 40; SEQ ID NO: 41, and SEQ ID NO: 42), or PP2 (SEQ ID NO: 43, SEQ ID NO: 44, SEQ ID NO: 45, and SEQ ID NO: 68).

41. The method of claim 37, further comprising the step of administering to said patient a composition comprising a GALC-expressing cell.

42. The method of claim 41 wherein said GALC-expressing cell is a macrophage within a donor bone marrow sample.

43-46. (canceled)

47. The method of claim 37 wherein said effector of psychosine-mediated axonopathy is selected from the group consisting of a kinase, a phosphatase, and a sodium/calcium exchange protein, and wherein said inhibitor is said small-molecule antagonist or said peptide antagonist.

48. The method of claim 47 wherein said effector of psychosine-mediated axonal degeneration is selected from the group consisting of CDK5 (SEQ ID NO:
24), GSK3β (SEQ ID NO: 25), PKC (SEQ ID NO: 26), PP1 (SEQ ID NO: 20 or SEQ ID NO: 22), PP1 α-isoform (SEQ ID NO: 20), PP1 β-isoform (SEQ ID NO: 22), PP2 α-isoform (SEQ ID NO: 55), PP2 β-isoform (SEQ ID NO: 69), NCX1 (SEQ ID NO: 27), P38 (SEQ ID NO: 46), jnk (SEQ ID NO: 47), CK2 (SEQ ID NO: 52, SEQ ID NO: 53, and SEQ ID NO: 54), src (SEQ ID NO: 48), PP2 (SEQ ID NO: 55, SEQ ID NO: 56, SEQ ID NO: 57, and SEQ ID NO: 59), caspase 3 (SEQ ID NO: 49), and calpain (SEQ ID NO: 50 and SEQ ID NO: 51).

49. (canceled)

50. The method of claim 37 wherein said effector of psychosine-mediated axonal degeneration is NCX1 and said inhibitor is flecainide.

51. The method of claim 37 wherein said effector of psychosine-mediated axonal degeneration is GSK3β (SEQ ID NO: 25) and wherein said inhibitor is a peptide that comprises the amino acid sequence Lys-Glu-Ala-Pro-Pro-Ala-Pro-Pro-Gln-pSer-Pro (SEQ ID NO: 60).

51-53. (canceled)
